(19) United States
(12) Patent Application Publication
(10) Pub. No.: US 2012/0178915 A1
(43) Pub. Date: Jul. 12, 2012

(54) PYRIMIDINE DERIVATIVES AND ANALOGS, PREPARATION METHOD AND USE THEREOF

(76) Inventor: Lifeng Xu, San Diego, CA (US)

(21) Appl. No.: 13/393,226

(22) PCT Filed: Dec. 24, 2009
(86) PCT No.: PCT/CN09/75920

§ 371 Date: Feb. 29, 2012

(30) Foreign Application Priority Data
Sep. 23, 2009 (CN) 200910177792.9

(51) Int. Cl. C07D 487/04; C07D 239/91; C07D 413/10; C07D 487/14; A61P 31/12; C07H 15/26; A61P 31/04; A61P 31/10; A61P 29/00; C07D 265/22; C07D 401/04

(52) U.S. Cl. 536/17.4; 544/281; 544/117; 544/92; 544/289; 544/116; 544/251; 544/247; 540/501; 540/473

(57) ABSTRACT

This invention relates with the arylheterocycle-fused pyrimidines, derivatives and analogs of formula I:

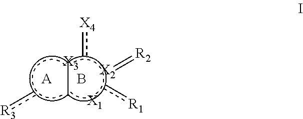

or stereoisomers, tautoers, prodrugs, pharmaceutically acceptable salts, complex salts or solvates thereof, wherein:

A-cycle is of 3-8 saturated or unsaturated arylheterocycles or aliheterocyclic, containing 1-4 heteroatoms, B-cycle 5-8 member saturated or unsaturated heterocycle containing 1-4 heteroatoms; X₁, X₂, X₃, X₄ are, independently at each occurrence, C, O, S, Se, N and P elements; R₁, R₂, R₃ is a substituent containing alicyclic group, arylcycle group, heterocyclic group, adamantane alkyl, adamantane heterocycle, adamantane analogs, sugar group, hydroxyl group, amino acid group or a combination of the above substituents.

This invention also relates with their preparative methods and applications.

### FIELD OF THE INVENTION

This invention relates to arylheterocycle-fused pyrimidines, derivatives and analogs to their use as antibacterial and antifungal agents, to pharmaceutical compositions containing these compounds and to methods for their preparation.

### BACKGROUND OF THE INVENTION

Since 1929 Alexander Fleming discovered penicillin, varieties of antibacterial drugs have been discovered. Antibacterial resistance is a global clinical and public health problem that has emerged with alarming rapidity in recent years and will undoubtedly increase in the near future. Multi-drug resistance, called “superbugs” methicillin-resistant Staphylococcus aureus (MRSA), vancomycin-resistant enterococci (VRE) have caused the morbidity, mortality, and financial costs of such infections pose an increasing burden for health care systems worldwide.

### DESCRIPTION OF BACKGROUND ART

The arylheterocycle-fused pyrimidines, derivatives and analogs of this invention have antibacterial and antifungal activity as structure I. So far there are no the antibacterial activity reports of arylheterocycle-fused pyrimidine compounds except the other pharmacological activities or single cycle of pyrimidine compounds. Other activity of patents are: the anticancer activity of pyrrolo-fused pyrimidinone was described by US 20060160831; the anticancer activity of pyrazolo-fused pyrimidinone was described by U.S. Pat. No. 6,531,477; the anticancer activity of pyridin-fused pyrimidinone was described by US20030100572; the anticancer activity of five membered alicyclic ketone-fused pyrimidines was described by U.S. Pat. No. 7,262,187; the anticancer activity of quinolin-fused pyrimidinone was described by US 20030100572 and 20030220345 and the paper (Literature 1: Kano et al. Chemical and Pharmaceutical Bulletin, 7, 1959, 903). The mental illness treatment of sulfur-containing arylhetero-fused pyrimidine was described by U.S. Pat. No. 4,581,171; The sexual function treatment of the imidazoarylcycle-fused pyrimidines was described by US 20060264624; the anti-Hypertension of isoquinolin-fused pyrimidines was describe red by U.S. Pat. No. 4,482,556 and U.S. Pat. No. 5,346,901; the mental illness treatment of piperidin-fused pyrimidinones was describe red by U.S. Pat. No. 5,158,952; the asthma and immune system disease treatment of five membered heterocycle-fused pyrimidine was describe red by U.S. Pat. No. 6,790,850; The antihistamine treatment of heterocycle-fused pyrimidine was described by U.S. Pat. No. 5,008,268; the nervous system disorders treatment of benzopyrimidine was described by US 20070167423; cancer treatment of benzoaminopyrimidine was describe red by U.S. Pat. No. 7,132,427; However, the patent of anti-bacterial and anti-fungal activities of arylheterocycle-fused pyrimidine, derivatives analogs have not been found. The anti-cancer treatment of arylheterocycle-fused triazole was described by U.S. Pat. No. 4,546,181, as well as other structural related reports (Ref 1; literature 2.: Allen et al., Journal of Organic Chemistry, 24, 1959, 787; literature 3: Sako, Magoichi, et al, Chemical and Pharmaceutical Bulletin, 42, 4, 1994, 806; literature 4: Lewin et al., J. Gen. Chem. USSR (Engl. Transl.), 33, 1963, 2603; literature 5: Zhurnal Obshchei Khimii, 33, 1963, 2673; literature 6: Guerret et al., Bulletin de la Societe Chimique de France, 1972, 3503, literature 7: Brady, Herbst. Journal of Organic Chemistry, 24, 1959, 922, 924).

In conclusion, so far there have not yet been any report related with the present invention represented by structural formula I, the arylheterocycle-fused pyrimidines, analogs and derivatives with antibacterial and antifungal activity.

### BRIEF DESCRIPTION OF THE DRAWINGS

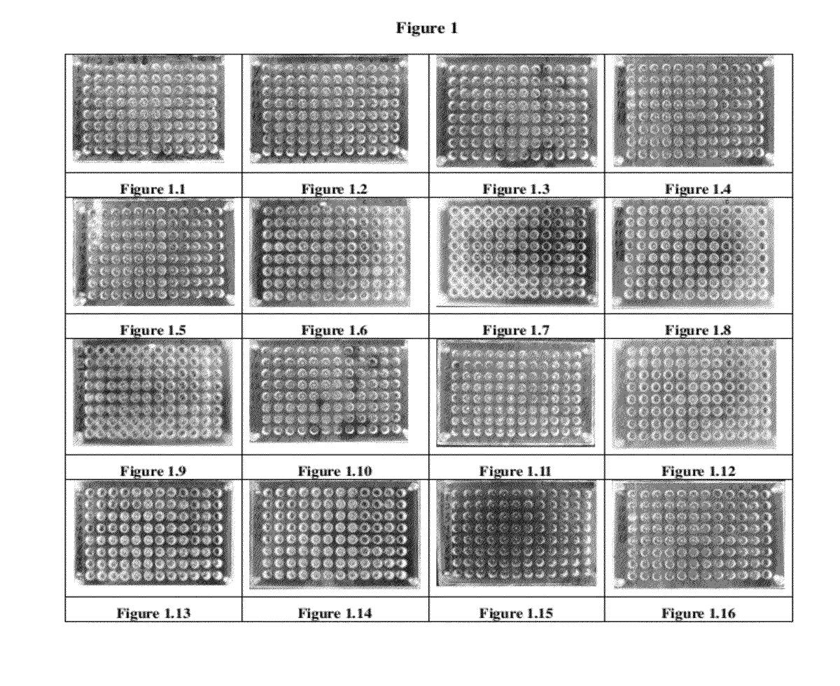

In FIG. 1, FIG. 1.1: shows the inhibition growth of Bacillus cereus 246; FIG. 1.2: shows the inhibition growth of Bacillus cereus 246; FIG. 1.3: Bacillus subtilis 168; FIG. 1.4: shows the inhibition growth of Bacillus subtilis 168; FIG. 1.5: shows the inhibition growth of Enterococcus faecalis 51299; FIG. 1.6: shows the inhibition growth of Enterococcus faecalis 51299; FIG. 1.7: shows the inhibition growth of Enterococcus faecalis 1513 (VRE); FIG. 1.8: shows the inhibition growth of Enterococcus faecalis 1513 (VRE); FIG. 1.9: shows the inhibition growth of Staphylococcus aureus 43300 (MRSA); FIG. 1.10: shows the inhibition growth of Staphylococcus aureus 43300 (MRSA); FIG. 1.11: shows the inhibition growth of Streptococcus pneumoniae 6303 (PRSP); FIG. 1.12: shows the inhibition growth of Streptococcus pneumoniae 6303 (PRSP); FIG. 1.13: shows the inhibition growth of Streptococcus pneumoniae purulent M2; FIG. 1.14: shows the inhibition growth of Streptococcus pneumoniae purulent M2; FIG. 1.15: shows the inhibition growth of Streptococcal 10342; FIG. 1.16: shows the inhibition growth of Streptococcal 10342.

Note: 1, 2, 3, 4, 5, 6, 7, 8, 9, 10, 11, 12 and 13 in first column FIG. 1-16 are the example compounds of 3, 5, 7, 8, 10, 14, 21, 23, 33, 38, 42, 70 and 78; 1, 2, 3, 4, 5 and 6 in the column FIG. 1-16 are the concentration gradient of correspondent example compounds of 3, 5, 7, 8, 10, 14, 21, 23, 33, 38, 42, 70 and 78; C is ciprofloxacin (positive control), V is vancomycin (positive control), NS is negative control (saline).

### DETAILED DESCRIPTION OF THE INVENTION

The purpose of the present invention is to provide an arylheterocycle-fused pyrimidines, derivatives and analogs to their use as antibacterial and antifungal agents, to pharmaceutical compositions containing these compounds and to methods for their preparation, which have the following general formula I prodrugs or pharmaceutical salts thereof and the prodrug, and to provide pharmacological activity of their preparation and experimental methods and pharmacological activity. This invention relates with the formula I,

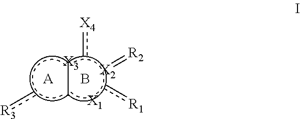

or stereoisomers, tautoers, prodrug, pharmaceutically acceptable salts, complex salts or solvates thereof, wherein:

The dotted lines are independently at each occurrence, optionally substituted single bonds, optionally substituted double bond or a optionally substituted heterocyclic group containing carbon, oxygen, sulfur or nitrogen element; A-cycle is of 3-8 saturated or unsaturated arylheterocycles or aliheterocyclic, containing 1-4 heteroatoms, B-cycle 5-8 member saturated or unsaturated heterocycle containing 1-4 heteroatoms; X₁, X₂, X₃, X₄ are, independently at each occurrence, C, O, S, Se, N and P elements, or contains a substituted C, O, S, Se, N, P elements existing independently or combination; R₁, R₂, R₃ is a substituent containing alicyclic group, arylcycle group, heterocyclic group, adamantane alkyl, adamantane heterocycle, substituent adamantane, adamantane analogs, sugar group, hydroxyl group, amino acid group, the substituent containing O, S, Se, N and/or P, the hydrocarbon chain containing O, S, Se, N or P atom, cycle group and one or a combination of the above substituents;

When X₁, X₂, X₃ or X₄ is C element, they are the same or different substituents of C═O, C═Rb—Ra, CHOH, CHORb or CHRb, X₁ and X₂; when X₁, X₂, X₃ or X₄ is O element, they are the same or different substituents of —O—, —O═Rb—Ra; when X₁, X₂, X₃ or X₄ is S element, they are the same or different substituents of divalent, tetravalent, hexavalent S, ═S═Rb—Ra; when X₁, X₂, X₃ or X₄ is N elements, they are the same or different substituents of —NH—, ═NH, ═N—Rb—Ra; X₁, X₂, X₃ or X₄ is P elements, they are the same or different substituents of trivalent phosphine, pentavalent phosphine, —PH2, ═NH, ═PRb—Ra; when the X₃ is heteroatoms, the A cycle can be formed by the combination of heteroatoms with the C, O, S, Se, N, P elements to form carbon-hetero bind or hetero-Hetero bind, wherein:
Ra and Rb are the same or different substituents; Rb is C, N, P atoms; Ra is hydrogen, halogen, hydroxyl, thiol, cyano, carbonyl, substituted carbonyl, aldehyde, ketone, nitro, carboxyl, substituted carboxyl, carboxylic ester, amino, substituted amino, alkyl, alkoxy, aryl alkoxy, aryloxy, heteroaryloxy, alkylthio, arylalkylthio, arylthio, heteroarylthio, amino, amino-alkoxy, saturated or unsaturated heterocyclic, heterocyclic alkoxy or alkyl amino heterocyclic group to form a variety of substituents with double bonds can also form a new straight chain, branched-chain alkane group or hydrocarbon substituents containing fat alkyl, double bond or triple bond of saturated or unsaturated fatty alkyl, alicycle, alicycle, aliheterocyclic, aromatic, arylheterocycle fused heterocyclic group or a combination thereof;
Substituent is, independently at each occurrence, optionally substituted C₁₋₁₂ optionally substituted saturated or unsaturated alkyl, 1-4 optionally substituted double bond, 1-4 optionally substituted triple bond, optionally substituted or saturated, unsaturated C₁₋₁₀ alicyclic, C₁₋₁₀ optionally substituted aryl group or C₁₋₁₀ optionally substituted heterocyclic, where contains optionally substituted one or combination of oxygen, sulfur, nitrogen, phosphorus element, halogen, saturated or unsaturated 3-7 membered alicyclic, aryl cyclic, multi-cyclic, aliphatic heterocyclic, aryl heterocyclic, fused heterocyclic, alkyl, cycle, aryl or heterocyclic group or a combination thereof;
   Cyclic group is, independently at each occurrence, optionally substituted alicyclic, arylcyclic, aliheterocyclic or arylheterocyclic group, 3-8 membered cycle; alkyl is, independently at each occurrence, alialkyl, arylalkyl;

Glycosyl group is, independently at each occurrence, optionally substituted D- and L-configuration with C—C or C-Hetero bond of glycoside, substituent of 1-8 glycosyl, multi-Hydroxyl, multi-hydroxyl, phenol, polyphenol, amino acid;

Substituent is, independently at each occurrence, optionally substituted chain alkyl containing hydroxyl, alkoxy, ester, acyloxy, phosphate oxy, sulfonyloxy, aryloxy heterocyclic oxy, thiol, alkyl thiol, alkthiol, thiolester, aryl thiol, heterocyclic thiol, ether group, 4-8 membered arylcyclic, 4-8 membered heterocyclic, 4-8 membered alicyclic, 4-8 membered arylheterocyclic, bridge cyclic, spiral cyclic, adamantane cyclic, amino, primaryl amine, secondaryl amino, tertiaryl amino, quaternaryl ammonium, amide, hydrazine, oxime, hydrazone-based, phosphide, phosphate, phosphate group, O, S, Se, N or P element;

R₁, R₂, R₃ is independently at each occurrence, optionally substituted the same or different substituents of hydrogen, halogen, hydroxyl, thiol, cyano, carbonyl, aldehyde, ketone, nitro, carboxyl, carboxylic ester, alkyl, alkoxy, aryloxy, alkylthio, arylthio, amino, aminoalkoxy, heterocyclic, alkylalkoxy, acyl (RaCO), carbamoyl (RbRcNCO) or sulfonyl (RdSO₂), wherein:

Ra, Rb, Rc and Rd is the same or different substituents of hydrogen, halogen, hydroxyl, thiol, cyano, carbonyl, aldehyde, ketone, nitro, carboxyl, carboxylic ester, alkyl, alkoxy, arylalkoxy, alkyl sulfur, arylalkylthio, arylthio, amino, aminoalkoxy, heterocyclic, heterocyclicoxy, heterocyclic alkyl amino or double bond containing substituents(═X₅), wherein:

X₅ is independently at each occurrence, optionally substituted the same or different substituents containing O, S, Se, N or P element; substituent is independently at each occurrence, optionally substituted the same or different R₁ and R₂ of two cycles to form a new cycle or combination of two cycles;

R₁, R₂, R₃ or X₄ is independently at each occurrence, optionally substituted the same or different substituents of H, halogen or XRa; wherein:

X is optionally substituted group containing C, O, S, Se, N, and/or the P element;

A compound of arylheterocycle-fused pyrimidines, derivatives and analogs is selected from the exemplified examples or stereoisomers, tautomers, pharmaceutically acceptable salts, inorganic acid salt, organic acid salt, organic basic salt, organic basic salt, complex salt, prodrug or solvates thereof in association with a pharmaceutically acceptable excipient or carrier.

A compound of arylheterocycle-fused pyrimidines, derivatives and analogs fused with three membered A-cycle, four membered A-cycle, five membered A-cycle, six membered A-cycle, eight membered A-cycle or five membered B-cycle is, independently at each occurrence, from the example 1 to example 536.

A process for the manufacture of a compound of formula I comprises: For the preparation of compounds of formula I and salts thereof in which the reaction of a pyrimidinylcycle fused arylcycle to form A-cycle, B-cycle, X₁, X₂, X₃, X₄, R₁, R₂, R₃ with a bond of C—C, C—O, C—S, C—N or C—P under catalysis at −78° C. to 90° C., wherein:

Preparation of o-amino-nitrogen containing heterocycle analogs, the key intermediates to form the pyrimidinyl fused arylcycle by a solvent selected from THF, 1,4-dioxane, N,N-dimethyl-formamide amide, N,N-dimethylacetamide or toluene, the reaction temperature from room temperature to 180° C., a catalyst selected from p-toluenesulfonate, Lewis acid, sulfuric acid, molecular sieves, alumina, silica gel or dehydration agent;

Preparation of arylcycle fused pyrimidine analogs: by a solvent selected from ethanol, THF, 1,4-dioxane, N,N-dimethylformamide amide, N,N-dimethylacetamide or toluene, the reaction temperature from room temperature to 180° C., a catalyst selected from p-toluenesulfonate, Lewis acid, sulfuric acid, molecular sieves, alumina, silica gel or dehydration agent;

preparation of Heterocyclic cycle fused pyrimidine analogs: by a solvent selected from ethanol, THF, 1,4-dioxane, N,N-dimethylformamide amide, N,N-dimethylacetamide or toluene, the reaction temperature from room temperature to 180° C., a catalyst selected from p-toluenesulfonate, Lewis acid, sulfuric acid, molecular sieves, alumina, silica gel or dehydrationagen.

A compound for treating, preventing or slowing the progression of bacteria, fungi and other infections caused by known antibacterial, antifungal, anti-inflammatory and anti-viral and immune compatibility of drug use, including inflammation associated with bacterial infections and inflammatory diseases, fungi and fungal diseases, viruses and viral diseases and immune system disease, alone or with the following drugs known to be used in conjunction dose of 0.02 mg/kg-250 mg/kg (intravenous, intramuscular, oral, topical and other routes of administration); means of various methods of treatment and therapy, where the bacteria are Gram-positive bacteria, Staphylococcus aureus, Streptococcus pneumoniae, Enterococcus faecalis, Streptococcus, Streptococcus bovis, Streptococcus pneumoniae, Peptostreptococcus, Streptococcus pneumonia, Streptococcus pneumoniae, Streptococcus pyogenes, Streptococcus agalactiae, Streptococcus bovis, Streptococcus agalactiae B, Clostridium tetani, Bacillus cereus, Bacillus subtilis, Clostridium perfcycleens, Bacillus cereus, Bacillus subtilis, Bacillus anthracis, Clostridium bacillus, Clostridium perfcycleens, Clostridium perfcycleens spirochetes, M. tuberculosis, suppurative, purulent, diphtheria, tetanus bacilli, erysipelas bacillus, Bacillus anthracis, actinomycetes, Gram-positive bacteria resistant to methicillin-resistant Staphylococcus aureus, vancomycin-resistant gold Pu bacteria, Staphylococcus inducible clindamycin resistance, vancomycin-resistant enterococci, aminoglycoside-resistant enterococci high-level penicillin-resistant Streptococcus pneumoniae, multi-drug resistant Acinetobacter baumannii and multiple drug resistance drug-resistant Mycobacterium tuberculosis and Mycobacterium tuberculosis, Streptococcus, Enterococcus faecalis, Pseudomonas aeruginosa, Escherichia coli and Acinetobacter spp, drug-resistant Haemophilus influenzae, Neisseria gonorrhoeae resistance, resistance Neisseria meningitidis, Enterobacteriaceae resistant or resistant Pseudomonas aeruginosa.

A compound of arylheterocycle-fused pyrimidines, derivatives and analogs for treating, preventing or slowing the progression of bacteria and fungi and other infections caused by various infections including bacterial infection associated with inflammation and inflammatory diseases, fungi and fungal diseases, viral and viral diseases and immune system complications: upper and lower respiratory tract infections, skin and soft tissue infections, urinary tract infections, septicemia and endocarditis infection induced by methicillin-sensitive Staphylococcus aureus, hemolytic streptococci and Streptococcus pneumoniae; the infections of urinary tract and pneumonia induced by Streptococcus, Streptococcus pneumoniae, Haemophilus influenzae, Proteus mirabilis, Escherichia coli and other Gram-positive cocci; the infections of respiratory tract, urinary tract, skin and soft tissue, sepsis, bone and joint, abdominal, pelvic viridans, endocarditis, anaerobic, anthrax, syphilis or gonorrhea induced by hemolytic streptococcus and enterococci.

A compound of arylheterocycle-fused pyrimidines, derivatives and analogs is administered together with at least one known antibacterial agents, antifungal agents or anti-inflammatory agents selected from penicillin, procaine penicillin, benzathine penicillin, methicillin, oxacillin, cloxacillin, dicloxacillin, hetacillin, sulbenicillin, temocillin, mecillinam, piperacillin, ticarcillin, ticarcillin, flucloxacillin, sultamicillin, phenoxymethylpenicillin, bacampicillin, ticarcillin, talampicillin, furbenicillin, aspoxicillim, pivampicilli, meticillin, nafcillin, pivmecillinam, lenampicillin varacillin, apalcillin, carindacillin, carbenicillin, ceftriaxone, cefpirome, cefuroxime, ceftazidime, cefotaxime, cephalothin, cefathiamidine, cephalosporin, cephalosporin, cefazolin, cefinenoxime, cefoperazone, cefaclor, ceftizoxime, cefdinir, cefixime, cephradine, cefpiramide, cephalosporin, cephalosporin, cefpodoxime, cefodizime, cefotiam, cefetamet, cefprozil, cephalosporin, cefepime, cephalexin, cephradine, cefadroxil, cefoxitin, cefinetazole, cefotetan, cefminox, cephalosporin, cefozopran, cephalosporin, cefalotin, cefaloglycine, cefalexin, cefradine, cefacetrile cefapirin, cefadril, cefroxadine, ceftezole, cefonicid, cefamandole, cefbuperazone, cefdinir, cefzon, cefcapene, cefotaxime, cefteram, cefsulodine, latamoxef, cefpimizole, cefuzonam, aztreonam, erythromycin, roxithromycin, adriamycin, clarithromycin, fluoerythromycin, azithromycin, kitasamycin, albomycin, leucomycin, josamycin, spiramycin, acetyl spiramycin, netilmicin, isepamicin, streptomycin, gentamicin, tobramycin, amikacin, sisomicin, neomycin, neomycin, spectinomycin, streptomycin, kanamycin, chloramphenicol, thiamphenicol, lincomycin, clindamycin, clindamycin, cyclosporine, teicoplanin, vancomycin, teicoplanin, bacitracin, polymyxin B, rifamycin, rifabutin, rifapentine, rifaximin, rifampicin, rifamycinn, enoxacin, norfloxacin, ciprofloxacin, lomefloxacin, sparfloxacin, pefloxacin, fleroxacin, moxifloxacin, ofloxacin, levofloxacin, rufloxacin, isoxazole, sulfamethoxazole, sulfadiazine, trimethoprim, pipemidic acid, nitrofurantoin, furazolidone, nalidixic acid, difloxacin, gatifloxacin, pazufloxacin, moxifloxacin acid, tetracycline, minocycline, chlortetracycline, doxycycline, oxytetracycline, doxycycline, metacycline, clavulanic acid, sulbactam, tazobactam, imipenem, cilastatin, panipenem, betamipron, meropenem, cephamycin, sulfamethoxazole, sulfamethazine, sulfonamides, sulfamonomethoxine, sulfamethoxazole, sulfathiazole, sulfamethoxazole, sulfathiazole, thienamycin, aztreonam, faropenem, cilastatin, tazobactam, streptomycin, neomycin, kanamycin, neomycin, clindamycin, fosfomycin, brodimoprim, metronidazole, aclarubicin, epirubicin, pirarubicin, mupirocin, timidazole, sulfasalazine, itraconazole, bifonazole, fluconazole, clotrimazole, econazole, miconazole, ketoconazole, naftifine, butenafine, ciclopirox, amorolfine, amphotericin B, flucytosine, terbinafine, nystatin, Gentamycin, nebramycin, micronomicin, ribostamycin, astromicin, dibekacin, etimicin, nofloxacin, cotrim, sulfamethoxazole, mafenide, brodimoprim clafalix, laurylin, azitromycin, midecamycin, acetylmidecamycin, rokitamycin, meleumycin, methacycline, doxycyclme, demeclocycline, nifuratel, methylmercadone, norvancomycim, colistin, gramicidin, isoniazid, ethambutol, pyrazinamide, rifamycin, rifandin, viomycin, rifampin, capreomycin, ethionamide, terconazole, fenticonazole, sulconazole, fluorocytosine, ciclopirox, mepartricin, exalamide, terbinafine, ribavirin, acyclovir, ganciclovir, indinavir, nelfinavir, ritonavir, cidofovir, penciclovir, buciclovir, penciclovir, famciclovir, valaciclovir, famotine, vidarabine, zidovudine, azidothymidine, foscarnet, delavirdine, moroxydine, idoxuridine, amantadine, interferons, rimantadine, clindamycim.

The administration of arylheterocycle-fused pyrimidines, derivatives and analogs may be by oral route, parenteral, subcutaneous, intravenous, intramuscular, intra-peritoneal, transdermal, buccal, intrathecal, intracranial, intranasal or topical routes.

### Chemical Synthesis

Arylheterocyclic fused-pyrimidine derivatives and analogues of the preparation method: including the structure of claim 1 of the A cycle and B cycle cyclization, and introduction of X₁, X₂, X₃, X₄, R₁ and R₂ and the preparation of the key intermediate, amino substituted heterocyclic A-cycle and B-cycle with the formation of C—C bond, C—O bond, C—S bond, C—N bond and C—P bond were catalyzed by below schemes with a catalyst selected from p-toluenesulfonate, Lewis acid, sulfuric acid, zeolite, alumina, silica or dehydration agent, a solvent selected from THF, 1,4-dioxane, acetonitrile, N,N-dimethylformamide, N,N-dimethylacetamide, n-Hexane, toluene or solvent-free reaction and reaction temperature at −40° C. to 180° C.:

1. Preparation of o-aminonitrogen-containing heterocycles: o-amino nitrogen-containing heterocycles, key intermediate was synthesized by the cyclization of acyled phenylacetonitrile with hydrazines.

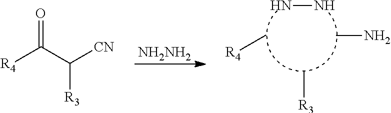

2. Preparation of arylpyrimidones: the cyclization of o-amino nitrogen-containing heterocycles with a carbonyl-containing ester.

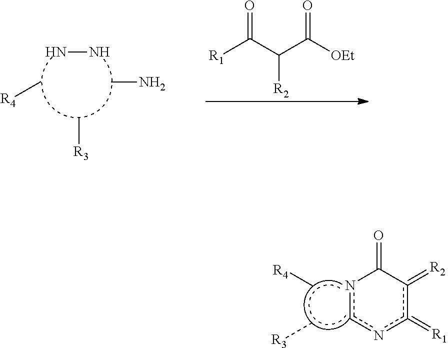

3. Preparation of pyrimidones fused heterocyclic: the cyclization of o-amino heterocycles or acyl-containing arylamides.

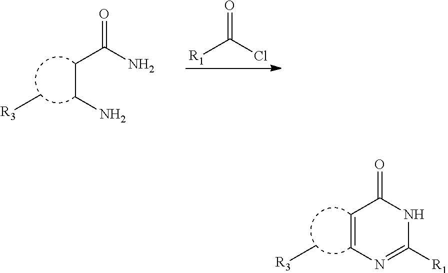

4. Preparation of pyrimidines fused polyheterocycles: the cyclization of 7-amino-3-substituted arylpyrazolo[1,5-a]pyrimidin-6-cyanide with arylisothiocyanates-containing compounds.

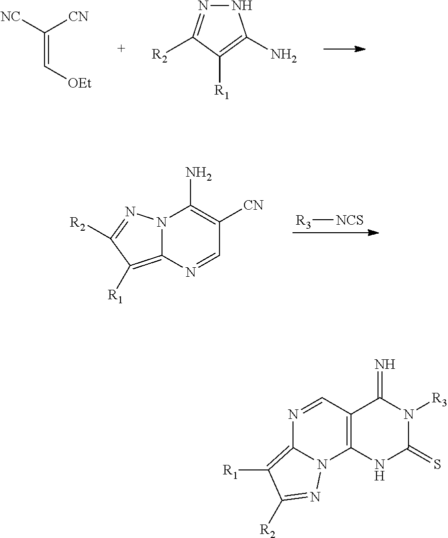

5. Preparation of pyrimidines fused heterocycles: the cyclization of amino-pyrazoles with aryldenemalononotriles.

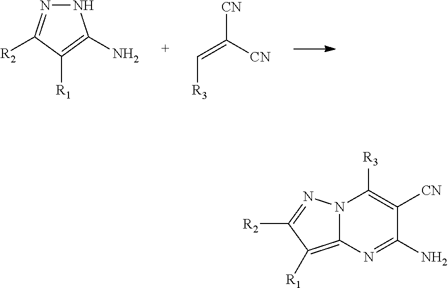

6. Preparation of heterocycles fused carbonylpyrimidone: the cyclization of 2-H-indene-1,3-diones with amino-pyrazoles.

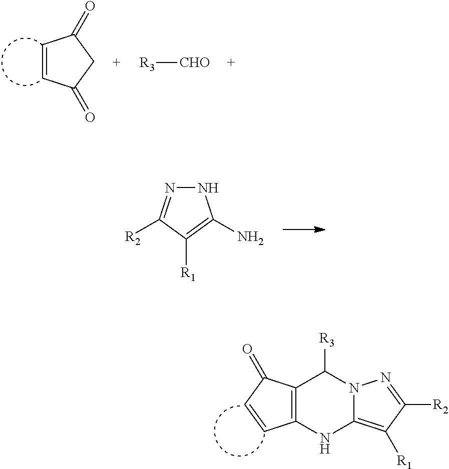

7. Preparation of heterocycles fused 3-arylpyrimidone: the cyclization of amino-pyrazoles with arylaldehydes.

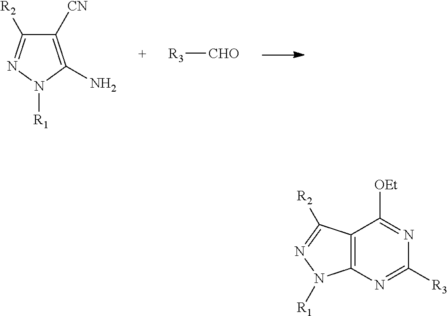

8. Preparation of heterocyclic dicarboxylate: the cyclization of cyano-substituted o-amino pyrazoles with dimethyl acetylenedicarboxylate.

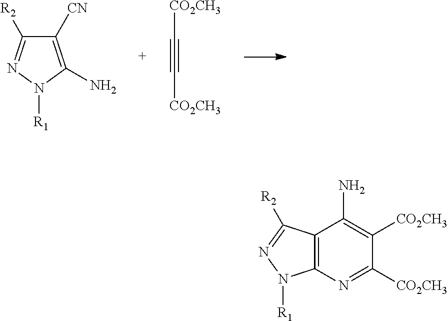

9. Preparation of 2-aryl pyrimidines fused heterocycle: the cyclization of cyano-substituted o-amino pyrazoles with triethoxy methane.

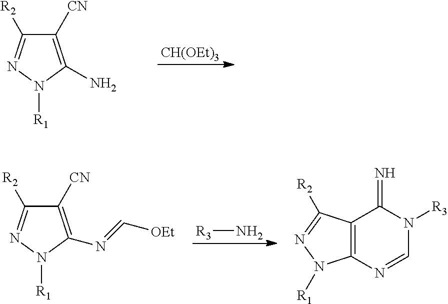

10. Preparation of 5-arylheterocycle fused hydroxyl-containing pyrimidones: the cyclization of amino-imidazoles with oxo-carboxylates.

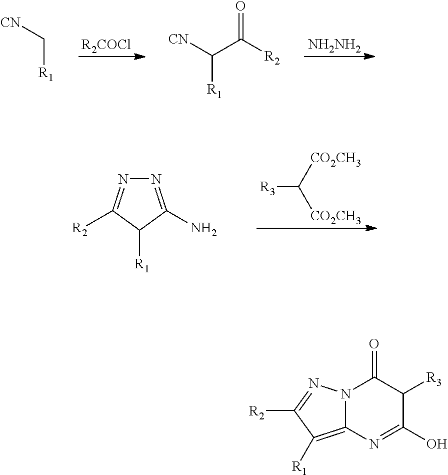

11. Preparation of pyrimidines fused diaryl-containing heterocycle: the cyclization of o-amino pyrazoles with diethyl malonate.

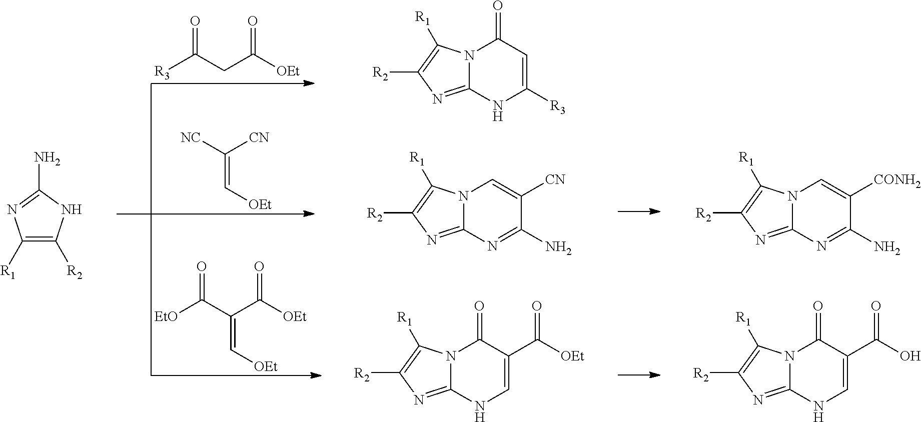

12. Preparation of pyrimidines fused 6-aryl-containing heterocycle: the cyclization of o-amino pyrazoles with diethyl arylmalonate.

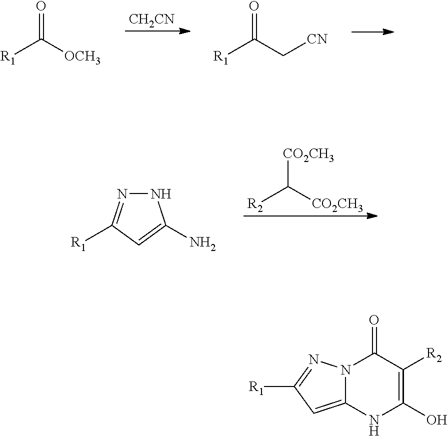

13. Preparation pyrimidones fused benzimidazole: the cyclization of o-amino pyrazoles with 3-oxo-phenylpropionates.

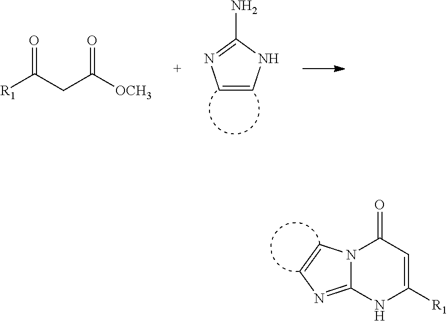

14. Preparation of polyheterocycle fused pyrimidone: the cyclization of cyanides with triethoxy methane and amino-compounds.

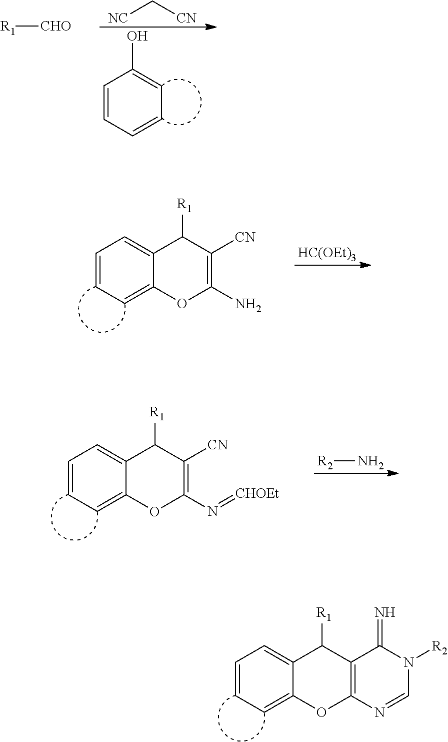

15. Preparation of pyrimidines fuzed triazole: the cyclization of amino- and imino-compounds with carbonyl-containing compounds.

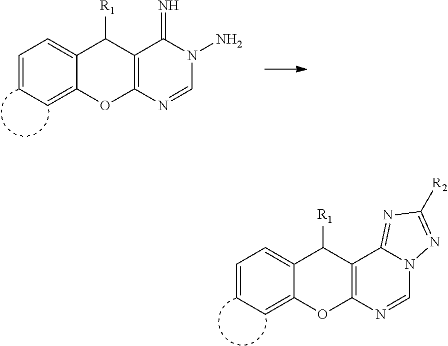

The invention compounds can be pharmaceutical salts are within the scope of this invention, the acid can react by salt and alkali, such as sodium carbonate, sodium hydride, potassium hydroxide, ammonium hydroxide. Structure containing a nitrogen atom and the acid by reaction of alkaline salt such as hydrochloric acid, fumaric acid, maleic acid, succinic acid, acetic acid, citric acid, tartaric acid, carbonic acid, phosphoric acid, oxalic or acid.

Prodrug compounds of this invention are also within the scope of this invention. Drugs of this invention can be modified into a prodrug, increases its water solubility and molecular size, and reduce its toxicity.

Pharmaceutical compounds of this invention can be administered by any means. For example, oral, parenteral, subcutaneous, intravenous, intramuscular, intraperitoneal, transdermal, buccal, intrathecal, intracranial, intranasal administration or local channels. Dose according to patient weight, age, condition, treatment, and to determine the compatibility of drugs, of which the effective dose range of 0.002 mg/kg-250 mg/kg.

### 2. Synthesis and Preparative Example

### Implementation of the Case and its Structure 1-536 Table 1)

### Example 1

### Compound 1 in Table 1

To a mixture of 5-methyl-4-(4-(trifluoromethyl)phenyl)-4H-pyrazol-3-amine 0.964 g (4.0 mmol) in ethanol 20 ml, was added diethyl 2-formylmalonate 750 mg (4.0 mmol) and sodium 0.55 g. The mixture was refluxed for 10 h. The reaction solution was filtered and distilled under reduced pressure. The crude was separated by silica gel column chromatography to give the compound 1, IR (KBr, cm⁻¹) 3446, 2986, 2933, 1701, 1617, 1595, 1557, 1468, 1326, 1261, 1191, 1164, 1108; ¹HNMR (DMSO-d6) δ 8.50 (s, 1H), 8.02 (d, J=8.1 Hz, 2H), 7.72 (d, J=8.4 Hz, 2H), 4.18 (q, J=6.9 Hz, 2H), 2.50 (m, 3H), 1.27 (t, J=7.2 Hz, 3H).

### Example 2

### Compound 2 in Table 1

To a mixture of 5-methyl-4-(4-(trifluoromethyl)phenyl)-4H-pyrazol-3-amine 723 mg (3 mmol) in ethanol 20 ml, was added ethyl 3-oxo-3-(4-(trifluoromethyl)phenyl)propanoate 882 mg (3.4 mmol) The mixture was reacted for 2 h, 100° C. The reaction solution was filtered and distilled under reduced pressure. The crude was separated by silica gel column chromatography to give the compound 2, IR (KBr, cm⁻¹) 3434, 3055, 2774, 1691, 1632, 1585, 1571, 1521, 1494, 1448, 1326, 1172, 1129, 1065; ¹HNMR (DMSO-d6) δ 12.30 (s, 1H), 7.93 (br, 4H), 7.56 (s, 1H), 7.53 (br, 2H), 6.10 (s, 1H), 2.19 (s, 3H).

### Example 3

### Compound 3 in Table 1

### To a mixture of 5-methyl-4-(4-(trifluoromethyl)phenyl)-4H-pyrazol-3-amine 482 mg (2 mmol) in toluene 20 ml, was added ethyl 3-oxo-3-(4-(trifluoromethyl)phenyl)propanoate 434 mg (1.67 mmol) The mixture was refluxed for 2 h. The reaction solution was filtered and distilled under reduced pressure. The crude was separated by silica gel column chromatography to give the compound 3, IR (KBr cm⁻¹) 3429, 3168, 3135, 3081, 2928, 1660, 1619, 1588, 1530, 1460, 1407, 1403, 1322, 1158, 1124, 1068; ¹H NMR (CDCl₃) δ 8.00-7.66 (m, 4H), 7.62-7.55 (m, 4H), 6.26 (s, 1H), 2.40 (s, 3H).

### Example 4

### Compound 4 in Table 1

To a mixture of 2-(2,4-dichlorophenyl)-3-phenyl-1H-pyrazol-5-amine 608 mg (2.5 mmol) in ethanol 20 ml, was added 3-(4-(cyano)phenyl)-3-oxo-propionate 434 mg (2 mmol) The mixture was refluxed for 2 h. The reaction solution was filtered. The crude was separated by silica gel column chromatography to give the compound 4, IR (KBr cm⁻¹) 3435, 3061, 2925, 2854, 2230, 1668, 1626, 1584, 1503, 1436, 1384, 1336; ¹HNMR (CDCl₃) δ 8.03 (d, J=8.1 Hz, 2H), 7.90 (d, J=8.1 Hz), 7.79 (s, 1H), 7.42 (m, 8H).

### Example 5

### Compound 5 in Table 1

To a mixture of 5-ethyl 4-(2,4-dichlorophenyl)-4H-pyrazol-3-amine 512 mg (2.1 mmol) in toluene 20 ml, was added 3-(4-(cyano)phenyl)-3-oxo-propionate 434 mg (2 mmol) The mixture was refluxed for 2 h. The reaction solution was filtered. The crude was separated by silica gel column chromatography to give the compound 5, IR (KBr cm⁻¹) 3445, 3167, 3090, 2972, 2931, 2228, 1685, 1626, 1583, 1500, 1441, 1322, 1200, 1129, 1100, 1065, 1015, 813; ¹HNMR (CDCl₃) δ 8.02 (d, J=8.4 Hz, 1H), 7.90 (d, J=7.8 Hz, 2H), 7.77 (s, 1H), 7.52 (m, 5H), 2.55 (q, J=7.8 Hz, 2H), 1.10 (t, J=7.2 Hz, 3H).

### Example 6

### Compound 6 in Table 1

To a mixture of 5-cyclopropyl 4-(2,4-dichloro phenyl)-4H-pyrazol-3-amine 536 mg (2 mmol) in toluene 20 ml, was added 3-(4-(cyano))phenyl)-3-oxo-propionate 434 mg (2 mmol). The mixture was refluxed for 2 h. The reaction solution was filtered and distilled under reduced pressure. The crude was separated by silica gel column chromatography to give the compound 6, IR (KBr cm⁻¹) 3435, 3090, 2230, 1667, 1628, 1583, 1504, 1445, 1397, 1321, 1255, 1229, 1189, 1090; ¹HNMR (CDCl₃) δ 8.02 (d, J=8.4 Hz, 2H), 7.90 (d, J=8.4 Hz, 2H), 7.79 (d, 1H), 7.55 (s, 2H), 6.25 (s, 1H), 4.45 (m, 1H), 0.91 (m, 4H).

### Example 7

### Compound 7 in Table 1

To a mixture of 5-methyl-4-(2,4-dichlorophenyl)-4H-pyrazol-3-amine 592 mg (2.4 mmol) in toluene 20 ml, was added 3-(4-(trifluoromethyl)phenyl)-3-oxo-propionate 520 mg (2 mmol) The mixture was refluxed for 2 h. The reaction solution was filtered. The crude was separated by silica gel column chromatography to give the compound 7, IR (KBr cm¹) 3434, 3055, 2774, 1691, 1632, 1585, 1571, 1521, 1494, 1448; ¹HNMR (CDCl₃) δ 12.30 (s, 1H), 7.93 (m, 4H), 7.56 (s, 1H), 7.53 (br, 2H), 6.10 (s, 1H), 2.19 (s, 3H).

### Example 8

### Compound 8 in Table 1

To a mixture of 5-methyl-4-(2,4-dichlorophenyl)-4H-pyrazol-3-amine 512 mg (2.1 mmol) in toluene 20 ml, was added 3-(4-(trifluoromethyl)pyridin 3-yl)-3-oxo-propionate 522 mg (2 mmol) The mixture was refluxed for 2 h. The reaction solution was filtered. The crude was separated by silica gel column chromatography to give the compound 8, IR (KBr cm⁻¹) 3433, 3164, 3065, 2953, 2849, 1697, 1675, 1633, 1607, 1585, 1519, 1497, 1447, 1407, 1376; ¹HNMR (CDCl₃) δ 12.34 (s, 1H), 9.09 (s, 1H), 8.40 (d, J=18.0 Hz, 1H), 8.10 (d, J=18.0 Hz, 1H), 7.79 (d, 1H), 7.54 (m, 2H), 6.22 (s, 1H), 2.21 (s, 3H).

### Example 9

### Compound 9 in Table 1

To a mixture of 5-methyl-4-(2,4-dichlorophenyl)-4H-pyrazol-3-amine 512 mg (2.1 mmol) in toluene 20 ml, was added 3-(4-nitro)phenyl)-3-oxo-propionate 474 mg (2 mmol) The mixture was reacted for 2 h.20° C. The reaction solution was filtered. The crude was separated by silica gel column chromatography to give the compound 9, IR (KBr cm⁻¹) 3420, 3161, 3062, 2925, 2850, 1690, 1633, 1606, 1577, 1520, 1498, 1448, 1407, 1376, 1345, 1238, 1199, 1128, 1101, 1066, 1006; ¹HNMR (CDCl₃) δ 12.32 (s, 1H), 8.37 (d, J=8.1 Hz, 2H), 8.00 (d, J=8.1 Hz, 2H), 7.78 (s, 1H), 7.53 (s, 2H), 6.14 (s, 1H), 2.20 (s, 3H).

### Example 10

### Compound 10 in Table 1

To a mixture of 5-methyl-4-(2,4-dichlorophenyl)-4H-pyrazol-3-amine 512 mg (2.1 mmol) in toluene 20 ml, was added 3-(4-cyano)phenyl)-3-oxo-propionate 434 mg (1.8 mmol) The mixture was refluxed for 2 h. The reaction solution was filtered and distilled under reduced pressure. The crude was separated by silica gel column chromatography to give the compound 10, IR (KBr cm⁻¹) 3435, 3160, 3067, 2924, 2232, 1693, 1628, 1582, 1551, 1519, 1502, 1445, 1412, 1394, 1372, 1336, 1206, 1127, 1101; ¹HNMR (CDCl₃) δ 12.22 (br, 1H), 8.02 (d, J=8.1 Hz, 2H), 7.92 (d, J=8.1 Hz, 2H), 7.77 (s, 1H), 7.53 (s, 2H), 6.11 (s, 1H), 2.20 (s, 3H).

### Example 11

### Compound 11 in Table 1

To a mixture of 3-(2,4-dichlorophenyl)-2-methyl-5-(morpholinomethyl)pyrazolo[1,5-a]pyrimidin-7(4H)one 684 mg (1.74 mmol) in toluene 20 ml was added morpholine 180 mg (2.1 mmol) The mixture was refluxted for 2 h. The reaction solution was filtered and distilled under reduced pressure. The crude was separated by silica gel column chromatography to give the compound 11, IR (KBr cm⁻¹) 3415, 2958, 2928, 2858, 1679, 1622, 1586, 1557, 1505, 1455, 1376, 1332, 1208, 1115, 1068; ¹HNMR (CDCl₃) δ 7.69 (d, 1H), 7.45 (m, 2H), 7.77 (s, 1H), 5.74 (s, 1H), 4.30 (br, 1H), 3.57 (br, 4H), 3.38 (m, 2H), 2.39 (br, 4H), 2.16 (s, 3H).

### Example 12

### Compound 12 in Table 1

To a mixture of 5-methyl-4-(2,4-dichlorophenyl)-4H-pyrazol-3-amine 512 mg (2.1 mmol) in ethanol 30 ml, was added 2-(ethoxycarbonyl methylene) malonate 432 mg (2.3 mmol) and sodium ethoxide 0.55 g (mmol). The mixture was refluxed for 12 h. The reaction solution was filtered and distilled under reduced pressure. The crude was separated by silica gel column chromatography to give the compound 12, IR (KBr cm⁻¹) 3410, 2950, 1701, 1606, 1570, 1549, 1496, 1442, 1356, 1325, 1280, 1196, 1122, 1101, 1066, 1026, 1007, 885; ¹HNMR (CDCl₃) δ 8.37 (s, 1H), 7.69 (d, 1H), 7.42 (m, 2H), 3.68 (s, 3H), 2.18 (s, 1H).

### Example 13

### Compound 13 in Table 1

To a mixture of p-trifluoromethyl-benzoic acid 3.8 g (20 mmol) in ethanol 20 ml, was added thionyl chloride 3.8 g (32.2 mmol) The mixture was refluxed for 1 h. and then added 4-chloro-2-amino benzoic acid 3.114 g in pyridine 60 ml. The mixture was reacted for 2 h, 100° C. The reaction solution was filtered. The crude was separated by silica gel column chromatography to give the compound 13, IR (KBr cm⁻¹) 3093, 1770, 1618, 1599, 1577, 1564, 1462, 1426, 1411, 1327, 1312, 1296, 1240, 1162, 1123; ¹HNMR (CDCl₃) δ 8.42 (d, J=8.1 Hz, 2H), 8.19 (d, J=6.1 Hz, 1H), 7.75 (m, 3H), 7.53 (br, 1H).

### Example 14

### Compound 14 in Table 1

To a mixture of trifluoromethylbenzoic acid 1.9 g (10 mmol) in thionyl chloride 15 ml and dichloromethane 30 ml, was added 4-chloro-2-aminobenzamide 1.76 g (10 mmol) in THF 20 ml. The mixture was refluxed for 20 minutes. The reaction solution was filtered and distilled under reduced pressure. The crude was separated by silica gel column chromatography to give the compound 14, IR (KBr cm⁻¹) 3447, 3178, 3088, 683, 1603, 1569, 1447, 1431, 1335, 1321, 1168, 1124, 1066, 943, 913, 859, 782, 694; ¹HNMR (CDCl₃) δ 12.85 (s, 1H), 8.34 (d, J=8.1 Hz, 2H), 8.14 (d, J=8.4 Hz, 1H), 7.92 (d, J=6.6 Hz, 2H), 7.80 (br, 1H), 7.57 (br, 1H).

### Example 15

### Compound 15 in Table 1

To a mixture of 7-chloro-2-(4-(trifluoromethyl)phenyl) quinazolin-4(3H)one 500 mg (1.5 mmol), was added morpholine 3 ml The mixture was reacted for 12 h, 100° C. The reaction solution was filtered. The crude was separated by silica gel column chromatography to give the compound 15, IR (KBr cm⁻¹) 3444, 3149, 3084, 3046, 2957, 2926, 2861, 1651, 1602, 1504, 1448, 1379, 1332, 1321, 1243, 1218, 1151, 1123, 1057, 2861; ¹HNMR (CDCl₃) δ 8.34 (d, J=8.4 Hz, 2H), 8.00 (d, J=9.3 Hz, 1H), 7.92 (d, J=8.4 Hz, 2H), 7.25 (m, 1H), 7.07 (d, J=2.4 Hz, 1H), 3.77 (br, 4H), 3.45 (br, 2H), 3.36 (br, 2H).

### Example 16

### Compound 16 in Table 1

To a mixture of 5-methyl-4-(2,4-dichlorophenyl)-4H-pyrazol-3-amine 484 mg (2.1 mmol) in toluene 30 ml, was added 4-(4-cyanophenyl)-3-oxo-butanoate 710 mg (3.5 mmol) The mixture was refluxed for 2 h. The reaction solution was filtered and distilled under reduced pressure. The crude was separated by silica gel column chromatography to give the compound 16, IR (KBr cm⁻¹) 3447, 3164, 3089, 2965, 2932, 2974, 1686, 1629, 1584, 1498, 1447, 1339, 1188, 1157, 1100, 1093, 1005, 841, 592; ¹HNMR (CDCl₃) δ 7.93 (br, 4H), 7.78 (s, 1H), 7.53 (br, 2H), 6.18 (s, 1), 3.03 (m, 4H), 2.20 (s, 3H), 1.49 (br, 4H), 0.82 (m, 6H).

### Example 17

### Compound 17 in Table 1

To a mixture of 5-methyl-4-(2,4-dichlorophenyl)-4H-pyrazol-3-amine 484 mg (2.1 mmol) in toluene 30 ml was added 4-(4-aminophenyl)-3-oxo-butanoate 236 mg (1.2 mmol). The mixture was reacted for 2 h.20° C. The reaction solution was filtered. The crude was separated by silica gel column chromatography to give the compound 17, IR (KBr cm⁻¹) 3435, 3081, 2888, 2216, 1672, 1628, 1610, 1585, 1527; ¹HNMR (CDCl₃) δ 7.81 (s, 1H), 7.52 (m, 4H), 6.70 (d, J=8.7 Hz, 2H), 5.62 (s, 1H), 4.03 (s, 2H), 2.16 (s, 3H).

### Example 18

### Compound 18 in Table 1

To a mixture of 5-((trifluoromethyl)phenyl)-4-(2,4-dichlorophenyl)-4H-pyrazol-3-amine 592 mg (2.0 mmol) in toluene 20 ml was added 3-(4-(cyano)phenyl)-3-oxo-propionate 434 mg (2.0 mmol) The mixture was reacted for 2 h. 20° C. The reaction solution was filtered and distilled under reduced pressure. The crude was separated by silica gel column chromatography to give the compound 18, IR (KBr cm⁻¹) 3446, 2228, 1699, 1625, 1580, 1552, 1494, 1457, 1306, 1181, 1143, 1060, 984; ¹HNMR (CDCl₃) δ 8.04 (d, J=7.2 Hz, 2H), 7.90 (d, J=6.9 Hz, 2H), 7.81 (s, 1H), 7.55 (br, 2H), 6.28 (s, 1H).

### Example 19

### Compound 19 in Table 1

To a mixture of 5-cyclopropyl-4-(2,4-dichlorophenyl)-4H-pyrazol-3-amine 1.07 g (4.0 mmol) in ethanol 30 ml, was added diethyl 2-(ethoxycarbonylmethylene) malonate 432 mg (2.3 mmol) The mixture was reacted 20° C. for 12 h. The reaction solution was filtered. The crude was separated by silica gel column chromatography to give the compound 19, IR (KBr cm⁻¹) 3444, 2979, 2925, 1706, 1610, 1570, 1543, 1442, 1311, 1269, 1178, 1092, 1053, 997, 795; ¹HNMR (CDCl₃) δ 8.35 (s, 1H), 7.68 (d, J=1.5 Hz, 2H), 7.45 (m, 2H), 4.16 (q, J=7.5 Hz, 2H), 1.66 (m, 1H), 1.25 (m, 4H), 0.84 (t, J=7.5 Hz, 3H).

### Example 20

### Compound 20 in Table 1

To a mixture of 5-methyl-4-(2,4-dichlorophenyl)-4H-pyrazol-3-amine 484 mg (2.1 mmol) in toluene 20 ml, was added ethyl 2-methyl-3-oxo-3-(4-trifluoromethyl)phenyl)propionate 544 mg (1.98 mmol) The mixture was reacted for 2 hr. 20° C. The reaction solution was filtered. The crude was separated by silica gel column chromatography to give the compound 20, IR (KBr cm⁻¹) 3435, 3170, 3060, 2928, 2799, 1677, 1638, 1586, 1515, 1454, 1379, 1326, 1172, 1127, 1068, 1009; ¹HNMR (CDCl₃) δ 7.91 (d, J=8.4 Hz, 2H), 7.75 (s, 1H), 7.72 (d, J=4.8 Hz, 2H), 7.47 (br, 2H), 2.18 (s, 3H), 1.87 (s, 3H).

### Example 21

### Compound 21 in Table 1

To a mixture of 5-cyclopropyl-4-(2,4-dichlorophenyl)-4H-pyrazol-3-amine 536 mg (2.0 mmol) in toluene 20 ml, was added 2-methyl-3-oxo-3-(4-trifluoromethyl)phenyl)propionate 544 mg (1.98 mmol) The mixture was refluxed for 2 h. The reaction solution was filtered and distilled under reduced pressure. The crude was separated by silica gel column chromatography to give the compound 21, IR (KBr cm⁻¹) 3432, 3183, 3076, 2927, 1659, 1625, 1587, 1514, 1460, 1382, 1324, 1243, 1169, 1131, 1068, 1017; ¹HNMR (CDCl₃) δ 7.91 (d, J=8.1 Hz, 2H), 7.74 (m, 3H), 7.50 (br, 2H), 1.85 (s, 3H), 1.70 (m, 1H), 0.91 (m, 4H).

### Example 22

### Compound 22 in Table 1

To a mixture of 5-phenyl-4-(2,4-dichlorophenyl)-4H-pyrazol-3-amine 608 mg (2.0 mmol) in toluene 20 ml, was added 2-methyl-3-oxo-3-(4-trifluoromethyl)phenyl)propionate 544 mg (1.98 mmol) The mixture was refluxed for 2 h. The reaction solution was filtered and distilled under reduced pressure. The crude was separated by silica gel column chromatography to give the compound 22, IR (KBr cm⁻¹) 3434, 3166, 3063, 2927, 1678, 1655, 1632, 1586, 1439, 1378, 1325, 1243, 1170, 1127, 1068, 1017, ¹HNMR (CDCl₃) δ 7.93 (d, J=7.8 Hz, 2H), 7.75 (m, 3H), 7.44 (br, 3H), 7.40 (s, 1H), 7.37 (m, 3H), 1.90 (s, 3H).

### Example 23

### Compound 23 in Table 1

To a mixture of 5-ethyl 4-(2,4-dichlorophenyl)-4H-pyrazol-3-amine 484 mg (2.0 mmol) in toluene 20 ml, was added 2-methyl-3-oxo-3-(4-trifluoromethyl)phenyl)propionate 544 mg (1.98 mmol). The mixture was refluxed for 2 h. The reaction solution was filtered and distilled under reduced pressure. The crude was separated by silica gel column chromatography to give the compound 23, IR (KBr cm⁻¹) 3444, 3066, 2929, 1674, 1634, 1588, 1514, 1447, 1381, 1324, 1241, 1167, 1129, 1068, 1012; ¹HNMR (CDCl₃) δ 7.91 (d, J=8.1 Hz, 2H), 7.73 (m, 3H), 7.46 (br, 2H), 2.57 (q, J=7.5 Hz, 2H), 1.86 (s, 3H), 1.09 (t, J=7.5 Hz, 3H).

### Example 24

### Compound 24 in Table 1

To a mixture of 5-methyl-4-(2,4-dichlorophenyl)-4H-pyrazol-3-amine 484 mg (2.1 mmol) in toluene 20 ml, was added 2-methyl-3-oxo-3-(4-trifluoromethyl)phenyl)propionate 544 mg (2.0 mmol) The mixture was reacted for 2 h., 20° C. The reaction solution was filtered and distilled under reduced pressure. The crude was separated by silica gel column chromatography to give the compound 24, IR (KBr cm⁻¹) 3434, 3051, 2927, 2859, 1683, 1634, 1620, 1591, 1535, 1466, 1380, 1324, 1165, 1131, 1068, ¹HNMR (CDCl₃) δ 7.91 (d, J=8.4 Hz, 2H), 7.77 (m, 4H), 7.66 (br, 2H), 2.18 (s, 3H), 1.56 (s, 3H).

### Example 25

### Compound 25 in Table 1

To a mixture of 5-methyl-4-(2,4-dichlorophenyl)-4H-pyrazol-3-amine 484 mg (2.1 mmol) in toluene 20 ml was added 2-methyl-3-oxo-3-(4-(N,N-dipropyl methylsulfonylamino)phenyl)propionate 738 mg (2.16 mmol) The mixture was reacted for 2 h. 20° C. The reaction solution was filtered. The crude was separated by silica gel column chromatography to give the compound 25, IR (KBr cm⁻¹) 3435, 3060, 2972, 2931, 2875, 1679, 1637, 1584, 1502, 1458, 1378, 1339, 1163; ¹HNMR (CDCl₃) δ 7.93 (d, J=8.4 Hz, 2H), 7.72 (m, 3H), 747 (br, 2H), 3.06 (m, 4H), 2.18 (s, 3H), 1.85 (s, 3H), 1.50 (m, 4H), 0.82 (m, 6H).

### Example 26

### Compound 26 in Table 1

To a mixture of 5-phenyl-4-(2,4-dichlorophenyl)-4H-pyrazol-3-amine 608 mg (2.0 mmol) in toluene 20 ml, was added 2-methyl-3-oxo-3-(4-(N,N-dipropylmethylsulfonylamino)phenyl)propionate 738 mg (2.16 mmol). The mixture was reacted for 2 h.20° C. The reaction solution was filtered and distilled under reduced pressure. The crude was separated by silica gel column chromatography to give the compound 26, IR (KBr cm⁻¹) 3437, 3063, 2964, 2930, 2874, 1663, 1629, 1584, 1497, 1438, 1377, 1339; ¹HNMR (CDCl₃) δ 7.95 (d, J=7.8 Hz, 2H), 7.73 (m, 3H), 7.40 (br, 7H), 3.06 (t, J=9.1 Hz, 4H), 1.88 (s, 3H), 1.51 (m, 4H), 0.83 (m, 6H).

### Example 27

### Compound 27 in Table 1

To a mixture of 5-cyclopropyl 4-(2,4-dichlorophenyl)-4H-pyrazol-3-amine 436 mg (1.63 mmol) in toluene 20 ml, was added 2-methyl-3-oxo-3-(4-cyano) phenyl)propionate 434 mg (2.0 mmol) in toluene 20 ml. The mixture was reacted for 2 h. 20° C. The reaction solution was filtered and distilled under reduced pressure. The crude was separated by silica gel column chromatography to give the compound 27, IR (KBr cm⁻¹) 3436, 3167, 3092, 2921, 2854, 2227, 1661, 1632, 1584, 1505, 1378, 1336, 1237, 1096, 1017; ¹HNMR (CDCl₃) δ 8.02 (d, J=8.4 Hz, 2H)), 7.71 (m, 3H), 7.50 (br, 2H), 1.83 (s, 3H), 1.60 (m, 1H), 0.92 (m, 4H).

### Example 28

### Compound 28 in Table 1

To a mixture of 5-phenyl-4-(2,4-dichlorophenyl)-4H-pyrazol-3-amine 964 mg (3.17 mmol) in ethanol 20 ml, was added 2-methyl-3-oxo-3-(4-cyano) phenyl)propionate 816 mg (3.76 mmol) and sodium ethoxide 550 mg. The mixture was reacted for 30 h. 20° C. The reaction solution was filtered and distilled under reduced pressure. The crude was separated by silica gel column chromatography to give the compound 28, IR (KBr cm⁻¹) 3446, 2986, 2933, 1701, 1617, 1595, 1557, 1468, 1326, 1261, 1191, 1164, 1108, 1070, 1009; ¹HNMR (CDCl₃) δ 8.21 (s, 1H), 7.93 (d, J=7.8 Hz, 2H), 7.75 (m, 3H), 7.55-7.24 (m, 6H), 2.85 (m, 3H).

### Example 29

### Compound 29 in Table 1

To a mixture of 5-methyl-4-(2,4-dichlorophenyl)-4H-pyrazol-3-amine-964 mg (4.19 mmol) in toluene 20 ml was added 2-methyl-3-oxo-3-(4-cyano) phenyl)propionate 816 mg (3.76 mmol) The mixture was reacted for 30 h.20° C. The reaction solution was filtered and to give 29, IR (KBr cm⁻¹) 3436, 3166, 3052, 2926, 2228, 1674, 1638, 1584; ¹HNMR (CDCl₃) δ 8.01 (d, J=8.4 Hz, 2H), 7.72 (m, 3H), 7.47 (br, 2H), 2.19 (s, 3H), 2.18 (s, 3H).

### Example 30

### Compound 30 in Table 1

To a mixture of 5-ethyl-4-(2,4-dichlorophenyl)-4H-pyrazol-3-amine 964 mg (4.16 mmol) in toluene 20 ml was added 2-methyl-3-oxo-3-(6-(trifluoromethyl)pyridin-3-yl)propionate 825 mg (3.8 mmol). The mixture was reacted for 30 h.20° C. The reaction solution was filtered. The crude was separated by silica gel column chromatography to give the compound 30, IR (KBr cm⁻¹) 3462, 3172, 3066, 2936, 2855, 1669, 1637, 1586, 1502, 1460, 1381, 1335; ¹HNMR (CDCl₃) δ 8.92 (s, 1H), 8.28 (d, J=6.9 Hz, 1H), 8.10 (d, J=8.4 Hz, 1H), 7.73 (s, 1H), 7.48 (s, 2H), 2.57 (m, 2H), 1.88 (s, 3H), 1.08 (t, J=6.2 Hz, 3H).

### Example 31

### Compound 31 in Table 1

To a mixture of 5-methyl-4-(2,4-dichlorophenyl)-4H-pyrazol-3-amine 4.36 g (20 mmol) in ethanol 50 ml, was added Ethoxymethylene malononitrile 2.85 g (24 mmol). The mixture was reacted for 3 h, 50° C. The reaction solution was filtered. The crude was separated by silica gel column chromatography to give the compound 31, IR (KBr, cm⁻¹) 3405, 3308, 3246, 3177, 2930, 2223, 1650, 1602, 1553, 1493, 1367, 1329, 1292, 1277, 1104, 1063, 1001, 857, 876, 816, 797, 610, 527, 507; ¹HNMR (DMSO-d6) δ 8.97 (s, 2H), 8.28 (s, 1H), 7.76 (s, J=1.8 Hz, 1H), 7.52 (s, 1H), 7.46 (d, J=8.4 Hz, 1H), 2.32 (s, 3H).

### Example 32

### Compound 32 in Table 1

To a mixture of o-aminonitrile 1.27 g (4.0 mmol) in pyridine 30 ml, was added PITC 4.86 g (36.00 mmol). The mixture was refluxed for 5 h. The reaction solution was filtered. The crude was separated by silica gel column chromatography to give the compound 32, IR (KBr, cm⁻¹) 3429, 3352, 3147, 1621, 1577, 1550, 1496, 1456, 1385, 1333, 1306, 1285, 1260, 1143, 1101, 1067; ¹H NMR (DMSO-d6) δ 9.53 (br, 1H), 9.03 (s, 1H), 7.79 (s, 1H), 7.62 (t, J=7.8 Hz, 1H), 7.54 (m, 2H), 7.51 (m, 1H), 7.36 (m, 2H), 2.32 (s, 3H).

### Example 33

### Compound 33 in Table 1

To a mixture of 5-methyl-4-(trifluoromethyl phenyl)-4H-pyrazol-3-amine 0.964 g (3.98 mmol) in toluene 20 ml, was added 2-methyl-3-oxo-3-(4-cyano)phenyl) propionate 816 mg (3.53 mmol). The mixture was refluxed for 30 h. The reaction solution was filtered and distilled under reduced pressure. The crude was separated by silica gel column chromatography to give the compound 33, IR (KBr, cm⁻¹) 3432, 3073, 2925, 2227 cm⁻¹, 1677, 1631, 1589, 1534, 1465, 1379, 1324, 1240, 1163, 1122, 1059; ¹H NMR (DMSO-d6) δ8.02 (d, J=7.8 Hz, 2H), 7.76 (d, J=8.4 Hz, 3H), 7.66 (J=7.8 Hz, 2H), 7.46 (br, 1H), 2.18 (s, 3H), 1.85 (s, 3H).

### Example 34

### Compound 34 in Table 1

To a mixture of 5-methyl-4-(2,4-dichlorophenyl)-4H-pyrazol-3-amine 1.2 g (4.96 mmol) in ethanol 20 ml, was added nitrocinnamonitrile 1.0 g (5.0 mmol) The mixture was reacted for 12 h, room temperature. The reaction solution was filtered and distilled under reduced pressure. The crude was separated by silica gel column chromatography to give the compound 34, IR (KBr, cm⁻¹) 3432, 3399, 3302, 3181, 2922, 2189, 1655, 1619, 1589, 1522, 1472, 1383, 1349, 1317, 1269, 1182, 1099, 1068, 1002, 728, 708; ¹H NMR (DMSO-d6) δ 8.25 (s, 1H), 8.19 (d, J=6.2 Hz, 1H), 7.74 (d, J=6.2 Hz, 1H), 7.56 (t, J=6.2 Hz, 1H), 7.46 (s, 1H), 7.30 (m, 1H), 7.19 (d, J=6.2 Hz, 1H), 5.52 (br, 2H), 5.42 (s, 1H), 4.53 (m, 1H), 2.13 (s, 3H).

### Example 35

### Compound 35 in Table 1

To a mixture of 4-(2,4-dichlorophenyl)-4H-pyrazol-3-amine 730 mg (3.2 mmol) in toluene 40 ml, was added 5-ethylindandione 750 mg (4.3 mmol) and m-nitrobenzyl formaldehyde 1.28 g (7.75 mmol). The mixture was refluxed for 6 h. The reaction solution was filtered. The crude was separated by silica gel column chromatography to give the compound 35, IR (KBr, cm⁻¹) 3435, 3218, 2965, 2927, 1661, 1611, 1572, 1528, 1490, 1463, 1438; ¹H HNMR (DMSO-d6) δ 11.38 (s, 1H), 8.14 (m, 1H), 7.98 (br, 1H), 7.84 (m, 1H), 7.74 (m, 3H), 7.56 (m, 2H), 7.41 (m, 2H), 7.30 (br, 1H), 6.50 (s, 1H), 2.33 (m, 2H), 0.91 (s, 3H).

### Example 36

### Compound 36 in Table 1

To a mixture of 5-amino-1-phenyl-1H-4-nitrile 1.84 g (10 mmol) in pyridine 25 ml, was added PITC 1.35 g (10 mmol). The mixture was refluxed for 20 h. The reaction solution was filtered and distilled under reduced pressure. The crude was separated by silica gel column chromatography to give the compound 36, (3 mmol) IR (KBr, cm⁻¹) 3358, 3063, 1615, 1582, 1558, 1531, 1498, 1459, 1425, 1355, 1313, 1272, 1242, 1165, 10871, 981, 9431, 779; ¹HNMR (DMSO-d6) δ 10.41 (s, 1H), 8.49 (br, 1H), 8.24-6.70 (m, 11H).

### Example 37

### Compound 37 in Table 1

To a mixture of 5-amino-1-phenyl-1H-pyrazole-4-carbonitrile 552 mg (3 mmol) in ethanol 8 ml, was added 2,4-dichlorobenzeneformaldehyde 630 mg (3.4 mmol) and NaOH 240 mg. The mixture was reacted for 2 h, 60° C. The reaction solution was filtered. The crude was separated by silica gel column chromatography to give the compound 37, IR (KBr, cm⁻¹) 3084, 1590, 1566, 1505, 1531, 1463, 1436, 1424, 1398, 1348, 1311, 1204 cm⁻¹, 1103, 1088, 933, 792; ¹HNMR (DMSO-d6) δ 8.31 (d, J=8.04 Hz, 2H), 8.25 (s, J=7.5 Hz, 1H), 7.94 (d, J=8.4 Hz, 1H), 7.52 (t, 3H), 7.38 (m, 2H), 4.42 (q, J=7.05 Hz, 2H), 1.53 (t, J=7.11 Hz, 3H).

### Example 38

### Compound 38 in Table 1

To a mixture of 5-amino-1-phenyl-1H-pyrazol-4-carbonitrile 1.00 g (3.0 mmol) in 1,4-dioxane 10 ml, was added benzonitrile 0.672 g (3.6 mmol) and sodium methoxide 0.578 g. The mixture was refluxed for 18 h. The reaction solution was filtered and distilled under reduced pressure. The crude was separated by silica gel column chromatography to give the compound 38, IR (KBr, cm⁻¹) 3485, 3302, 3098, 1649, 1587, 1567, 1506, 1477, 1425, 1393, 1290, 1211, 1063, 971, 933, 796, 774, 761, 710, 690, 628; ¹HNMR (DMSO-d6) δ 8.50 (m, 2H), 8.44 (s, 1H), 8.40 (br, 2H), 7.96 (br, 2H), 7.66 (m, 2H), 7.57 (m, 3H), 7.41 (m, 1H).

### Example 39

### Compound 39 in Table 1

To a mixture of 5-amino-1-phenyl-1H-pyrazol-4-carbonitrile 4.0 g (55.8 mmol) in DMSO 15 ml, was added dimethyl acetylenedicarboxylate 6.17 g (41 mmol) and potassium carbonate 6.0 g. The mixture was reacted for 5 h, 100° C. The reaction solution was filtered. The crude was separated by silica gel column chromatography to give the compound 39, IR (KBr, cm⁻¹) 3474, 3364, 2950, 1707, 1627, 1589, 1559, 1500, 1476, 1438, 1369, 1236, 1120, 1033, 959, 777; ¹HNMR (DMSO-d6) δ 8.67 (s, 1H), 8.50 (br, 1H), 8.12 (br, 3H), 7.56 (t, J=7.8 Hz, 2H), 7.36 (t, J=7.8 Hz, 1H), 3.85 (s, 3H), 3.82 (s, 3H).

### Example 40

### Compound 40 in Table 1

To a mixture of 2-(ethoxylmethyl)imino-1-phenyl-1H-pyrazol-4-cyanopyrazole 1.20 g (5.0 mmol) in ethanol 15 ml, was added aniline 0.50 g (5.0 mol). The mixture was refluxed for 5 h. The reaction solution was filtered. The crude was separated by silica gel column chromatography to give the compound 40, IR (KBr, cm⁻¹) 3435, 3188, 3056, 2917, 1605, 1585, 1504, 1439, 1367, 1327, 1313; ¹H NMR (DMSO-d6) δ 10.23 (s, 1H,), 8.54 (br, 2H), 8.21 (d, J=7.8 Hz, 2H), 7.94 (d, J=7.8 Hz, 2H), 7.57 (t, 2H), 7.41 (m, 3H), 7.17 (m, 1H).

### Example 41

### Compound 41 in Table 1

To a mixture of phenyl malonate 4.88 g (27 mmol) in tributylamine 6 ml, was added 5-methyl-4-(2,4-dichlorophenyl)-4H-pyrazol-3-amine 0.964 g (40.0 mmol). The mixture was reacted for 20 minutes, 110° C. The reaction solution was filtered and distilled under reduced pressure. The crude was separated by silica gel column chromatography to give the compound 41, IR (KBr, cm⁻¹) 3434, 2956, 2931, 2870, 1639, 1615, 1592, 1560, 1501, 1440, 1348, 1374, 1307, 1258, 1170, 1101, 1065, 1006, 902; ¹HNMR (DMSO-d6) δ 9.98 (br, 1H), 9.35 (br, 1H), 7.69 (d, J=7.2 Hz, 2H), 7.63 (d, J=1.8, 1H), 7.40 (dd, J=2.4 Hz, J=8.4, 1H), 7.34 (d, J=8.4 Hz, 1H), 7.14 (t, J=7.8, 2H), 6.94 (t, J=7.2, 1H), 2.05 (s, 3H).

### Example 42

### Compound 42 in Table 1

To a mixture of diethyl phenylmalonate 0.52 g (2.2 mmol) in tributylamine 20 ml, was added 3-(4-(trifluoromethyl)phenyl)-1-H-pyrazol-5-amine 0.50 g (19.4 mmol) The mixture was reacted for 1 h, 110° C. The reaction solution was filtered and distilled under reduced pressure. The crude was separated by silica gel column chromatography to give the compound 42, IR (KBr, cm⁻¹) 3415, 2962, 2933, 2874, 1641, 1533, 1579, 1616; ¹HNMR (DMSO-d6) δ 10.42 (br, 1H), 9.04 (br, 1H), 8.09 (s, 2H), 7.72 (s, 4H), 7.16 (s, 2H), 6.96 (s, 1H), 6.02 (s, 1H).

### Example 43

### Compound 43 in Table 1

To a mixture of 2-amino benzimidazole 0.510 g (3.8 mmol) in THF 20 ml, was added 3-oxo-3-(4-(trifluoromethyl)phenyl)propionate 1.0 g (4.3 mmol) The mixture was reacted for 5 h, 120° C. The reaction solution was filtered and distilled under reduced pressure. The crude was separated by silica gel column chromatography to give the compound 43, IR (KBr, cm⁻¹) 3435, 3058, 2926, 1673, 1557, 1468, 1487, 1443, 1332, 1247, 1154, 1114, 1071, 1017, 991, 810; ¹HNMR (DMSO-d6) δ 13.21 (br, 1H), 8.48 (d, J=7.8, 1H), 8.34 (d, J=7.8, 2H), 7.87 (d, J=8.4, 2H), 7.49 (m, 2H), 7.32 (t, J=7.2, 1H), 6.68 (s, 1H).

### Example 44

### Compound 44 in Tablet

To a mixture of 3-(2,4-dichlorophenyl) 6-iso cyano-2-methylpyrazolo[1,5-a]pyrimidin-7-amine 1.00 g (3.1 mmol) in ethylenediamine 20 ml was added p-toluene sulfonic acid 0.717 g (4.2 mmol) and TMF 20 ml. The mixture was reacted for 5 h, 120° C. The reaction solution was filtered. The crude was separated by silica gel column chromatography to give the compound 44, IR (KBr, cm⁻¹) 3357, 3297, 2926, 2857, 1639, 1606, 1429, 1470, 1359, 1334, 1330, 1141, 1067, 1093; ¹HNMR (DMSO-d6) δ 8.63 (br, 1H), 8.42 (s, 1H), 7.75 (d, J=1.8, 1H), 7.50 (dd, J=1.8, J=7.8, 2H), 3.43 (t, J=8.4, 3H), 3.17 (s, 2H), 1.06 (t, J=7.2, 2H).

### Example 45

### Compound 45 in Table 1

To a mixture of N-(benzoyl)methyl-1H-indole-2-cyanide 1.0 g (3.8 mmol), was added hydrazine hydrate 10.0 ml. The mixture was reacted for 2 h, 100° C. The reaction solution was filtered. The crude was separated by silica gel column chromatography to give the compound 45, IR (KBr, cm⁻¹) 3284, 3148, 3106, 3052, 1632, 1503, 1460, 1406, 1373, 1325, 1295, 1266, 1218, 1188, 1150, 1122, 1108, 1055, 1014, 974, 930, 900; ¹HNMR (DMSO-d6) δ 8.21 (s, 1H), 8.23 (d, J=8.4, 1H), 7.84 (d, J=7.8, 1H), 7.82 (d, J=7.8, 2H), 7.50 (t, J=7.8, 2H), 7.43 (q, J=7.2, 2H), 7.33 (d, J=7.2, 1H), 7.30 (s, 1H), 3.89 (br, 3H).

### Example 46

### Compound 46 in Table 1

To a mixture of isatoic anhydride 1.19 g (7.3 mmol) in glacial acetic acid 10 ml, was added isonicotinic acid hydrazide 1.0 g (7.3 mmol) The mixture was reacted for 20 minutes, 130° C. The reaction solution was filtered and distilled under reduced pressure. The crude was separated by silica gel column chromatography to give the compound 46, IR (KBr, cm⁻¹) 3461, 3350, 3214, 3040, 1665, 1632, 1585, 1552, 1533, 1487, 1448, 1410, 1384, 1294, 1265, 1160, 1068; ¹H NMR (DMSO-d6) δ 8.79 (dd, J=1.8, J=5.4, 2H), 7.82 (d, J=5.4, 2H), 7.61 (d, J=7.2, 1H), 7.21 (t, J=7.2, 1H), 6.75 (d, J=7.8, 1H), 6.56 (t, J=7.2, 1H), 6.42 (br, 1H).

### Example 47

### Compound 47 in Table 1

To a mixture of adding guanidine hydrochloride 1.67 g (2.8 mmol) in methanol 250 ml, was added benzil 3.0 g (1.4 mmol) and NaOH 0.68 g The mixture was reacted for 30 minutes, room temperature. The reaction solution was filtered. The crude was separated by silica gel column chromatography to give the compound 47, IR (KBr. cm⁻¹) δ 3347, 3176, 2718, 1672, 1599, 1565, 1493, 1445, 1384, 1321, 1170, 1122, 1070, 1011, 955, 850, 774; ¹HMR (DMSO-d6) δ 7.47-7.12 (m, 10H), 3.62 (br, 3H).

### Example 48

### Compound 48 in Table 1

To a mixture of 2-amino-4,5-diphenyl-4H-imidazole-4-ol 30.4 g (12 mmol) in methanol 200 ml, was added 5% Pd/C 4.0 g. The mixture was hydrogenation for 24 h. The reaction solution was filtered. The crude was separated by silica gel column chromatography to give the compound 48, IR (KBr, cm⁻¹) 3707, 3443, 3359, 3051, 2717, 1611, 1574, 1501, 1264, 1174, 1071, 760, 697, 599; ¹H MR (DMSO-d6) δ 7.42-7.19 (m, 10H), 3.64 (br, 3H).

### Example 49

### Compound 49 in Table 1

To a mixture of diphenyl imidazole 0.5 g (2.3 mmol) in, was added ethyl acetate 20 ml. The mixture was reacted for 4 h, 120° C. The reaction solution was filtered. The crude was separated by silica gel column chromatography to give the compound 49, IR (KBr, cm⁻¹) 3444, 3055, 2924, 2853, 1681, 1649, 1614, 1599, 1580, 1439; ¹HNMR (DMSO-d6) δ 7.38 (m, 7H), 7.21 (m, 3H), 5.49 (s, 1H), 2.28 (s, 3H).

### Example 50

### Compound 50 in Table 1

To a mixture of (E)-ethyl N-3-cyano-4-(3-nitrophenyl)-4H-benzo[h]chromen-2-ylformimidate 2.00 g (5.0 mmol) in 1,4-dioxane 20 ml, was added methylamine hydrochloride 0.40 g (6.0 mmol) The mixture was reacted for 10 h, 100° C. The reaction solution was filtered and distilled under reduced pressure. The crude was separated by silica gel column chromatography to give the compound 50, IR (KBr cm⁻¹) 3431, 2950, 2857, 1660, 1616, 1585, 1572, 1533, 1423, 1393, 1376, 1348, 1280, 1263, 1223, 1117, 1085, 930, 870; ¹HNMR (DMSO-d6) δ 12.72 (s, 1H), 8.30 (d, J=8.4 Hz, 1H), 8.23 (s, 1H), 8.21-8.20 (t, J=1.8 Hz, 1H), 8.05 (d, J=1.2 Hz, 1H,), 7.93 (d, J=7.8 Hz, 1H), 7.72 (m, 3H), 7.63 (t, J=1.2 Hz, 1H), 7.55 (t, J=4.8 Hz, 1H), 7.34 (d, J=8.4 Hz, 1H), 5.57 (s, 1H), 3.57 (s, 3H).

### Example 51

### Compound 51 in Table 1

To a mixture of (E)-ethyl N-3-cyano-4-(3-nitrophenyl)-4H-benzo[h]chromen-2-ylformimidate 5.00 g (12.5 mmol) in ethanol 50 ml was added anhydrous hydrazine 12 ml. The mixture was reacted for 2 h, 100° C. The reaction solution was filtered. The crude was separated by silica gel column chromatography to give the compound 51, IR (KBr cm⁻¹) 3428, 3328, 3173, 3081, 1651, 1624, 1520, 1419, 1381; ¹HNMR (DMSO-d6) δ 8.321 (br, 1H), 8.271 (d, J=8.4 Hz, 1H), 8.16-8.13 (br, 1H,), 8.03 (d, J=7.8 Hz, 1H), 7.914 (d, J=7.8 Hz, 1H), 7.790 (s, 1H), 7.676 (m, 1H), 7.676 (m, 1H), 7.608 (t, J=8.4 Hz, 1H), 7.555 (t, J=7.2 Hz, 1H), 7.333 (d, J=7.0 Hz, 1H), 6.877 (s, 1H), 5.701 (s, 2H), 5.591 (s, 1H).

### Example 52

### Compound 52 in Table 1

To a mixture of 24-amino-5-(3-nitrophenyl)-4H-benzo[h]chromeno[2,3-d]pyrimidin-3(5H)-amine 1.0 g (2.6 mmol) was added triethyl orthoformate 15 ml. The mixture was reacted for 2 h, 100° C. The reaction solution was filtered. The crude was separated by silica gel column chromatography to give the compound 52, IR (KBr cm⁻¹) 3444, 3080, 2924, 2854, 1619, 1602, 1536, 1506, 1444, 1420, 1397, 1379, 11348, 1333, 1298; ¹HNMR (DMSO-d6) δ 9.76 (s, 1H), 8.57 (s, 1H), 8.40-8.37 (m, 1H), 8.06-8.04 (d, J=9.6 Hz, 1H), 7.97-7.96 (d, J=8.4 Hz, 1H), 7.82 (d, J=7.8 Hz, 1H), 7.55 (t, J=8.4 Hz, 2H), 7.67 (t, J=7.2 Hz 1H), 7.56 (t, J=7.8 Hz, 1H), 7.42 (d, J=8.4 Hz, 1H), 7.40 (m, 1H), 6.21 (s, 1H)

### Example 53

### Compound 53 in Table 1

To a mixture of 24-amino-5-(3-nitrophenyl)-4H-benzo[h]chromeno[2,3-d]pyrimidin-3(5H)-amine 1.0 g (2.6 mmol) in glacial acetic acid 5 ml, was added acetyl chloride 256 mg (3.0 mmol) The mixture was refluxed for 2 h. The reaction solution was filtered. The crude was separated by silica gel column chromatography to give the compound 53, IR (KBr cm⁻¹) 3434, 3162, 3014, 2851, 1659, 1632, 1562, 1537, 1432, 1380, 134; ¹HNMR (DMSO-d6) δ 9.62 (s, 1H), 8.39-8.38 (d, J=8.4 Hz, 1H), 8.34-8.33 (t, J=1.8 Hz, 1H), 8.05 (m, J=7.8 Hz, 1H), 7.97 (d, J=8.4 Hz, 1H), 7.82 (t, J=6.6 Hz, 1H), 7.74 (m, 2H), 7.66 (t, J=1.2 Hz, 1H), 7.57 (t, J=7.8 Hz, 1H), 7.43 (d, J=8.4 Hz, 1H), 6.16 (s, 1H), 2.50 (s, 3H)

### Example 54

### Compound 54 in Table 1

To a mixture of 24-amino-5-(3-nitrophenyl)-4H-benzo[h]chromeno[2,3-d]pyrimidin-3(5H)-amine 2.0 g (5.2 mmol) in ethanol 30 ml was added 2,4-dichlorobenzene formaldehyde 1.09 g (6.3 mmol). The mixture was refluxed for 5 h. The reaction solution was filtered. The crude was separated by silica gel column chromatography to give the compound 54, IR (KBr cm⁻¹) 3412, 3362, 3070, 1645, 1571, 1526, 1446, 1380, 1345, 1239, 1180, 1097, 971, 829; ¹H NMR (DMSO-d6) δ 8.33 (s, 1H), 8.28 (s, 1H), 8.15 (s, 1H), 7.94 (d, J=7.2, 1H), 7.76 (d, J=7.8 Hz, 1H), 7.76 (d, J=7.8 Hz, 1H), 7.69 (t, J=7.8 Hz, 1H), 7.69 (t, J=7.8 Hz, 1H), 7.64 (m, 1H), 7.61 (m, 1H), 7.56 (s, 1H), 7.55 (d, J=1.8 Hz, 1H), 7.32 (d, J=8.4 Hz, 1H), 6.97 (d, J=8.4 Hz, 1H), 6.38 (s, 1H), 6.33 (d, J=8.4 Hz, 1H), 5.56 (s, 1H).

### Example 55

### Compound 55 in Table 1

To a mixture of 4-amino-5-(3-nitrophenyl)-4H-benzo[h]chromeno[2,3-d]pyrimidin-3(5H)-amine 1.0 g (2.6 mmol), was added diethyl oxalate 5.0 ml. The mixture was reacted for 5 h, 120° C. The reaction solution was filtered and distilled under reduced pressure. The crude was separated by silica gel column chromatography to give the compound 55, IR (KBr cm⁻¹) 3427, 2923, 1735, 1625, 1561, 1530, 1482, 1383, 1347, 1301, 1208, 1115, 1020, 833, 764; ¹HNMR (DMSO-d6) δ 9.86 (s, 1H), 8.40 (m, 2H), 8.06-8.04 (d, J=1.8 Hz, 1H), 7.98 (d, J=8.4 Hz, 1H,), 7.83 (d, J=7.8 Hz, 1H), 7.77 (m, 2H), 7.67 (t, J=7.8 Hz, 1H), 7.57 (t, J=7.8 Hz, 1H), 7.47 (d, J=8.4 Hz, 1H), 6.30 (s, 1H), 4.04-4.37 (m, 2H), 1.34-1.31 (t, J=7.2 Hz, 3H).

### Example 56

### Compound 56 in Table 1

To a mixture of 4-amino-5-(3-nitrophenyl)-4H-benzo[h]chromeno[2,3-d]pyrimidin-3(5H)-amine 1.0 g (2.6 mmol) in 1,4-dioxane 10.0 ml, was added PITC 2.0 ml. The mixture was reacted for 7 h, 90° C. The reaction solution was filtered. The crude was separated by silica gel column chromatography to give the compound 56, IR (KBr cm⁻¹) 3308, 2924, 1605, 1570, 1530, 1448, 1398, 1377, 1345, 1273, 1261, 1235, 1180, 1119, 1085, 1235; ¹HNMR (DMSO-d6) δ 9.90 (s, 1H), 9.48 (s, 1H), 8.40 (t, J=4.8 Hz, 2H), 8.08 (d, J=1.2 Hz, 1H), 7.96 (d, J=4.8 Hz, 1H), 7.83 (d, J=7.8 Hz, 1H), 7.73 (m, 2H), 7.66 (t, J=7.8 Hz, 3H), 7.58 (t, J=8.4 Hz, 1H), 7.45 (d, J=8.4 Hz, 1H), 7.30 (d, J=7.8 Hz, 2H), 6.94 (t, J=7.2 Hz, 1H), 6.09 (s, 1H).

### Example 57

### Compound 57 in Table 1

To a mixture of 4-amino-5-(3-nitrophenyl)-4H-benzo[h]chromeno[2,3-d]pyrimidin-3(5H)-amine 1.0 g (2.6 mmol), was added benzoyl chloride 30.0 ml. The mixture was reacted for 2 h, 130° C. The reaction solution was filtered and distilled under reduced pressure. The crude was separated by silica gel column chromatography to give the compound 57, IR (KBr cm⁻¹) 3457, 1628, 1604, 1566, 1530, 1498, 1455, 1446-9, 1412, 1399, 1377, 1345, 1318, 1345, 1318, 1274, 1236, 1185, 1127, 1115; ¹HNMR (DMSO-d6) δ9.79 (s, 1H), 8.49 (t, J=1.8 Hz, 1H), 8.41 (d, J=8.4 Hz, 1H), 8.19 (m, 2H), 8.07 (d, J=1.2 Hz, 1H), 7.99 (d, J=8.4 Hz, 1H), 7.84 (m, 1H), 7.78 (m, 1H), 7.68 (m, 1H), 7.58 (m, 5H), 7.44 (d, J=9 Hz, 1H), 6.285 (s, 1H).

### Example 58

### Compound 58 in Table 1

To a mixture of 2-indole nitrile 2.48 g (17 mmol) in acetone 20 ml, was added bromoacetophenone 3.47 g (17 mmol), TBAI90 mg (0.247 mmol) and anhydrous carbonate potassium 9.630 g (70 mmol). The mixture was refluxed for 2 h. The reaction solution was filtered and distilled under reduced pressure. The crude was separated by silica gel column chromatography to give the compound 58, IR (KBr, cm⁻¹) 3363, 3116, 3059, 2956, 2918, 2224, 1684, 1621, 1595, 1578, 1520, 1480, 1451, 1430, 1402, 1372, 1345, 1317, 1226, 1171, 1139.

### Example 59

### Compound 59 in Table 1

To a mixture of 2-(2,4-dichlorophenyl)-3-phenyl-1H-pyrazol-5-amine 1.0 g (4 mmol) in ethanol 20 ml, was added hydrazine hydrate The mixture was reacted for 2 h, 100° C. The reaction solution was filtered. The crude was separated by silica gel column chromatography to give the compound 59, IR (KBr, cm⁻¹) 3284, 3148, 3106, 3052, 1632, 1503, 1460, 1406, 1373, 1325, 1295, 1266, 1218, 1188, 1150, 1122, 1108, 1055, 1014; ¹HNMR (DMSO-d6) 8.21 (s, 1H), 8.23 (d, J=8.4, 1H), 7.84 (d, J=7.8, 1H), 7.82 (d, J=7.8, 2H), 7.50 (t, J=7.8, 2H), 7.43 (q, J=7.2, 2H), 7.33 (d, J=7.2, 1H), 7.30 (s, 1H).

### Example 60

### Compound 60 in Table 1

To a mixture of 4-(3-(2,4-dichlorophenyl)-2-methyl-7-oxo-4,7-dihydropyrazolo[1,5-a]pyrimidin-5-yl))benzoic acid 1.0 g (24 mmol) was added concentrated nitric acid 25 ml The mixture was reacted for 5 h, rt. The reaction solution was filtered. The crude was separated by silica gel column chromatography to give the compound 60, IR (KBr, cm⁻¹) 3444, 3088, 2924, 2647, 1692, 1599, 1584, 1542, 1468; ¹HNMR (DMSO-d6) δ7.75 (s, 1H), 7.69 (d, J=9.0 Hz, 2H), 7.51 (br, 2H), 7.09 (d, J=9.0 Hz, 2H), 2.18 (s, 3H).

### Example 61

### Compound 61 in Table 1

To a mixture of 3-(2,4-dichlorophenyl)-2-methyl-5-(4-trifluoromethyl)phenyl)pyrazolo[1,5-a]pyrimidin-7(4H)-one 1.0 g (2.3 mmol) was added nitric acid 25 ml The mixture was reacted for 5 h, room temperature. The reaction solution was filtered. The crude was separated by silica gel column chromatography to give the compound 61, IR (KBr, cm⁻¹) 3444, 3132, 2675, 2553, 1694, 1606, 1584, 1541, 1470, 141428, 1415, 1383, 1316, 1172, 1143, 1129, 1065; ¹HNMR (DMSO-d6), 67.97 (d, J=8.4 Hz, 1H), 7.91 (d, J=7.8 Hz, 2H), 7.84 (d, J=1.8 Hz, 1H), 7.68 (d, J=8.4 Hz, 2H), 7.64 (m, 1H), 2.40 (s, 3H).

### Example 62

### Compound 62 in Table 1

To a mixture of 3-(2,4-dichlorophenyl)-7-H-2-methyl pyrazolo[1,5-a]pyrimidin-7-one-5-yl)benzonitrile 790 mg (2.0 mmol) in N-dimethyl formamide 15 ml, was added methyl iodide 850 mg and sodium carbonate 636 mg (4.5 mmol). The mixture was reacted for 24 h, room temperature. The reaction solution was filtered and distilled under reduced pressure. The crude was separated by silica gel column chromatography to give the compound 62, IR (KBr, cm⁻¹) 3435, 3087, 2924, 2853, 2218, 1671, 1568, 1539, 1503, 1488, 1422, 1385, 1291, 1185, 1123, 1100, 941, 851, 807; ¹HNMR (DMSO-d6) 8.02 (d, J=8.1 Hz, 2H), 7.67 (d, J=8.7 Hz, 2H), 7.58 (s, J=1.5 Hz, 1H), 7.40 (s, 2H), 6.49 (s, 1H), 4.39 (s, 3H), 2.36 (s, 3H).

### Example 63

### Compound 63 in Table 1

To a mixture of 5-methyl-4-(4-(trifluoromethyl)phenyl)-4H— pyrazol-3-amine 482 mg (2.0 mmol) in toluene 20 ml, was added, ethyl 3-oxo-3-(6-(trifluoromethyl)pyridin-3-yl)propionate 522 mg (2.0 mmol) The mixture was refluxed for 2 h. The reaction solution was filtered and distilled under reduced pressure. The crude was separated by silica gel column chromatography to give the compound 63, IR (KBr, cm⁻¹) 3431, 3069, 3036, 2923, 2746, 1654, 1620, 1557, 1539, 1484, 1442, 1406, 1334, 1180, 1166, 1128; ¹HNMR (DMSO-d6) δ9.17 (s, 1H), 8.49 (d, J=6.0 Hz, 1H), 8.08 (d, J=7.8 Hz, 1H), 7.82 (m, 4H), 6.12 (s, 1H), 2.39 (s, 3H).

### Example 64

### Compound 64 in Table 1

To a mixture of 1-(2,4-dichlorophenyl)-5-phenyl-1H-pyrazol-3-amine 608 mg (2.0 mmol) in toluene 20 ml, was added ethyl 3-oxo-3-(4-(trifluoromethyl)phenyl)propionate 520 mg (2.34 mmol) The mixture was refluxed for 2 h. The reaction solution was filtered and distilled under reduced pressure. The crude was separated by silica gel column chromatography to give the compound 64, IR (KBr, cm⁻¹) 3434, 3166, 3063, 2927, 1678, 1655, 1632, 1586, 1439, 1378, 1325, 1243, 1170, 1127, 1068, 1017; ¹HNMR (DMSO-d6) 7.93 (d, J=7.8 Hz, 2H), 7.75 (m, 3H), 7.44 (br, 3H), 7.40 (s, 1H), 7.37 (m, 3H), 1.90 (s, 3H).

### Example 65

### Compound 65 in Table 1

To a mixture of 5-methyl-4-(2,4-dichlorophenyl)-4H— pyrazol-3-amine 512 mg (2.1 mmol) in toluene 20 ml was added 4-chloro-3-oxobutanonate 300 mg (2.2 mmol) The mixture was refluxed for 2 h. The reaction solution was filtered. The crude was separated by silica gel column chromatography to give the compound 65, IR (KBr, cm⁻¹) 3432, 3172, 3068, 2961, 2895, 1696, 1637, 1601, 1586, 1523, 1495, 1446, 1407, 1375, 1336, 1311, 1274, 1170, 1160; ¹HNMR (DMSO-d6) δ 7.80 (d, J=1.8 Hz, 1H), 7.55 (dd, J=1.8 Hz, J=8.4 Hz, 1H), 7.50 (d, J=8.4 Hz, 1H), 5.96 (s, 1H), 4.61 (s, 2H), 2.17 (s, 3H).

### Example 66

### Compound 66 in Table 1

To a mixture of 5-ethyl-4-(2,4-dichlorophenyl)-4H-pyrazol-3-amine 512 mg (2.0 mmol) in toluene 20 ml, was added ethyl 3-(4-(trifluoromethyl)phenyl)-3-oxo-propionate 520 mg (2.6 mmol) The mixture was refluxed for 2 h. The reaction solution was filtered and distilled under reduced pressure. The crude was separated by silica gel column chromatography to give the compound 66, IR (KBr, cm⁻¹) 3433, 3167, 3065, 2975, 2936, 2853, 1686, 1633, 1588, 1572, 1442, 1323, 1171, 1128, 1069, 1018; ¹HNMR (DMSO-d6) δ 7.92 (m, 4H), 7.77 (s, 1H), 7.52 (m, 2H), 6.16 (s, 1H), 2.58 (q, J=7.2 Hz, 2H), 1.09 (t, J=7.2 Hz, 3H).

### Example 67

### Compound 67 in Table 1

To a mixture of 5-(chloromethyl)-3-(2,4-dichlorophenyl)-2-chloromethylpyrazolo[1,5-a]pyrimidin-7(4H)-one 684 mg (2.0 mmol) in toluene 20 ml, was added, o-amino pyridine 164 mg (1.74 mmol). The mixture was refluxed for 2 h. The reaction solution was filtered. The crude was separated by silica gel column chromatography to give the compound 67, IR (KBr, cm⁻¹) 3418, 2958, 2924, 2852, 1618, 1594, 1555, 1511, 1460, 1416, 1376, 1341, 1253, 1237, 1120, 1101, 1072; ¹HNMR (DMSO-d6) δ 8.18 (d, J=6.0 Hz, 1H), 7.88 (m, 1H), 7.62 (d, J=1.2 Hz, 1H), 7.40 (dd, J=2.4 Hz, J=8.4 Hz, 1H), 7.33 (d, J=8.4 Hz, 1H), 7.06 (d, J=8.4 Hz, 1H), 6.93 (t, J=7.2 Hz, 1H), 5.46 (s, 1H), 5.11 (s, 2H), 2.17 (s, 3H).

### Example 68

### Compound 68 in Table 1

To a mixture of 7-chloro-2-(4-(trifluoromethyl)phenyl)-4H— benzo[d][1,3]oxazin-4-one 0.8 g (2.45 mmol), was added morpholine 8 ml. The mixture was reacted for 1 h, room temperature. The reaction solution was filtered. The crude was separated by silica gel column chromatography to give the compound 68, IR (KBr, cm⁻¹) 3068, 3008, 2971, 2921, 2866, 1678, 1610, 1596, 1530, 1510, 1486, 1466, 1430, 1329, 1313, 1288, 1155, 1118, 1067, 1019, 1004; ¹HNMR (DMSO-d6, 600 MHz) δ 8.10 (d, J=8.4 Hz, 2H), 7.94 (d, J=8.4 Hz, 2H), 7.69 (br, 1H), 7.43 (d, J=8.4 Hz, 1H), 7.39 (m, 1H) 3.58 (m, 4H), 3.39 (m, 4H).

### Example 69

### Compound 69 in Table 1

To a mixture of 7-chloro-2-(4-(trifluoromethyl)phenyl)-4H-benzo[d][1,3]oxazin-4-one 1.88 g (3.1 mmol) in DMF 100 ml was added phenol 800 mg (8.5 mmol) and potassium hydroxide 1.20 g. The mixture was reacted for 1 h, 100° C. The reaction solution was filtered. The crude was separated by silica gel column chromatography to give the compound 69, IR (KBr, cm⁻¹) 1662, 1638, 1579, 1523, 1506, 1440, 1425, 1366, 1329, 1244, 1163; ¹HNMR (DMSO-d6, 600 MHz) δ7.93 (br, 4H), 7.79 (s, 1H), 7.48 (br, 4H), 7.36 (br, 3H).

### Example 70

### Compound 70 in Table 1

To a mixture of 4-(2,4-dichlorophenyl)-3-phenyl-1 H— pyrazol-5-amine 484 mg (1.6 mmol) in toluene 20 ml was added 2-methyl-3-oxo-(4-cyano)phenyl)ethyl propionate 544 mg (2.35 mmol). The mixture was refluxed for 1 h. The reaction solution was filtered. The crude was separated by silica gel column chromatography to give the compound 70, IR (KBr, cm⁻¹) 3431, 3173, 3074, 2925, 2228, 1663, 1630, 1585, 1503, 1438, 1383, 1340; ¹HNMR (DMSO-d6) δ8.03 (d, J=8.4 Hz, 2H), 7.74 (m, 3H), 7.45 (m, 3H), 7.36 (m, 4H), 1.88 (s, 3H).

### Example 71

### Compound 71 in Table 1

To a mixture of 4-(2,4-dichlorophenyl)-3-ethyl-1H-pyrazol-5-amine 0.964 g (3.76 mmol) in toluene 20 ml was added ethyl 3-(4-cyanophenyl)-2-methyl-3-oxo-propionate 816 mg (2.0 mmol). The mixture was refluxed for 30 h. The reaction solution was filtered. The crude was separated by silica gel column chromatography to give the compound 71, IR (KBr, cm⁻¹) 3435, 3168, 3093, 2965, 2927, 2228, 1662, 1633, 1584, 1505, 1447, 1376, 1237, 1099, 1068, 1011; ¹HNMR (DMSO-d6, 600 MHz) δ8.01 (d, J=8.4 Hz, 2H), 7.71 (m, 3H), 7.46 (m, 2H), 2.56 (q, J=7.8 Hz, 2H), 1.85 (s, 3H), 1.08 (t, J=7.8 Hz, 3H).

### Example 72

### Compound 72 in Table 1

To a mixture of 3-methyl-4-((trifluoromethyl)phenyl)-1H— pyrazol-5-amine 0.964 g (4.0 mmol) in toluene 20 ml was added 2-methyl-3-oxo-(6-(trifluoromethyl)pyridin-3-yl)ethyl propionate 825 mg (3.0 mmol) and potassium hydroxide 1.20 g. The mixture was refluxed for 30 h. The reaction solution was filtered and distilled under reduced pressure. The crude was separated by silica gel column chromatography to give the compound 72 IR (KBr, cm⁻¹) 3436, 2925, 2854, 1685, 1634, 1590, 1536, 1455, 1382, 1335, 1324, 1170, 1144, 1087, 1011, 854; ¹HNMR (DMSO-d6, 600 MHz) δ 8.97 (s, 1H), 7.23 (m, 3H), 8.31 (m, 1H), 8.10 (d, J=7.8 Hz, 1H), 7.78 (d, J=7.8 Hz, 2H), 7.69 (d, J=7.8 Hz, 2H), 2.36 (s, 3H), 1.92 (s, 3H).

### Example 73

### Compound 73 in Table 1

To a mixture of 3-cyclopropyl-4-((2,4-dichlorophenyl)-1H-pyrazol-5-amine 536 mg (2.0 mmol) in toluene 20 ml was added ethyl 2-methyl-3-oxo-3-(4-(N,N-dipropylaminosulfonyl)phenyl)propionate 738 mg (8.5 mmol). The mixture was refluxed for 1 h. The reaction solution was filtered and distilled under reduced pressure. The crude was separated by silica gel column chromatography to give the compound 73, IR (KBr, cm⁻¹) 3447, 3082, 2964, 2928, 2874, 1661, 1629, 1584, 1498, 1460, 1381, 1336, 1156, 1094, 998; ¹HNMR (DMSO-d6) 67.93 (d, J=7.8 Hz, 2H), 7.23 (m, 3H), 7.50 (m, 2H), 3.06 (t, J=7.8 Hz, 4H), 1.83 (s, 3H), 1.66 (m, 1H), 1.48 (m, 4H), 0.95 (m, 1H), 0.89 (m, 3H), 0.82 (t, J=7.8 Hz, 6H).

### Example 74

### Compound 74 in Table 1

To a mixture of diphenyl-amino-imidazole 5.0 g (21.3 mmol) in methnol 20 ml was added sodium ethoxide 2.89 g (42.5 mmol) and ethoxy methylene diethyl malonate 30 ml. The mixture was reacted for 8 h, 70° C. The reaction solution was filtered and distilled under reduced pressure. The crude was separated by silica gel column chromatography to give the compound 74, IR (KBr, cm⁻¹) 3455, 2926, 1732, 1659, 1586, 1539 (s), 1505, 1460, 1426, 1332, 1106, 940, 822, 796, 776, 760; ¹HNMR (DMSO-d6) 8.58 (s, 1H), 7.39 (m, 8H), 7.26 (m, 4H).

### Example 75

### Compound 75 in Table 1

To a mixture of 2-amino-4-(3-nitrophenyl)-4H-benzo[h]chromene-3-carbonitrile 5.0 g (14.6 mmol) in toluene 20 ml was added triethyl orthoformate 20 ml and acetic anhydride 7 ml The mixture was refluxed for 1 h. The reaction solution was filtered. The crude was separated by silica gel column chromatography to give the compound 75, IR (KBr, cm⁻¹) 3447, 3061, 2956, 2208, 1750, 1655, 1617, 1575, 1529, 1472, 1445, 1395, 1349, 1298, 1272, 1184, 1148, 1115, 1095, 1081, 995; ¹HNMR (DMSO-d6) of δ 8.98 (s, 1H), 8.40 (d, J=8.4 Hz, 1H), 8.24 (m, 1H,), 8.17 (m, 1H), 7.92 (d, 1H), 7.83 (d, J=7.8 Hz, 1H,), 7.676 (m, 1H), 7.64 (m, 3H), 7.12 (d, J=8.4 Hz, 1H), 5.50 (s, 1H), 4.42 (q, J=7.2 Hz, 1H), 2.53 (s, 1H), 1.37 (t, J=7.2 Hz, 3H).

### Example 76

### Compound 76 in Table 1

To a mixture of (E)-ethyl-N-4-cyano-1-phenyl-1H-pyrazol-5-ylformimidate 0.68 g (2.8 mmol) in ethanol 40 ml was added (8.5 mmol). The mixture was reacted for 1 h, 100° C. The reaction solution was filtered. The crude was separated by silica gel column chromatography to give the compound 76, in 100 ml eggplant-shaped bottle to join, of 40 ml of ethanol, pyrimidine of diamines 1.13 g, 60° C. for 6 hours, the reaction solution, precipitation of solids, filter the product of the product may be the target. IR (KBr cm⁻¹) 3458, 3076, 2912, 1558, 1499, 1445, 1363, 1330, 1306, 1230, 1126, 1110, 1096; ¹HNMR (DMSO-d6) 8.62 (s, 1H), 8.49 (s, 1H), 8.22 (d, J=7.8 Hz, 2H), 8.19 (s, 1H), 7.92 (d, J=1.2 Hz, 1H), 7.58 (t, J=7.8 Hz, 2H), 7.38 (t, J=7.8 Hz, 1H), 7.05 (d, J=3.0 Hz, 1H), 6.69 (q, J=1.8 Hz, 1H), 7.05 (d, J=3.0 Hz, 1H).

### Example 77

### Compound 77 in Table 1

To a mixture of (Z)—N-benzylidene-4-imino-1-phenyl-1H-pyrazolo[3,4-d]pyrimidin-(4H)-5(4-(3,4,5,6-tetraacyl-oxo-tetrahydro-2H-pyran-2-yloxy)benzylidene) amine 0.60 g (0.93 mmol) in toluene 50 ml was added NaOH 10 ml (1.0 N). The mixture was reacted for 8 h, 60° C. The reaction solution was filtered. The crude was separated by silica gel column chromatography to give the compound 77, IR (KBr cm⁻¹) 3379, 2924, 1592, 1504, 1444, 1424, 1363, 1305, 1237, 1082, 1039, 974; ¹HNMR (DMSO-d6) 8.66 (s, 1H), 8.48 (s, 1H), 8.28 (s, 1H), 8.23 (d, J=7.8 Hz, 2H), 7.80 (d, J=8.4 Hz, 2H), 7.58 (t, J=7.8 Hz, 2H), 7.38 (t, J=7.8 Hz, 1H), 7.15 (d, J=8.4 Hz, 2H), 5.22 (d, J=7.8 Hz, 1H), 3.96 (br, 1H), 3.74 (m, 2H).

### Example 78

### Compound 78 in Table 1

To a mixture of 5-methyl-4-(2,4-dichlorophenyl)-4-H-pyrazol-3-amine 2.42 g (10 mmol) in ethanol 40 ml was added 2-(4-chlorobenzylidene) malononitrile 1.89 g (10 mmol). The mixture was reacted for 6 h, 75° C. The reaction solution was filtered and distilled under reduced pressure. The crude was separated by silica gel column chromatography to give the compound 78, IR (KBr, cm⁻¹) 3441, 3307, 2923, 2851, 2217, 1654, 1629, 1596, 1384, 13511, 1319, 1256, 1155; ¹HNMR (DMSO-d6) δ 8.99 (br, 2H), 7.77 (m, 3H), 7.58 (d, J=8.4 Hz, 2H), 7.51 (t, J=6.9 Hz, 1H), 2.35 (s, 3H).

### Example 79

### Compound 79 in Table 1

To a mixture of 4,5-diphenyl-1H-imidazol-2-amine 500 mg (2.1 mmol) in toluene 20 ml was added 2-(ethoxymethylene)malononitrile 0.5 g (4.1 mmol). The mixture was refluxed for 4 h. The reaction solution was filtered. The crude was separated by silica gel column chromatography to give the compound 79, IR (KBr, cm⁻¹) 3430, 3301, 3221, 3188, 3047, 2223, 1644, 1595, 1566, 1522, 1448, 1388, 1345; ¹HNMR (DMSO-d6) of δ 8.43 (s, 1H), 7.68 (m, 3H), 7.61 (m, 2H), 7.42 (m, 2H), 7.27 (m, 3H), 3.41 (s, 2H).

### Example 80

### Compound 80 in Table 1

To a mixture of 7-amino-2,3-diphenylimidazo[1,2-a]pyrimidin-6-carbonitrile 190 mg (0.6 mmol) in ethanol 2 ml was added NaOH 5 ml (1N). The mixture was reacted for 3 h, room temperature. The reaction solution was filtered and distilled under reduced pressure. The crude was separated by silica gel column chromatography to give the compound 80, IR (KBr. cm⁻¹) 3456, 3279, 3118, 1683, 1633, 1602, 1574, 1548, 1500, 1465, 1444, 1412, 1384, 1363, 1309, 1248, 1229, 1102, 1088, 1030, 971, 912, 803, 775, 699; ¹NMR (DMSO-d6) of δ 8.71 (s, 1H), 7.63 (m, 3H), 7.58 (m, 2H), 7.42 (m, 2H), 7.24 (m, 3H), 3.41 (s, 3H).

### Example 81

### Compound 81 in Table 1

To a mixture of 7-methyl-2,3-diphenylimidazo[1,2-a]pyrimidin-5(8H)-one 5.10 g (16.9 mmol) in toluene 20 ml was added phosphorus oxychloride 12 ml. The mixture was refluxed for 4 h. The reaction solution was filtered. The crude was separated by silica gel column chromatography to give the compound 81, IR (KBr, cm⁻¹) 3418, 2925, 1643, 1511, 1385, 1284, 1156, 1101, 1026, 772, 696, 526; ¹HNMR (DMSO-d6) δ 7.42 (m, 3H), 7.39 (m, 2H), 7.35 (m, 2H), 7.24 (m, 3H), 6.57 (s, 1H), 1.9 3 (s, 3H).

### Example 82

### Compound 82 in Table 1

To a mixture of N-(4-(2,4-dichlorophenyl)-3-(3-methoxy-phenyl)-1H-pyrazol-5-yl)acetamide 750 mg (2.0 mmol) in toluene 20 ml was added hydrochloric acid 50 ml (2 N). The mixture was refluxed for 8 h. The reaction solution was filtered and distilled under reduced pressure. The crude was separated by silica gel column chromatography to give the compound 82, IR (KBr, cm⁻¹) 3439, 3208, 2961, 1615, 1580, 1540, 1507, 1461, 1431, 1374, 1301, 1270, 1250, 1177, 1100, 1031, 966, 866, 833, 803.

### Example 83

### Compound 83 in Table 1

To a mixture of 4-(2,4-dichlorophenyl)-3-(3-methoxy-phenyl)-1H-pyrazole-5-amine 334 mg (1 mmol) in toluene 5.0 ml was added ethyl 3-(4-cyanophenyl)-2-methyl-3-oxo-propionate 280 mg (1.2 mmol). The mixture was refluxed for 50 h. The reaction solution was filtered and distilled under reduced pressure. The crude was separated by silica gel column chromatography to give the compound 83, IR (KBr, cm⁻¹) 3164, 2928, 2227, 1655, 1630, 1585, 1504, 1435, 1384, 1335, 1246, 1175, 1099; ¹HNMR (DMSO-d6) δ 8.03 (d, J=8.4 Hz, 2H), 7.74 (m, 3H), 7.45 (m, 1H), 7.36 (m, 3H), 6.92 (m, 2H), 3.75 (s, 3H), 1.87 (s, 3H).

### Example 84

### Compound 84 in Table 1

To a mixture of 5-amino-4-(2,4-dichlorophenyl)-1H-pyrazol-3-ol 488 mg (2.0 mmol) in toluene 20 ml was added 3-(4-cyanophenyl)-2-methyl-3-oxo-propionate 560 mg (2.8 mmol). The mixture was refluxed for 1.5 1 h. The reaction solution was filtered. The crude was separated by silica gel column chromatography to give the compound 84, IR (KBr, cm⁻¹) 3410, 3208, 3082, 2923, 2862, 2231, 1637, 1530, 1522, 1456, 1383, 1331, 1276, 1235, 1102, 1079, 1012, 849, 787; ¹HNMR (DMSO-d6) δ 11.87 (s, 1H), 8.01 (d, J=7.8 Hz, 2H), 7.71 (d, J=8.4 Hz, 2H), 7.66 (d, J=0.6 Hz, 1H), 7.43 (m, 2H), 1.84 (s, 3H).

### Example 85

### Compound 85 in Table 1

To a mixture of phthalic anhydride 1.53 g (3.1 mmol) in pyridine 50 ml was added o-aminobenzoyl hydrazine 1.42 g (9.4 mmol). The mixture was refluxed for 4 h. The reaction solution was filtered and distilled under reduced pressure. The crude was separated by silica gel column chromatography to give the compound 85, IR (KBr. cm⁻¹) 3194, 3085, 1687, 1669, 1605, 1590, 1556, 1485, 1463, 1438, 1372, 1332, 1264, 1256, 1235, 1168, 1312, 111; ¹HNMR (DMSO-d6,) δ12.95 (br, 1H), 8.83 (d, J=7.6 Hz, 1H), 8.30 (d, J=7.6 Hz, 1H), 8.20 (br, 1H), 8.00 (m, 2H), 7.90 (m, 2H), 7.60 (m, 1H).

### Example 86

### Compound 86 in Table 1

To a mixture of 2-amino-4-(4-chlorophenyl)-6-oxo-1,6-dihydropyrimidin-5-carbonitrile 5.65 g (22.9 mmol) in glacial acetic acid 40 ml was added (Z)-3-ethoxy-1-phenylprop-2-en-1-one 3.66 g (20.8 mmol). The mixture was refluxed for 5 h. The reaction solution was filtered. The crude was separated by silica gel column chromatography to give the compound 86, IR (KBr, cm⁻¹) 3274, 2960, 2925, 2853, 2217, 1735, 1684, 1593, 1581, 1563, 1519, 1488, 1387, 1260, 1091, 1014, 720; ¹HNMR (DMSO-d6) δ 7.88 (m, 2H), 7.63 (m, 2H), 7.37 (m, 5H), 6.47 (d, J=8.1 Hz, 1H), 6.22 (d, J=5.4 Hz, 1H).

### Example 87

### Compound 87 in Table 1

To a mixture of 7-chloro-2-mercapto-3-phenyl quinazolin-4(3H)-one 5.0 g (17.3 mmol) was added morpholine 10 ml. The mixture was reacted for 20 h, room temperature. The reaction solution was filtered and distilled under reduced pressure. The crude was separated by silica gel column chromatography to give the compound 87, IR (KBr, cm⁻¹) 3435, 3062, 2984, 2875, 1692, 1599, 1566, 1542, 1455, 1331, 1294, 1264, 1198, 1071, 969, 914, 830, 775, 697; ¹HNMR (DMSO-d6) δ 8.17 (d, J=8.7 Hz, 1H, Ph-H), 7.67 (d, J=1.2 Hz, 1H, Ph-H), 7.53 (t, J=3.9 Hz, 3H, Ph-H) 7.37-7.26 (m, 3H, Ph-H), 3.69 (br, 8H).

TABLE 1

| | Structure | M⁺/e |
|---|---|---|
| 1 | 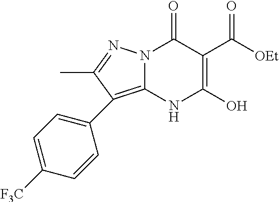 | 381.31 |
| 2 | 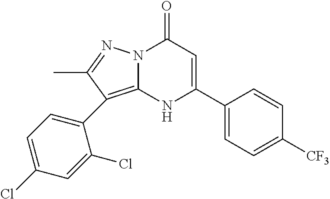 | 438.23 |
| 3 | 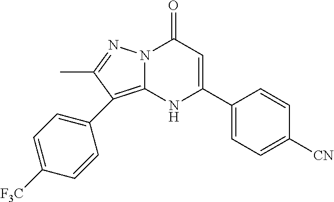 | 394.35 |
| 4 | 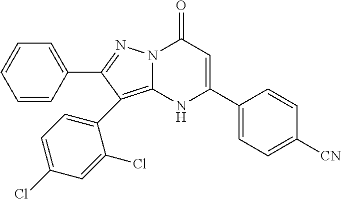 | 457.31 |
| 5 | 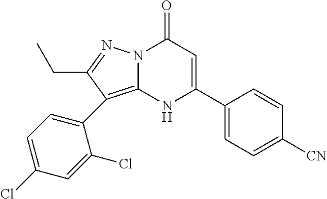 | 409.27 |
| 6 | 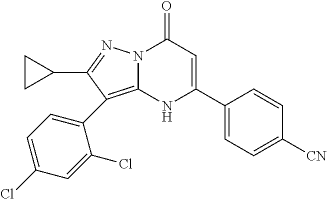 | 421.28 |
| 7 | 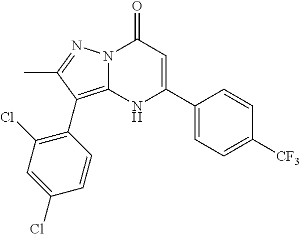 | 438.23 |
| 8 | 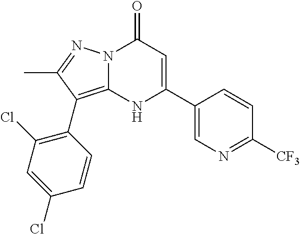 | 439.22 |
| 9 | 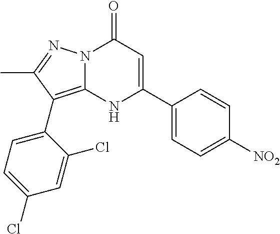 | 415.23 |
| 10 | 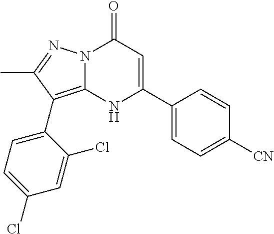 | 395.24 |
| 11 | 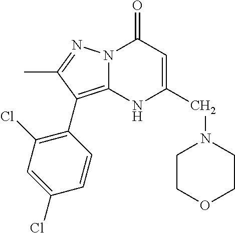 | 393.27 |
| 12 | 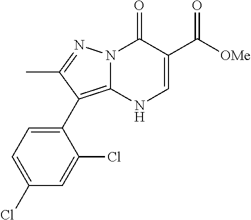 | 352.17 |
| 13 | 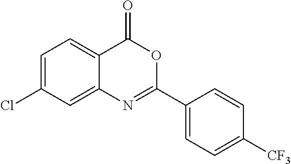 | 325.67 |
| 14 | 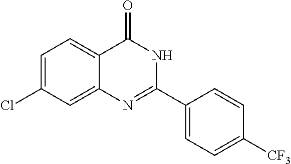 | 324.69 |
| 15 | 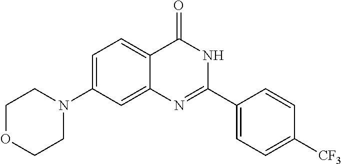 | 375.34 |
| 16 | 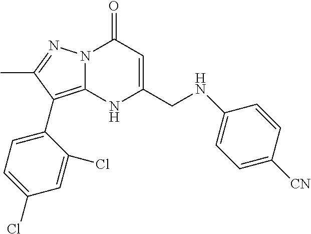 | 424.28 |
| 17 | 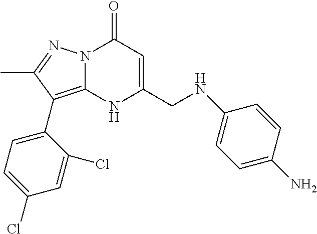 | 414.29 |
| 18 | 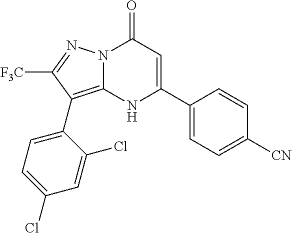 | 449.21 |
| 19 | 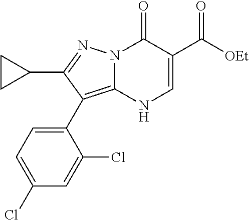 | 392.24 |
| 20 | 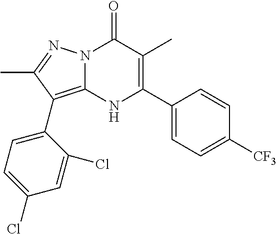 | 452.26 |
| 21 | 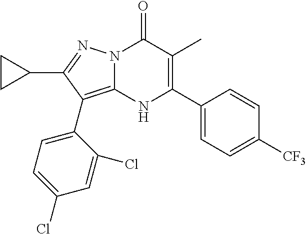 | 478.29 |
| 22 | 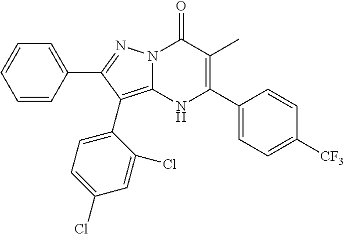 | 514.33 |
| 23 | 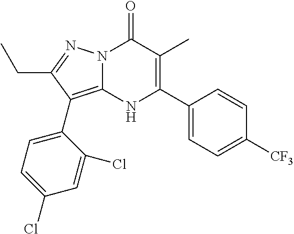 | 466.28 |
| 24 | 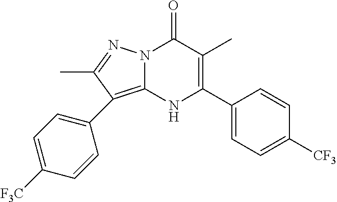 | 451.36 |
| 25 | 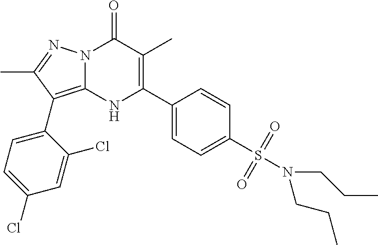 | 547.5 |
| 26 | 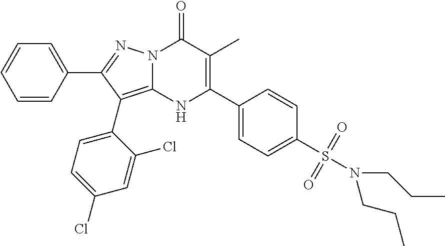 | 609.57 |
| 27 | 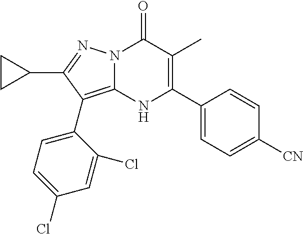 | 435.31 |
| 28 | 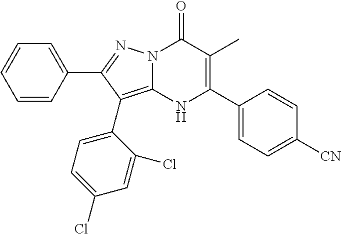 | 471.34 |
| 29 | 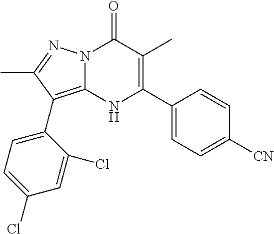 | 409.29 |
| 30 | 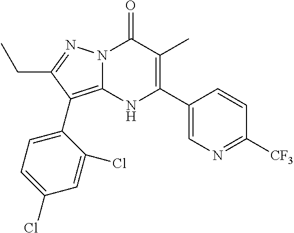 | 467.27 |
| 31 | 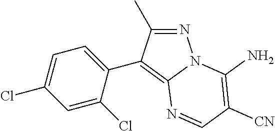 | 318.16 |
| 32 | 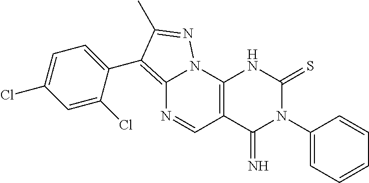 | 453.35 |
| 33 | 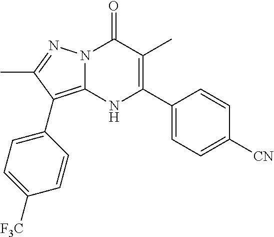 | 408.38 |
| 34 | 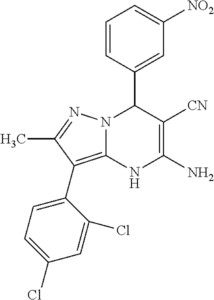 | 441.27 |
| 35 | 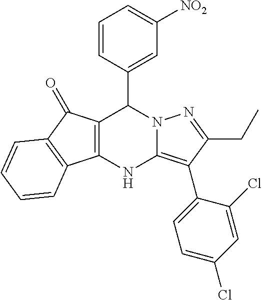 | 517.36 |
| 36 | 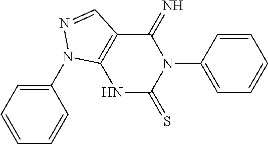 | 319.38 |
| 37 | 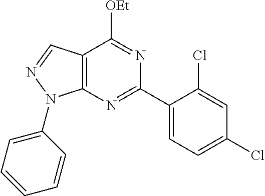 | 385.25 |
| 38 | 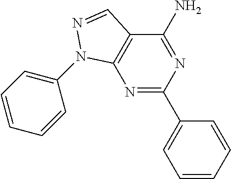 | 287.32 |
| 39 | 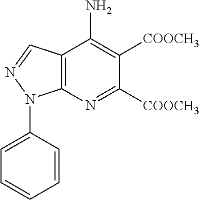 | 326.31 |
| 40 | 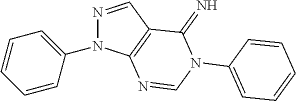 | 287.32 |
| 41 | 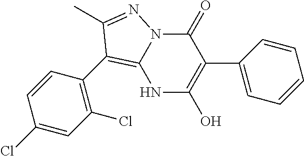 | 386.23 |
| 42 | 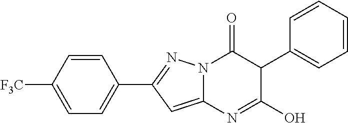 | 371.31 |
| 43 | 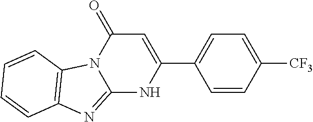 | 329.28 |
| 44 | 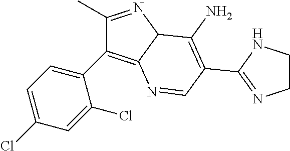 | 361.23 |
| 45 | 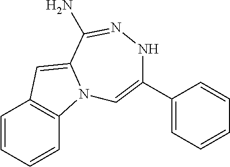 | 274.32 |
| 46 | 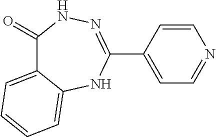 | 238.24 |
| 47 | 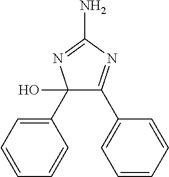 | 251.28 |
| 48 | 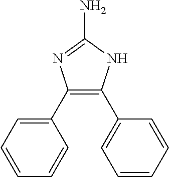 | 235.28 |
| 49 | 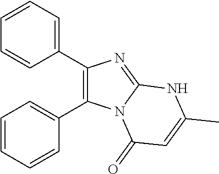 | 301.34 |
| 50 | 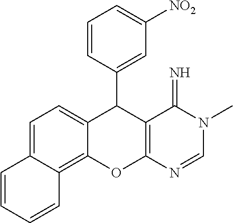 | 384.39 |
| 51 | 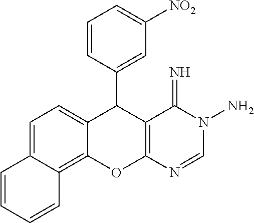 | 385.38 |
| 52 | 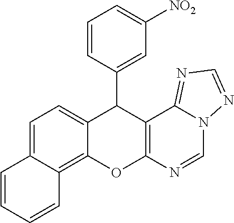 | 395.37 |
| 53 | 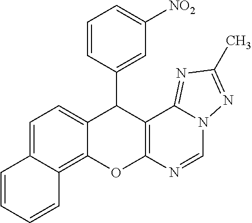 | 409.4 |
| 54 | 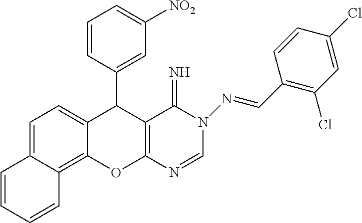 | 542.37 |
| 55 | 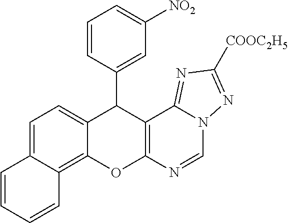 | 467.43 |
| 56 | 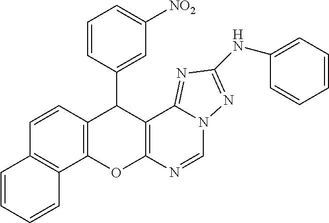 | 486.48 |
| 57 | 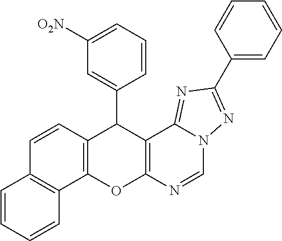 | 471.47 |
| 58 | 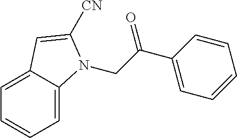 | 260.29 |
| 59 | 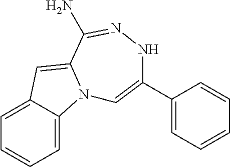 | 274.32 |
| 60 | 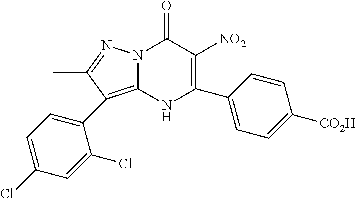 | 459.24 |
| 61 | 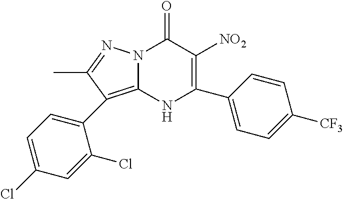 | 483.23 |
| 62 | 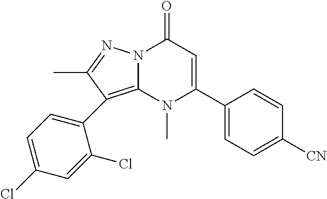 | 409.27 |
| 63 | 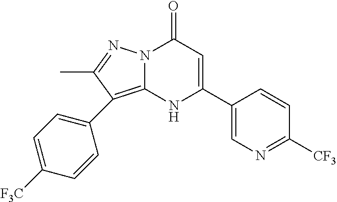 | 438.33 |
| 64 | 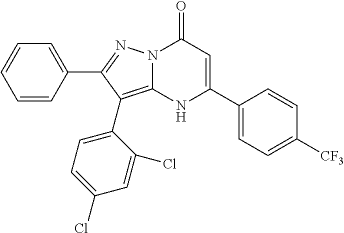 | 500.3 |
| 65 | 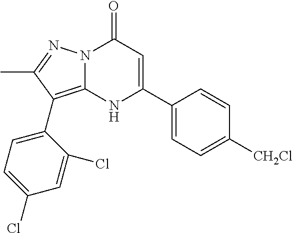 | 342.61 |
| 66 | 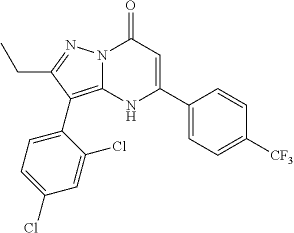 | 452.26 |
| 67 | 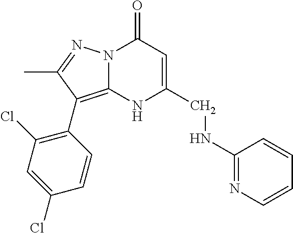 | 400.26 |
| 68 | 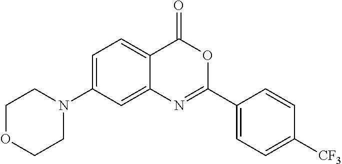 | 376.33 |
| 69 | 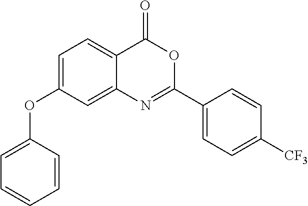 | 383.32 |
| 70 | 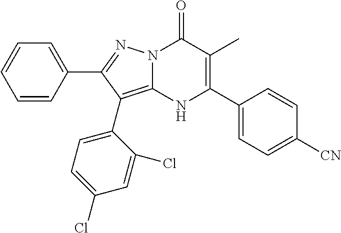 | 471.34 |
| 71 | 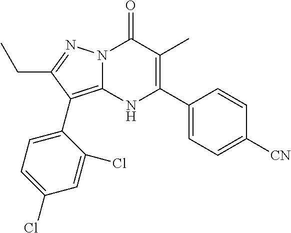 | 423.29 |
| 72 | 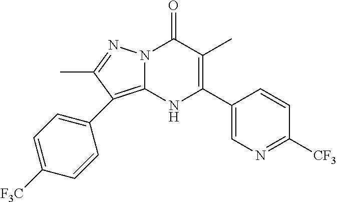 | 452.35 |
| 73 | 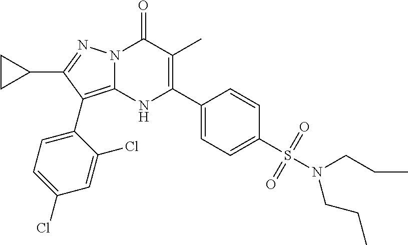 | 573.53 |
| 74 | 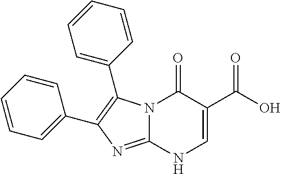 | 331.32 |
| 75 | 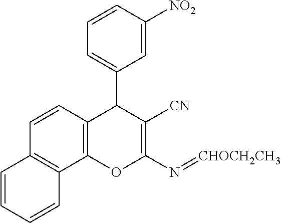 | 399.4 |
| 76 | 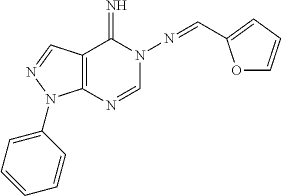 | 304.31 |
| 77 | 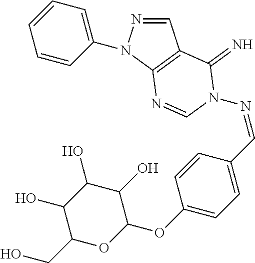 | 492.48 |
| 78 | 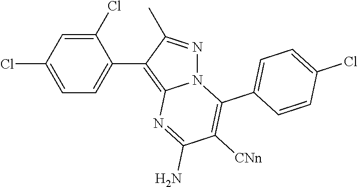 | 428.7 |
| 79 | 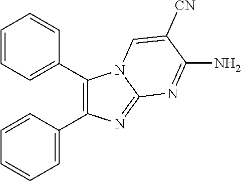 | 311.34 |
| 80 | 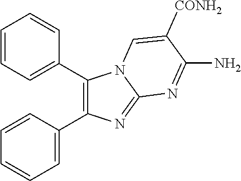 | 329.36 |
| 81 | 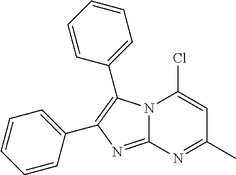 | 319.79 |
| 82 | 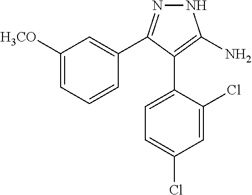 | 334.2 |
| 83 | 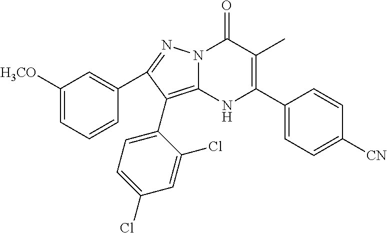 | 501.36 |
| 84 | 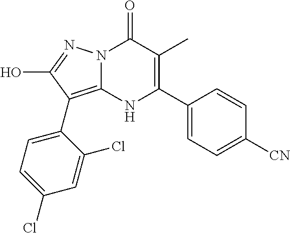 | 411.24 |
| 85 | 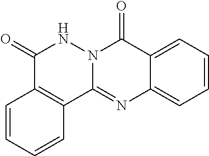 | 263.25 |
| 86 | 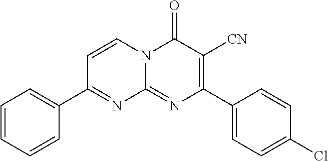 | 358.78 |
| 87 | 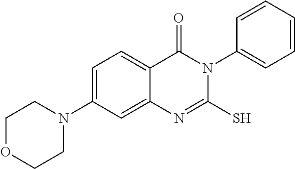 | 339.41 |
| 88 | 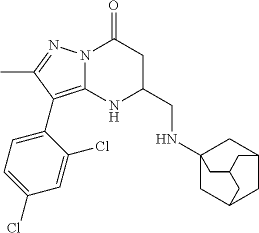 | 457.4 |
| 89 | 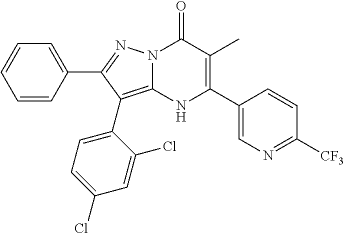 | 515.31 |
| 90 | 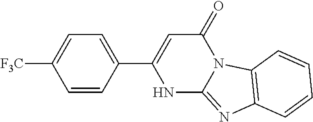 | 329.28 |
| 91 | 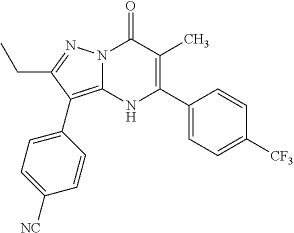 | 422.4 |
| 92 | 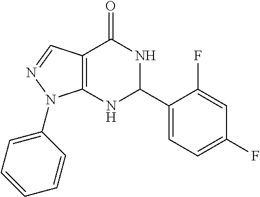 | 326.3 |
| 93 | 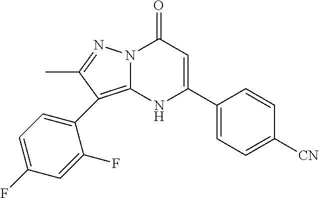 | 362.33 |
| 94 | 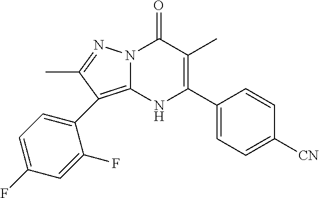 | 376.36 |
| 95 | 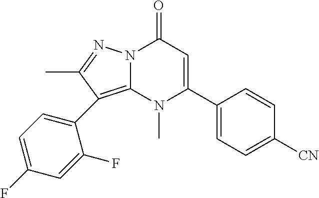 | 376.36 |
| 96 | 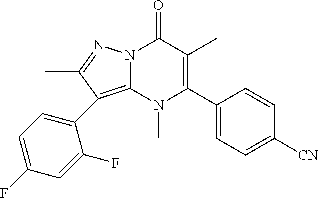 | 390.39 |
| 97 | 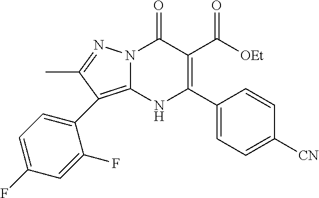 | 434.39 |
| 98 | 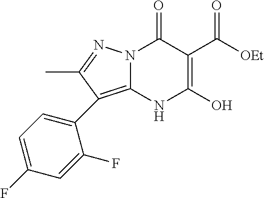 | 349.29 |
| 99 | 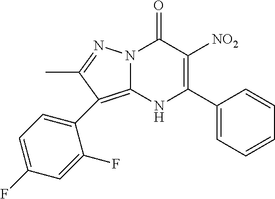 | 382.32 |
| 100 | 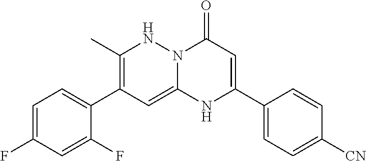 | 376.36 |
| 101 | 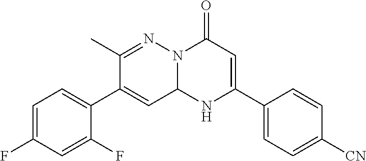 | 376.36 |
| 102 | 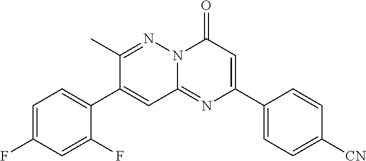 | 374.34 |
| 103 | 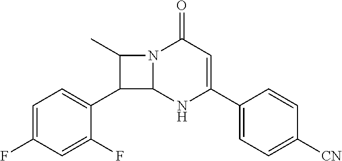 | 351.35 |
| 104 | 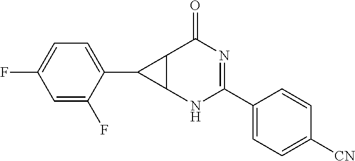 | 323.3 |
| 105 | 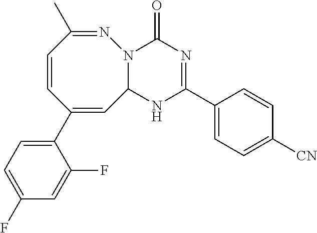 | 402.4 |
| 106 | 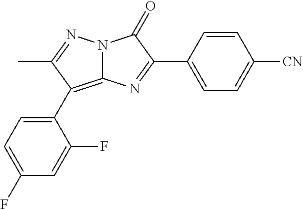 | 348.31 |
| 107 | 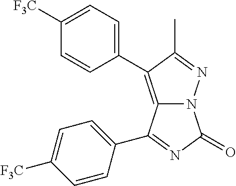 | 348.31 |
| 108 | 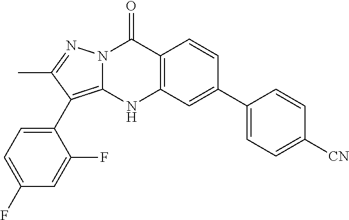 | 412.39 |
| 109 | 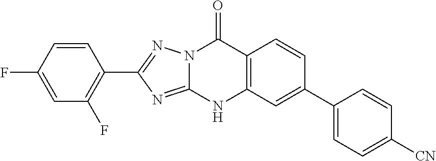 | 399.35 |
| 110 | 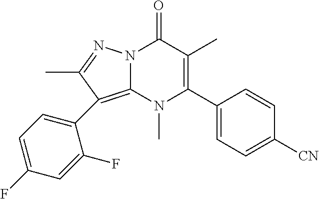 | 390.39 |
| 111 | 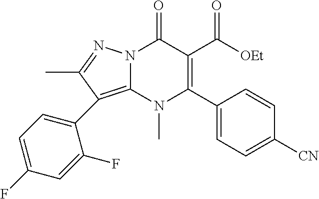 | 448.42 |
| 112 | 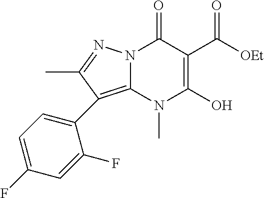 | 363.32 |
| 113 | 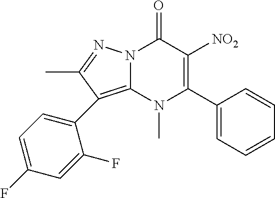 | 396.35 |
| 114 | 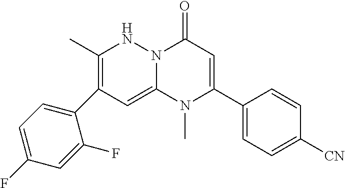 | 390.39 |
| 115 | 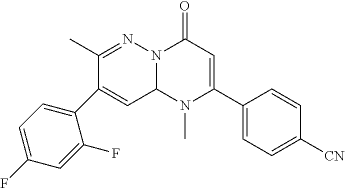 | 390.39 |
| 116 | 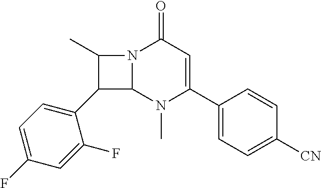 | 365.38 |
| 117 | 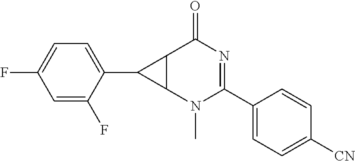 | 337.32 |
| 118 | 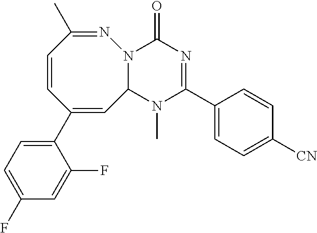 | 416.42 |
| 119 | 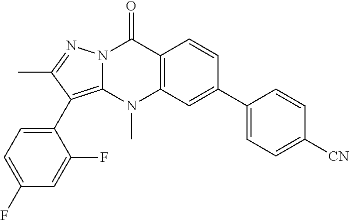 | 426.42 |
| 120 | 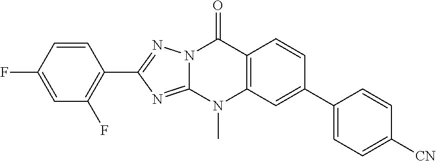 | 413.38 |
| 121 | 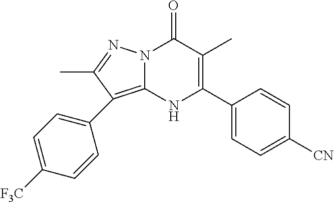 | 408.38 |
| 122 | 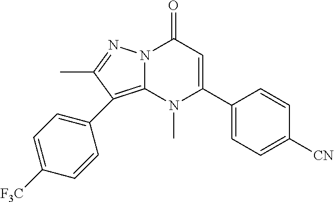 | 408.38 |
| 123 | 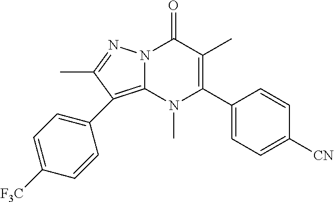 | 422.4 |
| 124 | 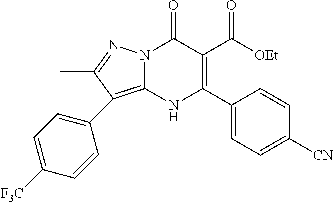 | 466.41 |
| 125 | 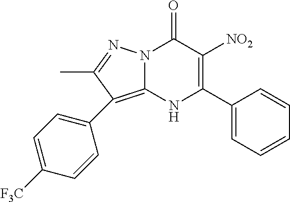 | 414.34 |
| 126 | 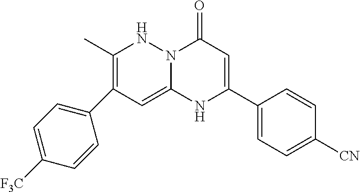 | 408.38 |
| 127 | 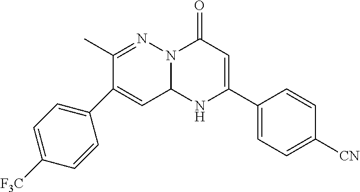 | 408.38 |
| 128 | 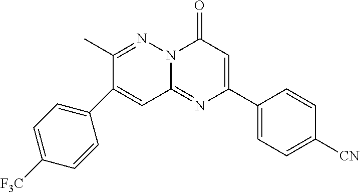 | 406.36 |
| 129 | 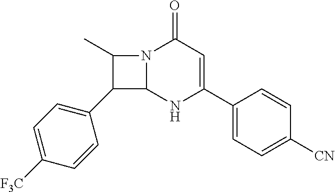 | 383.37 |
| 130 | 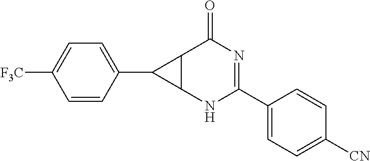 | 355.31 |
| 131 | 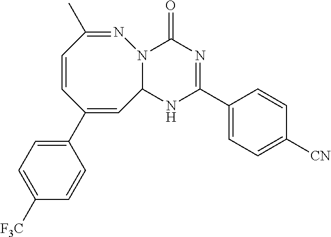 | 434.41 |
| 132 | 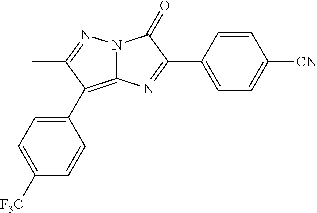 | 380.32 |
| 133 | 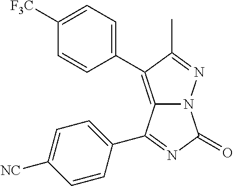 | 380.32 |
| 134 | 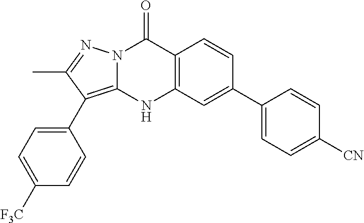 | 444.41 |
| 135 | 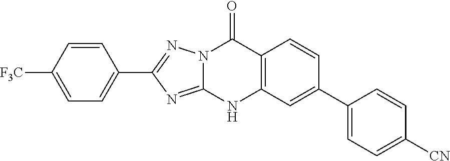 | 431.37 |
| 136 | 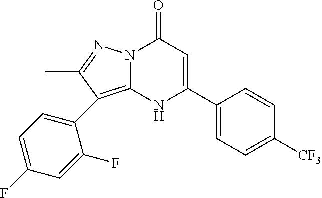 | 405.32 |
| 137 | 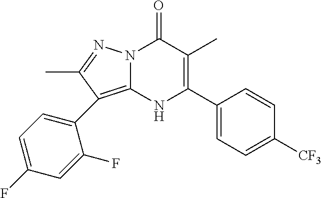 | 419.35 |
| 138 | 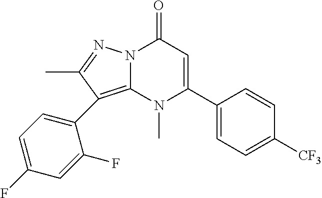 | 419.35 |
| 139 | 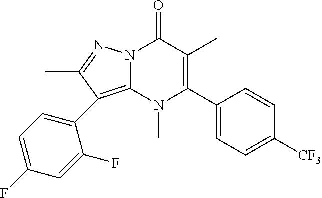 | 433.37 |
| 140 | 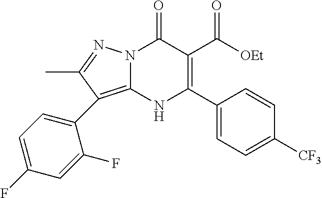 | 477.38 |
| 141 | 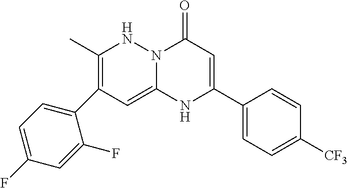 | 419.35 |
| 142 | 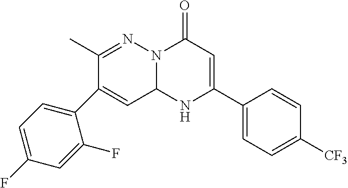 | 419.35 |
| 143 | 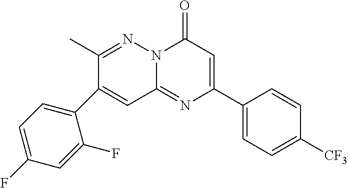 | 417.33 |
| 144 | 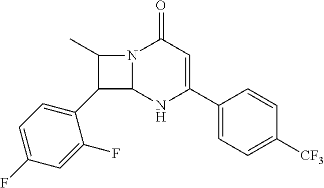 | 394.34 |
| 145 | 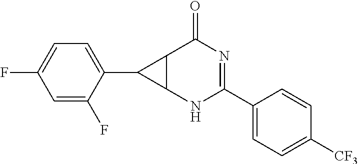 | 366.28 |
| 146 | 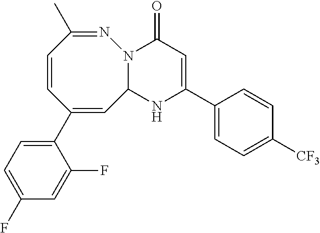 | 445.38 |
| 147 | 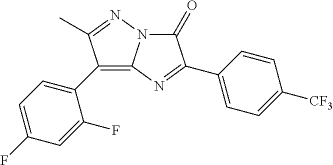 | 391.29 |
| 148 | 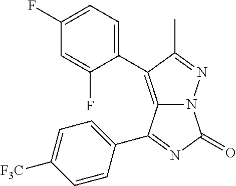 | 391.29 |
| 149 | 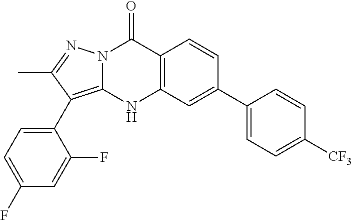 | 455.38 |
| 150 | 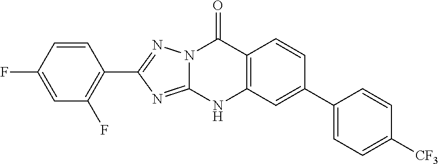 | 442.34 |
| 151 | 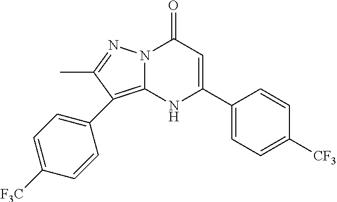 | 437.34 |
| 152 | 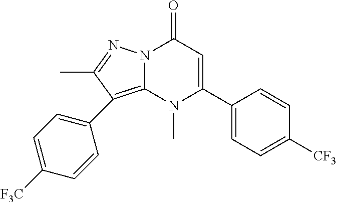 | 451.36 |
| 153 | 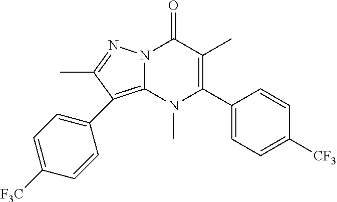 | 465.39 |
| 154 | 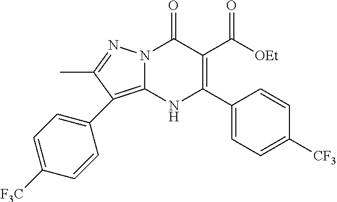 | 509.4 |
| 155 | 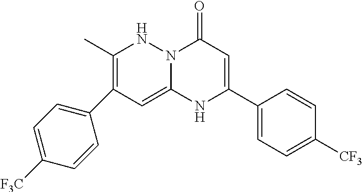 | 451.36 |
| 156 | 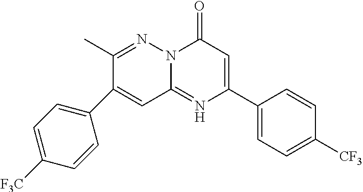 | 451.36 |
| 157 | 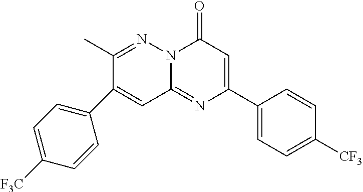 | 449.35 |
| 158 | 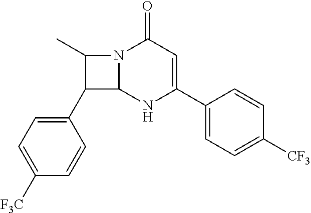 | 426.35 |
| 159 | 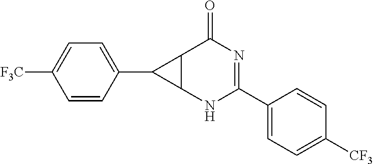 | 398.3 |
| 160 | 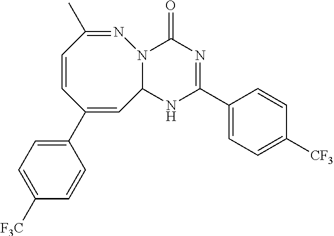 | 477.4 |
| 161 | 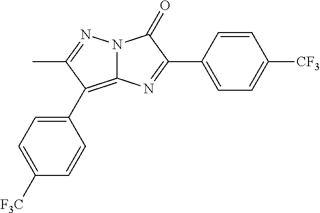 | 423.31 |
| 162 | 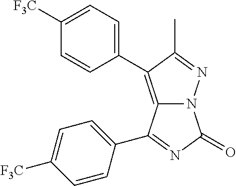 | 423.31 |
| 163 | 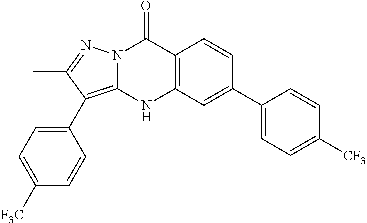 | 487.4 |
| 164 | 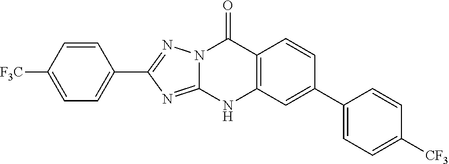 | 474.36 |
| 165 | 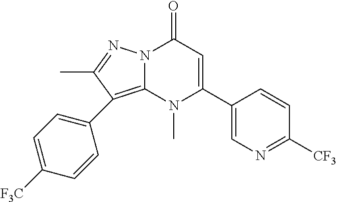 | 452.35 |
| 166 | 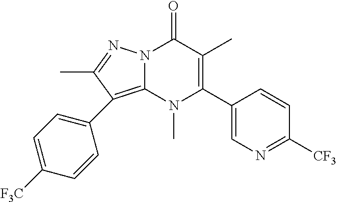 | 466.38 |
| 167 | 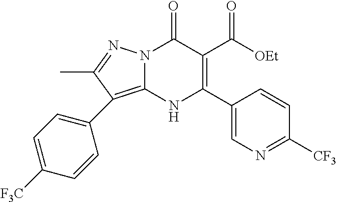 | 510.39 |
| 168 | 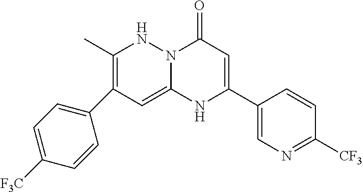 | 452.35 |
| 169 | 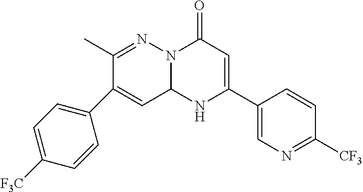 | 452.35 |
| 170 | 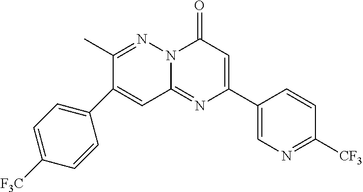 | 450.34 |
| 171 | 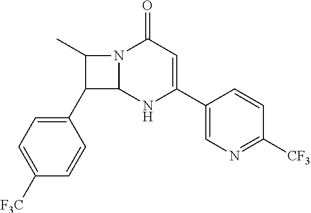 | 427.34 |
| 172 | 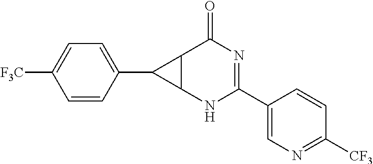 | 399.29 |
| 173 | 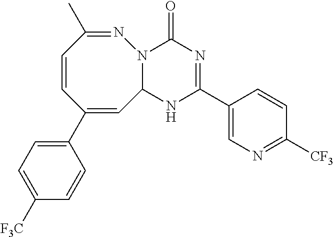 | 478.39 |
| 174 | 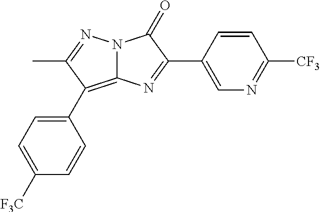 | 424.3 |
| 175 | 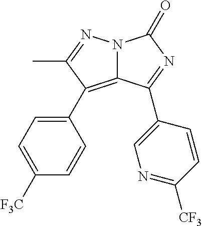 | 424.3 |
| 176 | 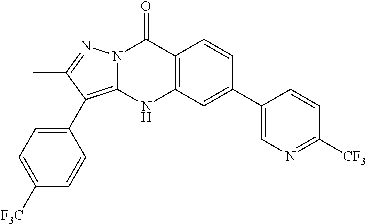 | 488.38 |
| 177 | 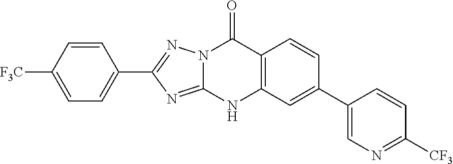 | 475.35 |
| 178 | 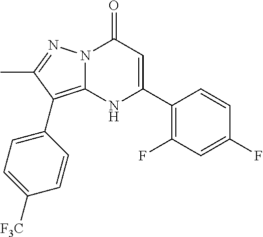 | 405.32 |
| 179 | 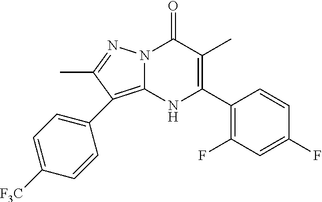 | 419.35 |
| 180 | 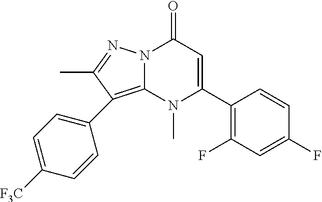 | 419.35 |
| 181 | 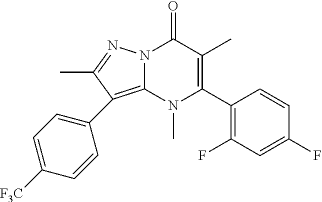 | 433.37 |
| 182 | 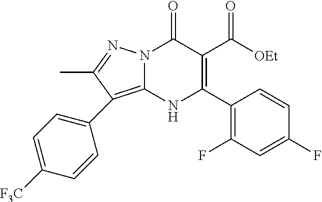 | 477.38 |
| 183 | 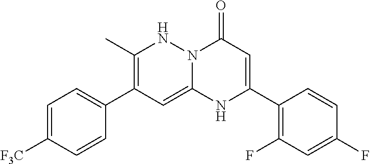 | 419.35 |
| 184 | 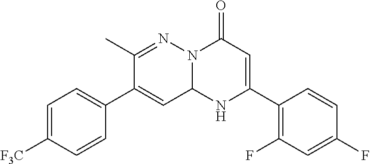 | 419.35 |
| 185 | 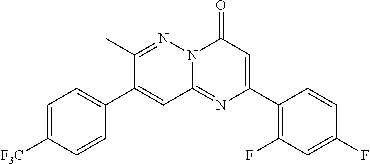 | 417.33 |
| 186 | 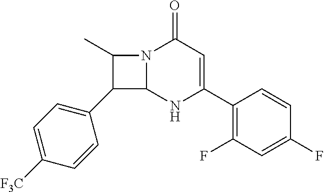 | 394.34 |
| 187 | 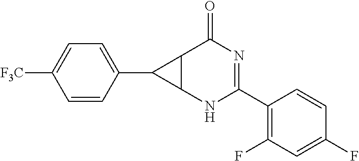 | 366.28 |
| 188 | 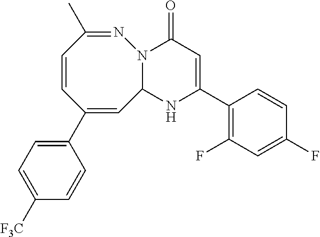 | 445.38 |
| 189 | 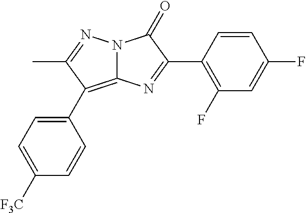 | 391.29 |
| 190 | 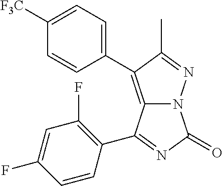 | 391.29 |
| 191 | 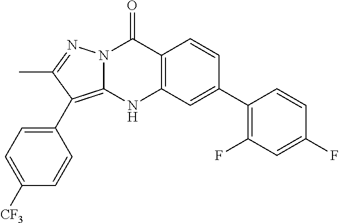 | 455.38 |
| 192 | 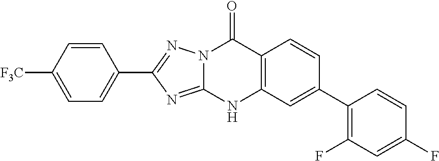 | 442.34 |
| 193 | 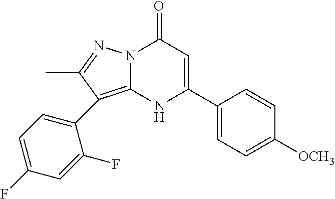 | 367.35 |
| 194 | 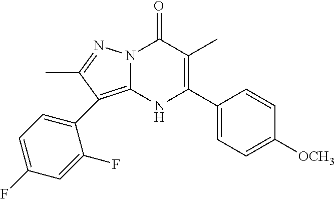 | 381.38 |
| 195 | 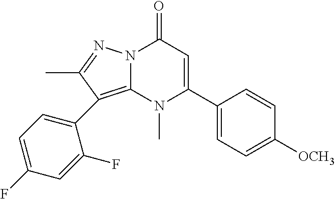 | 381.38 |
| 196 | 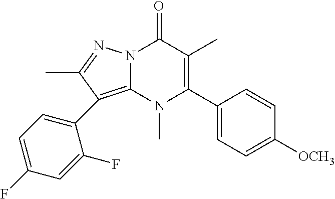 | 395.4 |
| 197 | 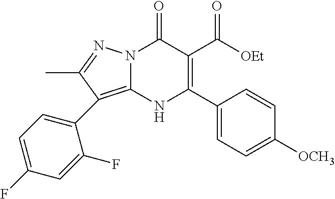 | 439.41 |
| 198 | 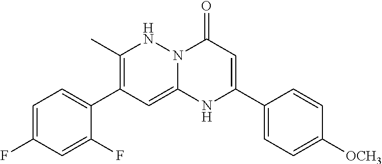 | 381.38 |
| 199 | 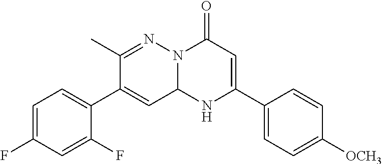 | 381.38 |
| 200 | 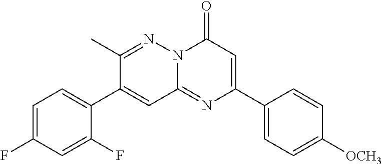 | 379.36 |
| 201 | 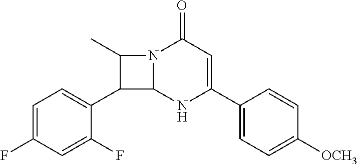 | 356.37 |
| 202 | 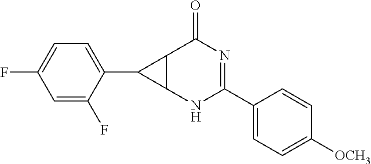 | 328.31 |
| 203 | 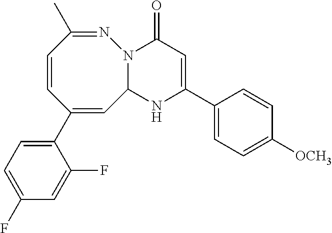 | 407.41 |
| 204 | 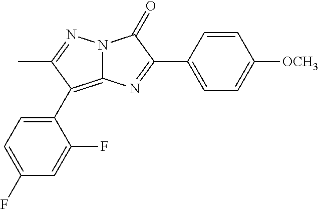 | 353.32 |
| 205 | 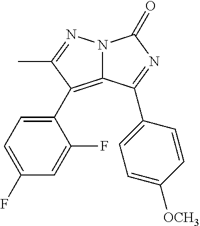 | 353.32 |
| 206 | 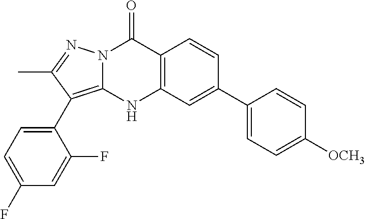 | 417.41 |
| 207 | 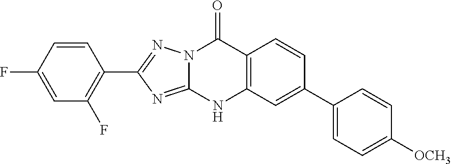 | 404.37 |
| 208 | 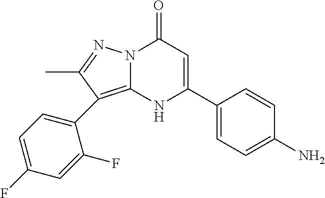 | 352.34 |
| 209 | 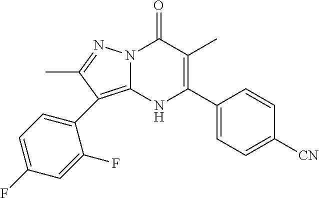 | 366.36 |
| 210 | 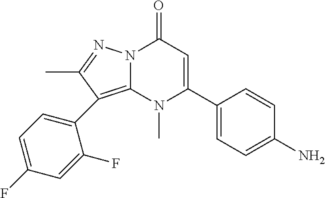 | 366.36 |
| 211 | 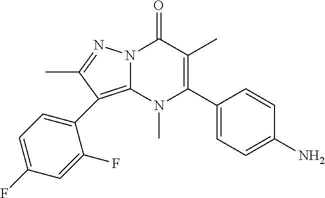 | 380.39 |
| 212 | 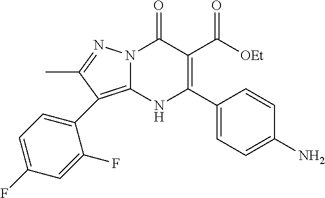 | 424.4 |
| 213 | 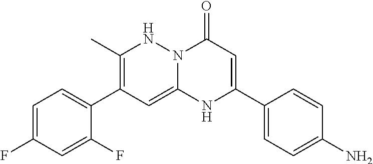 | 366.36 |
| 214 | 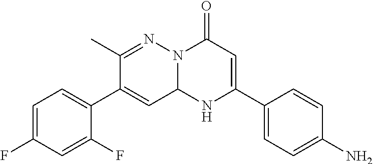 | 366.36 |
| 215 | 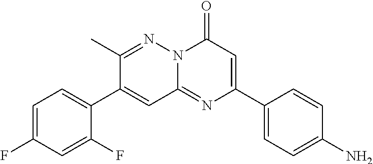 | 364.35 |
| 216 | 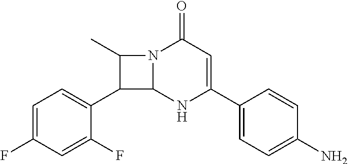 | 341.35 |
| 217 | 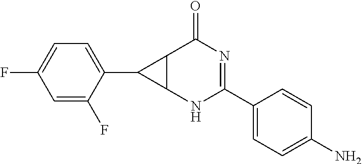 | 313.3 |
| 218 | 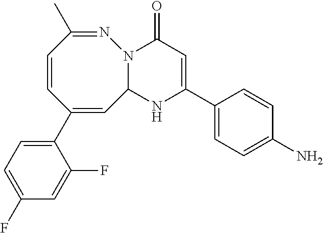 | 392.4 |
| 219 | 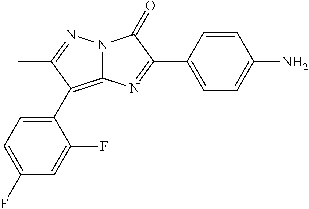 | 338.31 |
| 220 | 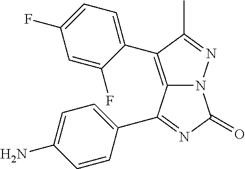 | 338.31 |
| 221 | 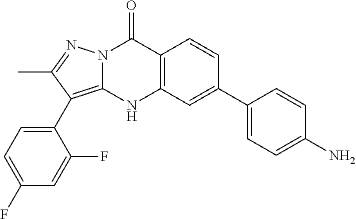 | 402.4 |
| 222 | 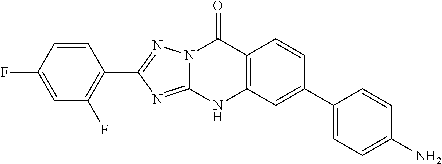 | 389.36 |
| 223 | 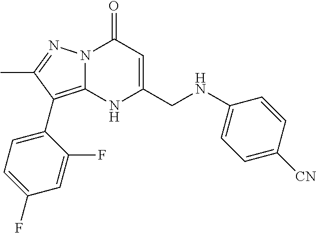 | 391.37 |
| 224 | 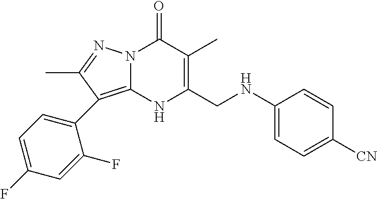 | 405.4 |
| 225 | 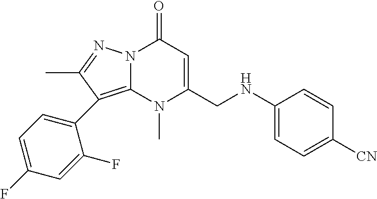 | 404.41 |
| 226 | 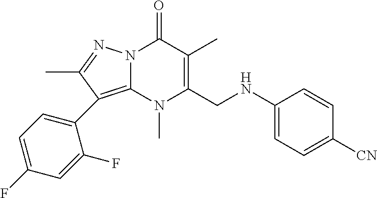 | 419.43 |
| 227 | 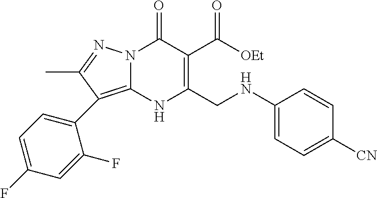 | 463.44 |
| 228 | 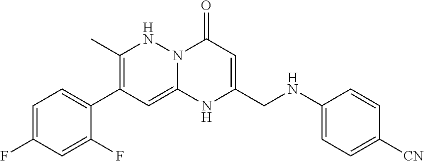 | 405.4 |
| 229 | 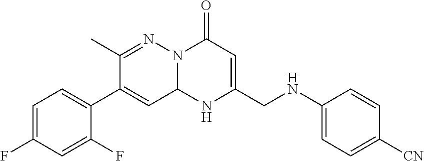 | 405.4 |
| 230 | 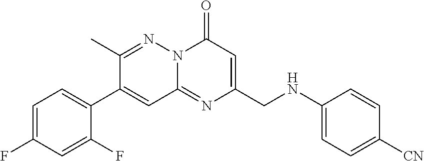 | 403.38 |
| 231 | 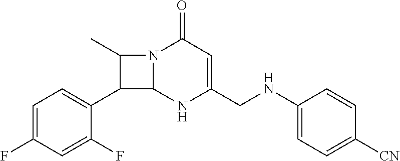 | 380.39 |
| 232 | 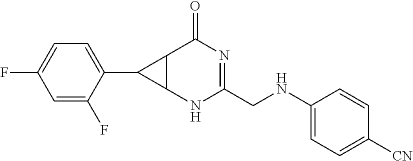 | 352.34 |
| 233 | 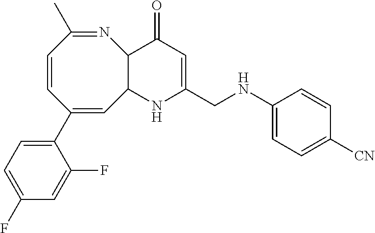 | 431.44 |
| 234 | 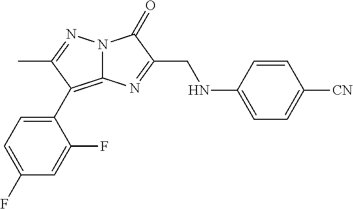 | 377.35 |
| 235 | 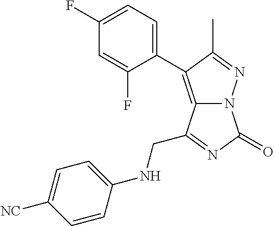 | 377.35 |
| 236 | 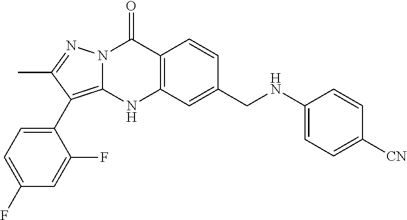 | 441.43 |
| 237 | 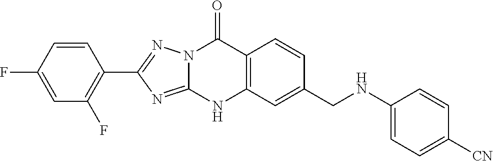 | 428.39 |
| 238 | 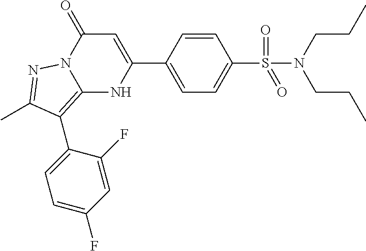 | 500.56 |
| 239 | 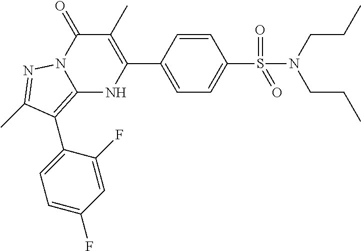 | 514.59 |
| 240 | 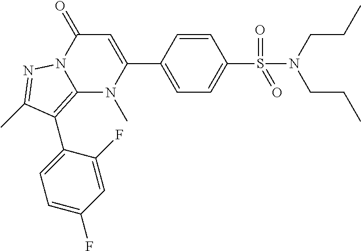 | 514.59 |
| 241 | 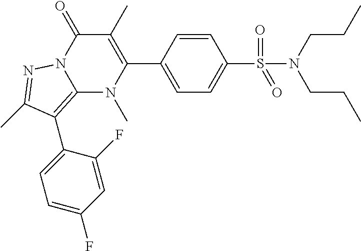 | 528.61 |
| 242 | 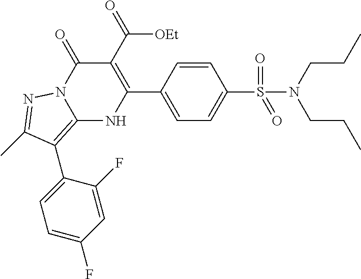 | 572.62 |
| 243 | 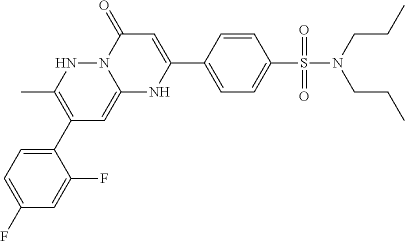 | 514.59 |
| 244 | 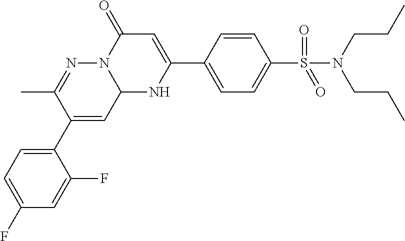 | 514.59 |
| 245 | 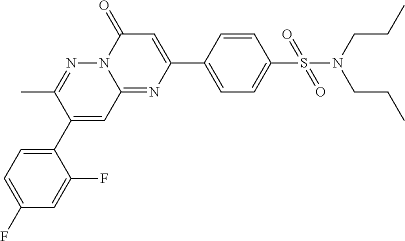 | 512.57 |
| 246 | 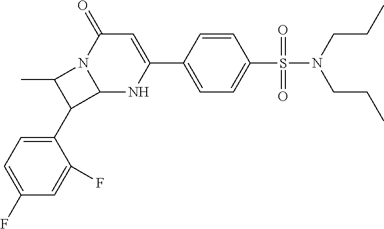 | 489.58 |
| 247 | 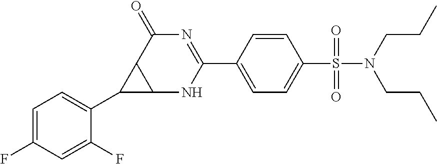 | 461.52 |
| 248 | 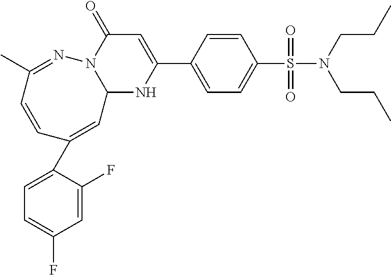 | 540.62 |
| 249 | 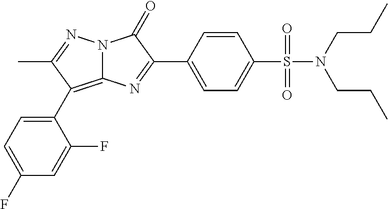 | 486.52 |
| 250 | 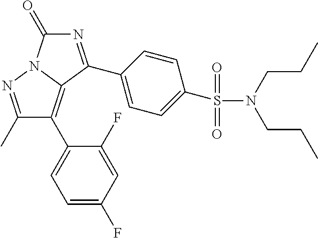 | 486.53 |
| 251 | 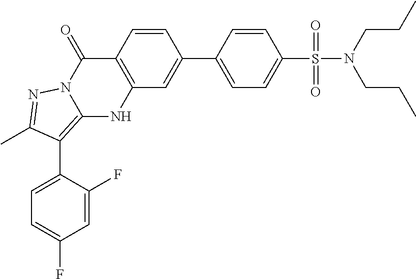 | 550.62 |
| 252 | 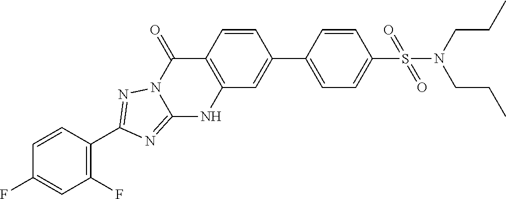 | 537.58 |
| 253 | 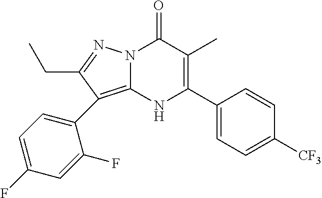 | 433.37 |
| 254 | 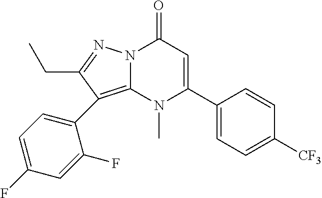 | 433.37 |
| 255 | 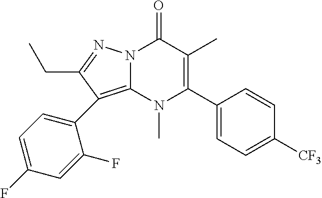 | 447.4 |
| 256 | 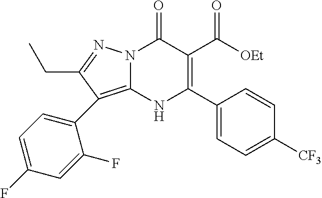 | 491.41 |
| 257 | 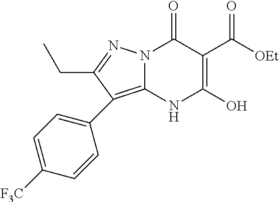 | 395.33 |
| 258 | 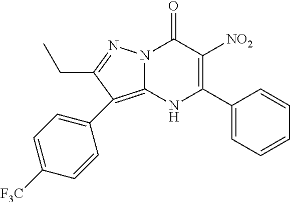 | 428.36 |
| 259 | 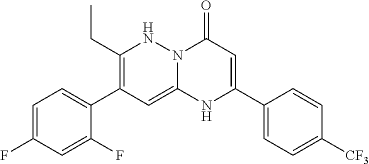 | 433.37 |
| 260 | 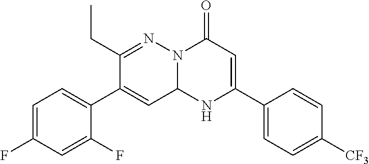 | 433.37 |
| 261 | 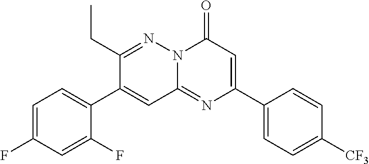 | 431.36 |
| 262 | 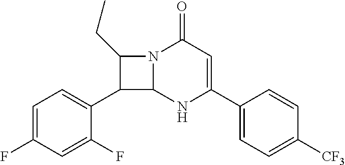 | 408.36 |
| 263 | 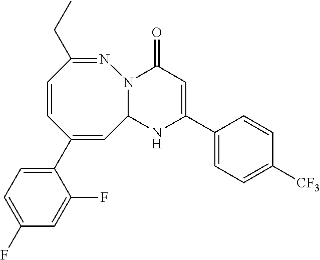 | 459.41 |
| 264 | 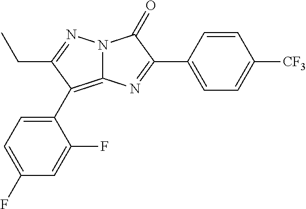 | 405.32 |
| 265 | 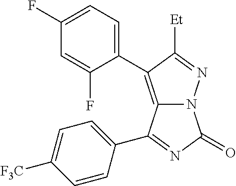 | 405.32 |
| 266 | 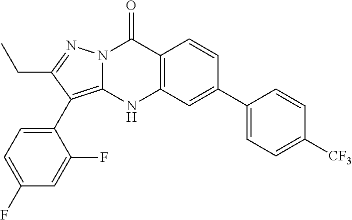 | 469.41 |
| 267 | 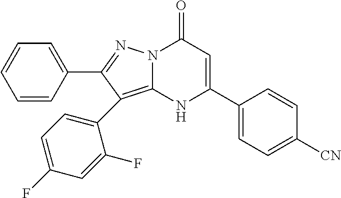 | 424.4 |
| 268 | 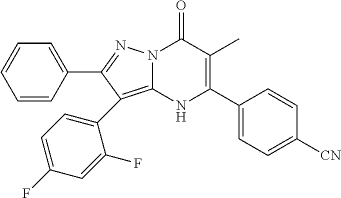 | 438.43 |
| 269 | 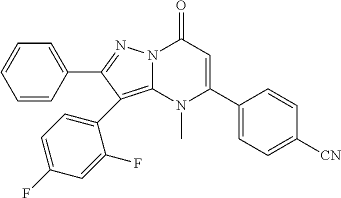 | 438.43 |
| 270 | 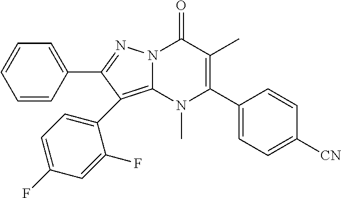 | 452.45 |
| 271 | 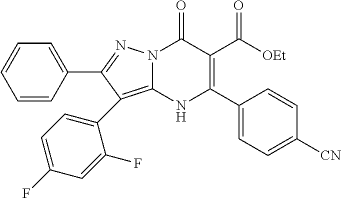 | 496.46 |
| 272 | 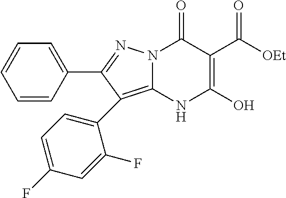 | 411.36 |
| 273 | 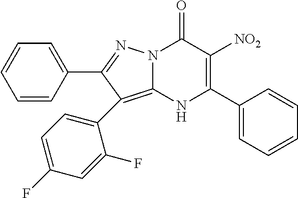 | 444.39 |
| 274 | 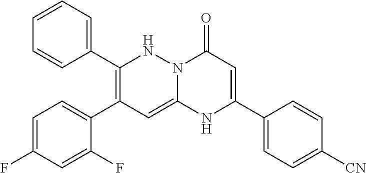 | 438.43 |
| 275 | 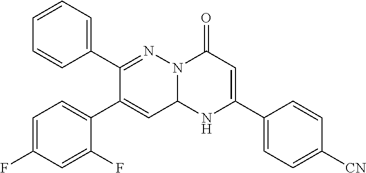 | 438.43 |
| 276 | 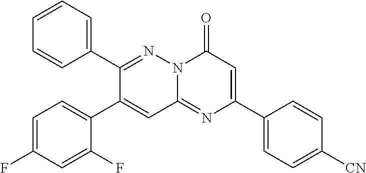 | 436.41 |
| 277 | 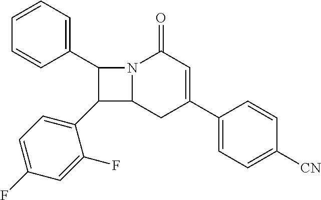 | 413.42 |
| 278 | 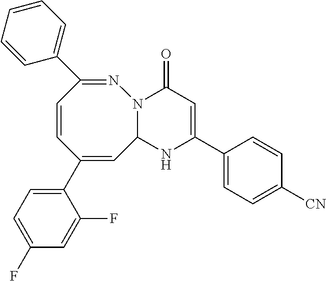 | 464.47 |
| 279 | 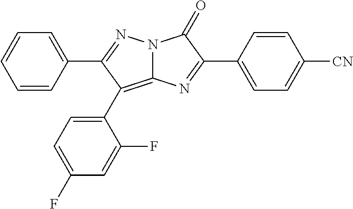 | 410.38 |
| 280 | 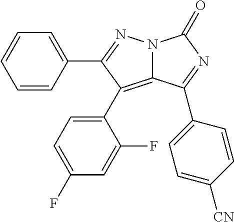 | 410.38 |
| 281 | 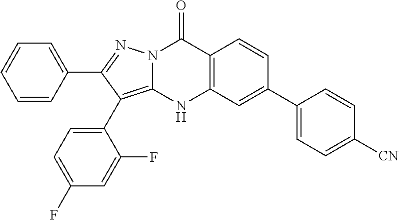 | 474.46 |
| 282 | 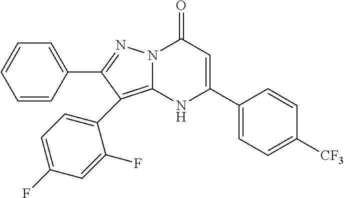 | 467.39 |
| 283 | 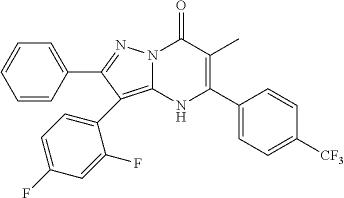 | 481.42 |
| 284 | 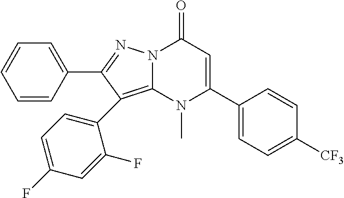 | 481.42 |
| 285 | 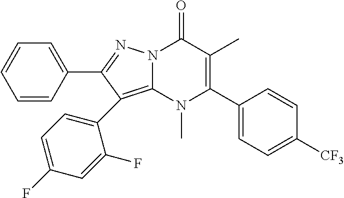 | 495.44 |
| 286 | 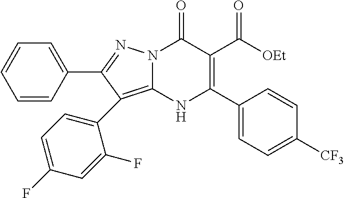 | 539.45 |
| 287 | 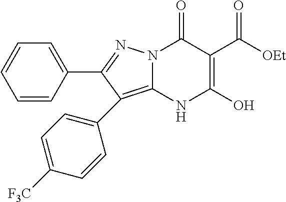 | 443.38 |
| 288 | 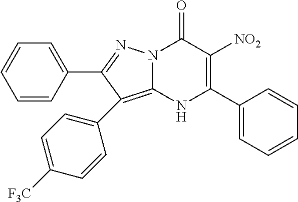 | 476.41 |
| 289 | 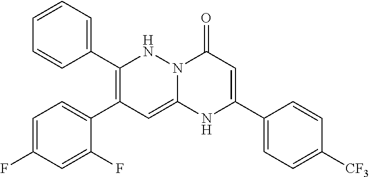 | 481.42 |
| 290 | 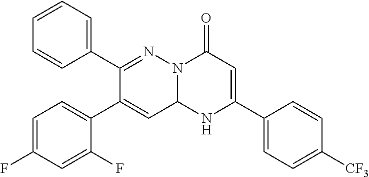 | 481.42 |
| 291 | 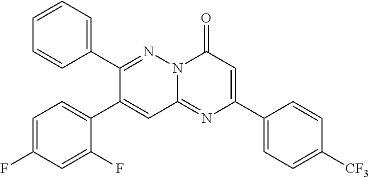 | 479.4 |
| 292 | 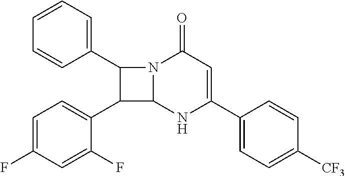 | 456.41 |
| 293 | 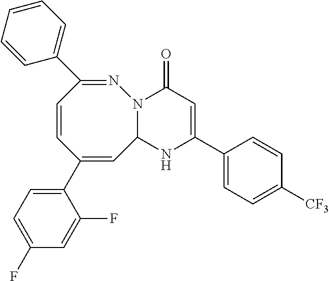 | 507.45 |
| 294 | 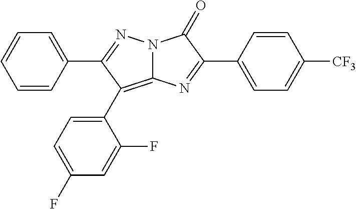 | 453.36 |
| 295 | 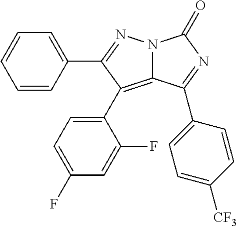 | 453.36 |
| 296 | 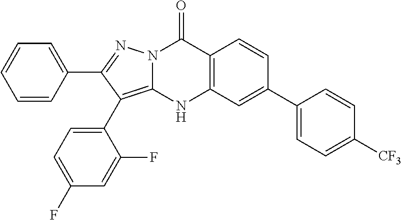 | 517.45 |
| 297 | 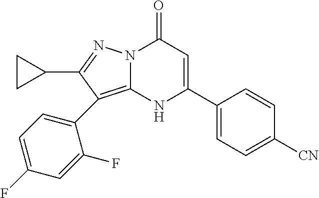 | 388.37 |
| 298 | 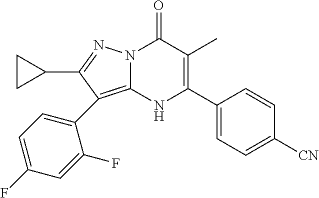 | 402.4 |
| 299 | 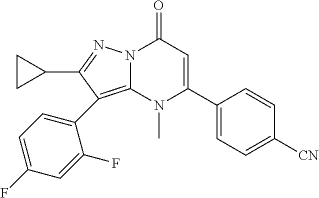 | 402.4 |
| 300 | 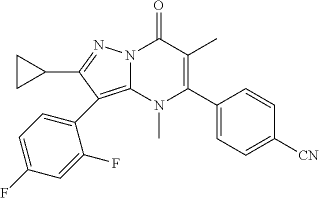 | 416.42 |
| 301 | 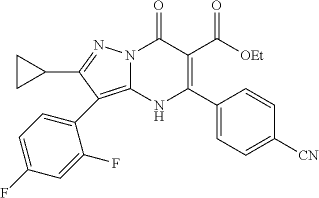 | 460.43 |
| 302 | 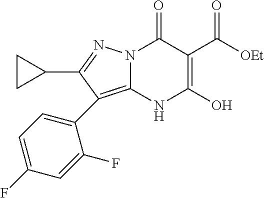 | 375.33 |
| 303 | 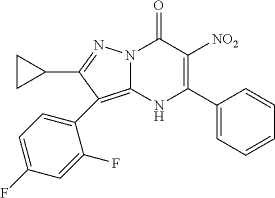 | 408.36 |
| 304 | 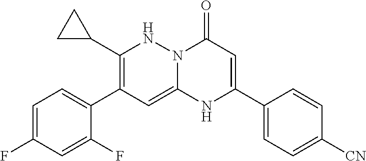 | 402.4 |
| 305 | 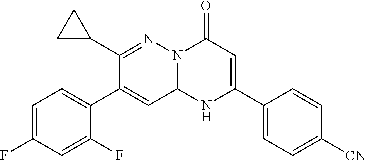 | 402.4 |
| 306 | 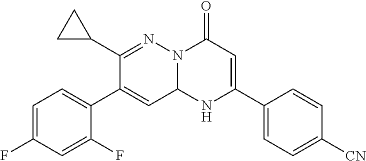 | 400.38 |
| 307 | 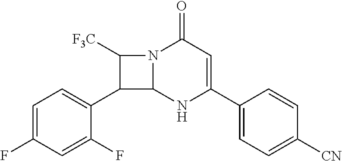 | 377.39 |
| 308 | 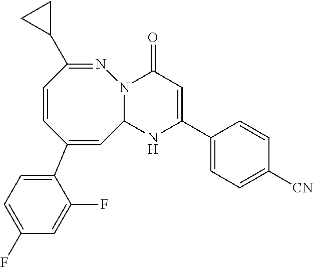 | 428.43 |
| 309 | 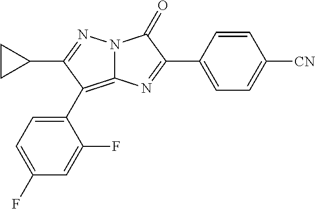 | 374.34 |
| 310 | 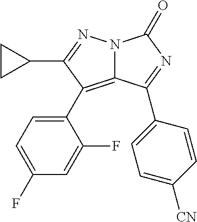 | 374.34 |
| 311 | 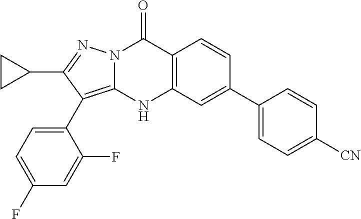 | 438.43 |
| 312 | 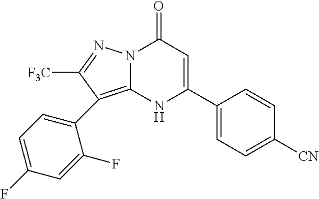 | 416.3 |
| 313 | 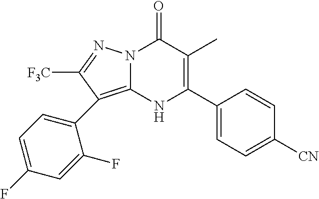 | 430.33 |
| 314 | 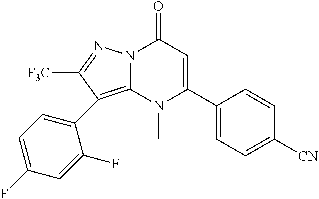 | 430.33 |
| 315 | 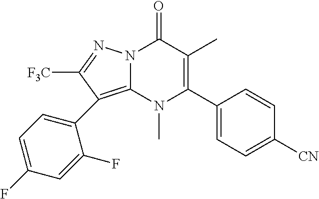 | 444.36 |
| 316 | 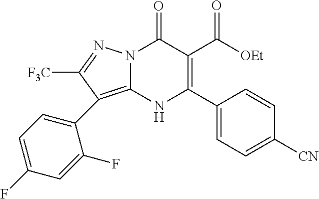 | 488.37 |
| 317 | 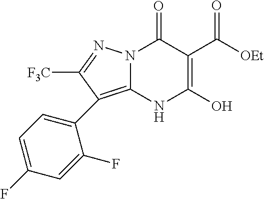 | 403.26 |
| 318 | 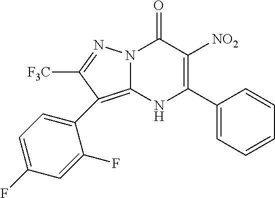 | 436.29 |
| 319 | 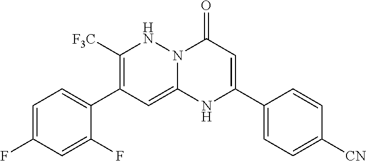 | 430.33 |
| 320 | 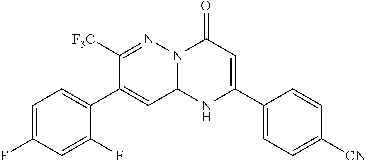 | 430.33 |
| 321 | 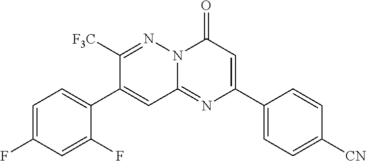 | 428.31 |
| 322 | 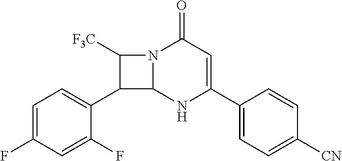 | 405.32 |
| 323 | 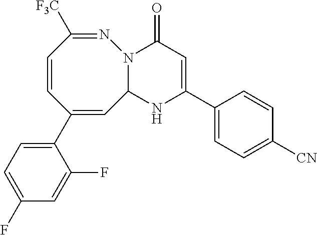 | 456.37 |
| 324 | 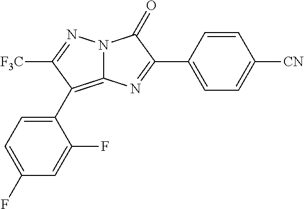 | 402.28 |
| 325 | 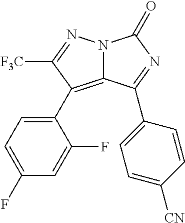 | 402.28 |
| 326 | 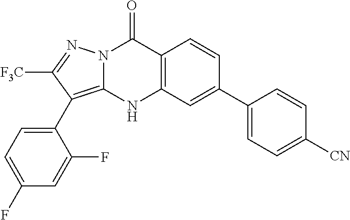 | 466.36 |
| 327 | 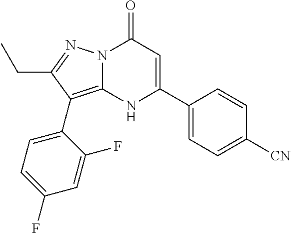 | 376.36 |
| 328 | 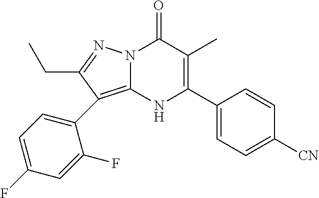 | 390.39 |
| 329 | 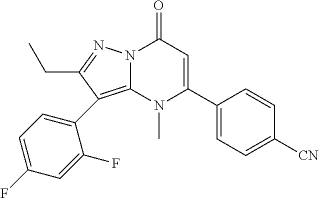 | 390.39 |
| 330 | 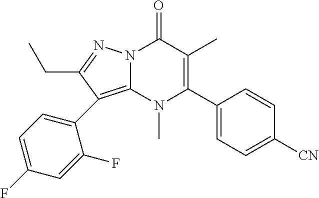 | 404.41 |
| 331 | 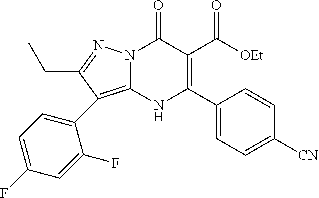 | 448.42 |
| 332 | 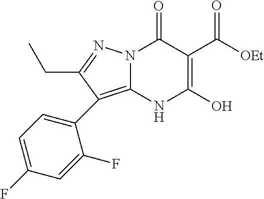 | 363.32 |
| 333 | 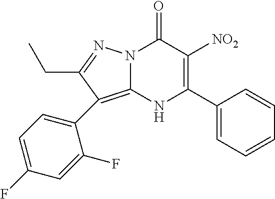 | 396.35 |
| 334 | 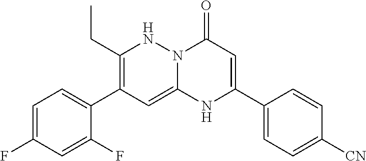 | 390.39 |
| 335 | 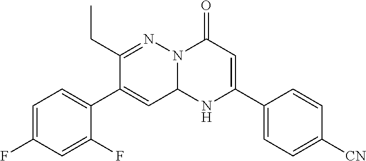 | 390.39 |
| 336 | 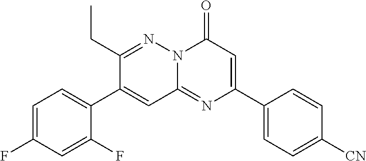 | 388.37 |
| 337 | 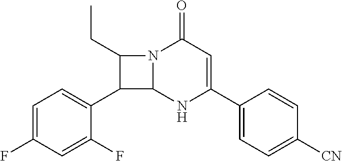 | 365.38 |
| 338 | 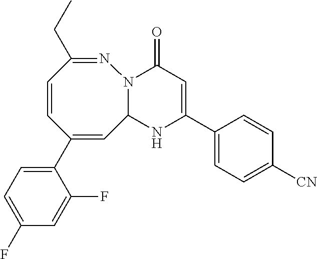 | 416.42 |
| 339 | 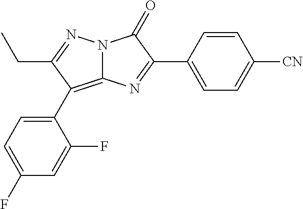 | 362.33 |
| 340 | 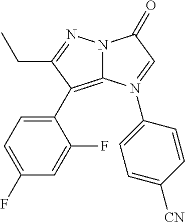 | 362.33 |
| 341 | 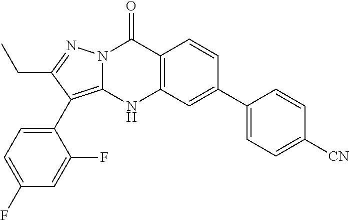 | 426.42 |
| 342 | 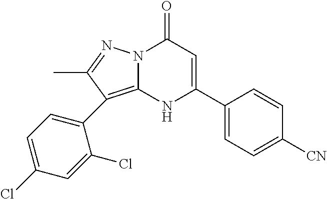 | 395.24 |
| 343 | 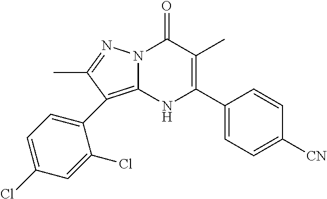 | 409.27 |
| 344 | 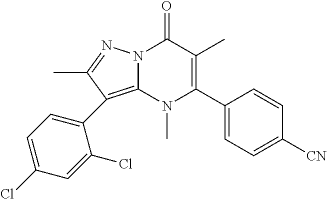 | 423.29 |
| 345 | 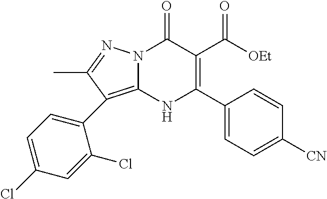 | 467.3 |
| 346 | 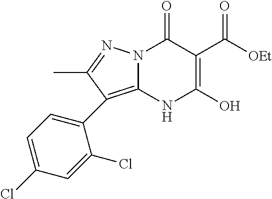 | 382.2 |
| 347 | 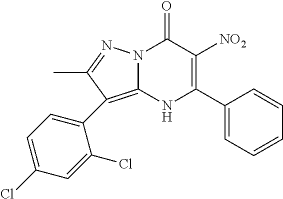 | 415.23 |
| 348 | 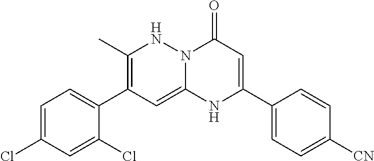 | 409.27 |
| 349 | 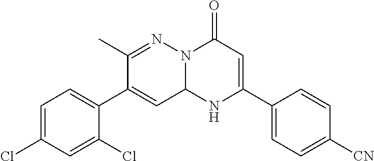 | 409.27 |
| 350 | 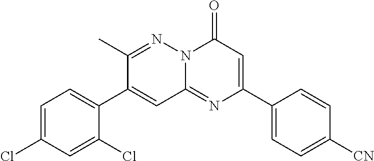 | 407.25 |
| 351 | 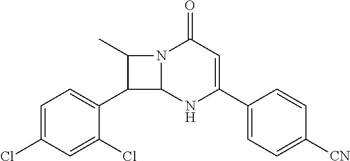 | 384.26 |
| 352 | 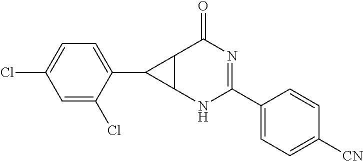 | 356.21 |
| 353 | 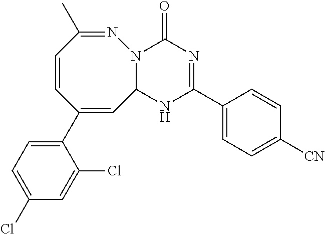 | 435.31 |
| 354 | 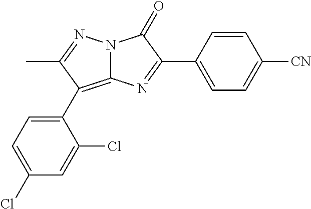 | 381.21 |
| 355 | 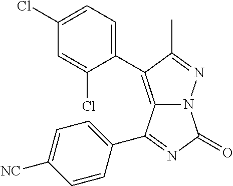 | 381.21 |
| 356 | 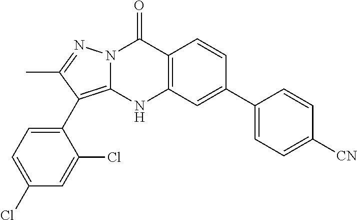 | 445.3 |
| 357 | 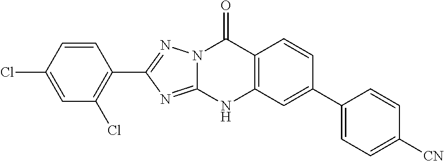 | 432.26 |
| 358 | 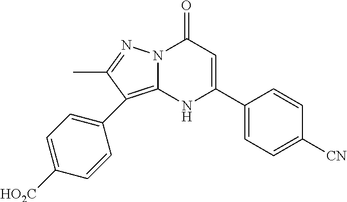 | 370.36 |
| 359 | 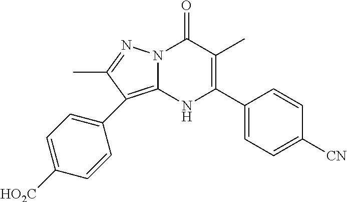 | 384.39 |
| 360 | 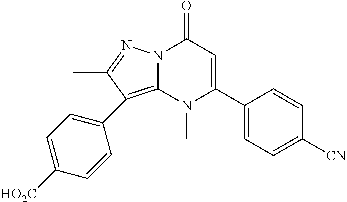 | 384.39 |
| 361 | 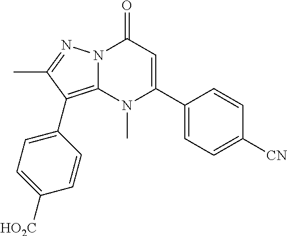 | 398.41 |
| 362 | 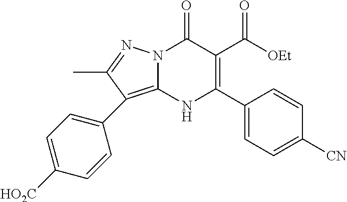 | 442.42 |
| 363 | 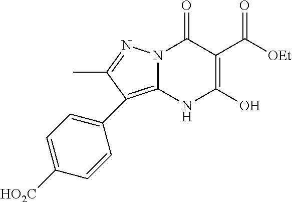 | 357.32 |
| 364 | 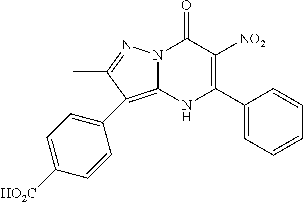 | 390.35 |
| 365 | 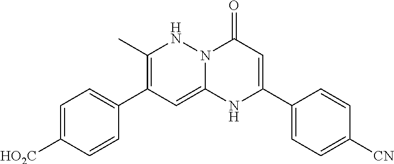 | 384.39 |
| 366 | 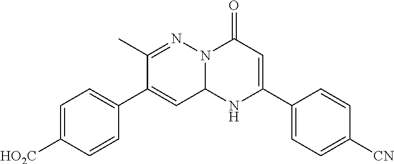 | 384.39 |
| 367 | 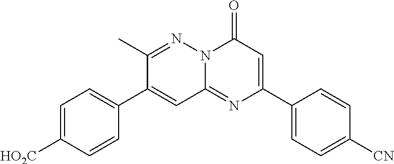 | 382.37 |
| 368 | 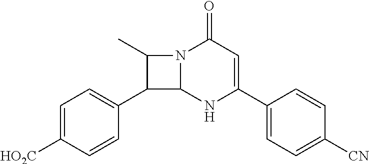 | 359.38 |
| 369 | 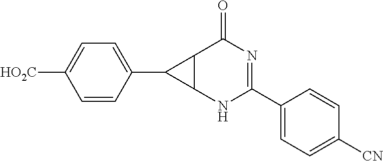 | 331.32 |
| 370 | 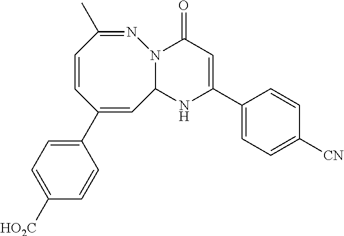 | 410.42 |
| 371 | 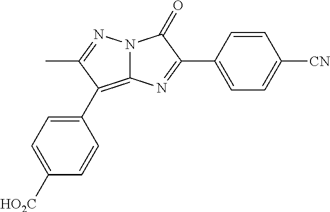 | 356.33 |
| 372 | 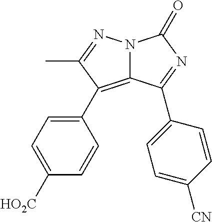 | 356.33 |
| 373 | 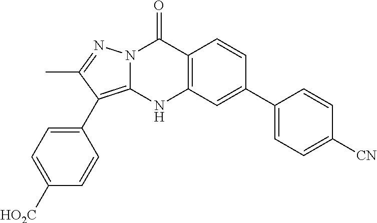 | 420.42 |
| 374 | 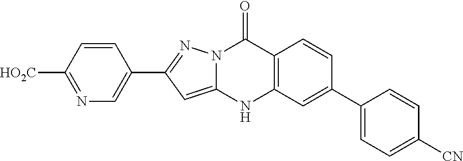 | 407.38 |
| 375 | 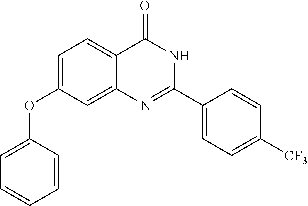 | 382.34 |
| 376 | 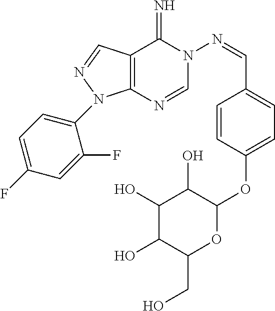 | 528.46 |
| 377 | 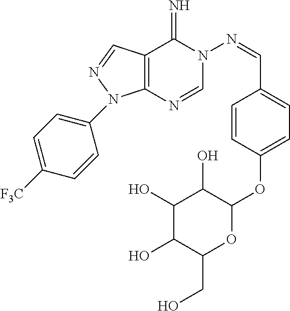 | 560.48 |
| 378 | 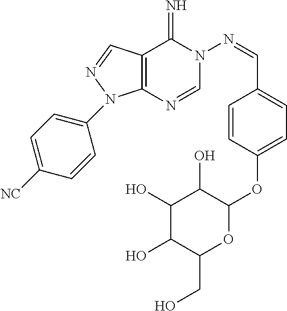 | 517.49 |
| 379 | 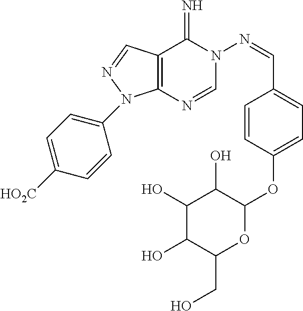 | 536.49 |
| 380 | 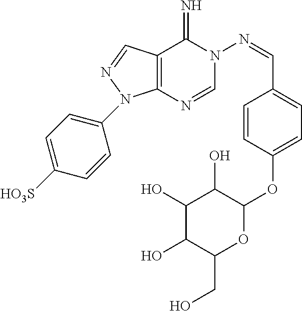 | 572.55 |
| 381 | 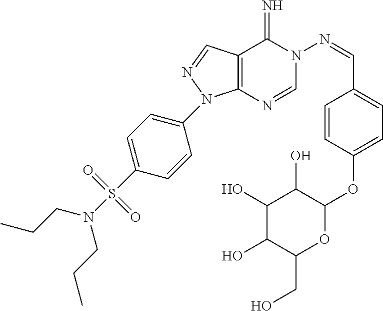 | 655.72 |
| 382 | 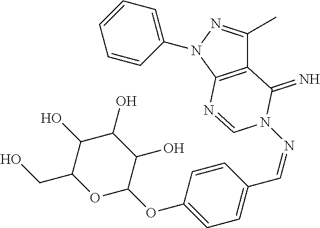 | 506.51 |
| 383 | 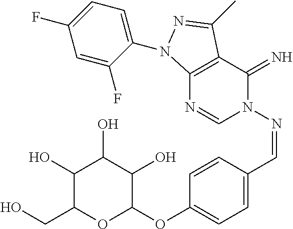 | 542.49 |
| 384 | 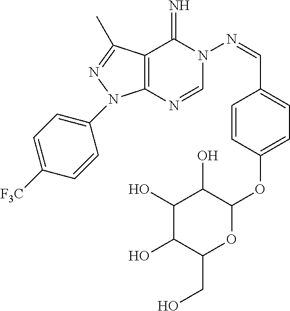 | 574.51 |
| 385 | 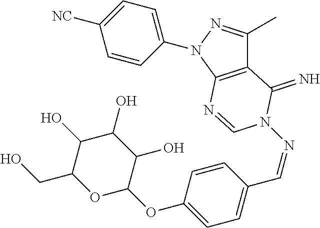 | 531.52 |
| 386 | 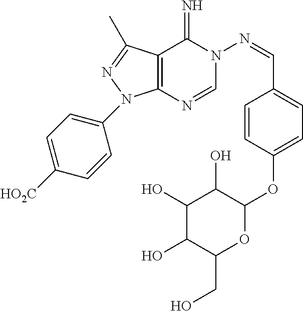 | 550.52 |
| 387 | 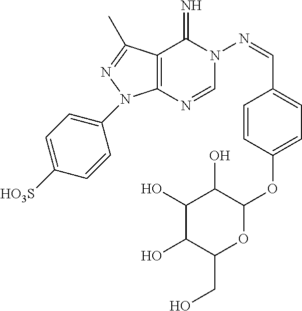 | 586.57 |
| 388 | 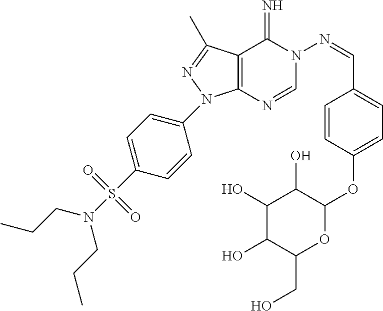 | 669.75 |
| 389 | 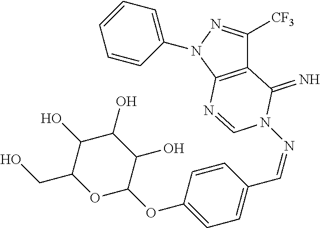 | 560.48 |
| 390 | 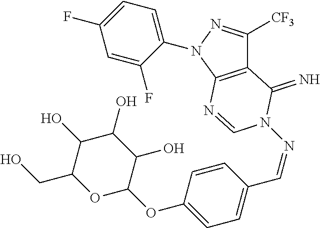 | 596.46 |
| 391 | 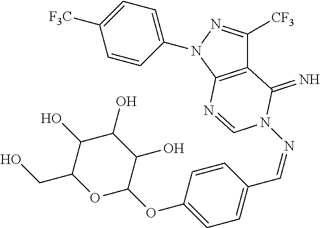 | 624.45 |
| 392 | 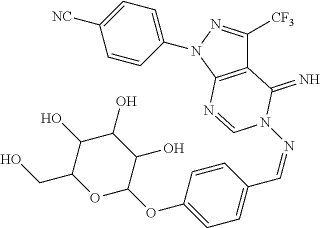 | 585.49 |
| 393 | 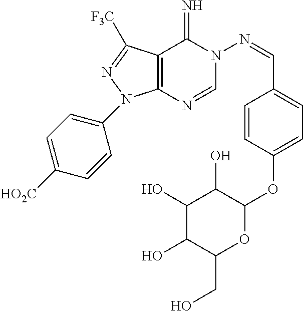 | 604.49 |
| 394 | 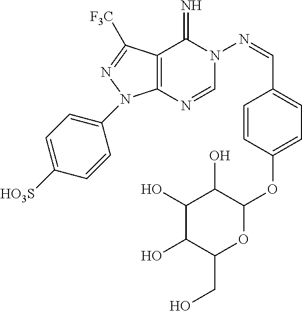 | 640.55 |
| 395 | 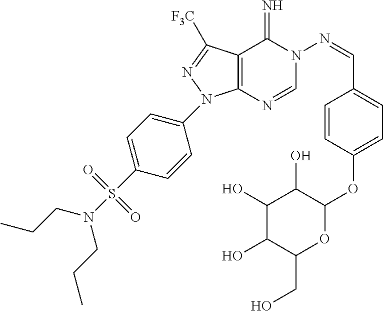 | 723.72 |
| 396 | 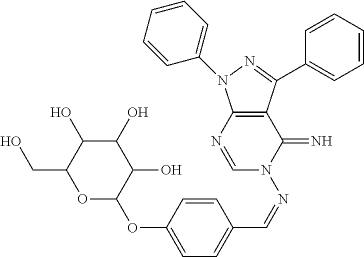 | 568.58 |
| 397 | 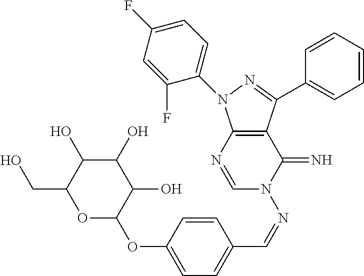 | 604.56 |
| 398 | 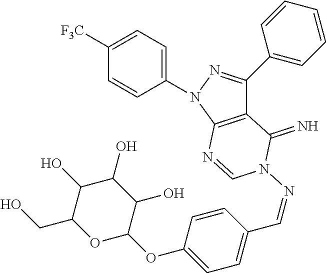 | 636.58 |
| 399 | 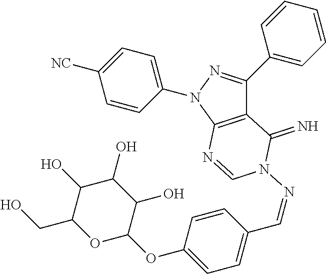 | 589.56 |
| 400 | 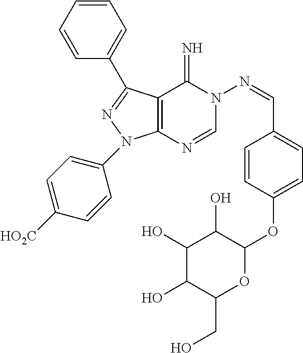 | 612.59 |
| 401 | 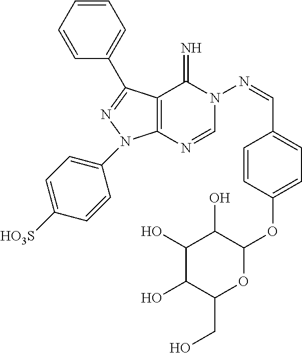 | 648.64 |
| 402 | 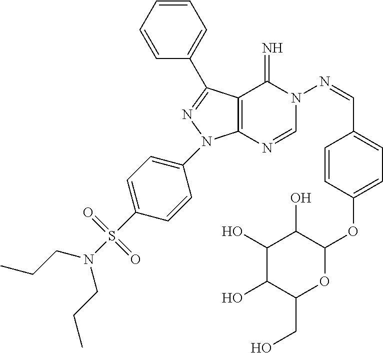 | 731.82 |
| 403 | 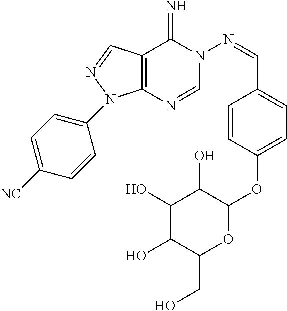 | 517.49 |
| 404 | 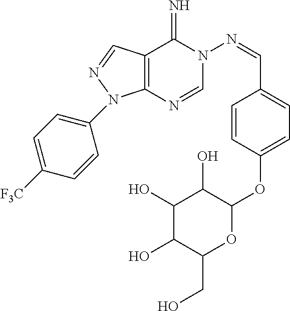 | 560.48 |
| 405 | 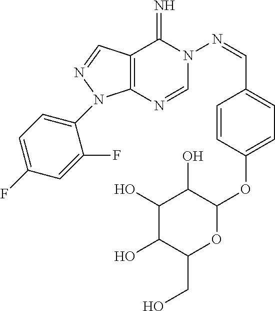 | 528.46 |
| 406 | 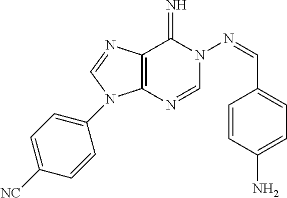 | 354.37 |
| 407 | 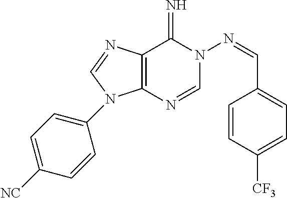 | 407.35 |
| 408 | 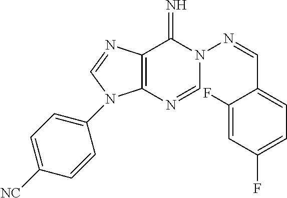 | 375.33 |
| 409 | 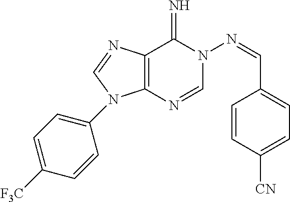 | 407.35 |
| 410 | 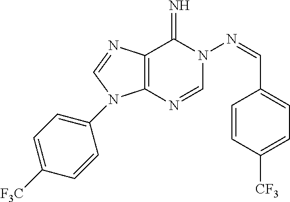 | 450.34 |
| 411 | 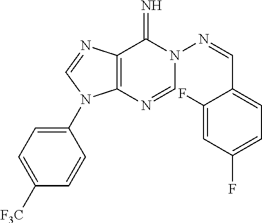 | 418.32 |
| 412 | 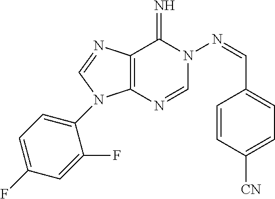 | 375.33 |
| 413 | 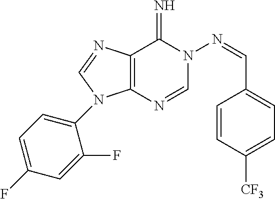 | 418.32 |
| 414 | 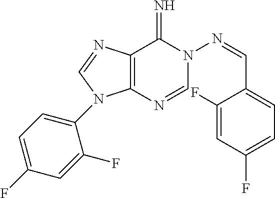 | 386.31 |
| 415 | 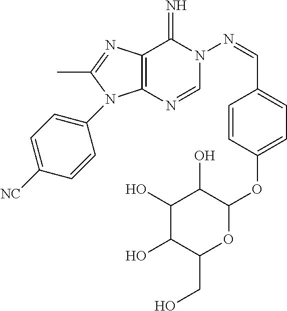 | 531.52 |
| 416 | 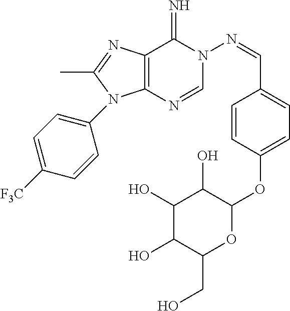 | 574.51 |
| 417 | 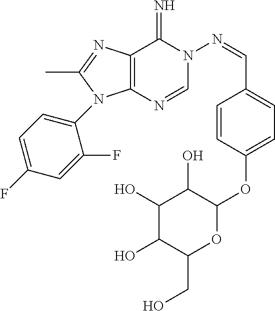 | 542.49 |
| 418 | 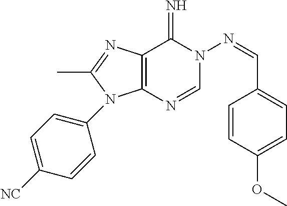 | 383.41 |
| 419 | 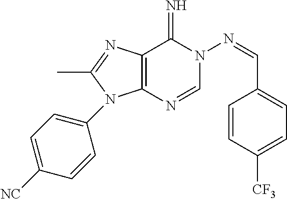 | 421.38 |
| 420 | 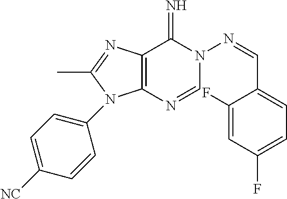 | 389.36 |
| 421 | 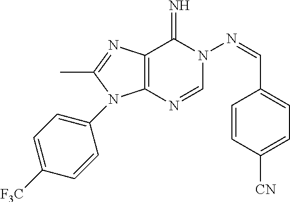 | 421.38 |
| 422 | 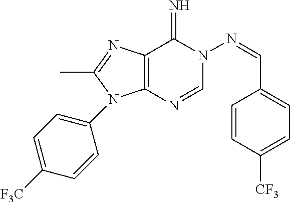 | 464.37 |
| 423 | 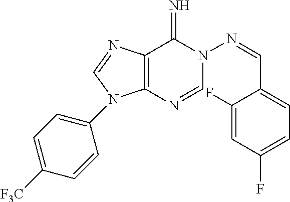 | 418.32 |
| 424 | 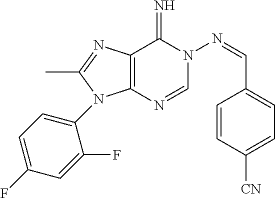 | 389.36 |
| 425 | 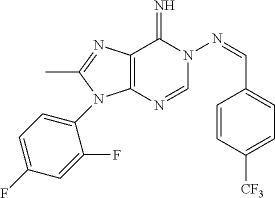 | 432.35 |
| 426 | 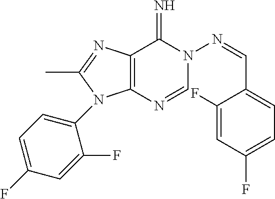 | 400.33 |
| 427 | 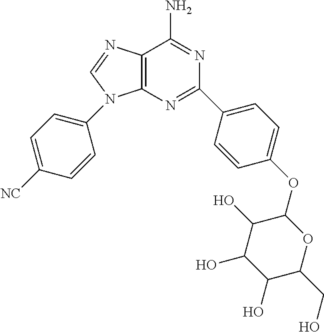 | 490.47 |
| 428 | 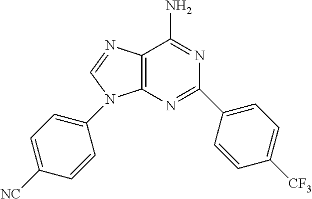 | 380.33 |
| 429 | 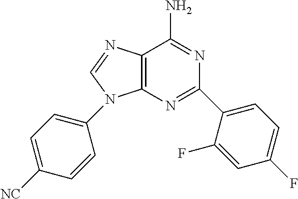 | 348.31 |
| 430 | 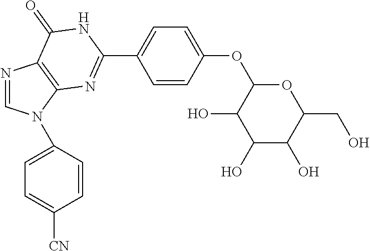 | 491.45 |
| 431 | 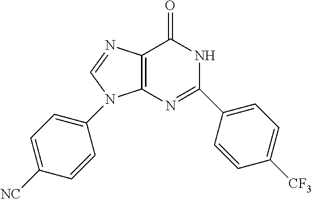 | 381.31 |
| 432 | 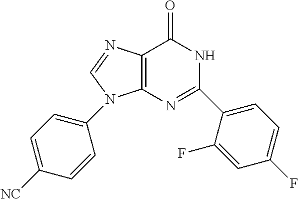 | 349.29 |
| 433 | 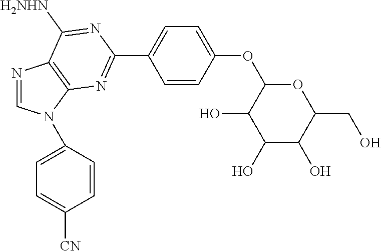 | 501.45 |
| 434 | 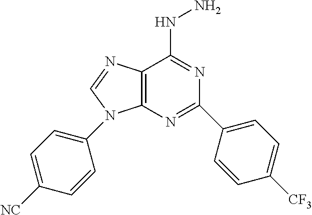 | 395.34 |
| 435 | 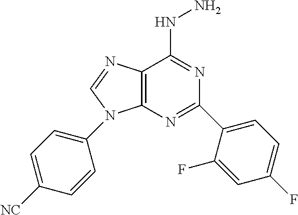 | 363.32 |
| 436 | 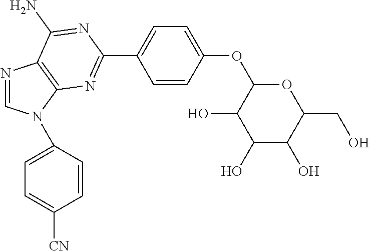 | 490.47 |
| 437 | 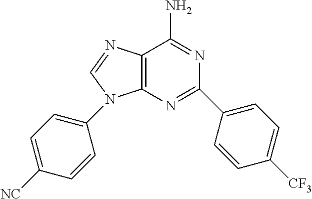 | 380.33 |
| 438 | 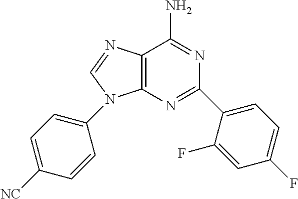 | 348.31 |
| 439 | 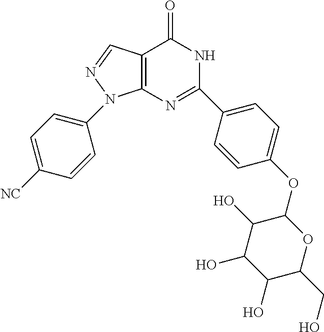 | 491.45 |
| 440 | 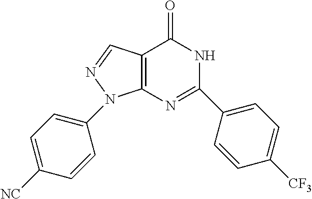 | 381.31 |
| 441 | 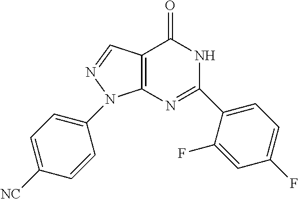 | 349.29 |
| 442 | 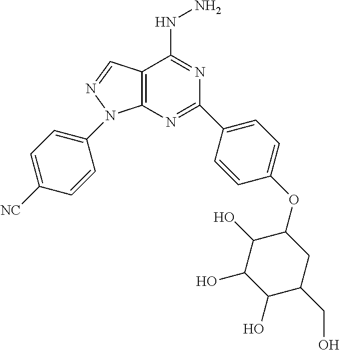 | 505.48 |
| 443 | 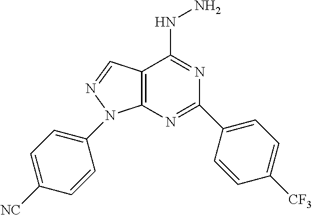 | 395.34 |
| 444 | 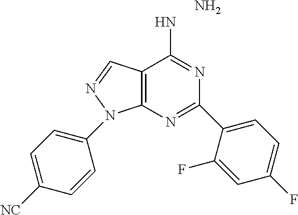 | 363.32 |
| 445 | 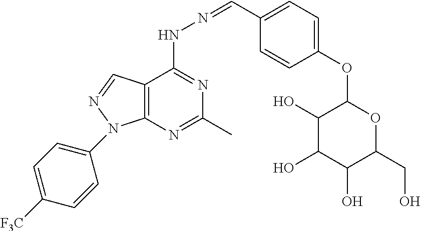 | 574.51 |
| 446 | 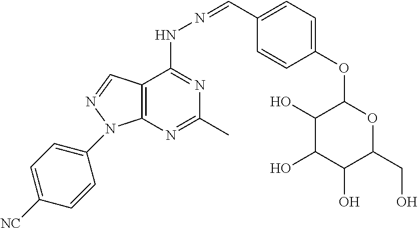 | 531.52 |
| 447 | 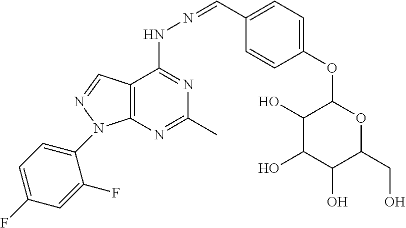 | 542.49 |
| 448 | 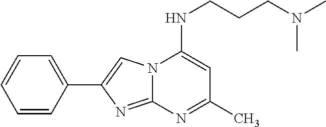 | 309.41 |
| 449 | 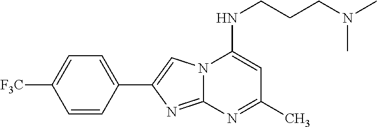 | 377.41 |
| 450 | 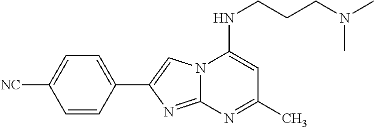 | 334.42 |
| 451 | 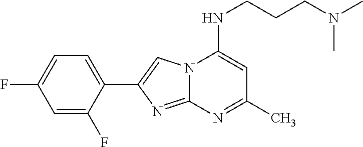 | 345.39 |
| 452 | 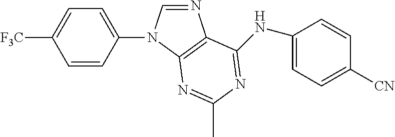 | 394.35 |
| 453 | 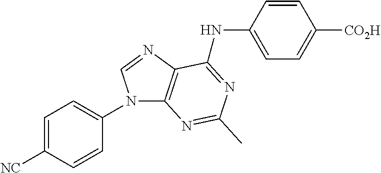 | 370.36 |
| 454 | 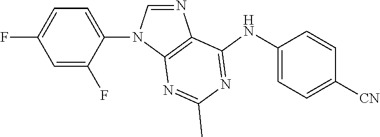 | 362.34 |
| 455 | 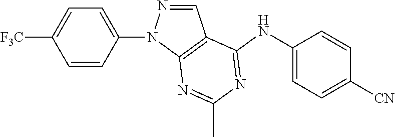 | 394.35 |
| 456 | 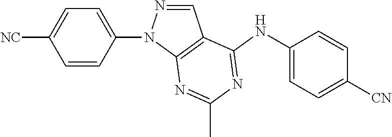 | 351.36 |
| 457 | 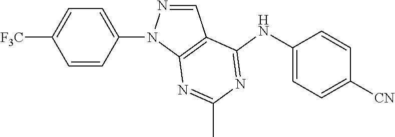 | 394.35 |
| 458 | 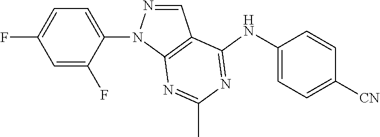 | 362.34 |
| 459 | 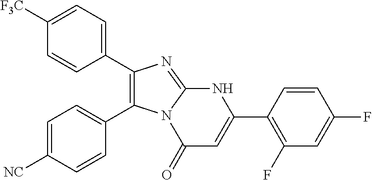 | 492.4 |
| 460 | 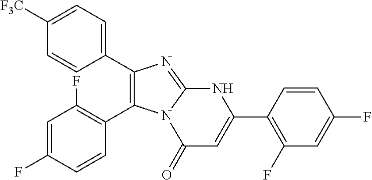 | 503.37 |
| 461 | 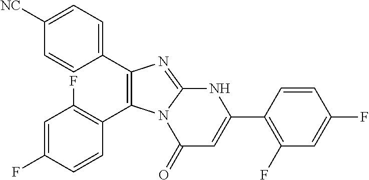 | 460.38 |
| 462 | 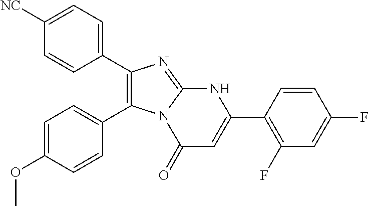 | 454.43 |
| 463 | 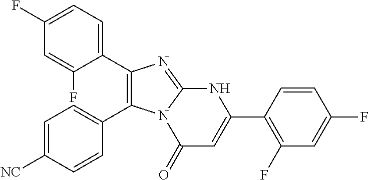 | 460.38 |
| 464 | 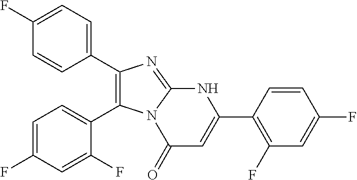 | 471.35 |
| 465 | 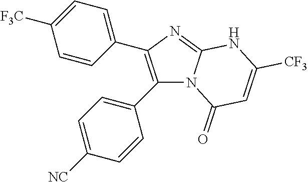 | 448.32 |
| 466 | 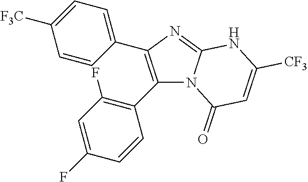 | 459.29 |
| 467 | 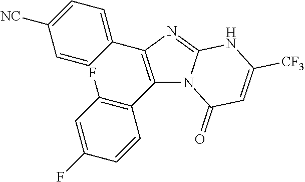 | 416.3 |
| 468 | 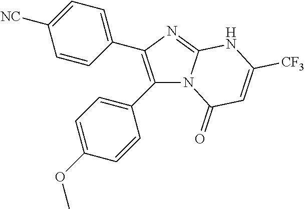 | 410.35 |
| 469 | 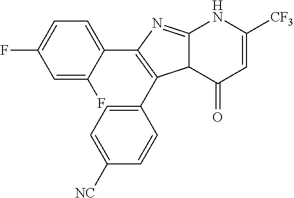 | 416.3 |
| 470 | 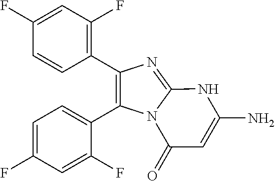 | 427.28 |
| 471 | 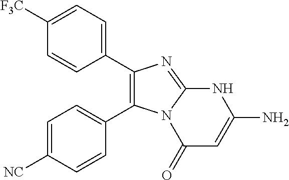 | 395.34 |
| 472 | 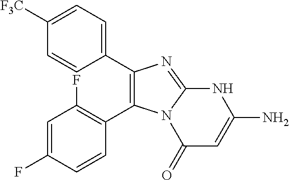 | 406.31 |
| 473 | 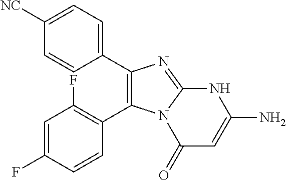 | 363.32 |
| 474 | 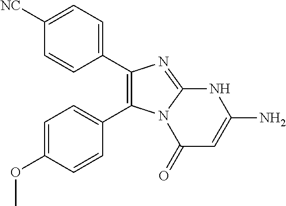 | 357.37 |
| 475 | 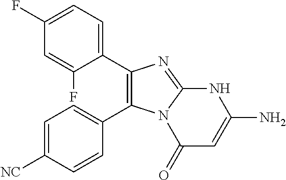 | 363.32 |
| 476 | 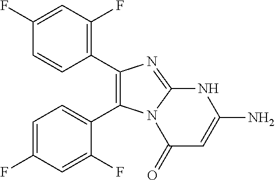 | 374.29 |
| 477 | 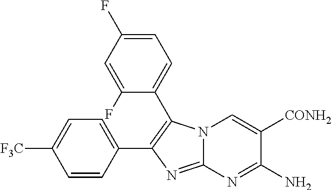 | 433.33 |
| 478 | 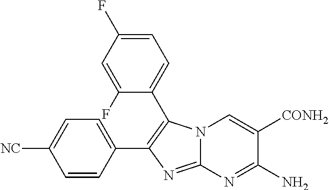 | 390.35 |
| 479 | 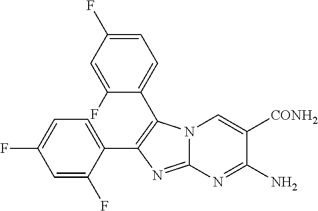 | 401.32 |
| 480 | 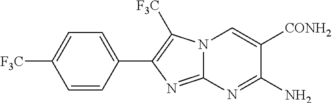 | 389.26 |
| 481 | 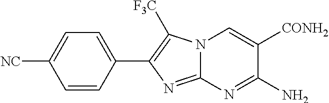 | 346.27 |
| 482 | 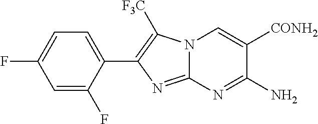 | 357.24 |
| 483 | 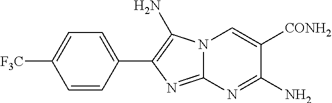 | 336.27 |
| 484 | 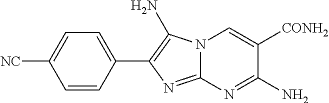 | 293.28 |
| 485 | 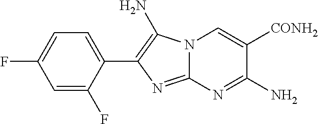 | 304.25 |
| 486 | 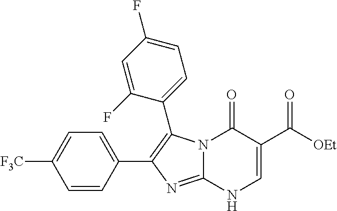 | 463.36 |
| 487 | 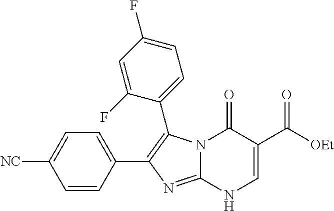 | 420.37 |
| 488 | 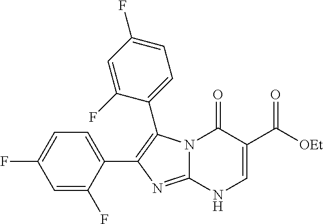 | 431.34 |
| 489 | 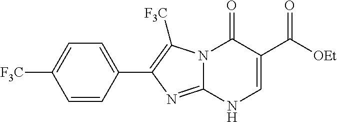 | 419.28 |
| 490 | 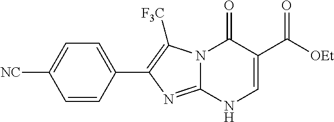 | 376.29 |
| 491 | 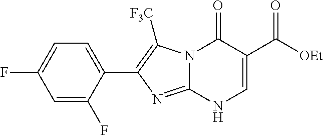 | 387.26 |
| 492 | 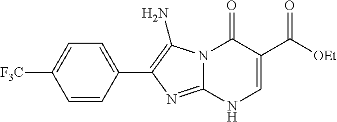 | 366.29 |
| 493 | 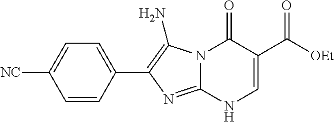 | 323.31 |
| 494 | 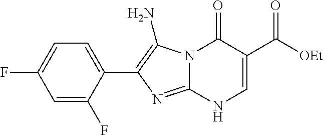 | 334.28 |
| 495 | 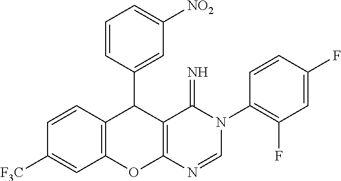 | 500.38 |
| 496 | 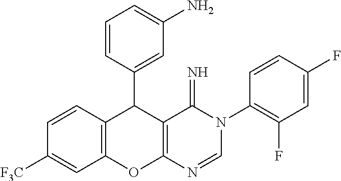 | 470.39 |
| 497 | 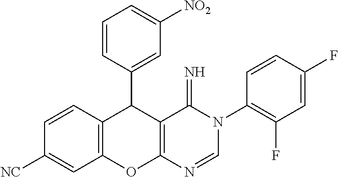 | 457.39 |
| 498 | 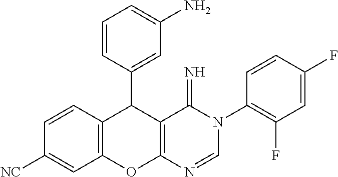 | 427.41 |
| 499 | 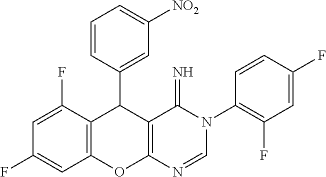 | 468.36 |
| 500 | 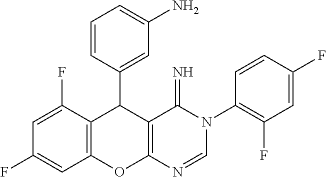 | 438.38 |
| 501 | 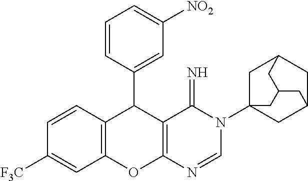 | 522.52 |
| 502 | 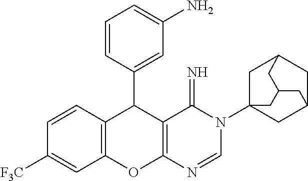 | 492.54 |
| 503 | 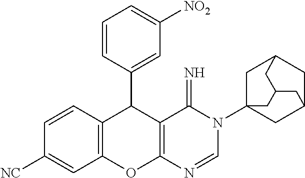 | 479.53 |
| 504 | 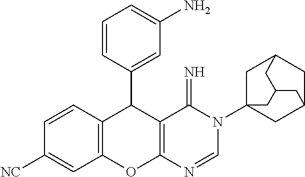 | 449.55 |
| 505 | 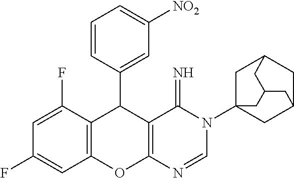 | 490.5 |
| 506 | 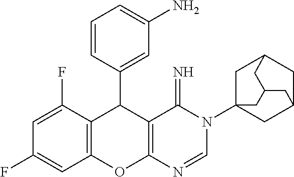 | 460.52 |
| 507 | 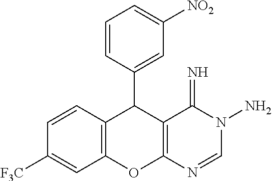 | 403.31 |
| 508 | 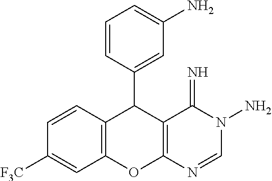 | 373.33 |
| 509 | 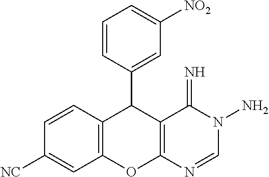 | 360.33 |
| 510 | 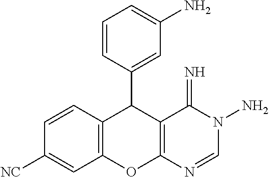 | 330.34 |
| 511 | 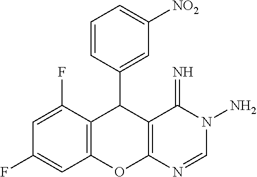 | 371.3 |
| 512 | 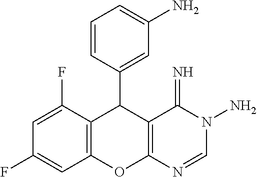 | 341.31 |
| 513 | 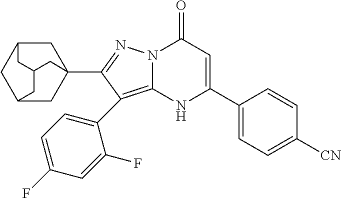 | 482.52 |
| 514 | 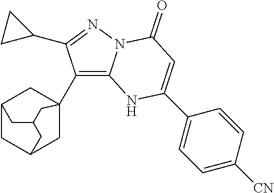 | 410.51 |
| 515 | 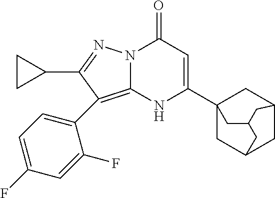 | 421.48 |
| 516 | 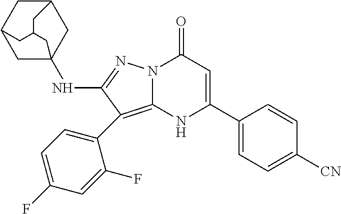 | 497.54 |
| 517 | 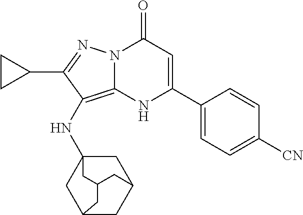 | 425.53 |
| 518 | 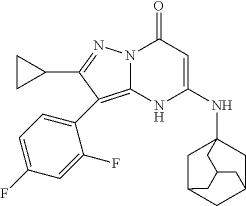 | 436.5 |
| 519 | 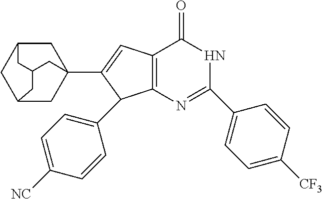 | 513.55 |
| 520 | 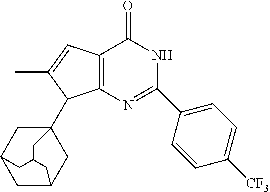 | 426.47 |
| 521 | 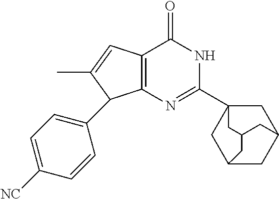 | 383.49 |
| 522 | 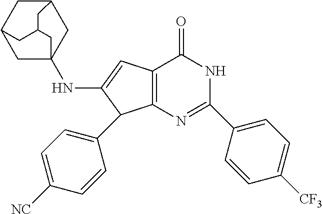 | 528.57 |
| 523 | 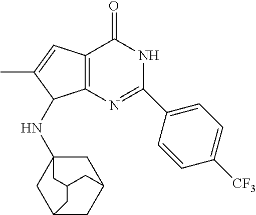 | 441.49 |
| 524 | 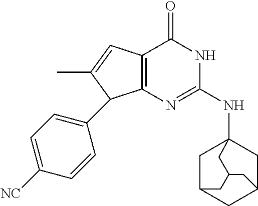 | 398.5 |
| 525 | 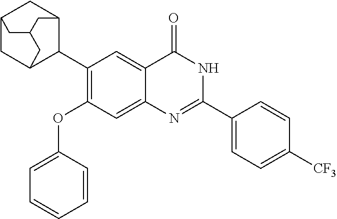 | 516.55 |
| 526 | 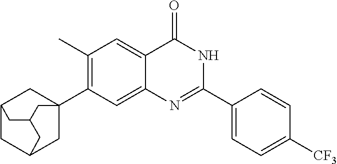 | 438.48 |
| 527 | 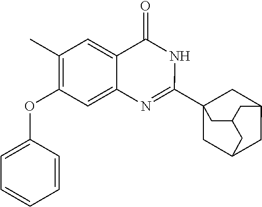 | 386.49 |
| 528 | 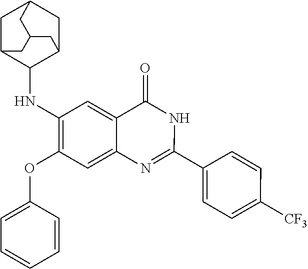 | 531.57 |
| 529 | 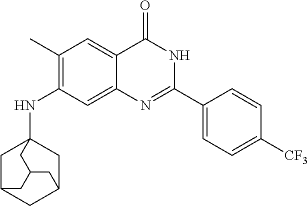 | 453.5 |
| 530 | 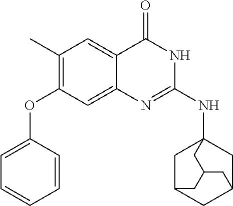 | 401.5 |
| 531 | 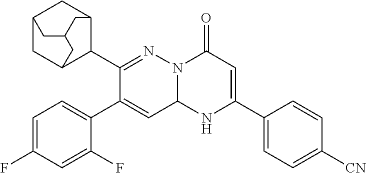 | 496.55 |
| 532 | 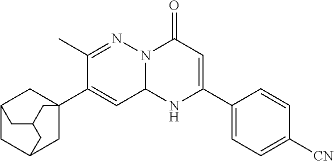 | 398.5 |
| 533 | 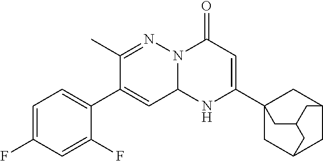 | 409.47 |
| 534 | 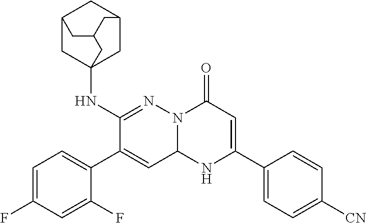 | 511.57 |
| 535 | 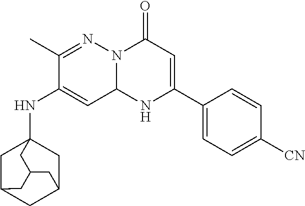 | 413.51 |
| 536 | 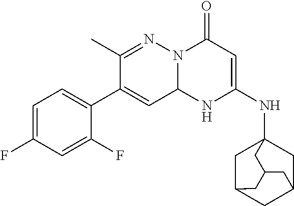 | 424.49 |

### Preparation of Injections

### Example 537

### Preparation of Injection 1

Compound 7, 5.0 g, ethanol 600 ml, 1,2-propanediol 600 ml and Tween (80) 100 ml were dissolved and the injection water was added up to total volume of 5000 ml. The solution was filtered with 0.22 μm membrane filter and sterilized for 30 min at 100° C. to obtain 1000 preparation of injection 5 mg/5 ml.

### Example 538

### Preparation of Injection 2

Compound 33 (example 2) 8.0 g, DMSO 50 ml, 1,2-propanediol 100 ml and Tween (80) 100 ml were dissolved and the injection water was added up to total volume of 5000 ml. The solution was filtered with 0.22 μm membrane filter and sterilized 30 min at 100° C. to obtain 1000 preparation of injection 8 mg/5 ml.

### Example 539

### Antibacterial Studies In Vitro

### Materials and Methods

1. STANDARD STRAINS: Bacillus cereus 2, Bacillus cereus 246, Bacillus subtilis 82, Bacillus subtilis 168, Enterococcus faecalis 29212, Enterococcus faecalis 51299, Enterococcus faecalis 19433, Enterococcus faecalis F2518 (VRE), Enterococcus fecal F631 (VRE), Enterococcus faecalis 1513 (VRE), Enterococcus faecalis 583 (VRE), Staphylococcus aureus 29231, Staphylococcus aureus 43300 (MRSA), Staphylococcus aureus 703 (MRSA), Staphylococcus aureus 704 (MRSA), Staphylococcus aureus 705 (MRSA), Streptococcus pneumoniae 6303 (PRSP), Streptococcus pneumoniae 62, Streptococcus pneumoniae 6301, Pyogenic Streptococcus pneumoniae M2, Pyogenic Streptococcus, Streptococcus agalactiae B, green Streptococcus 1009, Streptococcus bovis 10035, Streptococcus 10342, Streptococcus pneumoniae 10351, Bacillus anthracis 1, Corynebacterium diphtheriae, Clostridium tetani, Clostridium perfringens.

2. TEST SAMPLES: Compound 3, 5, 7, 8, 10, 14, 21, 23, 33, 38, 42, 70 and 78.

Bacterial culture identifications were confirmed by standard microbiological methods. Minimum Inhibitory Concentration (MIC) testing compounds of this invention were tested for antimicrobial activity by susceptibility testing in liquid media.

MICs for compounds against each strain were determined by a broth micro dilution method according to National Committee for Clinical Laboratory standards (NCCLS) guidelines. (NCCLS 2000 Method for dilution antimicrobial susceptibility tests for bacteria that grow aerobically-fifth edition) or followed the descriptions described below:

In this method, an aliquot of test compound from an 11-point series of 2-fold serial dilutions of compounds in 100% DMSO was added to wells in microdilution plates. The test organisms were prepared by adjusting the turbidity of actively broth cultures so that the final concentration of test organism after addition to well with or without compounds was approximately 1.07×10⁷ CFU/ml.

Following inoculation of the miocrodilution plates, the plates were incubated at 37° C. for 24 h and then scored for bacterial growth. The MIC is defined as the lowest concentration of the test compound that completely inhibits the visible growth of the test organism. The amount of growth in the wells containing the test compound was compared with the amount of growth in the growth-control wells (such as ciprofloxacin).

Compounds of the present invention were tested against several bacterial strains. The MIC value (in μm) are presented in Table 3

TABLE 3
Minimum Inhibitory Concentration, MIC (μM) Against Gram-positive Bacteria

| Micro- | Compd. | Compd. | Compd. | Compd. | Compd. | Compd | Compd. | Compd. | Compd. | Compd. | Compd. | Compd. | Compd. |
|---|---|---|---|---|---|---|---|---|---|---|---|---|---|
| organism | 33 | 7 | 21 | 10 | 3 | 70 | 8 | 78 | 42 | 14 | 5 | 23 | 38 |
| A | 1.25 | 0.63 | 0.36 | 0.31 | 2.5 | 0.31 | 0.65 | 5.19 | 0.25 | 20.25 | 10.5 | 110.5 | 123.5 |
| 2 | 1.25 | 1.25 | 1.55 | 1.32 | 0.53 | 1.32 | 2.6 | 1.0 | 0.23 | 30.16 | 0.3 | 200.2 | 154.1 |
| 3 | 0.31 | 0.02 | 0.68 | 0.63 | 5.0 | 1.25 | 1.35 | 1.53 | 0.02 | 10.31 | 0.04 | 110.3 | 120.1 |
| 4 | 1.25 | 0.22 | 0.46 | 0.63 | 2.5 | 1.25 | 0.66 | 1.62 | 1.23 | 12.5 | 0.12 | 101.3 | 212.5 |
| 5 | 2.5 | 0.31 | 0.32 | 1.25 | 0.23 | 2.5 | 0.66 | 10.22 | 20.5 | 15.0 | 0.16 | 220.2 | 145.0 |
| 6 | 2.5 | 2.5 | 10.5 | 2.3 | 0.55 | 2.5 | 0.36 | 11.2 | 21.2 | 21.5 | 0.30 | 130.3 | 106.5 |
| 7 | 5.0 | 0.61 | 12.3 | 0.02 | 0.42 | 0.23 | 11.5 | 10.2 | 18.2 | 13.5 | 2.01 | 140.2 | 108.5 |
| 8* | 2.5 | 0.23 | 2.5 | 0.31 | 0.48 | 0.55 | 0.32 | 121.2 | 18.2 | 122.2 | 31.3 | 215.2 | 115.5 |
| 9* | 0.63 | 0.63 | 0.33 | 0.56 | 0.33 | 0.55 | 0.04 | 221.2 | 21.2 | 221.2 | 19.2 | 80.5 | 151.2 |
| 10* | 5.0 | 0.65 | 2.3 | 0.61 | 1.3 | 2.5 | 0.16 | 152.5 | 20.1 | 130.2 | 35.2 | 152.2 | 150.5 |
| 11* | 2.5 | 0.3 | 0.11 | 0.99 | 0.63 | 1.25 | 0.31 | 16.2 | 30.3 | 120.3 | 32.1 | 131.5 | 160.3 |
| 12 | 0.63 | 5.0 | 0.60 | 0.63 | 0.22 | 0.55 | 10.3 | 0.34 | 0.28 | 6.31 | 5.31 | 281.6 | 100.3 |
| 13* | 0.55 | 2.5 | 0.35 | 1.23 | 0.36 | 0.56 | 0.60 | 122.1 | 150.3 | 120.5 | 213.4 | 120.5 | 210.3 |
| 14* | 0.42 | 2.50 | 0.42 | 0.34 | 1.25 | 1.25 | 1.23 | 150.2 | 172.0 | 125.1 | 135.3 | 130.6 | 215.1 |
| 15* | 0.28 | 0.32 | 0.28 | 0.23 | 0.63 | 0.35 | 10.3 | 250.3 | 230.2 | 160.3 | 150.3 | 180.2 | 160.3 |
| 16* | 0.33 | 0.55 | 0.30 | 0.31 | 0.46 | 0.55 | 50.6 | 122.1 | 180.3 | 210.3 | 123.2 | 150.2 | 210.3 |
| 17* | 1.30 | 0.21 | 1.33 | 0.04 | 0.32 | 0.04 | 1.23 | 120.2 | 203.0 | 250.1 | 105.3 | 230.6 | 125.1 |
| 18 | 0.63 | 0.19 | 0.25 | 0.25 | 10.5 | 10.5 | 0.63 | 18.19 | 0.25 | 100.2 | 10.5 | 160.5 | 140.5 |
| 19 | 2.5 | 5.0 | 0.23 | 0.16 | 0.3 | 0.2 | 2.5 | 15.0 | 0.23 | 140.6 | 0.3 | 170.2 | 140.6 |
| 20 | 1.25 | 2.5 | 0.02 | 0.31 | 0.04 | 0.63 | 1.25 | 12.5 | 0.02 | 120.1 | 0.04 | 200.3 | 156.1 |
| 21 | 0.63 | 2.5 | 1.23 | 2.5 | 0.12 | 1.33 | 0.63 | 21.5 | 1.23 | 132.5 | 20.12 | 231.3 | 126.3 |
| 22 | 0.56 | 0.22 | 0.55 | 5.0 | 0.16 | 0.25 | 0.56 | 35.2 | 0.55 | 225.0 | 0.16 | 200.5 | 105.0 |
| 23 | 0.3 | 1.35 | 1.55 | 1.45 | 0.31 | 0.23 | 0.3 | 10.2 | 1.25 | 125.5 | 0.31 | 210.3 | 101.3 |
| 24 | 10.5 | 2.25 | 1.25 | 1.05 | 2.01 | 0.02 | 10.5 | 12.5 | 1.28 | 100.5 | 2.01 | 153.0 | 121.4 |
| 25 | 0.3 | 0.25 | 1.35 | 1.15 | 1.33 | 1.23 | 0.3 | 13.5 | 15.5 | 170.5 | 1.33 | 123.1 | 131.2 |
| 26 | 0.04 | 1.25 | 1.25 | 1.35 | 1.25 | 0.55 | 0.04 | 11.25 | 1.25 | 103.5 | 1.25 | 155.2 | 136.5 |
| 27 | 35.6 | 80.3 | 13.5 | 35.6 | 25.6 | 12.5 | 56.8 | 68.6 | 32.5 | 122.5 | 52.4 | 166.5 | 265.8 |
| 28 | 80.31 | 71.2 | 30.3 | 70.3 | 62.1 | 45.2 | 30.3 | 125 | 32 | 135.2 | 26.1 | 161.5 | 150.5 |
| 29 | 60.3 | 50.3 | 30.2 | 60.3 | 50.3 | 80.2 | 70.3 | 50.3 | 30.2 | 160.3 | 50.3 | 80.2 | 160.3 |
| 30 | 50.6 | 22.1 | 80.3 | 20.3 | 23.2 | 50.2 | 50.6 | 22.1 | 80.3 | 120.3 | 23.2 | 50.2 | 120.3 |
| 31 | 31.2 | 50.2 | 32.01 | 25.1 | 55.3 | 30.6 | 61.3 | 60.21 | 2.01 | 125.1 | 55.32 | 30.6 | 125.1 |
| 32 | 125.5 | 35.6 | 88.5 | 99.2 | 53.1 | 120.4 | 20.3 | 50.4 | 21.3 | 133.2 | 52.1 | 122.1 | 150.2 |
| 33 | 53.6 | 36.5 | 33.7 | 55.9 | 59.1 | 125.3 | 31.8 | 64.8 | 52.1 | 121.4 | 33.5 | 56.1 | 121.1 |

Note
*is resistant bacteria
1. Bacillus cereus 2; 2. Bacillus cereus 246; 3. Bacillus subtilis 82; 4. Bacillus subtilis 168; 5. Enterococcus faecalis 29212; 6. Enterococcus faecalis 51299; 7. Enterococcus faecalis 19433; 8. Enterococcus faecalis F2518 (VRE); 9. Enterococcus faecalis F631 (VRE); 10*. Enterococcus faecalis 1 513 (VRE); 11*. Enterococcus faecalis 583 (VRE); 12. Staphylococcus aureus 29231; 13*. Staphylococcus aureus 43300 (MRSA); 14*. Staphylococcus aureus703(MRSA); 15*. Staphylococcus aureus 704 (MRSA); 16*. Staphylococcus aureus705 (MRSA); 17*. Streptococcus pneumoniae 6303 (PRSPs); 18. Streptococcus pneumoniae 62; 19. Streptococcus pneumoniae 6301; 20. Pyogenic Streptococcus pneumoniae (M2); 21. Pyogenic Streptococcus; 22. Streptococcus agalactiae group B; 23. green Streptococcus 1009; 24. Streptococcus bovis 10035; 25. Streptococcus 10342; 26. Streptococcus pneumoniae 10351; 27. Bacillus anthracis 1; 28. Corynebacterium diphtheriae; 29. Clostridium tetani; 30. Clostridium tetani; 31. Clostridium perfringens; 32. Oral candidiasis; 33. Dermatophytosis.

### Conclusion:

Table 3. showed the compounds of the present invention possess excellent antibacterial activity against gram-positive bacteria. It is especially noteworthy that the compounds of the invention are effective against Staphylococcus aureus (MRSA). The MICs of the Examples compound 7, 21, 10, 3, 70 and 8 significantly inhibited the growth of Enterococcus faecalis (VRE), Staphylococcus aureus (MRSA), Streptococcus pneumoniae 6303 (PRSP); Bacillus cereus, Bacillus subtilis, Enterococcus faecalis, Staphylococcus aureus, Streptococcus pneumoniae, Streptococcus agalactiae B and streptococci 4; Compounds 78 significantly inhibited the growth of Bacillus cereus and Enterococcus faecalis; Compound 42 and 14 significantly inhibited the growth of the majority of Bacillus cereus, Bacillus subtilis and Enterococcus faecalis; Compound 5 and 78 significantly inhibited the growth of the majority of Bacillus cereus, Bacillus subtilis and Enterococcus faecalis.

### Example 540

### Antibacterial Studies In Vivo

### 1 Materials

Test Samples: Compound 3, 7, 21, 33 and 70

Experimental animals: Kunming healthy mice (19˜21 g) provided by the Animal Center of Beijing Academy of Military Medical Sciences Institute of Materia Medica.

### Strains: MRSA 2152

### 2 Methods

Seventy mice were randomly divided into seven groups with half male and half female, blank control group, positive control group, test group (n=10). 0.2 ml/10 g of the cell suspension (MRSA 2152, concentration of 5.0×106 cfμ/ml) was administered into the abdominal cavity of each mouse. Then the test compound was administered by tail vein. The administration of test compound was repeated after six hours. After this inoculation, the survival rate and the morbidity rate (associated with body weight loss etc.) were determined on 30 day after inoculation.

Survival rate %=(survival days of test group−survival days of blank group)/survival days of blank group×100%

### 3 Results

TABLE 4
The average survival days and life prolongation rate

| | | Cipro- | Compd. | Compd. | Compd. | Compd. | Compd. |
|---|---|---|---|---|---|---|---|
| | saline | floxacin | 3 | 7 | 21 | 33 | 70 |
| Dose | — | 20 | 30 | 40 | 50 | 25 | 70 |
| (mg/kg) | | | | | | | |
| Survival | 1.3 | 3.4 | 4.3 | 5.6 | 3.2 | 3.5 | 4.6 |
| (day) | | | | | | | |
| Survival | — | 161.5 | 216.8 | 330.7 | 133.2 | 169.2 | 253.8 |
| rate (%) | | | | | | | |

The studies in vivo showed the inhibition of MRSA 2152 with life survival rate of over 50% by compound 70, 7, 21, 3 and 33.

1. A compound of the formula I: 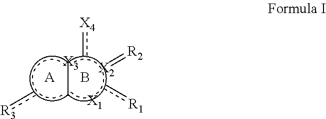
or stereoisomers, tautoers, prodrug, pharmaceutically acceptable salts, complex salts or solvates thereof, wherein:
The dotted lines are independently at each occurrence, optionally substituted single bonds, optionally substituted double bond or a optionally substituted heterocyclic group containing carbon, oxygen, sulfur or nitrogen element; A cycle is of 3-8 saturated or unsaturated arylheterocycles or aliheterocyclic, containing 1-4 heteroatoms, B cycle 5-8 member saturated or unsaturated X₁, X₂, X₃, X₄ are, independently at each occurrence, C, O, S, Se, N and P elements, or contains a substituted C, O, S, Se, N, P elements existing independently or combination; R₁, R₂, R₃ is a substituent containing alicyclic group, arylcycle group, heterocyclic group, adamantane alkyl, adamantane heterocycle, substituent adamantane, adamantane analogs, sugar group, hydroxyl group, amino acid group, the substituent containing O, S, Se, N and/or P, the hydrocarbon chain containing O, S, Se, N or P atom, cycle group and one or a combination of the above substituents.

2. A compound according to the claim 1, wherein:
When X₁, X₂, X₃ or X₄ is C element, they are the same or different substituents of C═O, C═Rb—Ra, CHOH, CHORb or CHRb, X₁ and X₂; When X₁, X₂, X₃ or X₄ is O element, they are the same or different substituents of —O—,—O═Rb—Ra; When X₁, X₂, X₃ or X₄ is S element, they are the same or different substituents of divalent, tetravalent, hexavalent S, ═S═Rb—Ra; when X₁, X₂, X₃ or X₄ is N elements, they are the same or different substituents of —NH—, ═NH, ═N—Rb—Ra; when X₁, X₂, X₃ or X₄ is P elements, they are the same or different substituents of trivalent phosphine, pentavalent phosphine, —PH2, ═NH, ═PRb—Ra; when the X₃ is heteroatoms, the A cycle can be formed by the combination of heteroatoms with the C, O, S, Se, N, P elements to form carbon-hetero bind or hetero-hetero bind, wherein:
Ra and Rb are the same or different substituents; Rb is C, N, P atoms; Ra is hydrogen, halogen, hydroxyl, thiol, cyano, carbonyl, substituted carbonyl, aldehyde, ketone, nitro, carboxyl, substituted carboxyl, carboxylic ester, amino, substituted amino, alkyl, alkoxy, aryl alkoxy, aryloxy, heteroaryloxy, alkylthio, arylalkylthio, arylthio, heteroarylthio, amino, amino-alkoxy, saturated or unsaturated heterocyclic, heterocyclic alkoxy or alkyl amino heterocyclic group to form a variety of substituents with double bonds can also form a new straight chain, branched-chain alkane group or hydrocarbon substituents containing fat alkyl, double bond or triple bond of saturated or unsaturated fatty alkyl, alicycle, alicycle, aliheterocyclic, aromatic, arylheterocyclic fused heterocyclic group or a combination thereof;
Substituent is, independently at each occurrence, optionally substituted C₁₋₁₂ optionally substituted saturated or unsaturated alkyl, 1-4 optionally substituted double bond, 1-4 optionally substituted triple bond, optionally substituted or saturated, unsaturated C₁₋₁₀ optionally alicyclic, C₁₋₁₀ substituted aryl group or C₁₋₁₀ optionally substituted heterocyclic, where contains optionally substituted one or combination of oxygen, sulfur, nitrogen, phosphorus element, halogen, saturated or unsaturated 3-7 membered alicyclic, aryl cyclic, multi-cyclic, aliphatic heterocyclic, aryl heterocyclic, fused heterocyclic, alkyl, cycle, aryl or heterocyclic group or a combination thereof;
Cyclic group is, independently at each occurrence, optionally substituted alicyclic, arylcyclic, aliheterocyclic or arylheterocyclic group, 3-8 membered cycle; alkyl is, independently at each occurrence, alialkyl, arylalkyl;
Glycosyl group is, independently at each occurrence, optionally substituted D- and L-configuration with C—C or C-Hetero bond of glycoside, substituent of 1-8 glycosyl, multi-hydroxyl, multi-hydroxyl, phenol, polyphenol, amino acid;
Substituent is, independently at each occurrence, optionally substituted chain alkyl containing hydroxyl, alkoxy, ester, acyloxy, phosphate oxy, sulfonyloxy, aryloxy heterocyclic oxy, thiol, alkyl thiol, alkthiol, thiolester, aryl thiol, heterocyclic thiol, ether group, 4-8 membered arylcyclic, 4-8 membered heterocyclic, 4-8 membered alicyclic, 4-8 membered arylheterocyclic, bridge cyclic, spiral cyclic, adamantane cyclic, amino, primaryl amine, secondaryl amino, tertiaryl amino, quaternaryl ammonium, amide, hydrazine, oxime, hydrazone-based, phosphide, phosphate, phosphate group, O, S, Se, N or P element;
R₁, R₂, R₃ is independently at each occurrence, optionally substituted the same or different substituents of hydrogen, halogen, hydroxyl, thiol, cyano, carbonyl, aldehyde, ketone, nitro, carboxyl, carboxylic ester, alkyl, alkoxy, aryloxy, alkylthio, arylthio, amino, aminoalkoxy, heterocyclic, alkylalkoxy, acyl(RaCO), carbamoyl (RbRcNCO) or sulfonyl (RdSO2), wherein:
Ra, Rb, Rc and Rd is the same or different substituents of hydrogen, halogen, hydroxyl, thiol, cyano, carbonyl, aldehyde, ketone, nitro, carboxyl, carboxylic ester, alkyl, alkoxy, arylalkoxy, alkyl sulfur, arylalkylthio, arylthio, amino, aminoalkoxy, heterocyclic, heterocyclicoxy, heterocyclic alkyl amino or double bond containing substituents(═X₅), wherein:
X₅ is independently at each occurrence, optionally substituted the same or different
substituents containing O, S, Se, N or P element;
substituent is independently at each occurrence, optionally substituted the same or different R₁ and R₂ of two cycles to form a new cycle or combination of two cycles;
R₁, R₂, R₃ or X₄ is independently at each occurrence, optionally substituted the same or different substituents of H, halogen or XRa; wherein:
X is optionally substituted group containing C, O, S, Se, N, and/or the P element;
A compound of arylheterocycle-fused pyrimidines, derivatives and analogs is selected from the exemplified examples or stereoisomers, tautomers, pharmaceutically acceptable salts, inorganic acid salt, organic acid salt, organic basic salt, organic basic salt, complex salt, prodrug or solvates thereof in association with a pharmaceutically acceptable excipient or carrier.

3. A compound according to the claims 1 and 2, wherein:
A process for the manufacture of a compound of formula I comprises:
For the preparation of compounds of formula I and salts thereof in which the reaction of a pyrimidinylcycle fused arylcycle to form A-cycle, B-cycle, X₁, X₂, X₃, X₄, R₁, R₂, R₃ with a bond of C—C, C—O, C—S, C—N or C—P under catalysis at −78° C. to 90° C., wherein;
preparation of o-amino-nitrogen containing heterocycle analogs, the key intermediates to form the pyrimidinyl fused arylcycle by a solvent selected from THF, 1,4-dioxane, N,N-dimethylformamide amide, N,N-dimethylacetamide or toluene, the reaction temperature from room temperature to 180° C., a catalyst selected from p-toluenesulfonate, Lewis acid, sulfuric acid, molecular sieves, alumina, silica gel or dehydration agent;
preparation of arylcycle fused pyrimidine analogs: by a solvent selected from ethanol, THF, 1,4-dioxane, N,N-dimethylformamide amide, N,N-dimethylacetamide or toluene, the reaction temperature from room temperature to 180° C., a catalyst selected from p-toluenesulfonate, Lewis acid, sulfuric acid, molecular sieves, alumina, silica gel or dehydration agent;
preparation of Heterocyclic cycle fused pyrimidine analogs: by a solvent selected from ethanol, THF, 1,4-dioxane, N,N-dimethylformamide amide, N,N-dimethylacetamide or toluene, the reaction temperature from room temperature to 180° C., a catalyst selected from p-toluenesulfonate, Lewis acid, sulfuric acid, molecular sieves, alumina, silica gel or dehydrationagen.

4. A method according to the claim 1 and 2, wherein:
a compound for treating, preventing or slowing the progression of bacteria, fungi and other infections caused by known antibacteria, antifungal, anti-inflammatory and anti-viral and immune compatibility of drug use, including inflammation associated with bacterial infections and inflammatory diseases, fungi and fungal diseases, viruses and viral diseases and immune system disease, alone or with the following drugs known to be used in conjunction dose of 0.02 mg/kg-250 mg/kg (intravenous, intramuscular, oral, topical and other routes of administration); means of various methods of treatment and therapy, where the bacteria are Gram-positive bacteria, Staphylococcus aureus, Streptococcus pneumoniae, Enterococcus faecalis, Streptococcus, Streptococcus bovis, Streptococcus pneumoniae, Peptostreptococcus, Streptococcus pneumonia, Streptococcus pneumoniae, Streptococcus pyogenes, Streptococcus agalactiae, Streptococcus bovis, Streptococcus agalactiae B, Clostridium tetani, Bacillus cereus, Bacillus subtilis, Clostridium perfcycleens, Bacillus cereus, Bacillus subtilis, Bacillus anthracis, Clostridium bacillus, Clostridium perfcycleens, Clostridium perfcycleens spirochetes, M. tuberculosis, suppurative, purulent, diphtheria, tetanus bacilli, erysipelas bacillus, Bacillus anthracis, actinomycetes, Gram-positive bacteria resistant to methicillin-resistant Staphylococcus aureus, vancomycin-resistant gold Pu bacteria, Staphylococcus inducible clindamycin resistance, vancomycin-resistant enterococci, aminoglycoside-resistant enterococci high-level penicillin-resistant Streptococcus pneumoniae, multi-drug resistant Acinetobacter baumannii and multiple drug resistance drug-resistant Mycobacterium tuberculosis and Mycobacterium tuberculosis, Streptococcus, Enterococcus faecalis, Pseudomonas aeruginosa, Escherichia coli and Acinetobacter spp, drug-resistant Haemophilus influenzae, Neisseria gonorrhoeae resistance, resistance Neisseria meningitidis, Enterobacteriaceae resistant or resistant Pseudomonas aeruginosa.

5. A method according to the claim 4, wherein:
a compound for treating, preventing or slowing the progression of bacteria and fungi and other infections caused by various infections including bacterial infection associated with inflammation and inflammatory diseases, fungi and fungal diseases, viral and viral diseases and immune system complications: upper and lower respiratory tract infections, skin and soft tissue infections, urinaryl tract infections, septicemia and endocarditis infection induced by methicillin-sensitive Staphylococcus aureus, hemolytic streptococci and Streptococcus pneumoniae; the infections of urinary tract and pneumonia induced by Streptococcus, Streptococcus pneumoniae, Haemophilus influenzae, Proteus mirabilis, Escherichia coli and other Gram-positive cocci; the infections of respiratory tract, urinary tract, skin and soft tissue, sepsis, bone and joint, abdominal, pelvic viridans, endocarditis, anaerobic, anthrax, syphilis or gonorrhea induced by hemolytic streptococcus and enterococci.

6. According to claim 1 and 2, wherein said compounds is administered together with at least one known antibacterial agents, antifungal agents or anti-inflammatory agents selected from penicillin, procaine penicillin, benzathine penicillin, methicillin, oxacillin, cloxacillin, dicloxacillin, hetacillin, sulbenicillin, temocillin, mecillinam, piperacillin, ticarcillin, ticarcillin, flucloxacillin, sultamicillin, phenoxymethylpenicillin, bacampicillin, ticarcillin, talampicillin, furbenicillin, aspoxicillim, pivampicilli, meticillin, nafcillin, pivmecillinam, lenampicillin varacillin, apalcillin, carindacillin, carbenicillin, ceftriaxone, cefpirome, cefuroxime, ceftazidime, cefotaxime, cephalothin, cefathiamidine, cephalosporin, cephalosporin, cefazolin, cefmenoxime, cefoperazone, cefaclor, ceftizoxime, cefdinir, cefixime, cephradine, cefpiramide, cephalosporin, cephalosporin, cefpodoxime, cefodizime, cefotiam, cefetamet, cefprozil, cephalosporin, cefepime, cephalexin, cephradine, cefadroxil, cefoxitin, cefmetazole, cefotetan, cefminox, cephalosporin, cefozopran, cephalosporin, cefalotin, cefaloglycine, cefalexin, cefradine, cefacetrile cefapirin, cefadril, cefroxadine, ceftezole, cefonicid, cefamandole, cefbuperazone, cefdinir, cefzon, cefcapene, cefotaxime, cefteram, cefsulodine, latamoxef, cefpimizole, cefuzonam, aztreonam, erythromycin, roxithromycin, adriamycin, clarithromycin, fluoerythromycin, azithromycin, kitasamycin, albomycin, leucomycin, josamycin, spiramycin, acetyl spiramycin, netilmicin, isepamicin, streptomycin, gentamicin, tobramycin, amikacin, sisomicin, neomycin, neomycin, spectinomycin, streptomycin, kanamycin, chloramphenicol, thiamphenicol, lincomycin, clindamycin, clindamycin, cyclosporine, teicoplanin, vancomycin, teicoplanin, bacitracin, polymyxin B, rifamycin, rifabutin, rifapentine, rifaximin, rifampicin, rifamycinn, enoxacin, norfloxacin, ciprofloxacin, lomefloxacin, sparfloxacin, pefloxacin, fleroxacin, moxifloxacin, ofloxacin, levofloxacin, rufloxacin, isoxazole, sulfamethoxazole, sulfadiazine, trimethoprim, pipemidic acid, nitrofurantoin, furazolidone, nalidixic acid, difloxacin, gatifloxacin, pazufloxacin, moxifloxacin acid, tetracycline, minocycline, chlortetracycline, doxycycline, oxytetracycline, doxycycline, metacycline, clavulanic acid, sulbactam, tazobactam, imipenem, cilastatin, panipenem, betamipron, meropenem, cephamycin, sulfamethoxazole, sulfamethazine, sulfonamides, sulfamonomethoxine, sulfamethoxazole, sulfathiazole, sulfamethoxazole, sulfathiazole, thienamycin, aztreonam, faropenem, cilastatin, tazobactam, streptomycin, neomycin, kanamycin, neomycin, clindamycin, fosfomycin, brodimoprim, metronidazole, aclarubicin, epirubicin, pirarubicin, mupirocin, timidazole, sulfasalazine, itraconazole, bifonazole, fluconazole, clotrimazole, econazole, miconazole, ketoconazole, naftifine, butenafine, ciclopirox, amorolfine, amphotericin B, flucytosine, terbinafine, nystatin, Gentamycin, nebramycin, micronomicin, ribostamycin, astromicin, dibekacin, etimicin, nofloxacin, cotrim, sulfamethoxazole, mafenide, brodimoprim clafalix, laurylin, azitromycin, midecamycin, acetylmidecamycin, rokitamycin, meleumycin, methacycline, doxycyclme, demeclocycline, nifuratel, methylmercadone, norvancomycim, colistin, gramicidin, isoniazid, ethambutol, pyrazinamide, rifamycin, rifandin, viomycin, rifampin, capreomycin, ethionamide, terconazole, fenticonazole, sulconazole, fluorocytosine, ciclopirox, mepartricin, exalamide, terbinafine, ribavirin, acyclovir, ganciclovir, indinavir, nelfinavir, ritonavir, cidofovir, penciclovir, buciclovir, penciclovir, famciclovir, valaciclovir, famotine, vidarabine, zidovudine, azidothymidine, foscarnet, delavirdine, moroxydine, idoxuridine, amantadine, interferons, rimantadine, clindamycim.

7. A compound according to the claim 1, wherein:
A compound of arylheterocycle-fused pyrimidines, derivatives and analogs fused by the formation of three-membered A-cycle, three membered cycle is, independently at each occurrence, selected from the example 1 to 536 and following list but not limiting of the method and composition of the present invention selected from:
4-(4-(2,4-dichlorophenyl)-2-oxo-3,5-diaza-bicyclo[4.1.0]hept-3-en-7-yl)benzonitrile, 4-(7-(2,4-difluorophenyl)-2-methyl-5-oxo-2,4-diazabicyclo[4.1.0]hept-3-en-3-yl)benzonitrile, 4-(5-oxo-7-(4-(trifluoromethyl)phenyl)2,4-diazabicyclo[4.1.0]hept-3-ene-3-yl)benzonitrile, 7-(2,4-dichlorophenyl)-4-(4-(trifluoromethyl)phenyl)-3,5-diazabicyclo[4.1.0]hept-3-en-2-one, 4,7-bis(4-(trifluoromethyl)phenyl)-3,5-diazabicyclo[4.1.0]hept-3-ene-2-one, 7-(4-(trifluoromethyl)phenyl)-4-(4-trifluoromethyl)pyridin-3-yl)-3,5-diazabicyclo[4.1.0]hept-3-ene-2-one, 4-(2,4-difluorophenyl)-7-(4-(trifluoromethyl)phenyl)-3,5-diazabicyclo[4.1.0]hept-3-en-2-one, 7-(2,4-difluorophenyl)-4-(4-methoxyphenyl)-3,5-diazabicyclo[4.1.0]hept-3-ene-2-one, 4-(4-aminophenyl)-7-(2,4-difluorophenyl)-3,5-diazabicyclo[4.1.0]hept-3-ene-2-one, 4-(7-(2,4-difluorophenyl)-5-oxo-2,4-diazabicyclo[4.1.0]hept-3-en-3-yl)-N,N-dipropylbenzene sulfonamide, 4-(7-(2,4-dichlorophenyl)-5-oxo-2,4-diazabicyclo[4.1.0]hept-3-en-3-yl)benzonitrile, 4-(4-(4-cyanophenyl)-2-oxo-3,5-diazabicyclo[4.1.0]hept-3-en-7)benzoic acid;
The formation of four-membered A-cycle, 4-(7-(2,4-difluorophenyl)-8-methyl-5-oxo-2,6-diazabicyclo[4.2.0]octa-1(8)-2-dien-3-yl)benzonitrile, 4-(8-(2,4-difluorophenyl)-2,7-dimethyl-5-oxo-2,6-diazabicyclo[4,2,0]oct-3-en-3-yl)benzonitrile, 4-(7-methyl-5-oxo-8-(4-trifluoromethyl)phenyl-1,6-diazabicyclo[4,2,0]oct-3-en-3-yl)benzonitrile, 7-(2,4-difluorophenyl)-8-methyl-4-(4-(trifluoromethyl)phenyl-1,5-diazabicyclo[4,2,0]oct-3-ene-2-one, 8-methyl-4,7-bis(4-(trifluoromethyl)phenyl-1,5-diazabicyclo[4,2,0]oct-3-ene-2-one, 8-methyl-7-(4-(trifluoromethyl)phenyl-4-(6-trifluoromethyl)pyridin-3-yl)-1,5-diazabicyclo[4,2,0]oct-3-ene-2-one, 4-(2,4-difluorophenyl)-8-methyl-7-(4-(trifluoromethyl)phenyl)-1,5-diazabicyclo[4,2,0]oct-3-ene-2-one, 7-(2,4-difluoro-4-(4-methoxyphenyl))-8-methyl-1,5-diazabicyclo[4,2,0]oct-3-ene-2-one, 4-(4-aminophenyl)-7-(2,4-difluorophenyl)-8-methyl-1,5-diazabicyclo[4,2,0]oct-3-ene-2-one, 4-(8-(2,4-difluorophenyl)-7-methyl-5-oxo-2,6-diazabicyclo[4,2,0]oct-3-en-3-yl)aminomethyl)benzonitrile, 4-(8-(2,4-difluorophenyl)-7-methyl-5-oxo-2,6-diazabicyclo[4,2,0]oct-3-en-3-yl)-N,N-dipropylbenzenesulfonamide, 4-(2,4-difluorophenyl)-4H-8-ethyl-7-(4-(trifluoromethyl)phenyl-1,5-diazabicyclo[4,2,0]oct-3-ene-2-one, 4-(8-(2,4-difluorophenyl)-5-oxo-7-phenyl-2,6-diazabicyclo[4,2,0]oct-3-en-3-yl)benzonitrile, 7-(2,4-difluorophenyl)-8-phenyl-4-(4-(trifluoromethyl)benzenyl)-1,5-diazabicyclo[4,2,0]oct-3-ene-2-one, 4-(7-(cyclopropyl-8-(2,4-difluorophenyl))-5-oxo-2,6-diazabicyclo[4,2,0]oct-3-en-3-yl)benzonitrile, 4-(8-(2,4-difluorophenyl)-5-oxo-7-(4-(trifluoromethyl)phenyl)-2,6-diazabicyclo[4,2,0]oct-3-en-3-yl)benzonitrile, 4-(8-(2,4-difluorophenyl)-7-ethyl-5-oxo-2,6-diazabicyclo[4,2,0]oct-3-ene-2-yl)benzonitrile, 4-(8-(2,4-dichlorophenyl)-7-methyl-5-oxo-2,6-diazabicyclo[4,2,0]oct-3-en-3-yl)benzonitrile, 4-(4-(4-cyanophenyl)-8-methyl-2-oxo-1,5-diazabicyclo[4,2,0]oct-3-en-7-yl)benzoic acid;
The formation of five-membered A-cycle, Ethyl2-methyl-7-oxo-3-(4-(trifluoromethyl)phenyl)-4,7-dihydropyrazolo[1,5-a]pyrimidinyl-6-carboxylate, 3-(2,4-dichlorobenzene)-2-methyl-5-(4-(trifluoromethyl)phenyl)pyrazolo[1,5-a]pyrimidin-7(4H)-one, 3-(2,4-difluorophenyl)-2-phenyl-4,7-dihydropyrazolo[1,5-a]pyrimidin(7-one-5-yl)benzonitrile, 4-(3-(2,4-dichlorophenyl)-2-ethyl-7-oxo-4,7-dihydropyrazolo[1,5-a]pyrimidin-5-yl)benzonitrile, 4-(2-cyclopropyl-3-(2,4-dichlorophenyl)-7-oxo-4,7-dihydropyrazolo[1,5-a]pyrimidin-5-yl)benzonitrile, 3-(2,4-dichlorophenyl)-2-methyl-5-(4-(trifluoromethyl)phenyl)pyrazolo[1,5-a]pyrimidin-7(4H)one, 3-(2,4-dichlorophenyl)-2-methyl-5-(6-(trifluoromethyl)pyridin-3-yl)pyrazolo[1,5-a]pyrimidin-7(4H)-one, 3-(2,4-dichlorophenyl)-2-methyl-5-(4-nitrophenyl)pyrazolo[1,5-a]pyrimidin-7(4H)-one, 3-(2,4-dichlorophenyl)-2-methyl-5-(morpholinomethyl)pyrazolo[1,5-a]pyrimidin-7(4H)-one, ethyl(3-(2,4-dichlorophenyl)-2-methyl-7-oxo-4,7-dihydropyrazolo[1,5-a]pyrimidine)-6-carboxylate, 4-(3-(2,4-dichlorophenyl)-2-methyl-7-oxo-4,7-dihydropyrazolo[1,5-a]pyrimidin-5-yl)-N,N-dipropylbenzoyl sulfonamide, 4-((3-(2,4-dichlorophenyl)-2-methyl-7-oxo-4,7-dihydropyrazolo[1,5-a]pyrimidin-5-yl)aminomethyl)benzonitrile, 4-(3-(2,4-dichlorophenyl)-7-oxo-2-((trifluoromethyl)phenyl)-4,7-dihydropyrazolo[1,5-a]pyrimidin-5-yl)benzonitrile, ethyl 2-cyclopropyl-3-(2,4-dichlorophenyl)-7-oxo-4,7-dihydropyrazolo[1,5-a]pyrimidinyl-6-carboxylate, 3-(2,4-dichlorophenyl)-2,6-dimethyl-4,7-dihydro-5-(4-(trifluoromethyl)phenyl)pyrazolo[1,5-a]pyrimidin-7-one, 2-cyclopropyl-3-(2,4-dichlorophenyl)-6-methyl-4,7-dihydro-5-(4-(trifluoromethyl)phenyl)pyrazolo[1,5-a]pyrimidin-7-one, 3-(2,4-dichlorophenyl)-6-methyl-2-phenyl-4,7-dihydro-5-(4-(trifluoromethyl)phenyl)pyrazolo[1,5-a]pyrimidin-7-one, 3-(2,4-dichlorophenyl)-2-ethyl-6-methyl-4,7-dihydro-5-(4-(trifluoromethyl)phenyl)pyrazolo[1,5-a]pyrimidin-7-one, 2,6-dimethyl-4,7-dihydro-3,5-bis(4-(tri-fluoromethyl)phenyl)pyrazolo[1,5-a]pyrimidin-7-one, 4-(3-(2,4-dichlorophenyl)-2,6-dimethyl-7-oxo-4,7-dihydropyrazolo-[1,5-a]pyrimidin-5-yl)-N,N-dipropyl benzenesulfonamide, 4-(3-(2,4-dichlorophenyl)-6-methyl-7-oxo-2-phenyl-4,7-dihydropyrazolo[1,5-a]pyrimidin-5-yl)-N,N-dipropyl benzene sulfonamide, 4-(2-cyclopropyl-3-(2,4-dichlorophenyl)-6-methyl-7-oxo-4,7-dihydropyrazolo[1,5-a]pyrimidin-5-yl)benzonitrile, ethyl 2-m-7-oxo-3-(4-(trifluoromethyl)phenyl)-4,7-dihydro pyrazolo[1,5-a]pyrimidin-6-carboxylate, 4-(3-(2,4-dichlorobenzene)-2,6-dimethyl-7-oxo-4,7-dihydro-pyrazolo[1,5-a]pyrimidin-5-yl)benzonitrile, 3-(2,4-dichlorobenzene)-2-ethyl-6-methyl-4,7-dihydro-5-(6-(trifluoromethyl)pyridin-3-yl)pyrazolo[1,5-a]pyrimidin-7-one, 7-amino-3-(2,4-dichlorophenyl)-2-methylpyrazolo[1,5-a]pyrimidin-6-nitrile, 1-imino-2-phenyl-4H-7-methyl-8-(2,4-dichloro phenyl)pyrazolo[1,5-1′,2′]pyrimido[5′,6′-d]pyrimidin-2-thione, 4-(2,6-dimethyl-7-oxo-3-(4-(trifluoromethyl)phenyl)-4,7-dihydropyrazolo[1,5-a]pyrimidin-5-yl)benzonitrile, 5-amino-3-(2,4-dichlorophenyl)-2-methyl-7-(3-nitrophenyl)-4,7-dihydropyrazolo[1,5-a]pyrimidinyl-6-nitrile, 3-(2,4-dichlorophenyl)-4-ethyl-7-(3-nitrophenyl)indanono-1[2,3-5′,6′]-1,2,6,7-tetrahydropyrimido[2′,3′-b]pyrazole, 4-imin-1,5-diphenyl-4,5-dihydro-1H-pyrazolo[3,4-d]pyrimidin-6(7H)thione, 6-(2,4-dichlorophenyl)-4-ethoxy-1-phenyl-1H-pyrazolo[3,4-d]pyrimidine, 1,6-diphenyl-4-amino-1H-pyrazolo[3,4-d]pyrimidine, diethyl 4-amino-1-phenyl-1H-pyrazolo[3,4-b]pyridin-5,6-dicarboxylate, 5-di-phenyl-1H-pyrazolo[3,4-d]pyrimidin-4(5H)imine, 3-(2,4-dichlorobenzene)-5-hydroxy-2-methyl-6-phenylpyrazolo[1,5-a]pyrimidin-7(4H)one, 5-hydroxy-6-phenyl-2-(4-(trifluoromethyl)phenyl)pyrazolo[1,5-a]pyrimidin-7(4H)one, 2-(4-(trifluoromethyl)phenyl)benzo[4,5]imidazo[2,1-b]-1,11-dihydropyrimidin-4-one, 3-(2,4-dichlorophenyl)-6-(4,5-dihydro-1H-imidazole-2-yl)-2-methylpyrazolo[1,5-a]pyrimidin-7-amine, 4-phenyl-3H-[1,2,5]triazoheptadieno[5,4-a]indole-1-amine, 2-amino-4,5-diphenyl-4H-imidazole-4-alcohol, 4,5-diphenyl-1H-imidazole-2-amine, 7-methyl-2,3-diphenylimidazo[1,2-a]pyrimidin-5(8H)one, 4-(3-(2,4-dichlorophenyl)-2-methyl-6-nitro-4,7-dihydropyrazolo[1,5-a]pyrimidin-7-one-5-yl)benzoic acid, 3-(2,4-dichlorophenyl)-2-methyl-4,7-dihydro-6-nitro-5-(4-(trifluoromethyl)phenyl)pyrazolo[1,5-a]pyrimidin-7-one, 4-(3-(2,4-dichlorophenyl)-7-H-2,4-dimethylpyrazolo[1,5-a]pyrimidino-7-one-5-yl)benzonitrile, 2-methyl-3-(4-(trifluoromethyl)phenyl)-5-(6-(trifluoromethyl)pyridin-3-yl), 3-(2,4-difluorophenyl)-4H-2-phenyl-5-(4-(trifluoromethyl)phenyl)pyrazolo[1,5-a]pyrimidin-7-one, 5-(chloromethyl)-3-(2,4-dichlorophenyl)-2-methyl pyrazolo[1,5-a]pyrimidin-7(4H)one, 3-(2,4-dichlorophenyl)-2-ethyl-4,7-dihydro-5-(4-(trifluoromethyl)phenyl)pyrazolo[1,5-a]pyrimidin-7-one, 3-(2,4-dichlorophenyl)-2-methyl-5-((pyridin-2-yl)aminomethyl)pyrazolo[1,5-a]pyrimidin-7(4H)one, 3-(2,4-dichlorophenyl)-6-methyl-7-oxo-2-phenyl-7-dihydropyrazolo[1,5-a]pyrimidin-5-ylbenzonitrile, 4-(3-(2,4-dichlorophenyl)-2-ethyl-6-methyl-7-oxo-4,7-dihydropyrazolo[1,5-a]pyrimidin-5-yl)benzonitrile, 2,6-dimethyl-4,7-dihydro-3-(4-(trifluoromethyl)phenyl)-5-(6-(trifluoromethyl)pyridin-3-yl)pyrazolo[1,5-a]pyrimidin-7-one, 4-(2-cyclopropyl-3-(2,4-dichlorophenyl)-6-methyl-7-oxo-4,7-dihydropyrazolo[1,5-a]pyrimidin-5-yl)-N,N-dipropyl benzene sulfonamide, 5-oxo-2,3-diphenyl-5,8-dihydroimidazo[1,2-a]pyrimidin-6-carboxylic acid, N-(furan-2-sulfoxidemethyl)-4-imin-1-phenyl-1H-pyrazolo[3,4-d]pyrimidin-5(4H)amine, (Z)-2-(hydroxymethyl)-6-(4-((4-imin-1-phenyl-1H-pyrazolo[3,4-d]pyrimidin-5(4H) amino)methyl)phenoxy)tetrahydro-2H-pyran-3,4,5-triol, 5-amino-7-(4-chlorophenyl)-3-(2,4-dichlorophenyl)-2-methylpyrazolo[1,5-a]pyrimidin-6-nitrile, 7-amino-2,3-diphenylimidazo[1,2-a]pyrimidin-6-cyanide, 7-amino-2,3-diphenylimidazo[1,2-a]pyrimidin-6-amide, 5-chloro-7-methyl-2,3-diphenylimidazo[1,2-a]pyrimidine, 4-(2,4-dichlorophenyl)-3-(3-methoxyphenyl)-1H-pyrazol-5-amine, 4-(3-(2,4-dichlorophenyl)-2-(3-methoxyl-phenyl)-6-methyl-7-oxo-4,7-dihydropyrazolo[1,5-a]pyrimidin-5-yl)benzonitrile, 4-(3-(2,4-chlorophenyl)-2-hydroxy-6-dimethyl-7-ox-4,7-dihydropyrazolo[1,5-a]pyrimidin-5-yl)benzonitrile, 3-(2,4-dichlorophenyl)-2-methyl-5-(amantadinylmethylene) pyrazolo[1,5-a]pyrimidin-7(4H)one, 4-(3-(2,4-difluorophenyl)-4H-2-methylpyrazolo[1,5-a]pyrimidin-7-oxo-5-yl)benzonitrile, 4-(3-(2,4-difluorophenyl)-4H-2,6-methylpyrazolo[1,5-a]pyrimidin-7-oxo-5-yl)benzonitrile, 4-(3-(2,4-difluorophenyl)-4H-2,4-methylpyrazolo[1,5-a]pyrimidin-7-oxo-5-yl)benzonitrile, 4-(3-(2,4-difluorophenyl)-7-H-2,4,6-trimethylpyrazolo[1,5-a]pyrimidin-7-oxo-5-yl)benzonitrile, 4-(3-(2,4-difluorophenyl)-4,7-dihydro-2-methylpyrazolo[1,5-a]pyrimidin-7-oxo-6-methyl-5-yl)benzonitrile, 4-(3-(2,4-difluorophenyl)-7-oxo-4,7-dihydro-2-methyl-5-hydroxypyrazolo[1,5-a]pyrimidin-6-one, ethyl 4-(3-(2,4-difluorophenyl)-4H-2-methyl-6-nitro-5-phenylpyrazolo[1,5-a]pyrimidin-7-one, 4-(3-(2,4-difluorophenyl)-4H-2-methyl-3-H-imidazo[1,2-b]pyrazolo-3-oxo) benzonitrile, 4-(3-(2,4-difluorophenyl)-4H-2-methyl-6-H-imidazo[1,5-b]pyrazolo-6-oxo)benzonitrile, 4-(3-(2,4-difluorophenyl)-4H-2-methylpyrazolo[5,1-b]quinazolin-9(4H)-oxo)benzonitrile, 4-(2-(2,4-difluorophenyl)-4H-[1,2,4]triazolo[5,1-b]quinazolin-9(4H)-oxo)benzonitrile, 4-(3-(2,4-difluorophenyl)-4H-4,6-trimethylpyrazolo[1,5-a]pyrimidin-7-oxo-5-yl)benzonitrile, 4-(3-(2,4-difluorophenyl)-7-dihydro-2,4-dimethylpyrazolo[1,5-a]pyrimidin-7-one-6-ethyl)benzonitrile, ethyl 4-(3-(2,4-difluorophenyl)-7-oxo-4,7-dihydro-2,4-dimethyl-5-hydroxypyrazolo[1,5-a]pyrimidin-6-acetate, 4-(3-(2,4-difluorophenyl)-4-dihydro-2,4-methyl-6-nitro-5-phenyl)pyrazolo[1,5-a]pyrimidin-7-one, 4-(3-(2,4-difluorophenyl)-2,4-dimethyl-9-oxo-4,9-dihydropyrazolo[5,1-b]quinazolin-6-yl)benzonitrile, 4-(2-(2,4-difluorophenyl)-4-methyl-9-oxo-4,9-dihydro[1,2,4]triazolo[5,1-b]quinazolin-6-yl)benzonitrile, 4-(3-(4-(trifluoromethyl)phenyl)-4H-2-methylpyrazolo[1,5-a]pyrimidin-7-oxo-5-yl)benzonitrile, 4-(3-(4-(trifluoromethyl)phenyl)-4H-2,6-dimethylpyrazolo[1,5-a]pyrimidin-7-oxo-5-yl)-benzonitrile, 4-(3-(4-(trifluoromethyl)phenyl)-4H-2,4-dimethylpyrazolo[1,5-a]pyrimidin-7-oxo-5-yl)-benzonitrile, 4-(3-(4-(trifluoromethyl)phenyl)-4H-2,4,6-trimethylpyrazolo[1,5-a]pyrimidin-7-oxo-5-yl)benzonitrile, 4-(3-(4-(trifluoromethyl)phenyl)-4,7-dihydro-2-methylpyrazolo[1,5-a]pyrimidin-7-oxo-6-ethyl carboxylate)benzonitrile, ethyl 3-(4-(trifluoromethyl)phenyl)-7-oxo-4,7-dihydro-2-methyl-5-hydroxypyrazolo[1,5-a]pyrimidin-6-acetate, 3-((4-(trifluoromethyl)phenyl)-4-dihydro-2-methyl)-6-nitro-5-phenylpyrazolo[1,5-a]pyrimidin-7-one, 4-(6-methyl-3-oxo-7-(4-(trifluoromethyl)phenyl)-3H-imidazo[1,2-b]pyridin-2-yl)benzonitrile, 4-(2-methyl-6-oxo-3-(4-(trifluoromethyl)phenyl)-6H-imidazo[1,5-b]pyrazol-4-yl)benzonitrile, 4-(2-methyl-9-oxo-3-(4-(trifluoromethyl)phenyl)-4,9-dihydropyrazolo[5,1-b]quinazolin-6-yl)benzonitrile, 4-(9-oxo-2-(4-(trifluoromethyl)phenyl)-4,9-dihydro[1,2,4]triazolo[5,1-b]quinazolin-6-yl)benzonitrile, 3-(2,4-difluorophenyl)-4H-2-methyl-5-((4-trifluoromethyl)phenyl)pyrazolo[1,5-a]pyrimidin-7-one, 3-(2,4-difluorophenyl)-4H-2,6-dimethyl-5-((4-trifluoromethyl)phenyl)pyrazolo[1,5-a]pyrimidin-7-one, 3-(2,4-difluorophenyl)-4H-2,4-dimethyl-54(4-trifluoromethyl)phenyl)pyrazolo[1,5-a]pyrimidin-7-one, 3-(2,4-difluorophenyl)-4H-4,6-trimethyl-54(4-trifluoromethyl)phenyl)pyrazolo[1,5-a]pyrimidin-7-one, 3-((2,4-fluorophenyl)-4,7-dihydro-2-methyl-54(4-trifluoromethyl)phenyl)pyrazolo[1,5-a]pyrimidin-7-one, 7-(2,4-difluorophenyl)-6-methyl-2-(4-(trifluoromethyl)phenyl)-3H-imidazo[1,2-b]pyrazol-3-one, 3-(2,4-difluorophenyl)-2-methyl-4-(4-(trifluoromethyl)phenyl)-6H-imidazo[1,5-b]pyrazol-6-one, 3-(2,4-difluorophenyl)-2-methyl-6-(4-(trifluoromethyl)phenyl)pyrazolo[5,1-b]quinazolin-9(4H)-one, 2-(2,4-difluorophenyl)-6-(4-(trifluoromethyl)phenyl)-1,2,4-triazolo[5,1-b]quinazolin-9(4H)-one, 3-((4-trifluoromethyl)phenyl)-4,7-dihydro-2-methyl-5-(4-trifluoromethyl)pyrazolo[1,5-a]pyrimidin-7-one, 3-((4-trifluoromethyl)phenyl)-4H-2,6-dimethyl-5-(4-trifluoromethyl)phenylpyrazolo[1,5-a]pyrimidin-7-one, 3-((4-trifluoromethyl)phenyl)-4H-2,4-dimethyl-5-(4-trifluoromethyl)phenyl pyrazolo[1,5-a]pyrimidin-7-one, 3-((4-trifluoromethyl)phenyl)-7-H-2,4,6-trimethyl-5-(4-trifluoro methyl)phenyl pyrazolo[1,5-a]pyrimidin-7-one, ethyl 3-((4-trifluoromethyl)phenyl)-4,7-dihydro-2-methyl-5-(4-trifluoromethyl)phenyl pyrazolo[1,5-a]pyrimidin-7-one-6-acetate-6-methyl-2,7-bis(4-(trifluoromethyl)phenyl)-3H-imidazo[1,2-b]pyrazol-3-one, 2-methyl-3,4-bis(4-(trifluoromethyl)phenyl)-6H-imidazo[1,5-b]pyrazol-6-one, 2-methyl-3,6-bis(4-(trifluoromethyl)phenyl)pyrazolo[5,1-b]quinazolin-9(4H)one, 2,6-bis(4-(trifluoromethyl)phenyl)-1,2,4-triazolo[5,1-b]quinazolin-9(4H)one, 2-methyl-3-(4-(trifluoromethyl)phenyl)-5-(4-trifluoromethyl)pyridin-3-yl-4,7-dihydropyrazolo[1,5-a]pyrimidin-7-one, 2,6-dimethyl-3-((4-(trifluoromethyl)phenyl)-5-(4-trifluoromethyl)pyridin-3-yl)-4,7-dihydropyrazolo[1,5-a]pyrimidin-7-one, 2,4-dimethyl-3-(4-trifluorophenyl-5-(4-(trifluoromethyl)pyridin-3-yl)-4,7-dihydropyrazolo[1,5-a]pyrimidin-7-one, 2,4,6-trimethyl-3-(4-(trifluoromethyl)phenyl)-5-(4-(trifluoromethyl)pyridin-3-yl)-4,7-dihydropyrazolo[1,5-a]pyrimidin-7-one, ethyl 2-methyl-3-((4-(trifluoromethyl)phenyl)-5-(4-(trifluoromethyl)pyridin-3-yl)-4,7-dihydropyrazolo[1,5-a]pyrimidin-7-one-6-formlate, 6-methyl(4-(trifluoromethyl)phenyl)-5-(6-(trifluoromethyl)pyridin-3-yl)-3-H-imidazo[1,2-b]imidazol-3-one, 2-methyl-3-(4-(trifluoromethyl)phenyl)-4-(6-(trifluoromethyl)pyridin-3-yl)-6H-imidazo[1,5-b]pyrazol-6-one, 2-methyl-3-(4-(trifluoromethyl)phenyl)-6-((6-trifluoromethyl)pyridin-3-yl)pyrazolo[5,1-b]quinazolin-9(4H)one, 2-(4-(trifluoromethyl)phenyl)-6-(6-(trifluoromethyl)pyridin-3-yl)-1,2,4-triazolo[5,1-b]quinazolin-9(4H)-one, 3-((4-trifluoromethyl)phenyl)-4,7-dihydro-2-methyl-5-(2,4-difluorophenyl)pyrazolo[1,5-a]pyrimidin-7-one, 3-((4-trifluoro-methyl)phenyl)-4,7-dihydro-2,6-dimethyl-5-(2,4-difluorophenyl)pyrazolo[1,5-a]pyrimidin-7-one, 3-((4(4-trifluoromethyl)phenyl)-4,7-dihydro-2,4-dimethyl-5-(2,4-difluorophenyl)pyrazolo[1,5-a]pyrimidin-7-one, 3-((4-trifluoromethyl)phenyl)-4,7-dihydro-2,4,6-trimethyl-5-(2,4-difluorophenyl)pyrazolo[1,5-a]pyrimidin-7-one, 3-((4-trifluoromethyl)phenyl), 7-dihydro-2-methyl-5-(2,4-difluorophenyl)pyrazolo[1,5-a]pyrimidin-7-one-6-ethyl-2-(2,4-difluorophenyl)-6-methyl-7-(4-(trifluoromethyl)phenyl)-3H-imidazo[1,2-b]pyrazol-3-one, 4-(2,4-difluorophenyl)-2-methyl-3-(4-(trifluoromethyl)phenyl)-6H-imidazo[1,5-b]pyrazol-6-one, 6-(2,4-difluorophenyl)-2-methyl-3-(4-(trifluoromethyl)phenyl)pyrazolo[5,1-b]quinazolin-9(4H) one, 5-(2,4-difluorophenyl)-2-(4-(trifluoromethyl)phenyl)-1,2,4-triazolo[5,1-b]quinolinylmorpholin-9(4H)-one, 3-(2,4-difluorophenyl)-4,7-dihydro-2-methyl-5-(4-methoxy)phenylpyrazolo[1,5-a]pyrimidin-7-one, 3-(2,4-difluorophenyl)-4,7-dihydro-2,6-dimethyl-5-(4-methoxy)phenylpyrazolo[1,5-a]pyrimidin-7-one, 3-(2,4-difluorophenyl)-4,7-dihydro-2,4-dimethyl-5-(4-methoxy)phenylpyrazolo[1,5-a]pyrimidin-7-one, 3-(2,4-difluorophenyl)-4,7-dihydro-2,4,6-trimethyl-5-(4-methoxy)phenylpyrazolo[1,5-a]pyrimidin-7-one, ethyl 3-(2,4-difluorophenyl)-4,7-dihydro-2-methyl-5-(4-methoxy)phenylpyrazolo[1,5-a]pyrimidin-7-one-6-carboxylate, 7-(2,4-difluorophenyl)-5-(4-methoxyphenyl)-6-methyl-3H-imidazo[1,2-b]pyrazol-3-one, 3-(2,4-difluorophenyl)-5-(4-methoxyphenyl)-2-methyl-6H-imidazo[1,5-b]pyrazol-6-one, 3-(2,4-difluorophenyl)-6-(4-methoxylphenyl)-2-methylpyrazolo[5,1-b]quinazolin-9(4H)-one, 2-(2,4-difluorophenyl)-6-(4-methoxylphenyl)triazolo[5,1-b]quinazolin-9(4H)-one, 3-(2,4-difluorophenyl)-4,7-dihydro-2-methyl-5-(4-amino)phenylpyrazolo[1,5-a]pyrimidin-7-one, 3-(2,4-difluorophenyl)-4,7-dihydro-2,6-dimethyl-5-(4-amino)phenylpyrazolo[1,5-a]pyrimidin-7-one, 3-(2,4-difluorophenyl)-4,7-dihydro-2,4-dimethyl-5-(4-amino)phenylpyrazolo[1,5-a]pyrimidin-7-one, 3-(2,4-difluorophenyl)-4,7-dihydro-2,4,6-trimethyl-5-(4-amino)phenylpyrazolo[1,5-a]pyrimidin-7-one, 3-(2,4-difluorophenyl)-4,7-dihydro-2-methyl-5-(4-amino)phenylpyrazolo[1,5-a]pyrimidin-7-keton-6-ethyl, 2-(4-aminophenyl)-7-(2,4-difluorophenyl)-6-methyl-3H-imidazo[1,2-b]pyrazol-3-one, 4-(4-amin phenyl)-3-(2,4-difluorophenyl)-2-methyl-6H-imidazo[1,5-b]pyrazol-6-one, 6-(4-aminophenyl)-3-(2,4-difluorophenyl)-2-methylpyrazolo[5,1-b]quinazolin-9(4H)-one, 6-(4-aminophenyl)-2-(2,4-difluorophenyl)-1,2,4-triazolo[5,1-b]quinazolin-9(4H)-one, 4-(3-(2,4-difluorophenyl)-2-methyl-7-oxo-4,7-dihydropyrazolo[1,5-a]pyrimidin-5-ylaminomethyl)benzonitrile, 4-(3-(2,4-difluorophenyl)-2,6-dimethyl-7-oxo-4,7-dihydropyrazolo[1,5-a]pyrimidin-5-ylaminomethyl)benzonitrile, 4-(3-(2,4-difluorophenyl)-2,4-dimethyl-7-oxo-4,7-dihydropyrazolo[1,5-a]pyrimidin-5-ylaminomethyl)benzonitrile, 4-(3-(2,4-difluorophenyl)-2,4,6-trimethyl-7-oxo-4,7-dihydropyrazolo[1,5-a]pyrimidin-5-ylaminomethyl)benzonitrile, 4-(3-(2,4-difluorophenyl)-2-methyl-7-oxo-4,7-dihydropyrazolo[1,5-a]pyrimidin-5-yl aminomethyl-6-ethyl)benzonitrile, 4-((7-(2,4-difluorophenyl)-6-methyl-3-oxo-3H-imidazo[1,2-b]pyrazol-2-yl)aminomethyl)benzonitrile, 4-((3-(2,4-difluorophenyl)-2-methyl-6-oxo-3H-imidazo[1,5-b]pyrazol-4-ylaminomethyl)benzonitrile, 4-((3-(2,4-difluorophenyl)-2-methyl-9-oxo-4,9-dihydropyrazolo[5,1-b]quinazolin-6-yl aminomethyl)benzonitrile, 4-(2-(2,4-difluorophenyl)-9-oxo-4,9-dihydro-1,2,4-triazolo[5,1-b]quinazolin-6-yl)aminomethyl)benzonitrile, 4-(3-(2,4-difluorophenyl)-2-methyl-7-oxo-4,7-dihydropyrazolo[1,5-a]pyrimidin-5-yl-N,N-dipropyl)benzene sulfonamide, 4-(3-(2,4-difluorophenyl)-2,6-dimethyl-7-oxo-4,7-dihydropyrazolo[1,5-a]pyrimidin-5-yl-N,N-dipropylbenzenesulfonamide, 3-(2,4-dichlorophenyl)-5-(dipropylamino)-2,4-dimethylpyrazolo[1,5-a]pyridin-7(4H)-one, 4-(3-(2,4-difluorophenyl)-2,4,6-triethyl-7-oxo-4,7-dihydropyrazolo[1,5-a]pyrimidin-5-yl-N,N-dipropyl)benzene sulfonamide, ethyl 4-(3-(2,4-difluorophenyl)-2-methyl-7-oxo-4,7-dihydropyrazolo[1,5-a]pyrimidin-5-yl-N,N-dipropyl)benzene sulfonamide-6-carboxylate, ethyl 4-(7-(2,4-difluorophenyl)-6-methyl-3-oxo-3H-imidazo[1,2-b]pyrazol-2-yl)-N,N-dipropyl benzene sulfonamide, 4-(3-(2,4-difluorophenyl)-2-methyl-6-oxo-6H-imidazo[1,5-b]pyrazol-4-yl)-N,N-dipropylbenzenesulfonamide, 4-(3-(2,4-difluorophenyl)-2-methyl-9-oxo-4,9-dihydropyrazolo[5,1-b]quinazolin-6-yl)-N,N-dipropylbenzenesulfonamide, 4-(2-(2,4-difluorophenyl)-9-oxo-4,9-dihydro-1,2,4-triazolo[5,1-b]quinazolin-6-yl)-N,N-dipropyl benzenesulfonamide, 3-(2,4-difluorophenyl)-4H-2-ethyl-5-(4-trifluoromethyl)phenyl)pyrazolo[1,5-a]pyrimidin-7-one, 3-(2,4-difluoro phenyl)-4H-2-ethyl-6-methyl-54(4-trifluoromethyl)phenyl)pyrazolo[1,5-a]pyrimidin-7-one, 3-(2,4-difluorophenyl)-4H-2-ethyl-4,6-dimethyl-5-(4-trifluoromethyl)phenyl)pyrazolo[1,5-a]pyrimidin-7-one, ethyl 3-(2,4-difluorophenyl)-4H-2-ethyl-54(4-trifluoromethyl)phenyl)pyrazolo[1,5-a]pyrimidin-7-one-6-carboxylate, ethyl 3-(2,4-difluorophenyl)-4,7-dihydro-2-methyl-5-hydroxy pyrazolo[1,5-a]pyrimidin-7-one-6-carboxylate, 3-((2,4-difluorophenyl)-4-dihydro-2-methyl-6-nitro-5-phenyl)pyrazolo[1,5-a]pyrimidin-7-one, 3-(2,4-difluorophenyl)-4H-2-ethyl-5-(4-(trifluoromethyl)phenyl)-3-H-imidazo[1,2-b]pyrazol-3-one, 3-(2,4-difluorophenyl)-4H-2-ethyl-5-(4-(trifluoromethyl phenyl))-6-H-imidazo[1,5-b]pyrazol-6-one, 3-(2,4-difluorophenyl)-4H-2-ethyl-5-(4-(trifluoromethyl)phenyl]pyrazolo[5,1-b]quinazolin-9(4H)one, 3-(2,4-difluorophenyl)-4,7-dihydro-2-phenylpyrazolo[1,5-a]pyrimidin-7-one-5-yl)benzonitrile, 4-(3-(2,4-difluorophenyl)-4,7-dihydro-2-phenyl-6-methylpyrazolo[1,5-a]pyrimidin-7-oxo-5-yl)benzonitrile, 4-(3-(2,4-difluorophenyl)-4,7-dihydro-2-phenyl-4-methylpyrazolo[1,5-a]pyrimidin-7-oxo-5-yl)benzonitrile, 4-(3-(2,4-difluorophenyl)-7-H-2-phenyl-4,6-dimethylpyrazolo[1,5-a]pyrimidin-7-oxo-5-yl)benzonitrile, 4-(3-(2,4-difluorophenyl)-4,7-dihydro-2-phenylpyrazolo[1,5-a]pyrimidin-7-oxo-6-ethyl carboxylate-5-yl)benzonitrile, ethyl 4-(3-(2,4-difluorophenyl)-7-oxo-4,7-dihydro-2-phenyl-5-hydroxy pyrazolo[1,5-a]pyrimidinyl)-6-carboxylate, 4-(3-(2,4-difluorophenyl)-4H-2-phenyl-6-nitro-5-phenylpyrazolo[1,5-a]pyrimidin-7-one, 4-(3-(2,4-difluorophenyl)-4H-2-phenylpyrimido[1,2-b]pyridazin-4(6H)-oxo-5-yl)benzonitrile, 4-(7-(2,4-difluorophenyl)-3-oxo-6-phenyl-3H-imidazo[1,2-b]pyrazol-2-yl)benzonitrile, 4-(3-(2,4-difluorophenyl)-6-oxo-2-phenyl-6H-imidazo[1,5-b]pyrazol-4-yl)benzonitrile, 4-(3-(2,4-difluorophenyl)-9-oxo-2-phenyl-4,9-dihydropyrazolo[5,1-b]quinazolin-6-yl)benzonitrile, 3-(2,4-difluorophenyl)-4,7-dihydro-2-phenyl-5-(4-(trifluoromethyl)pyrazolo[1,5-a]pyrimidinyl)-7-one, 3-(2,4-difluorophenyl)-4,7-dihydro-2-phenyl-6-methyl-5-(4-trifluoromethyl)phenyl pyrazolo[1,5-a]pyrimidin-7-one, 3-(2,4-difluorophenyl)-4,7-dihydro-2-phenyl-4-methyl-5-(4-trifluoromethyl)phenyl pyrazolo[1,5-a]pyrimidin-7-one, 3-(2,4-difluorophenyl)-4,7-dihydro-2-phenyl-4,6-dimethyl-5-(4-trifluoromethyl)phenyl pyrazolo[1,5-a]pyrimidin-7-one, 3-(2,4-difluorophenyl)-4,7-dihydro-2-phenyl-5-(4-trifluoromethyl)phenyl pyrazolo[1,5-a]pyrimidin-7-one, ethyl 3-(2,4-difluorophenyl)-7-oxo-4,7-dihydro-2-phenyl-5-hydroxypyrazolo[1,5-a]pyrimidin-6-carboxylate 3-(2,4-difluorophenyl)-4-dihydro-2-phenyl-6-nitro-5-phenyl pyrazolo[1,5-a]pyrimidin-7-one, 7-(2,4-difluorophenyl)-6-phenyl-2-(4-(trifluoromethyl)phenyl)-3H-imidazo[1,2-b]pyrazol-3-one, 3-(2,4-difluorophenyl)-2-phenyl-4-(4-(trifluoromethyl)phenyl)-6H-imidazo[1,5-b]pyrazol-6-one, 3-(2,4-difluorophenyl)-2-phenyl-6-(4-(trifluoromethyl)phenyl)pyrazolo[5,1-b]quinazolin-9(4H)-one, 4-(2-cyclopropyl-3-(2,4-difluorophenyl)-7-oxo-4,7-dihydropyrazolo[1,5-a]pyrimidin-(5-yl))benzonitrile, 4-(2-cyclopropyl-3-(2,4-difluorophenyl)-6-methyl-7-oxo-4,7-dihydropyrazolo[1,5-a]pyrimidin-5-yl)benzonitrile, 4-(2-cyclopropyl-3-(2,4-difluorophenyl)-4-methyl-7-oxo-4,7-dihydropyrazolo[1,5-a]pyrimidin-5-yl)benzonitrile, 4-(2-cyclo-propyl-3-(2,4-difluorophenyl)-4,6-dimethyl-7-oxo-4,7-dihydropyrazolo[1,5-a]pyrimidin-5-yl)benzonitrile, 4-((2-cyclopropyl-3-(2,4-difluorophenyl)-7-oxo-4,7-dihydropyrazolo[1,5-a]pyrimidin-6-ethyl-5-yl)benzonitrile, ethyl 4-(2-cyclopropyl)-3-(2,4-difluorophenyl)-7-oxo-4,7-dihydro-5-hydroxy pyrazolo[1,5-a]pyrimidin-6-acetate, 4-(2-cyclopropyl)-3-(2,4-difluorophenyl)-4H-6-nitro-5-phenyl pyrazolo[1,5-a]pyrimidin-7-one, 4-(6-cyclopropyl-7-(2,4-difluorophenyl)-3-oxo-3H-imidazo[1,2-b]pyrazol-2-yl)benzonitrile, 4-(2-cyclopropyl-3-(2,4-difluorophenyl)-6-oxo-6H-imidazo[1,5-b]pyridin-4-yl)benzonitrile, 4-(2-cyclopropyl-3-(2,4-difluorophenyl)-9-oxo-4,9-dihydropyrazolo[5,1-b]quinazolin-6-yl)benzonitrile, 4-(3-(2,4-difluorophenyl)-7-oxo-2-trifluoromethyl)methyl-4,7-dihydropyrazolo[1,5-a]pyrimidin-5-yl)benzonitrile, 4-(3-(2,4-difluorophenyl)-7-oxo-2-(trifluoromethyl))-4,7-dihydropyrazolo[1,5-a]pyrimidin-5-yl)benzonitrile, 4-((3-(2,4-difluorophenyl)-7-oxo-2-trifluoromethyl)-4,7-dihydropyrazolo[1,5-a]pyrimidin-5-yl)benzonitrile, 4-(3-(2,4-difluorophenyl)-7-oxo-2-trifluoromethyl)-4,6-dimethyl-4,7-dihydropyrazolo[1,5-a]pyrimidin-5-yl)benzonitrile, 4-(3-(2,4-difluorophenyl)-7-oxo-2-(trifluoromethyl))-4,7-dihydropyrazolo[1,5-a]pyrimidin-6-ethyl formate-5-yl)benzonitrile, ethyl 4-(3-(2,4-difluorophenyl)-7-oxo-2-(trifluoromethyl))-4,7-dihydro-5-hydroxyl pyrazolo[1,5-a]pyrimidin-6-carboxylate, 4-(3-(2,4-difluorophenyl)-7-oxo-2-(trifluoromethyl))-4H-6-nitro-5-phenyl pyrazolo[1,5-a]pyrimidin-7-one, 4-(7-(2,4-difluorophenyl)-3-oxo-6-(trifluoromethyl)-3H-imidazo[1,2-b]pyrazol-2-yl)benzonitrile, 4-(3-(2,4-difluorophenyl)-6-oxo-2-(trifluoromethyl)-6H-imidazo[1,5-b]pyrazol-4-yl)benzonitrile, 4-(3-(2,4-difluorophenyl)-9-oxo-2-(trifluoromethyl) 4,9-dihydropyrazolo[5,1-b]quinazolin-6-yl)benzonitrile, 4-(3-(2,4-difluorophenyl)-2-ethyl-7-oxo-4H-pyrazolo[1,5-a]pyrimidin-5-yl)benzonitrile, 4-(3-(2,4-difluorophenyl)-2-ethyl-6-methyl-7-oxo-4H-pyrazolo[1,5-a]pyrimidin-5-yl)benzonitrile, 4-(3-(2,4-difluorophenyl)-2-ethyl-4-methyl-7-oxo-4H-pyrazolo[1,5-a]pyrimidin-5-yl)benzene carbonitrile, 4-(3-(2,4-difluorophenyl)-2-ethyl-4,6-dimethyl-7-oxo-4H-pyrazolo[1,5-a]pyrimidin-5-yl)benzonitrile, 4-(3-(2,4-difluorophenyl)-2-7-oxo-4,7-dihydropyrazolo[1,5-a]pyrimidin-6-ethyl-5-yl)benzonitrile, ethyl 4-(3-(2,4-difluorophenyl)-2-ethyl-7-oxo-4,7-dihydro-5-hydroxyl)pyrazolo[1,5-a]pyrimidin-6-carboxylate, ethyl 3-(2,4-difluorophenyl)-2-ethyl-4H-6-nitro-5-phenylpyrazolo[1,5-a]pyrimidin-7-one, 4-(7-(2,4-difluorophenyl)-6-ethyl-3-oxo-3H-imidazo[1,2-b]pyrazol-2-yl)benzonitrile, 4-(3-(2,4-difluorophenyl)-2-ethyl-6-oxo-6H-imidazo[1,5-b]pyrazol-4-yl)benzonitrile, 4-(3-(2,4-difluorophenyl)-2-ethyl-9-oxo-4,9-dihydropyrazolo[5,1-b]quinazolin-6-yl)benzonitrile, 4-(3-(2,4-dichlorophenyl)-4H-2-methyl pyrazolo[1,5-a]pyrimidin-7-oxo-5-yl)benzonitrile, 4-(3-(2,4-dichlorophenyl)-4H-2,6-methyl pyrazolo[1,5-a]pyrimidin-7-oxo-5-yl)benzonitrile, 4-(3-(2,4-dichlorophenyl)-4H 2,4-methyl pyrazolo[1,5-a]pyrimidin-7-oxo-5-yl)benzonitrile, 4-(3-(2,4-dichlorophenyl)-7H-2,4,6-trimethyl pyrazolo[1,5-a]pyrimidin-7-oxo-5-yl)benzonitrile, 4-(3-(2,4-dichlorophenyl)-4,7-dihydro-2-methylpyrazolo[1,5-a]pyrimidin-6-ethyl formate-7-oxo-5-yl)benzonitrile, ethyl 4-(3-(2,4-dichlorophenyl)-7-oxo-4,7-dihydro-2-methyl-5-hydroxy-pyrazolo[1,5-a]pyrimidinyl)-6-carboxylate, 4-(3-(2,4-dichlorophenyl)-4-dihydro-2-methyl-6-nitro-5-phenyl pyrazolo[1,5-a]pyrimidinyl)-7-one, 4-(7-(2,4-dichlorophenyl)-6-methyl-3-oxo-3H-imidazo[1,2-b]pyrazol-2-yl)benzonitrile, 4-(3-(2,4-dichlorophenyl)-2-methyl-6-oxo-6H-imidazo[1,5-b]pyrazol-4-yl)benzonitrile, 4-(3-(2,4-dichlorophenyl)-2-methyl-9-oxo-4,9-dihydropyrazolo[5,1-b]quinazolin-6-yl)benzonitrile, 4-(2-(2,4-dichlorophenyl)-9-oxo-4,9-dihydro-1,2,4-triazolo[5,1-b]quinazolin-6-yl)benzonitrile, 4-(3-(4-benzo-yloxy)-4H-2-methylpyrazolo[1,5-a]pyrimidin-7-oxo(5-yl)benzonitrile, 4-(3-(4-benzoyloxy)-4H-2,6-methylpyrazolo[1,5-a]pyrimidin-7-oxo-5-yl)benzonitrile, 4-(3-(4-benzoyloxy)-4H-2,4-methylpyrazolo[1,5-a]pyrimidin-7-oxo-5-yl)benzonitrile, 4-(3-(4-benzoic acid)-7H-2,4,6-trimethylpyrazolo[1,5-a]pyrimidin-7-oxo-5-yl)benzonitrile, 4-(3-(4-benzoic acid-yl)-4,7-dihydro-2-methylpyrazolo[1,5-a]pyrimidin-6-ethyl-7-oxo-5-yl)benzonitrile, ethyl 3-(4-methoxyphenyl)-7-oxo-4,7-dihydro-2-methyl-5-hydroxypyrazolo[1,5-a]pyrimidinyl)-6-carboxylate, 3-(4-methoxyphenyl)-2-methyl-6-nitro-5-phenylpyrazolo[1,5-a]pyrimidin-7(4H)-one, 4-(2-(4-cyanophenyl)-6-methyl-3-oxo-3H-imidazo[1,2-b]pyrazol-7-yl)benzoic acid, 4-(4-(4-cyanophenyl)-2-methyl-6-oxo-6H-imidazo[1,5-b]pyrazol-3-yl)benzoic acid, 4-(6-(4-cyanophenyl)-2-methyl-9-oxo-pyrazolo[5,1-b]quinazolin-3-yl)benzoic acid, 4-(6-(4-cyanophenyl)-9-oxo-4,9-dihydro-1,2,4-triazolo[5,1-b]quinazolin-2-yl)benzoic acid, (Z)-2-(hydroxymethyl)-6-(4-(7-methyl-8-phenylpyrazolo[1,5-a][1,3,5]triazin-2(1H)-ylideneamino)phenoxy)-tetrahydro-2H-pyran-3,4,5-triol, (Z)-2-(hydroxymethyl)-6-(4-(7-methyl-8-(2,4-difluorophenyl)pyrazolo[1,5-a][1,3,5]triazin-2(1H)-ylideneamino)phenoxy)-tetrahydro-2H-pyran-3,4,5-triol, (Z)-2-(hydroxymethyl)-6-(4-(7-methyl-8-((4-trifluoromethyl)phenyl)pyrazolo[1,5-a][1,3,5]triazin-2(1H)-ylideneamino)phenoxy)-tetrahydro-2H-pyran-3,4,5-triol, (E)-4-(7-methyl-8-(4-(trifluoromethyl)phenyl)-2-(4-(3,4,5-trihydroxy-6-(hydroxymethyl)-tetrahydro-2H-pyran-2-yloxy)phenylimino)-1,2-dihydropyrazolo[1,5-a][1,3,5]triazin-4-yl)benzonitrile, (Z)-4-(7-methyl-8-(4-(trifluoromethyl)phenyl)-2-(4-(3,4,5-trihydroxy-6-(hydroxymethyl)-tetrahydro-2H-pyran-2-yloxy)phenylimino)-1,2-dihydropyrazolo[1,5-a][1,3,5]triazin-4-yl)benzoic acid, (Z)-4-(7-methyl-8-(4-(trifluoromethyl)phenyl)-2-(4-(3,4,5-trihydro-oxy-6-(hydroxymethyl)-tetrahydro-2H-pyran-2-yloxy)phenylimino)-1,2-dihydropyrazolo[1,5-a][1,3,5]triazin-4-yl)benzene sulfonic acid, (Z)-4-(7-methyl-8-(4-(trifluoromethyl)phenyl)-2-(4-(3,4,5-trihy-droxy-6-(hydroxymethyl)-tetrahydro-2H-pyran-2-yloxy)phenylimino)-1,2-dihydropyrazolo[1,5-a][1,3,5]triazin-4-yl))benzenesulfonamide, (Z)-4-(7-methyl-8-(4-(trifluoromethyl)phenyl)-2-(4-(3,4,5-tri-hydroxy-6-(hydroxymethyl)-tetrahydro-2H-pyran-2-yloxy)phenylimino)-1,2-dihydropyrazolo[1,5-a][1,3,5]triazin-4-yl) N,N-dipropyl benzenesulfonamide, (Z)-4-(2-methyl-3-(4-(trifluoromethyl)phenyl)-5-(4-(3,4,5-trihydroxy-6-(hydroxymethyl)-tetrahydro-2H-pyran-2-yloxy)phenylimino)-4,5-dihydropyrazolo[1,5-a]pyrimidin-7-yl)benzonitrile, (Z)-4-(3-(2,4-difluorophenyl)-2-methyl-5-oxo-6-(4-(3,4,5-trihydroxy-6-(hydroxymethyl)-tetrahydro-2H-pyran-2-yloxy)phenylimino)-4,5,6,7-tetrahydropyrazolo[1,5-a]pyrimidin-7-yl)benzonitrile, (Z)-4-(3-(2,4-difluorophenyl)-2-methyl-5-oxo-6-(4-(3,4,5-trihydroxy-6-(hydroxymethyl)-tetrahydro-2H-pyran-2-yloxy)phenylimino)-4,5,6,7-tetrahydropyrazolo[1,5-a]pyrimidin-7-yl)benzonitrile, (z)-4-(5-(4-(3,4,5-trihydroxy hydrogen-6-hydroxy methyl-6H-pyran-2-yl)oxybenzylideneamine)-4-imino-3-methyl-4,5-dihydropyrazolo[3,4-d]pyrimidin-1-yl)benzonitrile, 4-(4-imino-3-methyl-6-(4-(3,4,5-trihydroxy-6-(hydroxymethyl)-tetrahydro-2H-pyran-2-yloxy)phenyl)-4,5-dihydropyrazolo[3,4-d]pyrimidin-1-yl)benzenesulfonic acid(z)-4-(5-(4-(3,4,5-trihydrooxyl-6-hydroxymethyl-6H-pyran-2-yl)oxybenzylidene amine)-4-imino-3-methyl-4,5-dihydropyrazolo[3,4-d]pyrimidin-1-yl)-N,N-dipropylbenzene sulfonamide, 4-(3-cyclopropyl-4-imino-6-(4-(3,4,5-trihydroxy-6-(hydroxymethyl)-tetrahydro-2H-pyran-2-yloxy)phenyl)-4,5-dihydropyrazolo[3,4-d]pyrimidin-1-yl)-N,N-dipropylbenzenesulfonamide, 4-(3-cyclopropyl-4-imino-6-(4-(3,4,5-trihydroxy-6-(hydroxymethyl)-tetrahydro-2H-pyran-2-yloxy)phenyl)-4,5-dihydropyrazolo[3,4-d]pyrimidin-1-yl)benzenesulfonamide, 4-(3-cyclopropyl-4-imino-6-(4-(3,4,5-trihydro-oxy-6-(hydroxymethyl)-tetrahydro-2H-pyran-2-yloxy)phenyl)-4,5-dihydropyrazolo[3,4-d]pyrimidin-1-yl)benzenesulfonic acid, 4-(3-cyclopropyl-4-imino-6-(4-(3,4,5-trihydroxy-6-(hydroxy methyl)-tetrahydro-2H-pyran-2-yloxy)phenyl)-4,5-dihydropyrazolo[3,4-d]pyrimidin-1-yl)benzonitrile, 2-(4-(3-cyclopropyl-4-imino-1-(pyridin-4-yl)-4,5-dihydro-1H-pyrazolo[3,4-d]pyrimidin-6-yl)phenoxy)-6-(hydroxymethyl)-tetrahydro-2H-pyran-3,4,5-triol, (z)-4-(5-(4-(3,4,5-trihydroxyl-6-hydroxy methyl-6H-pyran-2-yl)oxy benzylidene amine)-4-imino-3-phenyl-4,5-dihydropyrazolo[3,4-d]pyrimidin-1-yl)benzonitrile, (z)-4-(5-(4-(3,4,5-trihydroxy hydrogen-6-hydroxylmethyl-6H-pyran-2-yl)oxybenzylidene amine)-4-imino-3-phenyl-4,5-dihydropyrazolo[3,4-d]pyrimidin-1-yl)benzoic acid, (z)-4-(5-(4-(3,4,5-trihydroxy hydrogen-6-hydroxymethyl-6H-pyran-2-yl)oxybenzylidene amine)-4-imino-3-phenyl-4,5-dihydropyrazolo[3,4-d]pyrimidin-1-yl)benzoic acid, (z)-4-(5-(4-(3,4,5-trihydroxyl-6-hydroxymethyl-6H-pyran-2-yl)oxybenzylidene amine)-4-imino-3-phenyl-4,5-dihydrogenpyrazolo[3,4-d]pyrimidin-1-yl)-N,N-dipropyl benzenesulfonamide, (Z)-2-(hydroxy methyl)-6-(4-((6-imino-9-(2,4-difluoro)phenyl)-6H-purin-1(9H)-ylimino)methyl)phenoxy)-tetrahydr o-2H-pyran-3,4,5-triol, (Z)-2-(hydroxymethyl)-6-(4-((6-imino-9-(4-(trifluoromethyl)phenyl)-6H-purin-1(9H)-ylimino)methyl)phenoxy)-tetrahydro-2H-pyran-3,4,5-triol, (z)-4-(1-(4-aminophenyl amino)-6-imino-1,6-dihydropurin-9-yl)benzonitrile, (Z)-4-(1-(4-(trifluoromethyl)benzylideneamino)-6-imino-1,6-dihydropurin-9-yl)benzonitrile, (Z)-4-(1-(2,4-difluorobenzylidene amino)-6-imino-1,6-dihydropurin-9-yl)benzonitrile, 4-((6-imino-9-(4-(trifluoromethyl)phenyl)-6,9-dihydro-1H-purin-2-yl)methyl)benzonitrile, (z)-N-(4-(trifluoromethyl)benzylidene)-6-imino-9-(4(trifluoromethyl)phenyl)-6H-purin-1(9H)amine, (Z)—N-(2,4-difluorobenzylidene)-6-imino-9-(4-(trifluoromethyl)phenyl)-6H-purin-1(9H)-amine, (Z)-4-((6-imino-9-(4-(trifluoromethyl)phenyl)-6H-purin-1(9 H)-ylimino)methyl)benzonitrile, (Z)—N-(4-(trifluoromethyl)benzylidene)-9-(2,4-difluorophenyl)-6-imino-6H-purin-1(9H)-amine, (z)—N4(2,4-difluorophenyl)methyl)-9-(2,4-difluorophenyl)-6-imino-6H-purin-1(9H)amine, (z)-2-(hydroxymethyl)-6-((4-(6-imino-8-methyl-9-((4-trifluoromethyl)phenyl)-6H-purin-1-imino)methyl)phenoxy)-tetrahydro-2H-pyran-3,4,5-triol, (z)-4-(1-(4-methoxyl benzyl)imino)-6-amino-8-methyl-1,6-dihydropurin-9-yl-benzonitrile, (z)-4-((1-(4-trifluoromethyl)benzylidene amino)-6-imino-8-methyl-1,6-dihydropurin-9-yl)benzonitrile, (z)-4-(1-(2,4-difluoro benzylideneamine)-6-imino-8-methyl-1,6-dihydropurin-9-yl)benzonitrile, (z)-4-(6-imino-8-methyl-9-((4-trifluoromethyl)phenyl)-6H-purin-1-iminomethyl)benzonitrile, (z)-N-(4-(trifluoromethyl)benzylidene)-6-imino-8-methyl-9((4-trifluoromethyl)phenyl)-6H-purin-1(9H)amine, (z)-N-(2,4-difluorophenyl methyl)-6-imino-9((4-trifluoromethyl)phenyl)-6H-purin-1(9H)amine, (z)-4-((9-(2,4-difluorophenyl)-6-imino-8-methyl-6H-purin-1-imino)methyl)benzonitrile, (z)-N-((4-(trifluoromethyl)phenyl)methyl)-9-(2,4-difluorophenyl)-6-imino-8-methyl-6H-purin-1-amine, (z)-N-((2,4-difluorophenyl)methyl)-9-(2,4-difluorophenyl)-6-imino-8-methyl-6H-purin-1-amine, (Z)-2-(hydroxymethyl)-6-(4-((6-imino-8-methyl-9-(4-(trifluoromethyl)phenyl)-6H-purin-1(9H)-ylimino)methyl)phenoxy)-tetrahydro-2H-pyran-3,4,5-triol, (Z)-4-(1-(4-(3,4,5-trihydroxy-6-(hydroxymethyl)tetrahydro-2H-pyran-2-yloxy)benzylideneamino)-6-imino-8-methyl-1,6-dihydropurin-9-yl)benzonitrile, 4-(6-amino-2-(4-(trifluoromethyl)phenyl)-9H-purin-9-yl)benzonitrile, 4-(6-amino-2-(2,4-difluorophenyl)-9H-purin-9-yl)benzonitrile, 4-(6-oxo-2-(4-(3,4,5-trihydroxy-6-(hydroxymethyl)-6H-pyran-2-yl)phenyl)-1,6-dihydropurin-9-yl)benzonitrile, 4-(6-oxo-2-(4-(trifluoromethyl)phenyl)-1,6-dihydropurin-9-yl)benzonitrile, 4-(2-(2,4-difluorophenyl)-6-oxo-1,6-dihydropurin-9-yl)benzonitrile, 4-(7-imino-2-methyl-6-(4-(3,4,5-trihydroxy-6-(hydroxymethyl)-tetrahydro-2H-pyran-2-yloxy)phenyl)-6,7-dihydroimidazo[4,5-b]pyridin-3-yl)benzonitrile, 2-(hydroxymethyl)-6-(4-(7-imino-2-methyl-3-(4-(trifluoromethyl)phenyl)-6,7-dihydro-3H-imidazo[4,5-b]pyridin-6-yl)phenoxy)tetrahydro-2H-pyran-3,4,5-triol, 2-(hydroxymethyl)-6-(4-(4-imino-4,9a-dihydro-3H-pyrido[1,2-a]pyrimidin-3-yl)phenoxy)-tetrahydro-2H-pyran-3,4,5-triol, 4-(4-amino-6-(4-(trifluoromethyl)phenyl)-1H-pyrazolo[3,4-d]pyrimidin-1-yl)benzonitrile, 4-(4-amino-6-(2,4-difluorophenyl)-1H-pyrazolo[3,4-d]pyrimidin-1-yl)benzonitrile, 4-(4-oxo-6-(4-(3,4,5-trihydroxy-6-(hydroxymethyl)-6H-pyran-2-yl)phenyl)-4,5-dihydropyrazolo[3,4-d]pyrimidin-1-yl)benzonitrile, 4-(4-oxo-6-(4-(trifluoromethyl)phenyl)-4,5-dihydropyrazolo[3,4-d]pyrimidin-1-yl)benzonitrile, 4-(6-(2,4-difluorophenyl)-4-oxo-4,5-dihydropyrazolo[3,4-d]pyrimidin-1-yl)benzonitrile, 4-(4Hydrazino-6-(4-(3,4,5-trihydroxy-6-(hydroxymethyl)-6H-pyran-2-yl)phenyl)-4H-pyrazolo[3,4-d]pyrimidin-1-yl)benzonitrile, 4-(5-amino-4-imino-7-(4-(trifluoromethyl)phenyl)-4,5,6,7-tetrahydropyrazolo[3,4-d]pyrimidin-1-yl)benzonitrile, 4-(5-(hydrazino) 7-(2,4-difluorophenyl)-1H-pyrazolo[3,4-d]pyrimidin-1-yl)benzonitrile, 4-(3,4,5-trihydroxy-6-(hydroxymethyl)-6H-pyran-2-yl)oxybenzyl)-hydrazin-6-methyl-1-(4-(trifluoromethyl)phenyl)-1-H-pyrazolo[3,4-d]pyrimidine, (Z)-4-(5-(4-(3,4,5-trihydroxy-6-(hydroxymethyl)-tetrahydro-2H-pyran-2-yloxy)benzylideneamino)-4-imino-4,5,6,7-tetrahydropyrazolo[3,4-d]pyrimidin-1-yl)benzonitrile, N-((3-di-methylamino)propyl)-7-methyl-2-phenylimidazo[1,2-a]pyrimidin-5-amine, N-((3-dimethylamino)propyl)-7-methyl-2-(4-(trifluoromethyl)phenyl)imidazo[1,2-a]pyrimidin-5-amine, 4-(5-(3-(dimethylamino)propylamino)-7-methylimidazo[1,2-a]pyrimidin-2-yl)benzonitrile, 2-(2,4-difluorophenyl)-N-(3-(dimethylamino)propyl)-7-methylimidazo[1,2-a]pyrimidin-5-amine, 4-(2-methyl-9-(4-(trifluoromethyl)phenyl)-9H-purin-6-amino)benzonitrile, 4-(9-(4-cyanophenyl)-2-methyl-9H-purin-6-amino) benzoic acid, 4-(9-(2,4-difluorophenyl)-2-methyl-9H-purin-6-amino)benzonitrile, 4-((6-methyl-1-(4-(trifluoromethyl)phenyl)-1H-pyrazolo[3,4-d]pyrimidin-4-yl)amino)benzonitrile, 4-(1-(4-cyanophenyl)-6-methyl-1H-pyrazolo[3,4-d]pyrimidin-4-yl-amino)benzoic acid, 4-((6-methyl-1-(4-(trifluoromethyl)phenyl)-1H-pyrazolo[3,4-d]pyrimidin-4-yl)amino)benzonitrile, 4-((1-(2,4-difluorophenyl)-6-methyl-1H-pyrazolo[3,4-d]pyrimidin-4-yl)amino)benzonitrile, 4-(5-oxo-1-(4-(trifluoromethyl)phenyl)-1,5,8,8a-tetrahydroimidazo[1,2-a]pyrimidin-7-yl) 3,7-bis(2,4-difluorophenyl)-2-(4-(trifluoromethyl)phenyl)imidazo[1,2-a]pyrimidin-5(8H)-one, 4-(1,7-bis(2,4-difluorophenyl)-5-oxo-imidazo[1,2-a]pyrimidin-2-yl)benzonitrile, 4-(7-(2,4-difluorophenyl)-3-(4-methoxy-phenyl)-5-oxo-5,8-dihydroimidazo[1,2-a]pyrimidin-2-yl)benzonitrile, 4-(2,7-bis(2,4-difluorophenyl)-5-oxo-5,8-dihydroimidazo[1,2-a]pyrimidin-3-yl)benzonitrile, 2,3,7-tris(2,4-difluorophenyl)imidazo[1,2-a]pyrimidin-5(8H)one, 4-(5-oxo-7-((trifluoromethyl)phenyl)-2-(4-(trifluoromethyl)phenyl)-5,8-dihydroimidazo[1,2-a]pyrimidin-3-yl)benzonitrile, 3-(2,4-difluorophenyl)-7-(4-(trifluoromethyl)phenyl)-2-(4-(trifluoromethyl)phenyl)imidazo[1,2-a]pyrimidin-5(8H)-one, 4-(3-(2,4-difluorophenyl)-5-oxo-7-(4-(trifluoromethyl)phenyl)-5,8-dihydroimidazo[1,2-a]pyrimidin-2-yl)benzonitrile, 4-(3-(4-methoxyphenyl)-5-oxo-7-((trifluoromethyl)phenyl)-5,8-dihydroimidazo[1,2-a]pyrimidin-2-yl)benzonitrile, 4-(2-(2,4-difluorophenyl)-5-oxo-7-(4-(trifluoromethyl)phenyl)-5,8-dihydroimidazo[1,2-a]pyrimidin-3-yl)benzonitrile, 2,3-bis(2,4-difluorophenyl)-7-(4-(trifluoromethyl)phenyl)imidazo[1,2-a]pyrimidin-5(1H)-one, 4-(7-amino-5-oxo-2-(4-(trifluoromethyl)phenyl)-5,8-dihydroimidazo[1,2-a]pyrimidin-3-yl)benzonitrile, 7-amino-3-(2,4-difluorophenyl)-2-(4-(trifluoromethyl)phenyl)imidazol-1,2-a]pyrimidin-5(8H)-one, 4-(7-amino-3-(2,4-difluorophenyl)-5-oxo-5,8-dihydroimidazo[1,2-a]pyrimidin-2-yl)benzonitrile, 4-(7-amino-3-(4-methoxyphenyl)-5-oxo-5,8-dihydroimidazo[1,2-a]pyrimidin-2-yl)benzonitrile, 4-(7-amino-2-(2,4-difluorophenyl)-5-oxo-5,8-dihydroimidazo[1,2-a]pyrimidin-3-yl)benzonitrile, 7-amino-2,3-bis(2,4-difluorophenyl)imidazo[1,2-a]pyrimidin-5(8H)-one, 7-amino-3-(2,4-difluorophenyl)-2-(4-(trifluoromethyl)phenyl)imidazo[1,2-a]pyrimidine-6-carboxamide, 7-amino-2-(4-cyanophenyl)-3-(2,4-difluorophenyl)imidazo[1,2-a]pyrimidine-6-carboxamide, 7-amino-2,3-bis(2,4-difluorophenyl)imidazo[1,2-a]pyrimidine-6-carboxamide, 7-amino-3-(trifluoromethyl)-2-(4-(trifluoromethyl)phenyl)imidazo[1,2-a]pyrimidine-6-carboxamide, 7-amino-2-(4-cyanophenyl)-7-((trifluoromethyl)phenyl)imidazo[1,2-a]pyrimidine-6-carboxamide, 7-amino-3-(2,4-difluorophenyl)-3-(trifluoromethyl)imidazo[1,2-a]pyrimidine-6-carboxamide, 3,7-diamino-2-(4-(trifluoromethyl)phenyl)imidazo[1,2-a]pyrimidine-6-carboxamide, 3,7-diamino-2-(4-cyanophenyl)-imidazo[1,2-a]pyrimidine-6-carboxamide, 3,7-diamino-2-(2,4-difluorophenyl)imidazo[1,2-a]pyrimidine-6-carboxamide, ethyl 3-(2,4-difluorophenyl)-5-oxo-2-(4-(trifluoromethyl)phenyl)-5,8-dihydroimidazo[1,2-a]pyrimidin-6-carboxylate, ethyl-2-(4-cyanophenyl)-3-(2,4-difluorophenyl)-5-oxo-5,8-dihydroimidazo[1,2-a]pyrimidin-6-carboxylate, ethyl 2-(4-cyanophenyl)-1-(2,4-dichlorophenyl)-6-oxo-6,7-dihydro-1H-imidazo[4,5-b]pyridine-5-carboxylate, ethyl-5-oxo-7-((trifluoromethyl)phenyl)-2-(4-(trifluoromethyl)phenyl)-imidazo[1,2-a]pyrimidine-6-carboxylate, ethyl 2-(4-cyanophenyl)-5-oxo-7-((trifluoromethyl)phenyl)-5,8-dihydroimidazo[1,2-a]pyrimidin-6-carboxylate, ethyl 2-(2,4-difluorophenyl)-5-oxo-7-((trifluoromethyl)phenyl)5,8-dihydroimidazo[1,2-a]pyrimidin-6-carboxylate, ethyl 3-amino-5-oxo-2-(4-(trifluoromethyl)phenyl)-5,8-dihydroimidazo[1,2-al pyrimidin-6-carboxylate, ethyl 3-amino-2-(4-cyanophenyl)-5-oxo-imidazo[1,2-a]pyrimidine-6-carboxylate, ethyl-3-amino-2-(2,4-difluorophenyl)-5-oxo-imidazo[1,2-a]pyrimidin-6-carboxylate, ethyl 3-amino-2-(4-cyanophenyl)-5-oxo-5,8-dihydro-imidazo[1,2-a]pyrimidin-6-carboxylate, 4-(3-(2,4-dichlorophenyl)-2-adamantyl-7-oxo-4,7-dihydropyrazolo[1,5-a]pyrimidin-5-yl)benzonitrile, 4-(3-adamantyl-2-cyclopropyl-7-oxo-4,7-dihydropyrazolo[1,5-a]pyrimidin-5-yl)benzonitrile, 2-cyclopropyl-3-(2,4-dichlorophenyl)-5-adamantyl pyrazolo[1,5-a]pyrimidin-7(4H)-one, 4-(3-(2,4-dichlorophenyl)-2-((adamantyl)amino)-7-oxo-4,7-dihydropyrazolo[1,5-a]pyrimidin-5-yl)benzonitrile, 4-(3-((adamantyl)amino)-2-cyclopropyl-7-oxo-4,7-dihydropyrazolo[1,5-a]pyrimidin-5-yl)benzonitrile, 2-cyclopropyl-3-(2,4-dichlorophenyl)-5-((adamantyl)amino)pyrazolo[1,5-a]pyrimidin-7(4H)-one, 4-(3-adamantyl-7-oxo-2-cyclopropyl-5-(4-(trifluoromethyl)phenyl)-4,7-dihydropyrazolo[1,5-a]pyrimidin-6-yl)benzonitrile, 7-adamantyl-6-methyl-2-(4-(trifluoromethyl)phenyl)-3H-cyclopenta[d]pyrimidin-4(4aH)-one, 4-(2-adamantyl-6-methyl-4-oxo-4,7-dihydro-3H-cyclopenta[d]pyrimidin-7-yl)benzonitrile, 4-(2-((adamantyl)amino)-6-methyl-4-oxo-4,7-dihydro-3H-cyclopenteno[d]pyrimidin-7-yl)benzonitrile, 7-(adamantylamino)-6-methyl-2-(4-(trifluoromethyl)phenyl)-3H-cyclopenteno[d]pyrimidin-4-one, 4-(6-(adamantyl)amino)-4-oxo-2-(4-(trifluoromethyl)phenyl)-4,7-dihydro-3H-cyclopenteno[d]pyrimidin-7-yl)benzonitrile, 3-((2,4-difluorophenyl)-6-methyl-2-phenyl-5-(6-(trifluoromethyl)pyridine)-3-yl)pyrazolo[1,5-a]pyrimidin-7(4H)one, 4-(2-ethyl-6-dimethyl-7-oxo-5-((4-trifluoromethyl)phenyl)-4,7-dihydropyrazolo[1,5-a]pyrimidin-3-yl)benzonitrile, 6-(2,4-difluorophenyl)-1-phenyl-6,7-dihydro-1H-pyrazolo[3,4-d]pyrimidin-4(5H)-one, 7-(4-(trifluoromethyl)phenyl)-1H-benzo[d]imidazo[1,2-a]pyrimidin-5(8H)-one;
The formation of six-membered A-cycle, 7-chloro-2-(2,4-dichlorophenyl)quinazolin-4(1H)-one, 7-chloro-2-(4-(trifluoromethyl)phenyl) quinazolin-4(1H)one, 7-morpholino-2-(4-(trifluoromethyl)phenyl)quinolin-4(1H)-one, 8-chloro-2-(pyridin-4-yl)-1H-pyrido[1,2-a][1,3,5]triazin-4(9aH)-one, 5-H-5-(3-nitrophenyl)chromenol-2,3-dl pyrimidin-4-imine, 4-imino-5-(3-nitrophenyl)-4H-chromeno[2,3-d]pyrimidin-3(5H)-amine, 8-H-8-(3-phenyl)benzeno[7,8]chromeno[2,3-4′,5′]pyrimido[1′,6′-e][1,2,4]triazole, 6-methyl-8-H-8-(3-nitro-phenyl)benzo[7,8]chromeno[2,3-4′,5′]pyrimido[1′,6′-e][1,2,4]triazole, 4-(2,4-dichlorobenzyl imino)-5-H-5-(3-nitrophenyl)benzo[7,8]chromene[2,3-d]pyrimidin-5-imine-6-ethoxy carbonyl-8-H-8-(3-nitrophenyl)benzo[7,8]chromeno[2,3-4′,5′]pyrimido[1′,6′-e]1,2,4-triazole, 6-phenylamino-8-H-8-(3-nitrophenyl)benzo[7,8]chromeno[2,3-4′,5′]pyrimido[1′,6′-e]-1,2,4-triazole, 6-phenyl-8-H-8-(3-nitrophenyl)benzo[7,8]chromeno[2,3-4′,5′]pyrimido[1′,6′-e]-1,2,4-triazole, N-benzoylmethyl-1H-indole-2-cyanide, 4-phenyl-3H-[1,2,5]triazino[5,4-a]indole-1-amine, 7-morpholino-2-(4-(trifluoro methyl)phenyl)-4H-benzo[d][1,3][1,3,5]triazin-4-one, 7-phenoxy-2-(4-(trifluoromethyl)phenyl)-4H-benzo[d][1,3]triazin-4-one, 2-(ethoxymethylamino)-4-(3-nitrophenyl)-4H-benzo[h]chromene-3-carbonitrile, 6H-phthalazine[1,2-b]quinazolin-5,8-dione, 2-(4-chlorophenyl)-4-oxo-8-phenyl-4H-pyrimido[1,2-a]pyrimidin-3-nitrile, 2-mercapto-7-morpholino-3-phenylquinolin-yl-4(3H)one, 4-(3-(2,4-difluorophenyl)-2-methyl-4-oxo-4H-pyrimido[1,2-b]pyridazin-8-yl)benzonitrile, 4-(3-(2,4-difluorophenyl)-4-oxo-4H-2-methylpyrimido[1,2-b]pyridazin-8-yl)benzonitrile, 4-(3-(2,4-difluorophenyl)-7-H-2-methylpyrimido[1,2-b]pyridazin-4-oxo)benzonitrile, 4-(3-(2,4-difluorophenyl)-4H-2,4-dimethylpyrimido[1,2-b]pyridazin-4(6H)-oxo)benzonitrile, 4-(8-(2,4-difluorophenyl)-1,7-dimethyl-4-oxo-4,9-dihydro-1H-pyrimido[1,2-b]pyridazin-2-yl)benzonitrile, 4-(7-methyl-4-oxo-8-(4-(trifluoromethyl)phenyl)-4,6-dihydro-1H-pyrimido[1,2-b]pyridazin-2-yl)benzonitrile, 4-(7-methyl-4-oxo-8-(4-(trifluoromethyl)phenyl)-4,9-dihydro-1H-pyrimido[1,2-b]pyridazin-2-yl)benzonitrile, 4-(7-methyl-4-oxo-8-(4-(trifluoromethyl)phenyl)-4H-pyrimido[1,2-b]pyridazin-2-yl)benzonitrile, 8-(2,4-difluorophenyl)-7-methyl-2-(4-(trifluoromethyl)phenyl)-1H-pyrimido[1,2-b]pyridazin-4(6H)-one, 8-(2,4-difluorophenyl)-7-methyl-2-(4-(trifluoromethyl)phenyl)-1H-pyrimido[1,2-b]pyridazin-4-one, 8-(2,4-difluorophenyl)-7-methyl-2-(4-(trifluoromethyl)phenyl)-4H-pyrimido[1,2-b]pyridazin-4-one, 7-methyl-2,8-bis(4-(trifluoromethyl)phenyl)-1H-pyrimido[1,2-b]pyridazin-4(6H)-one, 7-methyl-2,8-bis(4-(trifluoromethyl)phenyl)-1H-pyrimidine[1,2-b]pyridazin-4-one, 7-methyl-2,8-bis(4-(trifluoromethyl)phenyl)-4H-pyrimido[1,2-b]pyridazin-4-one, 7-methyl-8-(4-(trifluoromethyl)phenyl)-5-(6-trifluoromethyl)pyridin-3-yl)-1H-pyrimido[1,2-b]pyridazin-4(6H)-one, 7-methyl-8-(4-(trifluoromethyl)phenyl)-2-(6-trifluoromethyl)pyridin-3-yl)-1H-pyrimido[1,2-b]pyridazin-4(6H)-one, 7-methyl-8-(4-(trifluoromethyl)phenyl)-2-(6-trifluoromethyl)pyridin-3-yl)-4H-pyrimido[1,2-b]pyridazin-4-one, 2-(2,4-difluorophenyl)-7-methyl-8-(4-(trifluoromethyl)phenyl)-1H-pyrimido[1,2-b]pyridazin-4(6H)-one, 2-(2,4-difluorophenyl)-7-methyl-8-(4-(trifluoromethyl)phenyl)-1H-pyrimido[1,2-b]pyridazin-4-one, 2-(2,4-difluorophenyl)-7-methyl-8-(4-(trifluoromethyl)phenyl)-4H-pyrimido[1,2-b]pyridazin-4-one, 8-(2,4-difluorophenyl)-2-(4-methoxyphenyl)-7-methyl-1H-pyrimido[1,2-b]pyridazin-4(6H)-one, 8-(2,4-difluorophenyl)-2-(4-methoxyphenyl)-7-methyl-1H-pyrimido[1,2-b]pyridazin-4-one, 8-(2,4-difluorophenyl)-2-(4-methoxyphenyl)-7-methyl-7,8-dihydropyrazino[1,2-a]pyrimidin-6-one, 8-(2,4-difluorophenyl)-2-(4-methoxyphenyl)-7-methyl-4H-pyrimido[1,2-b]pyridazin-4-one, 2-(4-aminophenyl)-8-(2,4-difluorophenyl)-7-methyl-1H-pyrimido[1,2-b]pyridazin-4(6H)-one, 2-(4-aminophenyl)-8-(2,4-difluorophenyl)-7-methyl-1H-pyrimido[1,2-b]pyridazin-4(9aH)-one, 2-(4-aminophenyl)-8-(2,4-difluorophenyl)-7-methyl-4H-pyrimido[1,2-b]pyridazin-4-one, 4-(8-(2,4-difluorophenyl)-7-methyl-4-oxo-4,6-dihydro-1H-pyrimido[1,2-b]pyridazin-2-yl-aminomethyl)benzonitrile, 4-(8-(2,4-difluorophenyl)-7-methyl-4-oxo-4,9-dihydro-1H-pyrimido[1,2-b]pyridazin-2-yl-aminomethyl)benzonitrile, 4-(8-(2,4-difluorophenyl)-7-methyl-4-oxo-4H-pyrimido[1,2-b]pyridazin-2-yl-aminomethyl)benzonitrile, 4-(8-(2,4-fluorophenyl-4-(8-(2,4-difluorophenyl)-7-methyl-4-oxo-4H-pyrimido[1,2-b]pyridazin-2-yl)-N,N-dipropylbenzenesulfonamide, 4-(8-(2,4-difluorophenyl)-7-methyl-4-oxo-4,9-dihydro-1H-pyrimido[1,2-b]pyridazin-2-yl)-N,N-dipropylbenzenesulfonamide, 4-(8-(2,4-difluorophenyl)-7-methyl-4-oxo-4H-pyrimido[1,2-b]pyridazin-2-yl)-N,N-dipropylbenzene sulfonamide, 3-(2,4-difluorophenyl)-4H-2-ethyl-5-(4-(trifluoromethyl)phenyl)pyrimido[1,2-b]pyridazin-4(6H)-one, 3-(2,4-difluorophenyl)-4H-2-ethyl-5-(4-(trifluoromethyl)phenyl)pyrimido[1,2-b]pyridazino-4-one, 3-(2,4-difluorophenyl)-7-H-2-ethyl-5-(4-(trifluoromethyl)phenyl)pyrimido[1,2-b]pyridazin-4-one, 4-(3-(2,4-difluorophenyl)-4H-2-phenylpyrimido[1,2-b]pyridazin-4(6H)-oxo-5-yl)benzonitrile, 4-(8-(2,4-difluorophenyl)-4-oxy-7-phenyl-5,9-dihydro-1H-pyrimido[1,2-b]pyridazin-2-yl)benzonitrile, 4-(8-(2,4-difluorophenyl)-4-oxo-7-phenyl-4H-pyrimido[1,2-b]pyridazin-2-yl)benzonitrile, 8-(2,4-difluorophenyl)-7-phenyl-2-(4-(trifluoromethyl)phenyl)-1H-pyrimido[1,2-b]pyridazin-4(6H)-one, 8-(2,4-fluorophenyl)-7-phenyl-2-(4-(trifluoromethyl)phenyl)-1H-pyrimido[1,2-b]pyridazin-4-one, 8-(2,4-difluorophenyl)-7-phenyl-2-(4-(trifluoromethyl)phenyl)-4H-pyrimido[1,2-b]pyridazin-4-one, 4-(7-cyclopropyl-8-(2,4-difluorophenyl)-4-oxo-4,6-dihydro-1H-pyrimido[1,2-b]pyridazin-2-yl)benzonitrile, 4-(7-cyclopropyl-8-(2,4-difluorophenyl)-4-oxo-4,9-dihydro-1H-pyrimido[1,2-b]pyridazin-2-yl)benzonitrile, 4-(7-cyclopropyl-8-(2,4-difluorophenyl)-4-oxo-4H-pyrimido[1,2-b]pyridazin-2-yl)benzonitrile, 4-(8-(2,4-difluorophenyl)-4-oxo-7-(trifluoromethyl)-4,6-dihydro-1H-pyrimido[1,2-b]pyridazin-2-yl)benzonitrile, 4-(8-(2,4-difluorophenyl)-4-oxo-7-((trifluoromethyl)phenyl)-4-dihydro-1H-pyrimido[1,2-b]pyridazin-2-yl)benzonitrile, 4-(8-(2,4-difluorophenyl)-4-oxo-7-((trifluoromethyl)phenyl)-4H-pyrimido[1,2-b]pyridazin-2-yl)benzonitrile, 4-(8-(2,4-difluorophenyl)-7-ethyl-4-oxo-1H-pyrimido[1,2-b]pyridazin-2-yl)benzonitrile, 4-(8-(2,4-difluorophenyl)-7-ethyl-4-oxo-4,9-dihydro-1H-pyrimido[1,2-b]pyridazin-2-yl)benzonitrile, 4-(8-(2,4-difluorophenyl)-7-ethyl-4-oxo-pyrimidino(4H)[1,2-b]pyridazin-2-yl)benzonitrile, 4-(8-(2,4-dichlorophenyl)-7-methyl-4-oxo-4,6-dihydro-1H-pyrimido[1,2-b]pyridazin-2-yl)benzonitrile, 4-(8-(2,4-dichlorophenyl)-7-methyl-4-oxo-4,9-dihydro-1H-pyrimido[1,2-b]pyridazin-2-yl)benzonitrile, 4-(8-(2,4-dichlorophenyl)-7-methyl-4-oxo-4H-pyrimido[1,2-b]pyridazin-2-yl)benzonitrile, 4-(2-(4-cyanophenyl)-7-methyl-4-oxo-4,6-dihydro-1H-pyrimido[1,2-b]pyridazin-8-yl)benzoic acid, 4-(2-(4-cyanophenyl)-7-methyl-4-oxo-4,9-dihydro-1H-pyrimido[1,2-b]pyridazin-8-yl)benzoic acid, 4-(2-(4-cyanophenyl)-7-methyl-4-oxo-4H-pyrimido[1,2-b]pyridazin-8-yl)benzoic acid, 7-chloro-2-((4-trifluoromethyl)phenyl)quinolinyloxazolin-4(3H)-one, 7-morpholino-2-(4-(trifluoromethyl)phenyl)quinazolin-4(1H)one, 7-phenoxy-2-(4-(trifluoromethyl)phenyl)quinazolin-4(1H)one, 3-(2,4-difluorophenyl)-5-(3-nitrophenyl)-8-(trifluoromethyl)-3H-chromeno[2,3-d]pyrimidin-4(5H)imine, 3-(3-(2,4-difluorophenyl)-4-imino-8-((trifluoromethyl)phenyl)-4,5-dihydrochromeno[2,3-d]pyrimidin-5-yl)aniline, 3-(2,4-difluorophenyl)-4-imino-5-(3-nitrophenyl)-4,5-dihydro-3H-chromeno[2,3-d]pyrimidin-8-carbonitrile, 5-(3-aminophenyl)-3-(2,4-difluorophenyl)-4-imino-4,5-dihydro-3H-chromeno[2,3-d]pyrimidin-8-carbonitrile, 3-(2,4-difluorophenyl)-6,8-difluorophenyl-5-(3-nitrophenyl)-3H-chromeno[2,3-d]pyrimidin-4(5H) imine, 3-(3-(2,4-difluorophenyl)-6,8-difluoro-4-imino-4,5-dihydro-3H-chromeno[2,3-d]pyrimidin-5-yl)aniline, 5-(3-nitrophenyl)-3,8-bis(trifluoromethyl)-3H-chromeno[2,3-d]pyrimidin-4(5H)imine, 3-(4-imino-3,8-bis(trifluoromethyl)-4,5-dihydro-3H-chromeno[2,3-d]pyrimidin-5-yl)aniline, 4-imino-5-(3-nitrophenyl)-3-(trifluoromethyl)-4,5-dihydro-3H-chromeno[2,3-d]pyrimidin-8-carbonitrile, 5-(3-aminophenyl)-4-imino-3-((trifluoromethyl)phenyl)-4,5-dihydro-3H-chromeno[2,3-d]pyrimidin-8-carbonitrile, 6,8-difluoro-5-(3-nitrophenyl)-3-((trifluoromethyl)phenyl)-3H-chromeno[2,3-d]pyrimidin-4(5H)-imine, 3-(6,8-difluoro-4-imino-3-((trifluoromethyl)phenyl)-4,5-dihydro-3H-chromeno[2,3-d]pyrimidin-5-yl)aniline, 4-imino-5-(3-nitrate phenyl)-8-((trifluoromethyl)phenyl)-4H-chromeno[2,3-d]pyrimidin-3(5H)amine, 5-(3-aminophenyl)-4-imino-84 trifluoromethyl)-4H-chromeno[2,3-d]pyrimidin-3(5H)amine, 3-amino-4-imino-5-(3-nitrophenyl)-4,5-dihydro-3H-chromeno[2,3-d]pyrimidin-8-carbonitrile, 3-amino-5-(3-aminophenyl)-4-amino-4,5-iminodihydro-3H-chromeno[2,3-d]pyrimidin-8-carbonitrile, 6,8-difluoro-4-imino-5-(3-nitrophenyl)-4H-chromeno[2,3-d]pyrimidin-3(5H)amine, 5-(3-aminophenyl)-6,8-difluoro-4-imino-4H-chromeno[2,3-d]pyrimidin-3(5H)amine, 4-(3-(2,4-difluorophenyl)-4-oxo-2-methylpyrimido[1,2-b]pyridazinyl)benzonitrile, 4-(3-(4-aminophenyl)-4-oxo-2-methylpyrimido[1,2-b]pyridazin-9-yl)benzonitrile, 4-(3-(2,4-difluorophenyl)-4-oxo-2-methylpyrimido[1,2-b]pyridazin-9-yl)benzonitrile, 4-(3-(2,4-difluorophenyl)-2-methyl-4-oxo-4H-pyrimido[1,2-b]pyridazin-8-yl)benzonitrile, 4-(8-(2,4-difluorophenyl)-1,7-dimethyl-4-oxo-4,9-dihydro-1H-pyrimido[1,2-b]pyridazin-2-yl)benzonitrile, 4-(7-methyl-4-oxo-8-(4-(trifluoromethyl)phenyl)-4,6-dihydro-1H-pyrimido[1,2-b]pyridazin-2-yl)benzonitrile, 4-(7-methyl-4-oxo-8-(4-(trifluoromethyl)phenyl)-4,9-dihydro-1H-pyrimido[1,2-b]pyridazin-2-yl)benzonitrile, 4-(7-methyl-4-oxo-8-(4-(trifluoromethyl)phenyl)-4H-pyrimido[1,2-b]pyridazin-2-yl)benzonitrile, 8-(2,4-difluorophenyl)-7-methyl-2-(4-(trifluoromethyl)phenyl)-1H-pyrimido[1,2-b]pyridazin-4(6H)one, 8-(2,4-difluorophenyl)-7-methyl-2-(4-(trifluoromethyl)phenyl)-1H-pyrimido[1,2-b]pyridazin-4-one, 8-(2,4-difluorophenyl)-7-methyl-2-(4-(trifluoromethyl)phenyl)-4H-pyrimido[1,2-b]pyridazin-4-one, 7-methyl-2,8-bis(4-(trifluoromethyl)phenyl)-1H-pyrimido[1,2-b]pyridazin-4(6H)one, 7-methyl-2,8-bis(4-(trifluoromethyl)phenyl)-1H-pyrimido[1,2-b]pyridazin-4-one, 7-methyl-2,8-bis(4-(trifluoromethyl)phenyl)-4H-pyrimido[1,2-b]pyridazin-4-one, 7-methyl-8-(4-(trifluoromethyl)phenyl)-3-((6-trifluoromethyl)pyridin-3-yl)-1H-pyrimido[1,2-b]pyridazin-4(6H)-one, 7-methyl-8-(4-(trifluoromethyl)phenyl)-2-((6-trifluoromethyl)pyridin-3-yl)-1H-pyrimido[1,2-b]pyridazin-4(6H)one, 7-methyl-8-(4-(trifluoromethyl)phenyl)-2-((6-trifluoromethyl)pyridin-3-yl)-4H-pyrimido[1,2-b]pyridazin-4-one, 2-(2,4-difluorophenyl)-7-methyl-8-(4-(trifluoromethyl)phenyl)-1H-pyrimido[1,2-b]pyridazin-4(6H)-one, 2-(2,4-difluorophenyl)-7-methyl-8-(4-(trifluoromethyl)phenyl)-1H-pyrimido[1,2-b]pyridazin-4-one, 2-(2,4-difluorophenyl)-7-methyl-8-(4-(trifluoromethyl)phenyl)-4H-pyrimido[1,2-b]pyridazin-4-one, 2-(4-methoxyphenyl)-7-methyl-8-(2,4-difluorophenyl)-4H-pyrimido[1,2-b]pyridazin-4-one, 8-(2,4-difluorophenyl)-2-(4-methoxyphenyl)-7-methyl-1H-pyrimido[1,2-b]pyridazin-4-one, 8-(2,4-difluorophenyl)-2-(4-methoxyphenyl)-7-methyl-4H-pyrimido[1,2-b]pyridazin-4-one, 2-(4-aminophenyl)-8-(2,4-difluorophenyl)-7-methyl-1H-pyrimido[1,2-b]pyridazin-4(6H)-one, 2-(4-aminophenyl)-8-(2,4-difluorophenyl)-7-methyl-1H-pyrimido[1,2-b]pyridazin-4(9aH)-one, 2-(4-aminophenyl)-8-(2,4-difluorophenyl)-7-methyl-4H-pyrimido[1,2-b]pyridazin-4-one, 4-((8-(2,4-difluorophenyl)-7-methyl-4-oxo-4,6-dihydro-1H-pyrimido[1,2-b]pyridazin-2-yl)aminomethyl)benzonitrile, 4-((8-(2,4-difluorophenyl)-7-methyl-4-oxo-4,9-dihydro-1H-pyrimido[1,2-b]pyridazin-2-yl)aminomethyl)benzonitrile, 4-((8-(2,4-difluorophenyl)-7-methyl-4-oxo-4H-pyrimido[1,2-b]pyridazin-2-yl)aminomethyl)benzonitrile, 4-(7-methyl-4-oxo-8-(4-(trifluoromethyl)phenyl)-4H-pyrimido[1,2-b]pyridazin-2-yl)-N,N-dipropylbenzenesulfonamide, 4-(7-methyl-4-oxo-8-(2,4-difluorophenyl)-4H-pyrimido[1,2-b]pyridazin-2-yl)-N,N-dipropylbenzene sulfonamide, 4-(8-(2,4-difluorophenyl)-7-methyl-4-oxo-4H-pyrimido[1,2-b]pyridazin-2-yl)-N,N-dipropyl benzene sulfonamide, 3-(2,4-difluorophenyl)-4H-2-ethyl-5-(4-(trifluoromethyl)phenyl)pyrimido[1,2-b]pyridazin-4(6H)one, 3-(2,4-difluorophenyl)-4H-2-ethyl-5-(4-(trifluoromethyl)phenyl)pyrimido[1,2-b]pyridazin-4-one, 3-(2,4-difluorophenyl)-4H-2-ethyl-8-(4-(trifluoromethyl)phenyl)pyrimido[1,2-b]pyridazin-4-one, 4-(3-(2,4-difluorophenyl)-4H-2-phenyl-pyrimido[1,2-b]pyridazin-4-oxo-7-yl)benzonitrile, 4-(8-(2,4-difluorophenyl)-4-oxo-7-phenyl-4,9-dihydro-1H-pyrimido[1,2-b]pyridazin-2-yl)benzonitrile, 4-(8-(2,4-fluorophenyl)-4-oxo-7-phenylpyrimido[1,2-b]pyridazin-2-yl)benzonitrile, 8-(2,4-difluorophenyl)-7-phenyl-2-(4-(trifluoromethyl)phenyl)-1H-pyrimido[1,2-b]pyridazin-4(6H)one, 8-(2,4-difluorophenyl)-7-phenyl-2-(4-(trifluoromethyl)phenyl)-1H-pyrimido[1,2-b]pyridazin-4-one, 8-(2,4-difluorophenyl)-7-phenyl-2-(4-(trifluoromethyl)phenyl)-4H-pyrimido[1,2-b]pyridazin-4-one, 4-(7-cyclopropyl-8-(2,4-difluorophenyl)-4-oxo-4H-pyrimido[1,2-b]pyridazin-2-yl)benzonitrile, 4-(7-cyclopropyl-8-(2,4-difluorophenyl)-4-oxo-4,9-dihydro-1H-pyrimido[1,2-b]pyridazin-2-yl) benzonitrile, 4-(7-cyclopropyl-8-(2,4-difluorophenyl)-4-oxo-4H-pyrimido[1,2-b]pyridazin-2-yl)benzonitrile, 4-(8-(2,4-difluorophenyl)-4-oxo-7-((trifluoromethyl)phenyl)-4,6-dihydro-1H-pyrimido[1,2-b]pyridazin-2-yl)benzonitrile, 4-(8-(2,4-difluorophenyl)-4-oxo-7-((trifluoromethyl)phenyl)-4,6-dihydro-1H-pyrimido[1,2-b]pyridazin-2-yl)benzonitrile, 4-(8-(2,4-difluorophenyl)-4H-4-oxo-7-(trifluoromethyl)pyrimido[1,2-b]pyridazin-2-yl)benzonitrile, 4-(8-(2,4-difluorophenyl)-7-ethyl-4-oxo-1H-pyrimido[1,2-b]pyridazin-2-yl)benzonitrile, 4-(8-(2,4-difluorophenyl)-7-ethyl-4-oxo-4,9-dihydro-1H-pyrimido[1,2-b]pyridazin-2-yl)benzonitrile, 4-(8-(2,4-difluorophenyl)-7-ethyl-4-oxo-4H-pyrimido[1,2-b]pyridazin-2-yl)benzonitrile, 4-(8-(2,4-dichlorophenyl)-7-methyl-4-oxo-4,6-dihydro-1H-pyrimido[1,2-b]pyridazin-2-yl)benzonitrile, 4-(8-(2,4-dichlorophenyl)-7-methyl-4-oxo-4,9-dihydro-1H-pyrimido[1,2-b]pyridazin-2-yl)benzonitrile, 4-(8-(2,4-dichlorophenyl)-7-methyl-4-oxo-4H-pyrimido[1,2-b]pyridazin-2-yl)benzonitrile, 4-(2-(4-cyanophenyl)-7-methyl-4-oxo-4,6-dihydro-1H-pyrimido[1,2-b]pyridazin-8-yl)benzoic acid, 4-(2-(4-cyanophenyl)-7-methyl-4-oxo-4,9-dihydro-1H-pyrimido[1,2-b]pyridazin-8-yl)benzoic acid, 4-(2-(4-cyanophenyl)-7-methyl-4-oxo-4H-pyrimido[1,2-b]pyridazin-8-yl)benzoic acid, 7-chloro-2-((4-trifluoromethyl)phenyl)quinolin-4-one, 7-morpholino-2-(4-(trifluoromethyl)phenyl)quinazolin-4(1H)one, 7-phenoxy-2-(4-(trifluoromethyl)phenyl)quinazolin-4(3H)-one, 3-(2,4-difluorophenyl)-5-(3-nitro-phenyl)-8-(trifluoro methyl)-3H-chromeno[2,3-d]pyrimidin-4(5H)imine, 3-(3-(2,4-difluorophenyl)-4-imino-8-(trifluoromethyl)-4,5-dihydro-3H-chromeno[2,3-d]pyrimidin-5-yl)-aniline, 3-(2,4-di-fluorophenyl)-4-imino-5-(3-nitrophenyl)-4,5-dihydro-3H-chromeno[2,3-d]pyrimidin-8-carbonitrile, 5-(3-amino-phenyl)-3-(2,4-difluorophenyl)-4-imino-4,5-dihydro-3H-chromeno[2,3-d]pyrimidin-8-carbonitrile, 3-(2,4-difluorophenyl)-8-difluorophenyl-5-(3-nitrophenyl)-3H-chromeno[2,3-d]pyrimidin-4(5H)imine, 3-(3-(2,4-difluorophenyl)-6,8-difluoro-4-imino-4,5-dihydro-3H-chromeno[2,3-d]pyrimidin-5-yl)aniline, 5-(3-nitrophenyl)-3,8-bis(trifluoromethyl)-3H-chromeno[2,3-d]pyrimidin-4(5H) imine, 3-(4-imino-3,8-bis(trifluoromethyl)-4,5-dihydro-3H-chromeno[2,3-d]pyrimidin-5-yl)aniline, 5-(3-nitrophenyl)-8-cyano-3-(trifluoromethylphenyl)-4,5-dihydro-3H-chromeno[2,3-d]pyrimidine-4-imine, 5-(3-aminophenyl)-4-imino-3-(trifluoromethylphenyl)-4,5-dihydro-3H-chromeno[2,3-d]pyrimidin-8-carbonitrile, 6,8-difluoro-5-(3-nitrophenyl)-3-((trifluoromethyl)phenyl)-3H-chromeno[2,3-d]pyrimidin-4(5H)imine, 3-(6,8-difluoro-4-imino-7-((trifluoromethyl)phenyl)-4,5-dihydro-3H-chromeno[2,3-d]pyrimidin-5-yl)aniline, 4-imino-5-(3-nitrophenyl)-8-((trifluoromethyl)phenyl)-4H-chromeno[2,3-d]pyrimidin-3(5H)amine, 5-(3-aminophenyl)-4-imino-8-(trifluoromethylphenyl)-4H-chromeno[2,3-d]pyrimidin-3(5H)amine, 3-amino-4-imino-5-(3-nitrophenyl)-4,5-dihydro-3H-chromeno[2,3-d]pyrimidin-8-carbonitrile, 3-amino-5-(3-aminophenyl)-4-imino-4,5-dihydro-3H-chromeno[2,3-d]pyrimidin-8-carbonitrile, 6,8-bis(2,4-difluorophenyl)-4-imino-5-(3-nitrophenyl)-4,5-dihydrochromeno[2,3-d]pyrimidin-3-amine, 6,8-bis(2,4-difluorophenyl)-4-imino-5-(3-aminophenyl)-4,5-dihydrochromeno[2,3-d]pyrimidin-3-amine, 3-(2,4-difluorophenyl)-4,5-dihydro-5-(3-nitrophenyl)-8-((trifluoromethyl)phenyl)-3H-chromeno[2,3-d]pyrimidin-4-imine, 3-(3-(2,4-difluorophenyl)-4-imino-8-((trifluoromethyl)phenyl)-4,5-dihydro-3H-chromeno[2,3-d]pyrimidin-3-yl)aniline, 3-(2,4-difluorophenyl)-4-imino-5-(3-nitrophenyl)-4,5-dihydro-3H-chromeno[2,3-d]pyrimidin-8-carbonitrile, 5-(3-aminophenyl)-3-(2,4-difluorophenyl)-4-imino-4,5-dihydro-3H-chromeno[2,3-d]pyrimidin-8-carbonitrile, 3-(2,4-difluorophenyl)-6,8-dihydro-5-(3-nitrophenyl)-3H-chromeno[2,3-d]pyrimidin-4(5H)imine, 3-(3-(2,4-difluorophenyl)-6,8-difluoro-4-imino-4,5-dihydrochromeno[2,3-d]pyrimidin-3-yl)aniline, 5-(3-nitrophenyl)-3-adamantyl-8-(trifluoromethyl)-3H-chromeno[2,3-d]pyrimidin-4(5H)imine, 5-(3-aminophenyl)-3-adamantyl-8-((trifluoromethyl)phenyl)-3H-chromeno[2,3-d]pyrimidin-4(5H)imine, 5-(3-aminophenyl)-3-adamantyl-8-cyano-3H-chromeno[2,3-d]pyrimidin-4(5H)imine, 5-(3-nitrophenyl)-3-adamantyl-8-cyano-3H-chromeno[2,3-d]pyrimidin-4(5H)imine, 5-(3-nitrophenyl)-3-adamantyl-6,8-difluoro-3H-chromeno[2,3-d]pyrimidin-4(5H)imine, 5-(3-aminophenyl)-3-adamantyl-6,8-difluoro-3H-chromeno[2,3-d]pyrimidin-4(5H) imine, 5-(3-nitrophenyl)-3-amino-8-((trifluoromethyl)phenyl)-3H-chromeno[2,3-d]pyrimidin-4(5H) imine, 5-(3-aminophenyl)-3-amino-8-((trifluoromethyl)phenyl)-3H-chromeno[2,3-d]pyrimidin-4(5H)imine, 5-(3-nitrophenyl)-3-amino-8-cyano-3H-chromeno[2,3-d]pyrimidin-4(5H)imine, 5-(3-aminophenyl)-3-amino-8-cyano-3H-chromeno[2,3-d]pyrimidin-4(5H)imine, 5-(3-nitrophenyl)-3-amino-6,8-difluoro-3H-chromeno[2,3-d]pyrimidin-4(5H)imine, 5-(3-aminophenyl)-3-amino-6,8-difluoro-3H-chromeno[2,3-d]pyrimidin-4(5H) imine, 7-(adamantylamino)-6-methyl-2-(4-(trifluoromethyl)phenyl)-3H-cyclopenta[d]pyrimidin-4-one, 4-((2-adamantylamino)-6-methyl-4-oxo-4,7-dihydrocyclopenta[d]pyrimidin-7-yl)benzonitrile, 4-(2-adamantylamino)-6-methyl-4-oxo-4,7-dihydro-3H-cyclopenta[d]pyrimidin-7-yl)benzonitrile, 6-methyl-7-adamantyl-2-(4-(trifluoromethyl)phenyl)quinazolin-4(1H)one, 6-methyl-7-phenoxyl-2-adamantylquinazolin-4-one, 6-methyl-7-adamantylamino-2-(4-(trifluoromethyl)phenyl)quinazolin-4(3H)one, 6-dimethyl-7-phenoxy-2-(adamantyl amino)phenyl)quinazolin-4(1H)one, 4-(8-(2,4-difluorophenyl)-7-adamantyl-4-oxo-4H-1H-pyrimido[1,2-b]pyridazin-2-yl)benzonitrile, 4-(8-adamantyl-7-methyl-4-oxo-4,9-dihydro-1H-pyrimido[1,2-b]pyridazin-2-yl)benzonitrile, 4-(8-(2,4-difluorophenyl)-7-(adamantylamino)-4-oxo-4H-1H-pyrimido[1,2-b]pyridazin-2-yl)benzonitrile, 4-(8-(N-adamantylamino)-7-methyl-4-oxo-pyrimido[1,2-b]pyridazin-2-yl)benzonitrile, 8-(2,4-difluorophenyl)-2-(N-adamantylamino)-7-methyl-1H-pyrimido[1,2-b]pyridazin-4-one, ethyl 5-hydroxy-2-methyl-7-oxo-3-(4-(trifluoromethyl)phenyl)-4,7-dihydropyrazolo[1,5-a]pyrimidine-6-carboxylate, 3-(2,4-dichlorophenyl)-2-methyl-5-(4-(trifluoromethyl)phenyl)pyrazolo[1,5-a]pyrimidin-7(4H)-one, 4-(2-methyl-7-oxo-3-(4-(trifluoromethyl)phenyl)-4,7-dihydropyrazolo[1,5-a]pyrimidin-5-yl)benzonitrile, 4-(2-cyclopropyl-3-(2,4-dichlorophenyl)-7-oxo-4,7-dihydropyrazolo[1,5-a]pyrimidin-5-yl)benzonitrile, 4-(3-(2,4-dichlorophenyl)-7-oxo-2-phenyl-4,7-dihydropyrazolo[1,5-a]pyrimidin-5-yl)benzonitrile, 3-(2,4-dichlorophenyl)-2-methyl-5-(4-(trifluoromethyl)phenyl)pyrazolo[1,5-a]pyrimidin-7(4H)-one, 3-(2,4-dichlorophenyl)-2-methyl-5-(6-(trifluoromethyl)pyridin-3-yl)pyrazolo[1,5-a]pyrimidin-7(4H)-one, 4-(3-(2,4-dichlorophenyl)-2-methyl-7-oxo-4,7-dihydropyrazolo[1,5-a]pyrimidin-5-yl)benzonitrile, 3-(2,4-dichlorophenyl)-2-methyl-5-(morpholino methyl)pyrazolo[1,5-a]pyrimidin-7(4H)-one, methyl 3-(2,4-dichlorophenyl)-2-methyl-7-oxo-4,7-dihydropyrazolo[1,5-a]pyrimidine-6-carboxylate, 7-chloro-2-(4-(trifluoromethyl)phenyl)-4H-benzo[d][1,3]oxazin-4-one, 7-chloro-2-(4-(trifluoromethyl)phenyl)quinazolin-4(3H)-one, 7-morpholino-2-(4-(trifluoromethyl)phenyl)quinazolin-4(3H)-one, 4-((3-(2,4-dichlorophenyl)-2-methyl-7-oxo-4,7-dihydropyrazolo[1,5-a]pyrimidin-5-yl)methylamino)benzonitrile, 54(4-aminophenylamino)methyl)-3-(2,4-dichlorophenyl)-2-methylpyrazolo[1,5-a]pyrimidin-7(4H)-one, ethyl 2-cyclopropyl-3-(2,4-dichlorophenyl)-7-oxo-4,7-dihydropyrazolo[1,5-a]pyrimidine-6-carboxylate, 3-(2,4-dichlorophenyl)-2,6-dimethyl-5-(4-(trifluoromethyl)phenyl)pyrazolo[1,5-a]pyrimidin-7(4H)-one, 2-cyclopropyl-3-(2,4-dichlorophenyl)-6-methyl-5-(4-(trifluoromethyl)phenyl)pyrazolo[1,5-a]pyri-midin-7(4H)-one, 3-(2,4-dichlorophenyl)-6-methyl-2-phenyl-5-(4-(trifluoromethyl)phenyl)pyrazolo[1,5-a]pyrimidin-7(4H)-one, 3-(2,4-dichlorophenyl)-2-ethyl-6-methyl-5-(4-(trifluoromethyl)phenyl)pyrazolo[1,5-a]pyrimidin-7(4H)-one, 2,6-dimethyl-3,5-bis(4-(trifluoromethyl)phenyl)pyrazolo[1,5-a]pyrimidin-7(4H)-one, 4-(3-(2,4-dichlorophenyl)-2,6-dimethyl-7-oxo-4,7-dihydropyrazolo[1,5-a]pyrimidin-5-yl)-N,N-dipropylbenzenesulfonamide, 4-(3-(2,4-dichlorophenyl)-6-methyl-7-oxo-2-phenyl-4,7-dihydropyrazolo[1,5-a]pyrimidin-5-yl)-N,N-dipropylbenzenesulfonamide, 4-(3-(2,4-dichlorophenyl)-6-methyl-7-oxo-2-phenyl-4,7-dihydropyrazolo[1,5-a]pyrimidin-5-yl)benzonitrile, 4-(3-(2,4-dichlorophenyl)-2,6-dimethyl-7-oxo-4,7-dihydropyrazolo[1,5-a]pyrimidin-5-yl)benzonitrile, 3-(2,4-dichlorophenyl)-2-ethyl-6-methyl-5-(6-(trifluoromethyl)pyridin-3-yl)pyrazolo[1,5-a]pyrimidin-7(4H)-one, 7-amino-3-(2,4-dichlorophenyl)-2-methylpyrazolo[1,5-a]pyrimidine-6-carbonitrile, 5-amino-3-(2,4-dichlorophenyl)-2-methyl-7-(3-nitrophenyl)-4,7-dihydropyrazolo[1,5-a]pyrimidine-6-carbonitrile, 4-imino-1,5-diphenyl-4,5-dihydro-1H-pyrazolo[3,4-d]pyrimidine-6(7H)-thione, 1,6-diphenyl-1H-pyrazolo[3,4-d]pyrimidin-4-amine, dimethyl-4-amino-1-phenyl-1H-pyrazolo[3,4-b]pyridine-5,6-dicarboxylate, 1,5-diphenyl-1H-pyrazolo[3,4-d]pyrimidin-4(5H)-imine, 3-(2,4-dichlorophenyl)-5-hydroxy-2-methyl-6-phenylpyrazolo[1,5-a]pyrimidin-7(4H)-one, 5-hydroxy-6-phenyl-2-(4-(trifluoromethyl)phenyl)pyrazolo[1,5-a]pyrimidin-7(6H)-one, 3-(2,4-dichlorophenyl)-6-(4,5-dihydro-1H-imidazol-2-yl)-2-methylpyrazolo[1,5-a]pyrimidin-7-amine, (1E,4Z)-4-phenyl-3H-[1,2,5]triazepino[5,4-a]indol-1-amine, (Z)-2-(pyridin-4-yl)-1H-benzo[e][1,2,4]triazepin-5(4H)-one, 2-amino-4,5-diphenyl-4H-imidazol-4-ol, 4,5-diphenyl-1H-imidazol-2-amine, 7-methyl-2,3-diphenyl imidazo[1,2-a]pyrimidin-5(8H)-one, (1E,4Z)-4-phenyl-3H-[1,2,5]triazepino[5,4-a]indol-1-amine 4-(3-(2,4-dichlorophenyl)-2-methyl-6-nitro-7-oxo-4,7-dihydropyrazolo[1,5-a]pyrimidin-5-yl)benzoic acid, 4-(3-(2,4-dichlorophenyl)-2,4-dimethyl-7-oxo-4,7-dihydropyrazolo[1,5-a]pyrimidin-5-yl)benzo nitrile, 4-(3-(2,4-dichlorophenyl)-2,4-dimethyl-7-oxo-4,7-dihydropyrazolo[1,5-a]pyrimidin-5-yl)benzonitrile, 2-methyl-3-(4-(trifluoromethyl)phenyl)-5-(6-(trifluoromethyl)pyridin-3-yl)pyrazolo[1,5-a]pyrimidin-7(4H)-one, 3-(2,4-dichlorophenyl)-2-phenyl-5-(4-(trifluoromethyl)phenyl)pyrazolo[1,5-a]pyrimidin-7(4H)-one, 5-(chloromethyl)-3-(2,4-dichlorophenyl)-2-methylpyrazolo[1,5-a]pyrimidin-7(4H)-one, 3-(2,4-dichlorophenyl)-2-ethyl-5-(4-(trifluoromethyl)phenyl)pyrazolo[1,5-a]pyrimidin-7(4H)-one, 3-(2,4-dichlorophenyl)-2-methyl-5-((pyridin-2-ylamino)methyl)pyrazolo[1,5-a]pyrimidin-7(4H)-one, 7-morpholino-2-(4-(trifluoromethyl)phenyl)-4H-benzo[d][1,3]oxazin-4-one, 7-phenoxy-2-(4-(trifluoromethyl)phenyl)-4H-benzo[d][1,3]oxazin-4-one, 4-(3-(2,4-dichlorophenyl)-6-methyl-7-oxo-2-phenyl-4,7-dihydropyrazolo[1,5-a]pyrimidin-5-yl)benzonitrile, 4-(3-(2,4-dichlorophenyl)-2-ethyl-6-methyl-7-oxo-4,7-dihydropyrazolo[1,5-a]pyrimidin-5-yl)benzonitrile, 2,6-dimethyl-3-(4-(trifluoromethyl)phenyl)-5-(6-(trifluoromethyl)pyridin-3-yl)pyrazolo[1,5-a]pyrimidin-7(4H)-one, 4-(2-cyclopropyl-3-(2,4-dichlorophenyl)-6-methyl-7-oxo-4,7-dihydropyrazolo[1,5-a]pyrimidin-5-yl)-N,N-dipropylbenzenesulfonamide, ethyl N-3-cyano-4-(3-nitrophenyl)-4H-benzo[h]chromen-2-ylformimidate, (Z)—N-(furan-2-ylmethylene)-4-imino-1-phenyl-1H-pyrazolo[3,4-d]pyrimidin-5(4H)-amine, (Z)-2-(hydroxymethyl)-6-(4-((4-imino-1-phenyl-1H-pyrazolo[3,4-d]pyrimidin-5(4H)-ylimino)methyl)phenoxy)tetrahydro-2H-pyran-3,4,5-triol, 5-amino-7-(4-chlorophenyl)-3-(2,4-dichlorophenyl)-2-methylpyrazolo[1,5-a]pyrimidine-6-carbonitrile, 7-amino-2,3-diphenylimidazo[1,2-a]pyrimidine-6-carbonitrile, 7-amino-2,3-diphenylimidazo[1,2-a]pyrimidine-6-carboxamide, 4-(3-(2,4-dichlorophenyl)-2-(3-methoxyphenyl)-6-methyl-7-oxo-4,7-dihydropyrazolo[1,5-a]pyrimidin-5-yl)benzonitrile, 4-(3-(2,4-dichlorophenyl)-2-hydroxy-6-methyl-7-oxo-4,7-dihydropyrazolo[1,5-a]pyrimidin-5-yl)benzonitrile, 6H-phthalazino[1,2-b]quinazoline-5,8-dione, 2-(4-chlorophenyl)-4-oxo-8-phenyl-4H-pyrimido[1,2-a]pyrimidine-3-carbonitrile, 2-mercapto-7-morpholino-3-phenylquinazolin-4(3H)-one, 3-(2,4-dichlorophenyl)-6-methyl-2-phenyl-5-(6-(trifluoromethyl)pyridin-3-yl)pyrazolo[1,5-a]pyrimidin-7(4H)-one, 4-(2-ethyl-6-methyl-7-oxo-5-(4-(trifluoromethyl)phenyl)-4,7-dihydropyrazolo[1,5-a]pyrimidin-3-yl)benzonitrile, 4-(3-*2,4-difluorophenyl)-2-methyl-7-oxo-4,7-dihydropyrazolo[1,5-a]pyrimidin-5-yl)benzonitrile, 4-(3-(2,4-difluorophenyl)-2,6-dimethyl-7-oxo-4,7-dihydropyrazolo[1,5-a]pyrimidin-5-yl)benzonitrile, 4-(3-(2,4-difluorophenyl)-2,4-dimethyl-7-oxo-4,7-dihydropyrazolo[1,5-a]pyrimidin-5-yl)benzonitrile, 4-(3-(2,4-difluorophenyl)-2,4,6-trimethyl-7-oxo-4,7-dihydropyrazolo[1,5-a]pyrimidin-5-yl)benzonitrile, ethyl5-(4-cyanophenyl)-3-(2,4-difluorophenyl)-2-methyl-7-oxo-4,7-dihydropyrazolo[1,5-a]pyrimidine-6-carboxylate, ethyl 3-(2,4-difluorophenyl)-5-hydroxy-2-methyl-7-oxo-4,7-dihydropyrazolo[1,5-a]pyrimidine-6-carboxylate, 3-(2,4-difluorophenyl)-2-methyl-6-nitro-5-phenylpyrazolo[1,5-a]pyrimidin-7(4H)-one, 4-(8-(2,4-difluorophenyl)-7-methyl-4-oxo-4,6-dihydro-1H-pyrimido[1,2-b]pyridazin-2-yl)benzonitrile, 4-(8-(2,4-difluorophenyl)-7-methyl-4-oxo-4,9a-dihydro-1H-pyrimido[1,2-b]pyridazin-2-yl)benzonitrile, 4-(8-(2,4-difluorophenyl)-7-methyl-4-oxo-4H-pyrimido[1,2-b]pyridazin-2-yl)benzonitrile, 4-(8-(2,4-difluorophenyl)-7-methyl-5-oxo-2,6-diaza-bicyclo[4.2.0]oct-3-en-3-yl)benzonitrile, 4-(7-(2,4-difluorophenyl)-5-oxo-2,4-diaza-bicyclo[4.1.0]hept-3-en-3-yl)benzonitrile, 4-((6Z,8Z,10E)-10-(2,4-difluorophenyl)-7-methyl-4-oxo-4,11a-dihydro-1H-pyrimid[1,2-b][1,2]diazocin-2-yl)benzonitrile, 4-(7-(2,4-difluorophenyl)-6-methyl-3-oxo-3H-imidazo[1,2-b]pyrazol-2-yl)benzonitrile, 4-(3-(2,4-difluorophenyl)-2-methyl-6-oxo-6H-imidazo[1,5-b]pyrazol-4-yl)benzonitrile, 4-(3-(2,4-difluorophenyl)-2-methyl-9-oxo-4,9-dihydropyrazolo[5,1-b]quinazolin-6-yl)benzonitrile, 4-(2-(2,4-difluorophenyl)-9-oxo-4,9-dihydro-[1,2,4]triazolo[5,1-b]quinazolin-6-yl)benzonitrile, 4-(3-(2,4-difluorophenyl)-2,4,6-trimethyl-7-oxo-4,7-dihydropyrazolo[1,5-a]pyrimidin-5-yl)benzonitrile, ethyl 5-(4-cyanophenyl)-3-(2,4-difluorophenyl)-2,4-dimethyl-7-oxo-4,7-dihydropyrazolo[1,5-a]pyrimidine-6-carboxylate, ethyl 3-(2,4-difluorophenyl)-5-hydroxy-2,4-dimethyl-7-oxo-4,7-dihydropyrazolo[1,5-a]pyrimidine-6-carboxylate, 3-(2,4-difluorophenyl)-2,4-dimethyl-6-nitro-5-phenylpyrazolo[1,5-a]pyrimidin-7(4H)-one, 4-(8-(2,4-difluorophenyl)-1,7-dimethyl-4-oxo-4,6-dihydro-1H-pyrimido[1,2-b]pyridazin-2-yl)benzonitrile, 4-(8-(2,4-difluorophenyl)-1,7-dimethyl-4-oxo-4,9a-dihydro-1H-pyrimido[1,2-b]pyridazin-2-yl)benzonitrile, 4-(8-(2,4-difluorophenyl)-2,7-dimethyl-5-oxo-2,6-diaza-bicyclo[4.2.0]oct-3-en-3-yl)benzonitrile, 4-(7-(2,4-difluorophenyl)-2-methyl-5-oxo-2,4-diaza-bicyclo[4.1.0]hept-3-en-3-yl)benzonitrile, 4-((6Z,8Z,10E)-10-(2,4-difluorophenyl)-1,7-dimethyl-4-oxo-4,11a-dihydro-1H-pyrimido[1,2-b][1,2]diazocin-2-yl)benzonitrile, 4-(2-(2,4-difluorophenyl)-4-methyl-9-oxo-4,9-dihydro-[1,2,4]triazolo[5,1-b]quinazolin-6-yl)benzonitrile, 4-(2,6-dimethyl-7-oxo-3-(4-(trifluoromethyl)phenyl)-4,7-dihydropyrazolo[1,5-a]pyrimidin-5-yl)benzonitrile, 4-(2,4-dimethyl-7-oxo-3-(4-(trifluoromethyl)phenyl)-4,7-dihydropyrazolo[1,5-a]pyrimidin-5-yl)benzonitrile, 4-(2,4,6-trimethyl-7-oxo-3-(4-(trifluoromethyl)phenyl)-4,7-dihydropyrazolo[1,5-a]pyrimidin-5-yl)benzonitrile, 2-methyl-6-nitro-5-phenyl-3-(4-(trifluoromethyl)phenyl)pyrazolo[1,5-a]pyrimidin-7(4H)-one, 4-(7-methyl-4-oxo-8-(4-(trifluoromethyl)phenyl)-4,6-dihydro-1H-pyrimido[1,2-b]pyridazin-2-yl)benzonitrile, 4-(7-methyl-4-oxo-8-(4-(trifluoromethyl)phenyl)-4,9a-dihydro-1H-pyrimido[1,2-b]pyridazin-2-yl)benzonitrile, 4-(7-methyl-4-oxo-8-(4-(trifluoromethyl)phenyl)-4H-pyrimido[1,2-b]pyridazin-2-yl)benzonitrile, 4-(7-methyl-5-oxo-8-(4-(trifluoromethyl)phenyl)-2,6-diaza-bicyclo[4.2.0]oct-3-en-3-yl)benzonitrile, 4-(5-oxo-7-(4-(trifluoromethyl)phenyl)-2,4-diaza-bicyclo[4.1.0]hept-3-en-3-yl)benzonitrile, 4-((6Z,8Z,10E)-7-methyl-4-oxo-10-(4-(trifluoromethyl)phenyl)-4,11a-dihydro-1H-pyrimido[1,2-b][1,2]diazocin-2-yl)benzonitrile, 4-(9-oxo-2-(4-(trifluoromethyl)phenyl)-4,9-dihydro-[1,2,4]triazolo[5,1-b]quinazolin-6-yl)benzonitrile, 3-(2,4-difluorophenyl)-2-methyl-5-(4-(trifluoromethyl)phenyl)pyrazolo[1,5-a]pyrimidin-7(4H)-one, 3-(2,4-difluorophenyl)-2,6-dimethyl-5-(4-(trifluoromethyl)phenyl)pyrazolo[1,5-a]pyrimidin-7(4H)one, 3-(2,4-difluorophenyl)-2,4-dimethyl-5-(4-(trifluoromethyl)phenyl)pyrazolo[1,5-a]pyrimidin-7(4H)-one, 3-(2,4-difluorophenyl)-2,4,6-trimethyl-5-(4-(trifluoromethyl)phenyl)pyrazolo[1,5-a]pyrimidin-7(4H)-one, ethyl 3-(2,4-difluorophenyl)-2-methyl-7-oxo-5-(4-(trifluoromethyl)phenyl)-4,7-dihydropyrazolo[1,5-a]pyrimidine-6-carboxylate, 8-(2,4-difluorophenyl)-7-methyl-2-(4-(trifluoromethyl)phenyl)-1H-pyrimido[1,2-b]pyridazin-4(6H)-one, 8-(2,4-difluorophenyl)-7-methyl-2-(4-(trifluoromethyl)phenyl)-1H-pyrimido[1,2-b]pyridazin-4(9aH)-one, 8-(2,4-difluorophenyl)-7-methyl-2-(4-(trifluoromethyl)phenyl)-4H-pyrimido[1,2-b]pyridazin-4-one, 7-(2,4-difluorophenyl)-8-methyl-4-(4-(trifluoromethyl)phenyl)-1,5-diaza-bicyclo[4.2.0]oct-3-en-2-one, 7-(2,4-difluorophenyl)-4-(4-(trifluoromethyl)phenyl)-3,5-diaza-bicyclo[4.1.0]hept-3-en-2-one, (6Z,8Z,10E)-10-(2,4-difluorophenyl)-7-methyl-2-(4-(trifluoromethyl)phenyl)-1H-pyrimido[1,2-b][1, 2]diazocin-4(11aH)-one, 2-(2,4-difluorophenyl)-6-(4-(trifluoromethyl)phenyl)-[1,2,4]triazolo[5,1-b]quinazolin-9(4H)-one, 2-methyl-3,5-bis(4-(trifluoromethyl)phenyl)pyrazolo[1,5-a]pyrimidin-7(4H) one, 2,4-dimethyl-3,5-bis(4-(trifluoromethyl)phenyl)pyrazolo[1,5-a]pyrimidin-7(4H)one, 2,4,6-trimethyl-3,5-bis(4-(trifluoromethyl)phenyl)pyrazolo[1,5-a]pyrimidin-7(4H)-one, ethyl 2-methyl-7-oxo-3,5-bis(4-(trifluoromethyl)phenyl)-4,7-dihydropyrazolo[1,5-a]pyrimidine-6-carboxylate, 7-methy 1-2,8-bis(4-(trifluoromethyl)phenyl)-1H-pyrimido[1,2-b]pyridazin-4(6H)-one, 7-methyl-2,8-bis(4-(trifluoromethyl)phenyl)-1H-pyrimido[1,2-b]pyridazin-4(9aH)-one, 7-methyl-2,8-bis(4-(trifluoromethyl)phenyl)-4H-pyrimido[1,2-b]pyridazin-4-one, 8-methyl-4,7-bis(4-(trifluoromethyl)phenyl)-1,5-diazabicyclo[4.2.0]oct-3-en-2-one, 7-bis(4-(trifluoromethyl)phenyl)-3,5-diaza-bicyclo[4.1.0]hept-3-en-2-one, (6Z,8Z,10E)-7-methyl-2,10-bis(4-(trifluoromethyl)phenyl)-1H-pyrimido[1,2-b][1,2]diazocin-4(11aH)-one, 2-methyl-3,6-bis(4-(trifluoromethyl)phenyl)pyrazolo[5,1-b]quinazolin-9(4H)-one, 2,6-bis(4-(trifluoromethyl)phenyl)-[1,2,4]triazolo[5,1-b]quinazolin-9(4H)-one, 2,4-dimethyl-3-(4-(trifluoromethyl)phenyl)-5-(6-(trifluoromethyl)pyridin-3-yl)pyrazolo[1,5-a]pyrimidin-7(4H)-one, 2,4,6-trimethyl-3-(4-(trifluoromethyl)phenyl)-5-(6-(trifluoromethyl)pyridin-3-yl)pyrazolo[1,5-a]pyrimidin-7(4H)-one, ethyl 2-methyl-7-oxo-3-(4-(trifluoromethyl)phenyl)-5-(6-(trifluoromethyl)pyridin-3-yl)-4,7-dihydropyrazolo[1,5-a]pyrimidine-6-carboxylate, 7-methyl-8-(4-(trifluoromethyl)phenyl)-2-(6-(trifluoromethyl)pyridin-3-yl)-1H-pyrimido[1,2-b]pyridazin-4(6H)-one, 7-methyl-8-(4-(trifluoromethyl)phenyl)-2-(6-(trifluoromethyl)pyridin-3-yl)-1H-pyrimido[1,2-b]pyridazin-4(9aH)-one, 7-methyl-8-(4-(trifluoromethyl)phenyl)-2-(6-(trifluoromethyl)pyridin-3-yl)-4H-pyrimido[1,2-b]pyridazin-4-one, 8-methyl-7-(4-(trifluoromethyl)phenyl)-4-(6-(trifluoromethyl)pyridin-3-yl)-1,5-diaza-bicyclo[4.2.0]oct-3-en-2-one, 7-(4-(trifluoromethyl)phenyl)-4-(6-(trifluoromethyl)pyridin-3-yl)-3,5-diaza-bicyclo[4.1.0]hept-3-en-2-one, (6Z,8Z,10E)-7-methyl-10-(4-(trifluoromethyl)phenyl)-2-(6-(trifluoromethyl)pyridin-3-yl)-1H-pyrimido[1,2-b][1,2]diazocin-4(11a H)-one, 6-methyl-7-(4-(trifluoromethyl)phenyl)-2-(6-(trifluoromethyl)pyridin-3-yl)-3H-imidazo[1,2-b]pyrazol-3-one, 2-methyl-3-(4-(trifluoromethyl)phenyl)-6-(6-(trifluoromethyl)pyridin-3-yl)pyrazolo[5,1-b]quinazolin-9(4H)-one, 2-(4-(trifluoromethyl)phenyl)-6-(6-(trifluoromethyl)pyridin-3-yl)-[1,2,4]triazolo[5,1-b]quinazolin-9(4H)-one, 4-(2-(2,4-difluorophenyl)-6-oxo-1,6-dihydropurin-9-yl)benzonitrile, 5-(2,4-difluorophenyl)-2-methyl-3-(4-(trifluoromethyl)phenyl)pyrazolo[1,5-a]pyrimidin-7(4H)-one, 5-(2,4-difluorophenyl)-2,6-dimethyl-3-(4-(trifluoromethyl)phenyl)pyrazolo[1,5-a]pyrimidin-7(4H)-one, 5-(2,4-difluorophenyl)-2,4-dimethyl-3-(4-(trifluoromethyl)phenyl)pyrazolo[1,5-a]pyrimidin-7(4H)-one, 5-(2,4-difluorophenyl)-2,4,6-trimethyl-3-(4-(trifluoromethyl)phenyl)pyrazolo[1,5-a]pyrimidin-7(4H)-one, ethyl 5-(2,4-difluorophenyl)-2-methyl-7-oxo-3-(4-(trifluoromethyl)phenyl)-4,7-dihydropyrazolo[1,5-a]pyrimidine-6-carboxylate, 2-(2,4-difluorophenyl)-7-methyl-8-(4-(trifluoromethyl)phenyl)-1H-pyrimido[1,2-b]pyridazin-4(6H)-one, 2-(2,4-difluorophenyl)-7-methyl-8-(4-(trifluoromethyl)phenyl)-1H-pyrimido[1,2-b]pyridazin-4(9aH)-one, 2-(2,4-difluorophenyl)-7-methyl-8-(4-(trifluoromethyl)phenyl)-4H-pyrimido[1,2-b]pyridazin-4-one, 4-(2,4-difluorophenyl)-8-methyl-7-(4-(trifluoromethyl)phenyl)-1,5-diazabicyclo[4.2.0]oct-3-en-2-one 4-(2,4-difluorophenyl)-7-(4-(trifluoromethyl)phenyl)-3,5-diaza-bicyclo[4.1.0]hept-3-en-2-one, (6Z, 8Z,10E)-2-(2,4-difluorophenyl)-7-methyl-10-(4-(trifluoromethyl)phenyl)-1H-pyrimido[1,2-b][1,2]diazocin-4(11aH)-one, 3-(2,4-difluorophenyl)-5-(4-methoxyphenyl)-2,4,6-trimethylpyrazolo[1,5-a]pyrimidin-7(4H)-one, 3-(2,4-difluorophenyl)-5-(4-methoxyphenyl)-2-methyl-7-oxo-4,7-dihydropyrazolo[1,5-a]pyrimidine-6-carboxylate, (6Z,8Z,10E)-10-(2,4-difluorophenyl)-2-(4-methoxyphenyl)-7-methyl-1H-pyrimido[1,2-b][1,2]diazocin-4(11aH)-one, 5-(4-aminophenyl)-3-(2,4-difluorophenyl)-2-methyl-7-oxo-4,7-dihydropyrazolo[1,5-a]pyrimidine-6-carboxylate, (6Z,8Z,10E)-2-(4-aminophenyl)-10-(2,4-difluorophenyl)-7-methyl-1H-pyrimido[1,2-b][1,2]diazocin-4(11aH)-one, ethyl 6-(2,4-difluorophenyl)-2-methyl-3-(4-(trifluoromethyl)phenyl)pyrazolo[5,1-b]quinazolin-9(4H)-one, 6-(2,4-difluorophenyl)-2-(4-(trifluoromethyl)phenyl)-[1,2,4]triazolo[5,1-b]quinazolin-9(4H)-one, 3-(2,4-difluorophenyl)-5-(4-methoxyphenyl)-2-methylpyrazolo[1,5-a]pyrimidin-7(4H)-one, 3-(2,4-difluorophenyl)-5-(4-methoxyphenyl)-2,6-dimethylpyrazolo[1,5-a]pyrimidin-7(4H)-one, 3-(2,4-difluorophenyl)-5-(4-methoxyphenyl)-2,4-dimethylpyrazolo[1,5-a]pyrimidin-7(4H)-one, 8-(2,4-difluorophenyl)-2-(4-methoxyphenyl)-7-methyl-1H-pyrimido[1,2-b]pyridazin-4(6H)-one, 8-(2,4-difluorophenyl)-2-(4-methoxyphenyl)-7-methyl-1H-pyrimido[1,2-b]pyridazin-4(9aH)-one, 8-(2,4-difluorophenyl)-2-(4-methoxyphenyl)-7-methyl-4H-pyrimido[1,2-b]pyridazin-4-one, 4-((3-(2,4-difluorophenyl)-2-methyl-7-oxo-4,7-dihydropyrazolo[1,5-a]pyrimidin-5-yl)methylamino) benzonitrile, 4-(4-aminophenyl)-7-(2,4-difluorophenyl)-3,5-diaza-bicyclo[4.1.0]hept-3-en-2-one, 7-(2,4-difluorophenyl)-4-(4-methoxyphenyl)-8-methyl-1,5-diaza-bicyclo[4.2.0]oct-3-en-2-one, 7-(2,4-difluorophenyl)-4-(4-methoxyphenyl)-3,5-diaza-bicyclo[4.1.0]hept-3-en-2-one, 7-(2,4-difluorophenyl)-2-(4-methoxyphenyl)-6-methyl-3H-imidazo[1,2-b]pyrazol-3-one, 3-(2,4-difluorophenyl)-4-(4-methoxyphenyl)-2-methyl-6H-imidazo[1,5-b]pyrazol-6-one, 3-(2,4-difluorophenyl)-6-(4-methoxyphenyl)-2-methylpyrazolo[5,1-b]quinazolin-9(4H)-one, 2-(2,4-difluorophenyl)-6-(4-methoxyphenyl)-[1,2,4]triazolo[5,1-b]quinazolin-9(4H)-one, 5-(4-aminophenyl)-3-(2,4-difluorophenyl)-2-methylpyrazolo[1,5-a]pyrimidin-7(4H)-one, 4-(3-(2,4-difluorophenyl)-2,6-dimethyl-7-oxo-4,7-dihydropyrazolo[1,5-a]pyrimidin-5-yl)benzonitrile 5-(4-aminophenyl)-3-(2,4-difluorophenyl)-2,4-dimethylpyrazolo[1,5-a]pyrimidin-7(4H)-one, 5-(4-aminophenyl)-3-(2,4-difluorophenyl)-2,4,6-trimethylpyrazolo[1,5-a]pyrimidin-7(4H)-one, ethyl 2-(4-aminophenyl)-8-(2,4-difluorophenyl)-7-methyl-1H-pyrimido[1,2-b]pyridazin-4(6H)-one, 2-(4-aminophenyl)-8-(2,4-difluorophenyl)-7-methyl-1H-pyrimido[1,2-b]pyridazin-4(9aH)-one, 2-(4-aminophenyl)-8-(2,4-difluorophenyl)-7-methyl-4H-pyrimido[1,2-b]pyridazin-4-one, 4-(4-aminophenyl)-7-(2,4-difluorophenyl)-8-methyl-1,5-diaza-bicyclo[4.2.0]oct-3-en-2-one, 4-(3-(2,4-difluorophenyl)-2,6-dimethyl-7-oxo-4,7-dihydropyrazolo[1,5-a]pyrimidin-5-yl)methylamino)benzonitrile, 4-(3-(2,4-difluorophenyl)-2,4,6-trimethyl-7-oxo-4,7-dihydropyrazolo[1,5-a]pyrimidin-5-yl)methylamino)benzonitrile, ethyl 54(4-cyanophenylamino)methyl)-3-(2,4-difluorophenyl)-2-methyl-7-oxo-4,7-dihydropyrazolo[1,5-a]pyrimidine-6-carboxylate, 4-(8-(2,4-difluorophenyl)-7-methyl-4-oxo-4,6-dihydro-1H-pyrimido[1,2-b]pyridazin-2-yl)methylamino)benzonitrile, 4-((8-(2,4-difluorophenyl)-7-methyl-4-oxo-4,9a-dihydro-1H-pyrimido[1,2-b]pyridazin-2-yl)methyl amino)benzonitrile, 4-((8-(2,4-difluorophenyl)-7-methyl-4-oxo-4H-pyrimido[1,2-b]pyridazin-2-yl)methylamino)benzonitrile, 4-((8-(2,4-difluorophenyl)-7-methyl-5-oxo-2,6-diaza-bicyclo[4.2.0]oct-3-en-3-yl)methylamino)benzonitrile, 4-((7-(2,4-difluorophenyl)-5-oxo-2,4-diaza-bicyclo[4.1.0]hept-3-en-3-yl)methylamino)benzonitrile, 4-(((6Z,8Z,10E)-10-(2,4-difluorophenyl)-7-methyl-4-oxo-4,1a-dihydro-1H-pyrimido[1,2-b][1,2]diazocin-2-yl)methylamino)benzonitrile, 4-((7-(2,4-difluorophenyl)-6-methyl-3-oxo-3H-imidazo[1,2-b]pyrazol-2-yl)methylamino)benzonitrile, 4-((3-(2,4-difluorophenyl)-2-methyl-6-oxo-6H-imidazo[1,5-b]pyrazol-4-yl)methylamino)benzonitrile, 4-((3-(2,4-difluorophenyl)-2-methyl-9-oxo-4,9-dihydropyrazolo[5,1-b]quinazolin-6-yl)methylamino)benzo nitrile, 4-((2-(2,4-difluorophenyl)-9-oxo-4,9-dihydro-[1,2,4]triazolo[5,1-b]quinazolin-6-yl)methylamino)benzonitrile, 4-(3-(2,4-difluorophenyl)-2-methyl-7-oxo-4,7-dihydropyrazolo[1,5-a]pyrimidin-5-yl)-N,N-dipropylbenzenesulfonamide, 4-(3-(2,4-difluorophenyl)-2,6-dimethyl-7-oxo-4,7-dihydropyrazolo[1,5-a]pyrimidin-5-yl)-N,N-dipropylbenzenesulfonamide, 0.4-(3-(2,4-difluorophenyl)-2,4-dimethyl-7-oxo-4,7-dihydropyrazolo[1,5-a]pyrimidin-5-yl)-N,N-dipropylbenzenesulfonamide, 4-(3-(2,4-difluorophenyl)-2,4,6-trimethyl-7-oxo-4,7-dihydropyrazolo[1,5-a]pyrimidin-5-yl)-N,N-dipropylbenzenesulfonamide, 4-(8-(2,4-difluorophenyl)-7-methyl-4-oxo-4,6-dihydro-1H-pyrimido[1,2-b]pyridazin-2-yl)-N,N-dipropylbenzenesulfonamide, 4-(8-(2,4-difluorophenyl)-7-methyl-4-oxo-4,9a-dihydro-1H-pyrimido[1,2-b]pyridazin-2-yl)-N,N-dipropylbenzenesulfonamide, 4-(8-(2,4-difluorophenyl)-7-methyl-4-oxo-4H-pyrimido[1,2-b]pyridazin-2-yl)-N,N-dipropylbenzenesulfonamide, 4-(8-(2,4-difluorophenyl)-7-methyl-5-oxo-2,6-diaza-bicyclo[4.2.0]oct-3-en-3-yl)-N,N-dipropylbenzenesulfonamide, 4-(7-(2,4-difluorophenyl)-5-oxo-2,4-diaza-bicyclo[4.1.0]hept-3-en-3-yl)-N,N-dipropyl benzenesulfonamide, 4-((6Z,8Z,10E)-10-(2,4-difluorophenyl)-7-methyl-4-oxo-4,11a-dihydro-1H-pyrimido[1,2-b][1,2]diazocin-2-yl)-N,N-dipropylbenzenesulfonamide, 4-(7-(2,4-difluorophenyl)-6-methyl-3-oxo-3H-imidazo[1,2-b]pyrazol-2-yl)-N,N-dipropylbenzenesulfonamide, 4-(3-(2,4-difluorophenyl)-2-methyl-9-oxo-4,9-dihydropyrazolo[5,1-b]quinazolin-6-yl)-N,N-dipropyl benzenesulfonamide, 4-(2-(2,4-difluorophenyl)-9-oxo-4,9-dihydro-[1,2,4]triazolo[5,1-b]quinazolin-6-yl)-N,N-dipropylbenzenesulfonamide, 3-(2,4-difluorophenyl)-2-ethyl-6-methyl-5-(4-(trifluoromethyl)phenyl)pyrazolo[1,5-a]pyrimidin-7(4H)-one, 3-(2,4-difluorophenyl)-2-ethyl-4-methyl-5-(4-(trifluoromethyl)phenyl)pyrazolo[1,5-a]pyrimidin-7(4H)-one, 3-(2,4-difluorophenyl)-2-ethyl-4,6-dimethyl-5-(4-(trifluoromethyl)phenyl)pyrazolo[1,5-a]pyrimidin-7(4H)-one, ethyl 3-(2,4-difluorophenyl)-2-ethyl-7-oxo-5-(4-(trifluoromethyl)phenyl)-4,7-dihydropyrazolo[1,5-a]pyrimidine-6-carboxylate, ethyl 2-ethyl-5-hydroxy-7-oxo-3-(4-(trifluoromethyl)phenyl)-4,7-dihydropyrazolo[1,5-a]pyrimidine-6-carboxylate, 2-ethyl-6-nitro-5-phenyl-3-(4-(trifluoromethyl)phenyl)pyrazolo[1,5-a]pyrimidin-7(4H)-one, 8-(2,4-difluorophenyl)-7-ethyl-2-(4-(trifluoromethyl)phenyl)-1H-pyrimido[1,2-b]pyridazin-4(6H)-one, 8-(2,4-difluorophenyl)-7-ethyl-2-(4-(trifluoromethyl)phenyl)-1H-pyrimido[1,2-b]pyridazin-4(9aH)-one, 8-(2,4-difluorophenyl)-7-ethyl-2-(4-(trifluoromethyl)phenyl)-4H-pyrimido[1,2-b]pyridazin-4-one, 7-(2,4-difluorophenyl)-8-ethyl-4-(4-(trifluoromethyl)phenyl)-1,5-diaza-bicyclo[4.2.0]oct-3-en-2-one, (6Z,8Z,10E)-10-(2,4-difluorophenyl)-7-ethyl-2-(4-(trifluoromethyl)phenyl)-1H-pyrimido[1,2-b][1,2]diazocin-4(11aH)-one, 7-(2,4-difluorophenyl)-6-ethyl-2-(4-(trifluoromethyl)phenyl)-3H-imidazo[1,2-b]pyrazol-3-one, 3-(2,4-difluorophenyl)-2-ethyl-4-(4-(trifluoromethyl)phenyl)-6H-imidazo[1,5-b]pyrazol-6-one, 3-(2,4-difluorophenyl)-2-ethyl-6-(4-(trifluoromethyl)phenyl)pyrazolo[5,1-b]quinazolin-9(4H)-one, 4-(3-(2,4-difluorophenyl)-7-oxo-2-phenyl-4,7-dihydropyrazolo[1,5-a]pyrimidin-5-yl)benzonitrile, 4-(3-(2,4-difluorophenyl)-6-methyl-7-oxo-2-phenyl-4,7-dihydropyrazolo[1,5-a]pyrimidin-5-yl)benzonitrile, 4-(3-(2,4-difluorophenyl)-4,6-dimethyl-7-oxo-2-phenyl-4,7-dihydropyrazolo[1,5-a]pyrimidin-5-yl)benzonitrile, ethyl 5-(4-cyanophenyl)-3-(2,4-difluorophenyl)-7-oxo-2-phenyl-4,7-dihydropyrazolo[1,5-a]pyrimidine-6-carboxylate, ethyl 3-(2,4-difluorophenyl)-5-hydroxy-7-oxo-2-phenyl-4,7-dihydropyrazolo[1,5-a]pyrimidine-6-carboxylate, 3-(2,4-difluorophenyl)-6-nitro-2,5-diphenylpyrazolo[1,5-a]pyrimidin-7(4H)-one, 4-(8-(2,4-difluorophenyl)-4-oxo-7-phenyl-4,6-dihydro-1H-pyrimido[1,2-b]pyridazin-2-yl)benzonitrile, 4-(8-(2,4-difluorophenyl)-4-oxo-7-phenyl-4,9a-dihydro-1H-pyrimido[1,2-b]pyridazin-2-yl)benzonitrile, 4-(8-(2,4-difluorophenyl)-4-oxo-7-phenyl-4H-pyrimido[1,2-b]pyridazin-2-yl)benzonitrile, 4-(8-(2,4-difluorophenyl)-5-oxo-7-phenyl-2,6-diaza-bicyclo[4.2.0]oct-3-en-3-yl)benzonitrile, 4-((6E,8Z,10E)-10-(2,4-difluorophenyl)-4-oxo-7-phenyl-4,11a-dihydro-1H-pyrimido[1,2-b][1,2]diazocin-2-yl)benzonitrile, 3-(2,4-difluorophenyl)-2-phenyl-5-(4-(trifluoromethyl)phenyl)pyrazolo[1,5-a]pyrimidin-7(4H)-one, 3-(2,4-difluorophenyl)-6-methyl-2-phenyl-5-(4-(trifluoromethyl)phenyl)pyrazolo[1,5-a]pyrimidin-7(4H)-one, 3-(2,4-difluorophenyl)-4-methyl-2-phenyl-5-(4-(trifluoromethyl)phenyl)pyrazolo[1,5-a]pyrimidin-7(4H)-one, 3-(2,4-difluorophenyl)-4,6-dimethyl-2-phenyl-5-(4-(trifluoromethyl)phenyl)pyrazolo[1,5-a]pyrimidin-7(4H)-one, ethyl 3-(2,4-difluorophenyl)-7-oxo-2-phenyl-5-(4-(trifluoromethyl)phenyl)-4,7-dihydropyrazolo[1,5-a]pyrimidine-6-carboxylate, ethyl5-hydroxy-7-oxo-2-phenyl-3-(4-(trifluoromethyl)phenyl)-4,7-dihydropyrazolo[1,5-a]pyrimidine-6-carboxylate, 6-nitro-2,5-diphenyl-3-(4-(trifluoromethyl)phenyl)pyrazolo[1,5-a]pyrimidin-7(4H)-one, 8-(2,4-difluorophenyl)-7-phenyl-2-(4-(trifluoromethyl)phenyl)-1H-pyrimido[1,2-b]pyridazin-4(6H)-one. 8-(2,4-difluorophenyl)-7-phenyl-2-(4-(trifluoromethyl)phenyl)-1H-pyrimido[1,2-b]pyridazin-4(9aH)-one, 8-(2,4-difluorophenyl)-7-phenyl-2-(4-(trifluoromethyl)phenyl)-4H-pyrimido[1,2-b]pyridazin-4-one, 7-(2,4-difluorophenyl)-8-phenyl-4-(4-(trifluoromethyl)phenyl)-1,5-diaza-bicyclo[4.2.0]oct-3-en-2-one, (6E,8Z,10E)-10-(2,4-difluorophenyl)-7-phenyl-2-(4-(trifluoromethyl)phenyl)-1H-pyrimido[1,2-b][1,2]diazocin-4(11aH)-one, 3-(2,4-difluorophenyl)-2-phenyl-6-(4-(trifluoromethyl)phenyl)pyrazolo[5,1-b]quinazolin-9(4H)-one, 4-(2-cyclopropyl-3-(2,4-difluorophenyl)-7-oxo-4,7-dihydropyrazolo[1,5-a]pyrimidin-5-yl)benzonitrile, 4-(2-cyclopropyl-3-(2,4-difluorophenyl)-6-methyl-7-oxo-4,7-dihydropyrazolo[1,5-a]pyrimidin-5-yl)benzonitrile, 4-(2-cyclopropyl-3-(2,4-difluorophenyl)-4,6-dimethyl-7-oxo-4,7-dihydropyrazolo[1,5-a]pyrimidin-5-yl)benzonitrile, ethyl 5-(4-cyanophenyl)-2-cyclopropyl-3-(2,4-difluorophenyl)-7-oxo-4,7-dihydropyrazolo[1,5-a]pyrimidine-6-carboxylate, ethyl 2-cyclopropyl-3-(2,4-difluorophenyl)-5-hydroxy-7-oxo-4,7-dihydropyrazolo[1,5-a]pyrimidine-6-carboxylate, 2-cyclopropyl-3-(2,4-difluorophenyl)-6-nitro-5-phenylpyrazolo[1,5-a]pyrimidin-7(4H)-one, 4-(7-cyclopropyl-8-(2,4-difluorophenyl)-4-oxo-4,6-dihydro-1H-pyrimido[1,2-b]pyridazin-2-yl)benzonitrile, 4-(7-cyclopropyl-8-(2,4-difluorophenyl)-4-oxo-4,9a-dihydro-1H-pyrimido[1,2-b]pyridazin-2-yl)benzonitrile, 4-(8-(2,4-difluorophenyl)-5-oxo-7-(trifluoromethyl)-2,6-diaza-bicyclo[4.2.0]oct-3-en-3-yl)benzonitrile, 4-((6Z,8Z,10E)-7-cyclopropyl-10-(2,4-difluorophenyl)-4-oxo-4,11a-dihydro-1H-pyrimido[1,2-b][1,2]diazocin-2-yl)benzonitrile, 4-(3-(2,4-difluorophenyl)-7-oxo-2-(trifluoromethyl)-4,7-dihydropyrazolo[1,5-a]pyrimidin-5-yl)benzonitrile, 4-(3-(2,4-difluorophenyl)-6-methyl-7-oxo-2-(trifluoromethyl)-4,7-dihydropyrazolo[1,5-a]pyrimidin-5-yl)benzonitrile, 4-(3-(2,4-difluorophenyl)-4-methyl-7-oxo-2-(trifluoromethyl)-4,7-dihydropyrazolo[1,5-a]pyrimidin-5-yl)benzonitrile, 4-(3-(2,4-difluorophenyl)-4,6-dimethyl-7-oxo-2-(trifluoromethyl)-4,7-dihydropyrazolo[1,5-a]pyrimidin-5-yl)benzonitrile ethyl 5-(4-cyanophenyl)-3-(2,4-difluorophenyl)-7-oxo-2-(trifluoromethyl)-4,7-dihydropyrazolo[1,5-a]pyrimidine-6-carboxylate, ethyl 3-(2,4-difluorophenyl)-5-hydroxy-7-oxo-2-(trifluoromethyl)-4,7-dihydropyrazolo[1,5-a]pyrimidine-6-carboxylate, 3-(2,4-difluorophenyl)-6-nitro-5-phenyl-2-(trifluoromethyl)pyrazolo[1,5-a]pyrimidin-7(4H)-one, 4-(8-(2,4-difluorophenyl)-4-oxo-7-(trifluoromethyl)-4,6-dihydro-1H-pyrimido[1,2-b]pyridazin-2-yl)benzonitrile, 4-(8-(2,4-difluorophenyl)-4-oxo-7-(trifluoromethyl)-4,9a-dihydro-1H-pyrimido[1,2-b]pyridazin-2-yl)benzonitrile, 4-(8-(2,4-difluorophenyl)-4-oxo-7-(trifluoromethyl)-4H-pyrimido[1,2-b]pyridazin-2-yl)benzonitrile, 4-((6E,8Z,10E)-10-(2,4-difluorophenyl)-4-oxo-7-(trifluoromethyl)-4,11a-dihydro-1H-pyrimido[1,2-b][1,2]diazocin-2-yl)benzonitrile, 4-(3-(2,4-difluorophenyl)-9-oxo-2-(trifluoromethyl)-4,9-dihydropyrazolo[5,1-b]quinazolin-6-yl)benzonitrile, 4-(3-(2,4-difluorophenyl)-2-ethyl-7-oxo-4,7-dihydropyrazolo[1,5-a]pyrimidin-5-yl)benzonitrile, 4-(3-(2,4-difluorophenyl)-2-ethyl-6-methyl-7-oxo-4,7-dihydropyrazolo[1,5-a]pyrimidin-5-yl)benzonitrile, 4-(3-(2,4-difluorophenyl)-2-ethyl-4-methyl-7-oxo-4,7-dihydropyrazolo[1,5-a]pyrimidin-5-yl)benzonitrile, 4-(3-(2,4-difluorophenyl)-2-ethyl-4,6-dimethyl-7-oxo-4,7-dihydropyrazolo[1,5-a]pyrimidin-5-yl)benzonitrile, ethyl 5-(4-cyanophenyl)-3-(2,4-difluorophenyl)-2-ethyl-7-oxo-4,7-dihydropyrazolo[1,5-a]pyrimidine-6-carboxylate, ethyl 3-(2,4-difluorophenyl)-2-ethyl-5-hydroxy-7-oxo-4,7-dihydropyrazolo[1,5-a]pyrimidine-6-carboxylate, 3-(2,4-difluorophenyl)-2-ethyl-6-nitro-5-phenylpyrazolo[1,5-a]pyrimidin-7(4H)-one, 4-(8-(2,4-difluorophenyl)-7-ethyl-4-oxo-4,6-dihydro-1H-pyrimido[1,2-b]pyridazin-2-yl)benzonitrile, 4-(8-(2,4-difluorophenyl)-7-ethyl-4-oxo-4,9a-dihydro-1H-pyrimido[1,2-b]pyridazin-2-yl)benzonitrile, 4-(8-(2,4-difluorophenyl)-7-ethyl-4-oxo-4H-pyrimido[1,2-b]pyridazin-2-yl)benzonitrile, 4-(8-(2,4-difluorophenyl)-7-ethyl-5-oxo-2,6-diaza-bicyclo[4.2.0]oct-3-en-3-yl)benzonitrile, 4-((6Z,8Z,10E)-10-(2,4-difluorophenyl)-7-ethyl-4-oxo-4,11a-dihydro-1H-pyrimido[1,2-b][1,2]diazocin-2-yl)benzonitrile, 4-(7-(2,4-difluorophenyl)-6-ethyl-3-oxo-3H-imidazo[1,2-b]pyrazol-2-yl)benzonitrile, 4-(3-(2,4-difluorophenyl)-2-ethyl-9-oxo-4,9-dihydropyrazolo[5,1-b]quinazolin-6-yl)benzonitrile, 4-(3-(2,4-dichlorophenyl)-2,4,6-trimethyl-7-oxo-4,7-dihydropyrazolo[1,5-a]pyrimidin-5-yl)benzonitrile, ethyl 5-(4-cyanophenyl)-3-(2,4-dichlorophenyl)-2-methyl-7-oxo-4,7-dihydropyrazolo[1,5-a]pyrimidine-6-carboxylate, ethyl 3-(2,4-dichlorophenyl)-5-hydroxy-2-methyl-7-oxo-4,7-dihydropyrazolo[1,5-a]pyrimidine-6-carboxylate, 3-(2,4-dichlorophenyl)-2-methyl-6-nitro-5-phenyl pyrazolo[1,5-a]pyrimidin-7(4H)-one, 4-(8-(2,4-dichlorophenyl)-7-methyl-4-oxo-4,6-dihydro-1H-pyrimido[1,2-b]pyridazin-2-yl)benzonitrile, 4-(8-(2,4-dichlorophenyl)-7-methyl-4-oxo-4,9a-dihydro-1H-pyrimido[1,2-b]pyridazin-2-yl)benzonitrile, 4-(8-(2,4-dichlorophenyl)-7-methyl-4-oxo-4H-pyrimido[1,2-b]pyridazin-2-yl)benzonitrile, 4-(8-(2,4-dichlorophenyl)-7-methyl-5-oxo-2,6-diaza-bicyclo[4.2.0]oct-3-en-3-yl)benzonitrile, 4-(7-(2,4-dichlorophenyl)-5-oxo-2,4-diaza-bicyclo[4.1.0]hept-3-en-3-yl)benzonitrile, 4-((6Z,8Z,10E)-10-(2,4-dichlorophenyl)-7-methyl-4-oxo-4,11a-dihydro-1H-pyrimido[1,2-b][1,2]diazocin-2-yl)benzonitrile, 4-(5-(4-cyanophenyl)-2-methyl-7-oxo-4,7-dihydropyrazolo[1,5-a]pyrimidin-3-yl)benzoic acid, 4-(5-(4-cyanophenyl)-2,6-dimethyl-7-oxo-4,7-dihydropyrazolo[1,5-a]pyrimidin-3-yl)benzoic acid, 4-(5-(4-cyanophenyl)-2,4,6-trimethyl-7-oxo-4,7-dihydropyrazolo[1,5-a]pyrimidin-3-yl)benzoic acid, 4-(5-(4-cyanophenyl)-6-(ethoxycarbonyl)-2-methyl-7-oxo-4,7-dihydropyrazolo[1,5-a]pyrimidin-3-yl)benzoic acid, 4-(6-(ethoxycarbonyl)-5-hydroxy-2-methyl-7-oxo-4,7-dihydropyrazolo[1,5-a]pyrimidin-3-yl)benzoic acid, 4-(2-methyl-6-nitro-7-oxo-5-phenyl-4,7-dihydropyrazolo[1,5-a]pyrimidin-3-yl)benzoic acid, 4-(2-(4-cyanophenyl)-7-methyl-4-oxo-4,6-dihydro-1H-pyrimido[1,2-b]pyridazin-8-yl)benzoic acid, 4-(2-(4-cyanophenyl)-7-methyl-4-oxo-4,9a-dihydro-1H-pyrimido[1,2-b]pyridazin-8-yl)benzoic acid, 4-(2-(4-cyanophenyl)-7-methyl-4-oxo-4H-pyrimido[1,2-b]pyridazin-8-yl)benzoic acid, 4-(4-(4-cyanophenyl)-8-methyl-2-oxo-1,5-diaza-bicyclo[4.2.0]oct-3-en-7-yl)benzoic acid, 4-(4-(4-cyanophenyl)-2-oxo-3,5-diaza-bicyclo[4.1.0]hept-3-en-7-yl)benzoic acid, 4-((6Z,8Z,10E)-2-(4-cyanophenyl)-7-methyl-4-oxo-4,11a-dihydro-1H-pyrimido[1,2-b][1,2]diazocin-10-yl)benzoic acid, 4-(2-(4-cyanophenyl)-6-methyl-3-oxo-3H-imidazo[1,2-b]pyrazol-7-yl)benzoic acid, 4-(6-(4-cyanophenyl)-2-methyl-9-oxo-4,9-dihydropyrazolo[5,1-b]quinazolin-3-yl)benzoic acid, 4-(6-(4-cyanophenyl)-9-oxo-4,9-dihydro-[1,2,4]triazolo[5,1-b]quinazolin-2-yl)benzoic acid, 7-phenoxy-2-(4-(trifluoromethyl)phenyl)quinazolin-4(3H)-one, (Z)-2-(hydroxymethyl)-6-(4-((4-imino-1-(4-(trifluoromethyl)phenyl)-H-pyrazolo[3,4-d]pyrimidin-5(4H)-ylimino)methyl)phenoxy)-tetrahydro-2H-pyran-3,4,5-triol, (Z)-4-(5-(4-(3,4,5-trihydroxy-6-(hydroxymethyl)-tetrahydro-2H-pyran-2-yloxy)benzylideneamino)-4-imino-4,5-dihydropyrazolo[3,4-d]pyrimidin-1-yl)benzonitrile, (Z)-4-(5-(4-(3,4,5-trihydroxy-6-(hydroxymethyl)-tetrahydro-2H-pyran-2-yloxy)benzylideneamino)-4-imino-4,5-dihydropyrazolo[3,4-d]pyrimidin-1-yl)benzoic acid, (Z)-4-(5-(4-(3,4,5-trihydroxy-6-(hydroxymethyl)-tetrahydro-2H-pyran-2-yloxy)benzylideneamino)-4-imino-4,5-dihydropyrazolo[3,4-d]pyrimidin-1-yl)benzene sulfonic acid, (Z)-4-(5-(4-(3,4,5-trihydroxy-6-(hydroxymethyl)-tetrahydro-2H-pyran-2-yloxy)benzylideneamino)-4-imino-4,5-dihydropyrazolo[3,4-d]pyrimidin-1-yl)-N,N-dipropylbenzenesulfonamide, (Z)-2-(hydroxymethyl)-6-(4-((4-imino-3-methyl-1-phenyl-1H-pyrazolo[3,4-d]pyrimidin-5(4H)-yl-amino)methyl)phenoxy)tetrahydro-2H-pyran-3,4,5-triol, (Z)-2-(4-((1-(2,4-difluorophenyl)-4-imino-3-methyl-1H-pyrazolo[3,4-d]pyrimidin-5(4H)-ylimino)methyl)phenoxy)-6-(hydroxymethyl)-tetrahydro-2H-pyran-3,4,5-triol, (Z)-2-(hydroxymethyl)-6-(4-((4-imino-3-methyl-1-(4-(trifluoromethyl)phenyl)-1H-pyrazolo[3,4-d]pyrimidin-5(4H)-ylimino)methyl)phenoxy)-tetrahydro-2H-pyran-3,4,5-triol, (Z)-4-(5-(4-(3,4,5-trihydroxy-6-(hydroxymethyl)-tetrahydro-2H-pyran-2-yloxy)benzylidene amino)-4-imino-3-methyl-4,5-dihydropyrazolo[3,4-d]pyrimidin-1-yl)benzonitrile, (Z)-4-(5-(4-(3,4,5-trihydroxy-6-(hydroxymethyl)-tetrahydro-2H-pyran-2-yloxy)benzylideneamino)-4-imino-3-methyl-4,5-dihydropyrazolo[3,4-d]pyrimidin-1-yl)benzoic acid, (Z)-4-(5-(4-(3,4,5-trihydroxy-6-(hydroxymethyl)tetrahydro-2H-pyran-2-yloxy)benzylideneamino)-4-imino-3-methyl-4,5-dihydropyrazolo[3,4-d]pyrimidin-1-yl)benzenesulfonic acid, (Z)-4-(5-(4-(3,4,5-trihydroxy-6-(hydroxymethyl)tetrahydro-2H-pyran-2-yloxy)benzylideneamino)-4-imino-3-methyl-4,5-dihydropyrazolo[3,4-d]pyrimidin-1-yl)N,N-dipropylbenzenesulfonamide, (Z)-2-(hydroxymethyl)-6-(4-((4-imino-1-phenyl-3-(trifluoromethyl)-1H-pyrazolo[3,4-d]pyrimidin-5(4H)-ylimino)methyl)phenoxy)-tetrahydro-2H-pyran-3,4,5-triol, (Z)-2-(4-((1-(2,4-difluorophenyl)-4-imino-3-(trifluoromethyl)-1H-pyrazolo[3,4-d]pyrimidin-5(4H)-ylimino)methyl)phenoxy)-6-(hydroxymethyl)-tetrahydro-2H-pyran-3,4,5-triol, (Z)-2-(hydroxymethyl)-6-(4-((4-imino-3-(trifluoromethyl)-1-(4-(trifluoromethyl)phenyl)-1H-pyrazolo[3,4-d]pyrimidin-5(4H)-ylimino)methyl)phenoxy)-tetrahydro-2H-pyran-3,4,5-triol, (Z)-4-(5-(4-(3,4,5-trihydroxy-6-(hydroxymethyl)-tetrahydro-2H-pyran-2-yloxy)benzylideneamino)-4-imino-3-(trifluoromethyl)-4,5-dihydropyrazolo[3,4-d]pyrimidin-1-yl)benzonitrile, (Z)-4-(5-(4-(3,4,5-trihydroxy-6-(hydroxymethyl)tetrahydro-2H-pyran-2-yloxy)benzylideneamino)-4-imino-3-(trifluoromethyl)-4,5-dihydropyrazolo[3,4-d]pyrimidin-1-yl)benzoic acid, (Z)-4-(5-(4-(3,4,5-trihydroxy-6-(hydroxymethyl)-tetrahydro-2H-pyran-2-yloxy)benzylideneamino)-4-imino-3-(trifluoromethyl)-4,5-dihydropyrazolo[3,4-d]pyrimidin-1-yl)benzenesulfonic acid, (Z)-4-(5-(4-(3,4,5-trihydroxy-6-(hydroxymethyl)-tetrahydro-2H-pyran-2-yloxy)benzylideneamino)-4-imino-3-(trifluoromethyl)-4,5-dihydropyrazolo[3,4-d]pyrimidin-1-yl)-N,N-dipropylbenzenesulfonamide, (Z)-2-(hydroxymethyl)-6-(4-((4-imino-1,3-diphenyl-1H-pyrazolo[3,4-d]pyrimidin-5(4H)-ylimino)methyl)phenoxy)-tetrahydro-2H-pyran-3,4,5-triol, (Z)-2-(4-((1-(2,4-difluorophenyl)-4-imino-3-phenyl-1H-pyrazolo[3,4-d]pyrimidin-5(4H)-ylimino)methyl)phenoxy)-6-(hydroxymethyl)-tetrahydro-2H-pyran-3,4,5-triol, (Z)-2-(hydroxymethyl)-6-(4-((4-imino-3-phenyl-1-(4-(trifluoromethyl)phenyl)-1H-pyrazolo[3,4-d]pyrimidin-5(4H)-ylimino)methyl)phenoxy)tetrahydro-2H-pyran-3,4,5-triol, (Z)-4-(5-(4-(3,4,5-trihydroxy-6-(hydroxymethyl)tetrahydro-2H-pyran-2-yl oxy)benzylideneamino)-4-imino-3-phenyl-4,5-dihydropyrazolo[3,4-d]pyrimidin-1-yl)benzonitrile, (Z)-4-(5-(4-(3,4,5-trihydroxy-6-(hydroxymethyl)-tetrahydro-2H-pyran-2-yloxy)benzylideneamino)-4-imino-3-phenyl-4,5-dihydropyrazolo[3,4-d]pyrimidin-1-yl)benzoic acid, (Z)-4-(5-(4-(3,4,5-trihydroxy-6-(hydroxymethyl)-tetrahydro-2H-pyran-2-yloxy)benzylideneamino)-4-imino-3-phenyl-4,5-dihydropyrazolo[3,4-d]pyrimidin-1-yl)benzenesulfonic acid, (Z)-4-(5-(4-(3,4,5-trihydroxy-6-(hydroxymethyl)-tetrahydro-2H-pyran-2-yloxy)benzylideneamino)-4-imino-3-phenyl-4,5-dihydropyrazolo[3,4-d]pyrimidin-1-yl)-N,N-dipropylbenzenesulfonamide, (Z)-2-(4-((9-(2,4-difluorophenyl)-6-imino-6H-purin-1(9H)-ylimino)methyl)phenoxy)-6-(hydroxymethyl)-tetrahydro-2H-pyran-3,4,5-triol, (Z)-4-(1-(4-aminobenzylideneamino)-6-imino-1,6-dihydropurin-9-yl)benzonitrile, (Z)-4-(1-(4-(trifluoromethyl)benzylideneamino)-6-imino-1,6-dihydropurin-9-yl)benzonitrile, (Z)-4-(1-(2,4-difluorobenzylideneamino)-6-imino-1,6-dihydropurin-9-yl)benzonitrile, (Z)-4-((6-imino-9-(4-(trifluoromethyl)phenyl)-6H-purin-1(9H)-ylimino)methyl)benzonitrile, (Z)—N-(4-(trifluoromethyl)benzylidene)-6-imino-9-(4-(trifluoromethyl)phenyl)-6H-purin-1(9H)-amine, (Z)-4-((9-(2,4-difluorophenyl)-6-imino-6H-purin-1(9H)-ylimino)methyl)benzonitrile, (Z)—N-(4-(trifluoromethyl)benzylidene)-9-(2,4-difluorophenyl)-6-imino-6H-purin-1(9H)-amine, (Z)-4-(1-(4-(3,4,5-trihydroxy-6-(hydroxymethyl)-tetrahydro-2H-pyran-2-yloxy)benzylideneamino)-6-imino-8-methyl-1,6-dihydropurin-9-yl)benzonitrile, (Z)-2-(hydroxymethyl)-6-(4-((6-imino-8-methyl-9-(4-(trifluoromethyl)phenyl)-6H-purin-1(9H)-ylimino)methyl)phenoxy)-tetrahydro-2H-pyran-3,4,5-triol, (Z)-2-(4-((9-(2,4-difluorophenyl)-6-imino-8-methyl-6H-purin-1(9H)-ylimino)methyl)phenoxy)-6-(hydroxymethyl)-tetrahydro-2H-pyran-3,4,5-triol, (Z)-4-(1-(4-methoxybenzylideneamino)-6-imino-8-methyl-1,6-dihydropurin-9-yl)benzonitrile, (Z)-4-(1-(4-(trifluoromethyl)benzylideneamino)-6-imino-8-methyl-1,6-dihydropurin-9-yl)benzonitrile, (Z)-4-(1-(2,4-difluorobenzylideneamino)-6-imino-8-methyl-1,6-dihydropurin-9-yl)benzonitrile, (Z)-4-((6-imino-8-methyl-9-(4-(trifluoromethyl)phenyl)-6H-purin-1(9H)-ylimino)methyl)benzonitrile, (Z)—N-(4-(trifluoromethyl)benzylidene)-6-imino-8-methyl-9-(4-(trifluoromethyl)phenyl)-6H-purin-1(9H)-amine, (Z)—N-(2,4-difluorobenzylidene)-6-imino-9-(4-(trifluoromethyl)phenyl)-6H-purin-1(9H)-amine, (Z)-4-((9-(2,4-difluorophenyl)-6-imino-8-methyl-6H-purin-1(9H)-ylimino)methyl)benzonitrile, (Z)—N-(4-(trifluoromethyl)benzylidene)-9-(2,4-difluorophenyl)-6-imino-8-methyl-6H-purin-1(9H)-amine, (Z)—N-(2,4-difluorobenzylidene)-9-(2,4-difluorophenyl)-6-imino-8-methyl-6H-purin-1(9H)-amine, 4-(6-amino-2-(4-(3,4,5-trihydroxy-6-(hydroxymethyl)-tetrahydro-2H-pyran-2-yloxy)phenyl)-9H-purin-9-yl)benzonitrile, 4-(6-oxo-2-(4-(3,4,5-trihydroxy-6-(hydroxymethyl)-tetrahydro-2H-pyran-2-yloxy)phenyl)-1,6-dihydropurin-9-yl)benzonitrile, 4-(6-oxo-2-(4-(trifluoromethyl)phenyl)-1,6-dihydropurin-9-yl)benzonitrile, 4-(4-amino-6-(4-(3,4,5-trihydroxy-6-(hydroxymethyl)-tetrahydro-2H-pyran-2-yloxy)phenyl)-1H-pyrazolo[3,4-d]pyrimidin-1-yl)benzonitrile, 4-(4-oxo-6-(4-(3,4,5-trihydroxy-6-(hydroxymethyl)tetrahydro-2H-pyran-2-yloxy)phenyl)-4,5-dihydropyrazolo[3,4-d]pyrimidin-1-yl)benzonitrile, 4-(4-oxo-6-(4-(trifluoromethyl)phenyl)-4,5-dihydropyrazolo[3,4-d]pyrimidin-1-yl)benzonitrile, 4-(6-(2,4-difluorophenyl)-4-oxo-4,5-dihydropyrazolo[3,4-d]pyrimidin-1-yl)benzonitrile, N-(3-(dimethylamino) propyl)-7-methyl-2-phenylimidazo[1,2-a]pyrimidin-5-amine, N-(3-(dimethylamino)propyl)-7-methyl-2-(4-(trifluoromethyl)phenyl)imidazo[1,2-a]pyrimidin-5-amine, 4-(5-(3-(dimethylamino)propylamino)-7-methylimidazo[1,2-a]pyrimidin-2-yl)benzonitrile, 2-(2,4-difluorophenyl)-N-(3-(dimethylamino)propyl)-7-methylimidazo[1,2-a]pyrimidin-5-amine, 4-(2-methyl-9-(4-(trifluoromethyl)phenyl)-9H-purin-6-ylamino)benzonitrile, 4-(9-(4-cyanophenyl)-2-methyl-9H-purin-6-ylamino)benzoic acid, 4-(9-(2,4-difluorophenyl)-2-methyl-9H-purin-6-ylamino)benzonitrile, 4-(6-methyl-1-(4-(trifluoromethyl)phenyl)-1H-pyrazolo[3,4-d]pyrimidin-4-ylamino)benzonitrile, 457. 4-(6-methyl-1-(4-(trifluoromethyl)phenyl)-1H-pyrazolo[3,4-d]pyrimidin-4-ylamino)benzonitrile 4-(1-(2,4-difluorophenyl)-6-methyl-1H-pyrazolo[3,4-d]pyrimidin-4-ylamino)benzonitrile, 4-(7-(2,4-difluorophenyl)-5-oxo-2-(4-(trifluoromethyl)phenyl)-5,8-dihydroimidazo[1,2-a]pyrimidin-3-yl)benzonitrile, 3,7-bis(2,4-difluorophenyl)-2-(4-(trifluoromethyl)phenyl)imidazo[1,2-a]pyrimidin-5(8H)one, 4-(3,7-bis(2,4-difluorophenyl)-5-oxo-5,8-dihydroimidazo[1,2-a]pyrimidin-2-yl)benzonitrile, 4-(7-(2,4-difluorophenyl)-3-(4-methoxyphenyl)-5-oxo-5,8-dihydroimidazo[1,2-a]pyrimidin-2-yl)benzonitrile, 4-(2,7-bis(2,4-difluorophenyl)-5-oxo-5,8-dihydroimidazo[1,2-a]pyrimidin-3-yl)benzonitrile, 2,3,7-tris(2,4-difluorophenyl)imidazo[1,2-a]pyrimidin-5(8H)-one, 4-(5-oxo-7-(trifluoromethyl)-2-(4-(trifluoromethyl)phenyl)-5,8-dihydroimidazo[1,2-a]pyrimidin-3-yl)benzonitrile, 3-(2,4-difluorophenyl)-7-(trifluoromethyl)-2-(4-(trifluoromethyl)phenyl)imidazo[1,2-a]pyrimidin-5(8 H)-one, 4-(3-(2,4-difluorophenyl)-5-oxo-7-(trifluoromethyl)-5,8-dihydroimidazo[1,2-a]pyrimidin-2-yl)benzonitrile, 4-(3-(4-methoxyphenyl)-5-oxo-7-(trifluoromethyl)-5,8-dihydroimidazo[1,2-a]pyrimidin-2-yl)benzonitrile, 2,3-bis(2,4-difluorophenyl)-7-(trifluoromethyl)imidazo[1,2-a]pyrimidin-5(8H)-one, 4-(7-amino-5-oxo-2-(4-(trifluoromethyl)phenyl)-5,8-dihydroimidazo[1,2-a]pyrimidin-3-yl)benzonitrile, 7-amino-3-(2,4-difluorophenyl)-2-(4-(trifluoromethyl)phenyl)imidazo[1,2-a]pyrimidin-5(8H)-one, 4-(7-amino-3-(2,4-difluorophenyl)-5-oxo-5,8-dihydroimidazo[1,2-a]pyrimidin-2-yl)benzonitrile, 4-(7-amino-2-(2,4-difluorophenyl)-5-oxo-5,8-dihydroimidazo[1,2-a]pyrimidin-3-yl)benzonitrile, 7-amino-2,3-bis(2,4-difluorophenyl)imidazo[1,2-a]pyrimidin-5(8H)-one, 7-amino-3-(2,4-difluorophenyl)-2-(4-(trifluoromethyl)phenyl)imidazo[1,2-a]pyrimidine-6-carboxamide, 7-amino-2-(4-cyanophenyl)-3-(2,4-difluorophenyl)imidazo[1,2-a]pyrimidine-6-carboxamide, 7-amino-2,3-bis(2,4-difluorophenyl)imidazo[1,2-a]pyrimidine-6-carboxamide, 7-amino-3-(trifluoromethyl)-2-(4-(trifluoromethyl)phenyl)imidazo[1,2-a]pyrimidine-6-carboxamide, 7-amino-2-(4-cyanophenyl)-3-(trifluoromethyl)imidazo[1,2-a]pyrimidine-6-carboxamide, 7-amino-2-(2,4-difluorophenyl)-3-(trifluoromethyl)imidazo[1,2-a]pyrimidine-6-carboxamide, 3,7-diamino-2-(4-(trifluoromethyl)phenyl)imidazo[1,2-a]pyrimidine-6-carboxamide, 3,7-diamino-2-(4-cyanophenyl)imidazo[1,2-a]pyrimidine-6-carboxamide, 3,7-diamino-2-(2,4-difluorophenyl)imidazo-[1,2-a]pyrimidine-6-carboxamide, ethyl 3-(2,4-difluorophenyl)-5-oxo-2-(4-(trifluoromethyl)phenyl)-5,8-dihydroimidazo[1,2-al pyrimidine-6-carboxylate, ethyl 2-(4-cyanophenyl)-3-(2,4-difluorophenyl)-5-oxo-5,8-dihydroimidazo[1,2-al pyrimidine-6-carboxylate, ethyl 2,3-bis(2,4-difluorophenyl)-5-oxo-5,8-dihydroimidazo[1,2-a]pyrimidine-6-carboxylate, ethyl 5-oxo-3-(trifluoromethyl)-2-(4-(trifluoromethyl)phenyl)-5,8-dihydroimidazo[1,2-a]pyrimidine-6-carboxylate, ethyl 2-(4-cyanophenyl)-5-oxo-3-(trifluoromethyl)-5,8-dihydroimidazo[1,2-a]pyrimidine-6-carboxylate, ethyl 2-(2,4-difluorophenyl)-5-oxo-3-(trifluoromethyl)-5,8-dihydroimidazo[1,2-a]pyrimidine-6-carboxylate, ethyl 3-amino-5-oxo-2-(4-(trifluoromethyl)phenyl)-5,8-dihydroimidazo[1,2-a]pyrimidine-6-carboxylate, ethyl 3-amino-2-(4-cyanophenyl)-5-oxo-5,8-dihydroimidazo[1,2-a]pyrimidine-6-carboxylate, ethyl 3-amino-2-(2,4-difluorophenyl)-5-oxo-5,8-dihydroimidazo[1,2-a]pyrimidine-6-carboxylate, 3-(2,4-difluorophenyl)-5-(3-nitrophenyl)-8-(trifluoromethyl)-3H-chromeno[2,3-d]pyrimidin-4(5H)-imine, 3-(3-(2,4-difluorophenyl)-4-imino-8-(trifluoromethyl)-4,5-dihydro-3H-chromeno[2,3-d]pyrimidin-5-yl)benzenamine, 3-(2,4-difluorophenyl)-4-imino-5-(3-nitrophenyl)-4,5-dihydro-3H-chromeno[2,3-d]pyrimidine-8-carbonitrile, 5-(3-aminophenyl)-3-(2,4-difluorophenyl)-4-imino-4,5-dihydro-3H-chromeno[2,3-d]pyrimidine-8-carbonitrile, 3-(2,4-difluorophenyl)-6,8-difluoro-5-(3-nitrophenyl)-3H-chromeno[2,3-d]pyrimidin-4(5H)-imine, 3-(3-(2,4-difluorophenyl)-6,8-difluoro-4-imino-4,5-dihydro-3H-chromeno[2,3-d]pyrimidin-5-yl)benzenamine, 4-imino-5-(3-nitrophenyl)-8-(trifluoromethyl)-4H-chromeno[2,3-d]pyrimidin-3(5H)-amine, 5-(3-aminophenyl)-4-imino-8-(trifluoromethyl)-4H-chromeno[2,3-d]pyrimidin-3(5H)-amine, 3-amino-4-imino-5-(3-nitrophenyl)-4,5-dihydro-3H-chromeno[2,3-d]pyrimidine-8-carbonitrile, 3-amino-5-(3-aminophenyl)-4-imino-4,5-dihydro-3H-chromeno[2,3-d]pyrimidine-8-carbonitrile, 6,8-difluoro-4-imino-5-(3-nitrophenyl)-4H-chromeno[2,3-d]pyrimidin-3(5H)-amine, 5-(3-aminophenyl)-6,8-difluoro-4-imino-4H-chromeno[2,3-d]pyrimidin-3(5H)-amine;
The formation of eight-membered A-cycle, 4-((6Z,8Z,10E)-7-methyl-4-oxo-10-(2,4-difluorophenyl)-4,11a-dihydro-1H-pyrimido[1,2-b][1,2]diazocin-2-yl)benzonitrile, 4-((6Z,8Z,10E)-1,7-dimethyl-4-oxo-10-(2,4-difluorophenyl)-4,11a-dihydro-1H-pyrimido[1,2-b][1,2]diazocin-2-yl)benzonitrile, (6Z,8Z,10E)-1,7-dimethyl-10-(4-(trifluoromethyl)phenyl)-1H-pyrimido[1,2-b][1,2]diazocin-4(11aH)one, (6Z, 8Z,10E)-7-methyl-2,10-bis(4-(trifluoromethyl)phenyl)-1H-pyrimido[1,2-b][1,2]diazocin-4-one, (6Z,8Z,10E)-7-methyl-10-(4-(trifluoromethyl)phenyl)-2-(6-(trifluoromethyl)pyridin-3-yl)-2,3-dihydro-1-H-pyrimido[1,2-b][1,2]diazocin-4-one, (6Z,8Z,10E)-2-(2,4-difluorophenyl)-7-methyl-10-(4-(trifluoromethyl)phenyl)-1H-pyrimido[1,2-b][1,2]diazocin-4-one, (6Z,8Z,10E)-10-(2,4-difluorophenyl)-2-(4-methoxyphenyl)-7-methyl-1H-pyrimido[1,2-b][1,2]diazocin-4-one, (6Z,8Z,10E)-2-(4-aminophenyl)-10-(2,4-difluorophenyl)-7-methyl-1H-pyrimido[1,2-b][1,2]diazocin-4-one, 4-(((6Z,8Z,10E)-10-(2,4-difluorophenyl)-7-methyl-4-oxo-4,11-dihydro-1H-pyrimido[1,2-b][1,2]diazocin-2-yl)aminomethyl)benzonitrile, 4-((6Z,8Z,10E)-10-(2,4-difluorophenyl)-7-methyl-4-oxo-4,11-dihydro-1H-pyrimido[1,2-b][1,2]diazocin-2-yl)-N,N-dipropyl benzene sulfonamide, 2-(2,4-difluorophenyl)-4,11-dihydro-4H-10-(4-(trifluoromethyl)phenyl)(6Z,8Z,10Z)-1H-pyrimido[1,2-b][1,2]diazocin-4-one, 4-((6Z,8Z,10E)-10-(2,4-difluorophenyl)-4-oxo-7-phenyl-4,11-dihydro-1H-pyrimido[1,2-b][1,2]diazocin-2-yl)benzonitrile, (6Z,8Z,0E)-10-(2,4-difluorophenyl)-7-phenyl-2-(4-(trifluoromethyl)phenyl)-1H-pyrimido[1,2-b][1,2]diazocin-4-one, 4-((6Z,8Z,10E)-7-cyclopropyl-10-(2,4-difluorophenyl)-4-oxo-4,11-dihydro-1H-pyrimido[1,2-b][1,2]diazocin-2-yl)benzonitrile, 4-((6Z,4-((6Z,8Z,10E)-10-(2,4-difluorophenyl)-4-oxo-7-((trifluoromethyl)phenyl)-4,11-dihydro-1H-pyrimido[1,2-b][1,2]diazocin-2-yl)benzonitrile, 4-((6Z,8Z,10E)-10-(2,4-difluorophenyl)-7-ethyl-4-oxo-4,1-dihydro-1H-pyrimido[1,2-b][1,2]diazocin-2-yl)benzonitrile, 4-((6Z,8Z,10E)-10-(2,4-dichlorophenyl)-7-methyl-4-oxo-4,11-dihydro-1H-pyrimido[1,2-b][1,2]diazocin-2-yl)benzonitrile, 4-((6Z,8Z,10E)-2-(4-cyanophenyl)-7-methyl-4-oxo-4,11-dihydro-1H-pyrimido[1,2-b][1,2]diazocin-10-yl)benzoic acid;
The formation of five-membered B-cycle, 4-(2-(2,4-difluorophenyl)-3-oxo-imidazo[1,2-b]pyrazol-7-yl)benzonitrile, 4-(2-(2,4-difluorophenyl)-6-oxo-imidazo[1,5-b]pyrazol-3-yl)benzonitrile, 4-(2-methyl-6-oxo-3-(4-(trifluoromethyl)phenyl)-6H-imidazo[1,5-b]pyridin-4-yl)benzonitrile, 3-(2,4-difluorophenyl)-2-methyl-4-(4-(trifluoromethyl)phenyl)6H-imidazo[1,5-b]pyrazol-6-one, 3-(2,4-difluorophenyl)-2-phenyl-4-(4-(trifluoromethyl)phenyl)-6H-imidazo[1,5-b]pyrazol-6-one, 4-(2-cyclopropyl-3-(2,4-difluorophenyl)-6-oxo-6H-imidazo[1,5-b]pyrazol-4-yl)benzonitrile, 3-(2,4-difluorophenyl)-2-ethyl-4-(4-(trifluoromethyl)phenyl)-1H-imidazo[1,5-b]pyrazol-6(3aH)-one, 4-(3-(2,4-difluorophenyl)-6-oxo-2-phenyl-6H-imidazo[1,5-b]pyrazol-4-yl)benzonitrile, 4-(3-(2,4-difluorophenyl)-6-oxo-2-((trifluoromethyl)phenyl)-6H-imidazo[1,5-b]pyrazol-4-yl)benzonitrile, 4-(3-(2,4-difluorophenyl)-2-ethyl-6-oxo-6H-imidazo[1,5-b]pyrazol-4-yl)benzonitrile, 4-(4-(4-cyanophenyl)-2-methyl-6-oxo-6H-imidazo[1,5-b]pyrazol-3-yl)benzoic acid, 4-(3-(2,4-dichlorophenyl)-2-methyl-6-oxo-6H-imidazo[1,5-b]pyrazol-4-yl)benzonitrile, 4-(3-(2,4-difluorophenyl)-2-methyl-6-oxo-6H-imidazo[1,5-b]pyrazol-4-yl)-N,N-dipropylbenzenesulfonamide, 4-((3-(2,4-difluorophenyl)-2-methyl-6-oxo-6H-imidazo[1,5-b]pyrazol-4-yl)aminomethyl)benzonitrile, 4-(4-aminophenyl)-3-(2,4-difluorophenyl)-2-methyl-6H-imidazo[1,5-b]pyrazol-6-one, 3-(2,4-difluorophenyl)-5-(4-methoxyphenyl)-2-methyl-6H-imidazo[1,5-b]pyrazol-6-one, 4-(2,4-difluorophenyl)-2-methyl-3-(4-(trifluoromethyl)phenyl)-6H-imidazo[1,5-b]pyrazol-6-one, 2-methyl-3-(4-(trifluoromethyl)phenyl)-4-(6-(trifluoromethyl)pyridin-3-yl)-6H-imidazo[1,5-b]pyridin-6-one, 2-methyl-3,4-bis(4-(trifluoromethyl)phenyl)-6H-imidazo[1,5-b]pyrazol-6-one, 7-(2,4-difluorophenyl)-6-methyl-2-(4-(trifluoromethyl)phenyl)-3H-imidazo[1,2-b]pyrazol-3-one, 4-(6-methyl-3-oxo-7-(4-(trifluoromethyl)phenyl)-3H-imidazo[1,2-b]pyrazol-2-yl)benzonitrile, 6-methyl-2,7-bis(4-(trifluoromethyl)phenyl)-3H-imidazo[1,2-b]pyrazol-3-one, 6-methyl-7-(4-(trifluoromethyl)phenyl)-5-(6-(trifluoromethyl)pyridin-3-yl)-3-H-imidazo[1,2-b]imidazol-3-one, 2-(2,4-difluorophenyl)-6-methyl-7-(4-(trifluoromethyl)phenyl)-3H-imidazo[1,2-b]pyrazol-3-one, 7-(2,4-difluorophenyl)-5-(4-methoxyphenyl)-6-methyl-3H-imidazo[1,2-b]pyridin-3-one, 2-(4-aminophenyl)-7-(2,4-difluorophenyl)-6-methyl-3H-imidazo[1,2-b]pyrazol-3-one, 4-((7-(2,4-difluorophenyl)-6-methyl-3-oxo-3H-imidazo[1,2-b]pyrazol-2-yl)amino methyl)benzonitrile, 4-(7-(2,4-difluorophenyl)-6-methyl-3-oxo-3H-imidazo[1,2-b]pyrazol-2-yl)-N,N-dipropylbenzene sulfonamide, 2-(2,4-difluorophenyl)-6-ethyl-7-(4-(trifluoromethyl)phenyl)-3H-imidazo[1,2-b]pyrazol-3-one, 4-(7-(2,4-difluorophenyl)-3-oxo-6-phenyl-3H-imidazo[1,2-b]pyrazol-2-yl)benzonitrile, 7-(2,4-difluorophenyl)-6-phenyl-2-(4-(trifluoromethyl)phenyl)-3H-imidazo[1,2-b]pyrazol-3-one, 4-(6-cyclopropyl-7-(2,4-difluorophenyl)-3-oxo-3H-imidazo[1,2-b]pyrazol-2-yl)benzonitrile, 4-(7-(2,4-difluorophenyl)-3-oxo-6-((trifluoromethyl)phenyl)-3H-imidazo[1,2-b]pyrazol-2-yl)benzonitrile, 4-(7-(2,4-difluorophenyl)-6-ethyl-3-oxo-3H-imidazo[1,2-b]pyrazol-2-yl)benzonitrile, 4-(7-(2,4-dichlorophenyl)-6-methyl-3-oxo-3H-imidazo[1,2-b]pyrazol-2-yl)benzonitrile, 4-(2-(4-cyanophenyl)-6-methyl-3-oxo-3H-imidazo[1,2-b]pyrazol-7-yl)benzoic acid.

8. A compound according to the claim 1 and 2, wherein:
the administration may be by oral route, parenteral, subcutaneous, intravenous, intramuscular, intra-peritoneal, transdermal, buccal, intrathecal, intracranial, intranasal or topical routes.
